(19) United States
(12) Patent Application Publication
(10) Pub. No.: US 2010/0159486 A1
(43) Pub. Date: Jun. 24, 2010

(54) BIOMARKERS FOR NEUROLOGICAL CONDITIONS

(75) Inventors: Lance A. Liotta, Bethesda, MD (US); Wediong Zhou, Manassas, VA (US); Wolff Kirsch, Redlands, CA (US); Virginia Espina, Rockville, MD (US); Emanuel Petricoin, III, Gainesville, VA (US); Mark Ross, Charlottesville, VA (US); Claudius Mueller, Loma Linda, CA (US); Shino Magaki, Redlands, CA (US)

Correspondence Address: FOLEY AND LARDNER LLP;SUITE 500, 3000 K STREET NW, WASHINGTON, DC, 20007, US

(73) Assignees: George Mason Intellectual Properties, Inc.; Loma Linda University

(21) Appl. No.: 12/513,032

(22) PCT Filed: Nov. 1, 2007
(86) PCT No.: PCT/US2007/023026

§ 371 Date: Feb. 17, 2010

Related U.S. Application Data
(60) Provisional application No. 60/855,749, filed on Nov. 1, 2006.

(51) Int. Cl. G01N 33/53; C12Q 1/02; G01N 33/566; G01N 33/00; C07K 16/18

(52) U.S. Cl. 435/7.92; 435/7.1; 435/29; 436/501; 436/89; 530/387.3; 530/387.9

(57) ABSTRACT

Low molecular weight (LMW) peptides have been discovered that are indicative of neurological conditions, such as Alzheimer's Disease (AD), cognitive impairment and brain microhemmorhages. Evaluating patient samples for the presence of such LMW peptides is an effective means of detecting neurological conditions and monitoring the progression of the disease. The LMW peptides are particularly useful in detecting neurological conditions during the early stages without invasive procedures.

### CROSS-REFERENCE TO RELATED APPLICATIONS

This application claims priority to U.S. Provisional Application No. 60/855,749, filed Nov. 1, 2006, which is hereby incorporated by reference.

### BACKGROUND

Alzheimer's disease (AD) is a progressive degenerative disease of the brain primarily associated with aging. AD is one of several disorders that cause the gradual loss of brain cells and is one of and possibly the leading cause of dementia. Clinical presentation of AD is characterized by loss of memory, cognition, reasoning, judgment, and orientation. Mild cognitive impairment (MCI) is often the first identified stage of AD. As the disease progresses, motor, sensory, and linguistic abilities also are affected until there is global impairment of multiple cognitive functions. These cognitive losses occur gradually, but typically lead to severe impairment and eventual death in the range of three to twenty years.

An early diagnosis of AD has many advantages including additional time to make choices that maximize quality of life, less anxiety about unknown problems, a better chance of benefiting from treatment and more time to plan for the future. However, reliable noninvasive methods for diagnosing AD are not available.

Alzheimer's disease is characterized by two major pathologic observations in the brain: neurofibrillary tangles (NFT) and beta-amyloid plaques, comprised predominantly of an aggregate of fragments known as Aβ peptides. Individuals with AD exhibit characteristic beta-amyloid deposits in the brain (beta-amyloid plaques) and in cerebral blood vessels (beta-amyloid angiopathy) as well as neurofibrillary tangles. Neurofibrillary tangles occur not only in Alzheimer's disease but also in other dementia-inducing disorders. On autopsy, presently the only definitive method of diagnosing AD, large numbers of these lesions are generally found in areas of the human brain important for memory and cognition.

There is an urgent clinical need to develop diagnostic markers that can detect early stage AD, particularly at the stage of MCI. While advances have been made in imaging beta-amyloid, (Lopresti et al. J. Nucl. Med. (2005) 46:1959-1972), no serum biomarkers for AD are clinically available. To date there are no validated biomarkers for confirming the diagnosis of a major neurodegenerative disorder or to monitor progression (Castano et al. Neurol. Res. (2006) 28:1155-163).

Despite the enthusiasm for the use of proteomic technology to discover blood markers of AD, and decades of effort, progress towards identifying useful markers has been slow, possibly because putative high specificity AD markers are assumed to be in very low abundance because they are shed from small volumes of diseased tissue and are expected to be rapidly cleared and metabolized. In addition, researchers have avoided studying blood because the blood proteome is dominated by, and complicated by, resident proteins such as albumin that can exist at a concentration many millions of times greater than the target low abundance biomarker. For this reason, researchers have focused on cerebrospinal fluid (CSF) as the target fluid for AD biomarkers (see Zhang et al., J. Alzheimer's Disease (2005) 8:377-3386). The CSF approach, however, has limited clinical application to routine screening. Moreover, the blood brain vascular circulation perfuses AD lesions with a higher efficiency, particularly in the case for amyloid angiopathy.

### SUMMARY

In one aspect, methods are provided for diagnosing a neurological condition in a patient comprising obtaining a biological sample from the patient and evaluating the sample for the abundance of at least one biomarker selected from the group consisting of a peptide having the amino acid sequence of SEQ ID NOs:1-440, wherein the abundance of said at least one biomarker is indicative of a neurological condition. In one embodiment, the abundance of the biomarker is greater than that of a control sample. In another embodiment, the abundance of the biomarker is less than that of a control sample.

The method also can comprise, prior to the evaluation step, harvesting low molecular weight peptides from said sample to generate at least one fraction comprising said peptides. The biomarker can be a low molecular weight protein complexed with a carrier protein. In a further embodiment, the low molecular weight protein is further purified from said carrier protein. In another embodiment, the low molecular weight protein is digested and optionally sequenced. In one embodiment, the biological sample is blood, serum or plasma. In another embodiment, the evaluation step comprises an assay selected from the group consisting of mass spectrometry, such as tandem mass spectrotrometry (MS MS), immunoassay, such as enzyme-linked immunosorbent assay (ELISA), immuno-mass spectrometry and suspension bead array. The method also can comprise obtaining a neuroimage of the brain microvasculopathy, which can be optionally obtained using susceptibility weighted imaging, perfusion weighted imaging and magnetic resonance spectroscopy.

The neurological condition can be Alzheimer's disease (AD), mild cognitive impairment (MCI), stable mild cognitive impairment (stable MCI), progressive mild cognitive impairment (PMCI), vascular dementia (VD), angiopathy black holes, cerebral amyloid angiopathy (CAA) and brain microhemorrages. In one embodiment, methods are provided for diagnosing Alzheimer's disease in a patient comprising obtaining a biological sample from said patient, and evaluating said sample for the abundance of at least one biomarker selected from the group consisting of a peptide having the amino acid sequence of SEQ ID NOs:1, 3-13, 15, 16, 21, 22, 24-28, 31-33, 37-44, 56-59, 66-68, 93-101, 111-128, 143-153, 156-1170, 172-183, 263-279, 310-335, 348, 355-359, 362, 363, 365, 372, 373, 376-402, 406-426 and 436-44, wherein the abundance of said at least one biomarker is indicative of Alzheimer's disease. In another aspect, the biomarker is a peptide associated with a metabolic pathway or cellular process. In others aspects, the biomarker is a peptide associated with inflammation, estrogen activity, pigment epithelium-derived factor (PEDF), vitamin D metabolism and bone mineralization, coagulation and platelet activity, the complement cascade, acyl-peptide hydrolase (APH) activity, vitamin A and thyroxine, phospholipase activity, globin activity, glycosylation or is glycosylated, protease inhibition, keratins and related proteins, heme degradation, pyruvate metabolism, calcium related proteins, defensin, gelsolin, vitronectin, profilin, thrombospondin, peroxiredoxin, alcohol dehydrogenase, apolipoproteins, iron and copper metabolism, or NMDA receptor-related proteins.

In another aspect, methods are provided for diagnosing mild cognitive impairment in a patient comprising obtaining a biological sample from the patient and evaluating the sample for the abundance of at least one biomarker selected from the group consisting of a peptide having the amino acid sequence of SEQ ID NOs: 2, 4, 14, 17, 23, 29, 34, 45-55, 60-65, 69-92, 102-110, 129-142, 154, 155, 171, 184-191, 193-226, 248-279, 281-320, 333, 336-347, 349-354, 360, 361, 364, 366-371, 374, 375, 403-405 and 427-435, wherein the abundance of said at least one biomarker is indicative of mild cognitive impairment.

In yet another aspect, methods are provided for diagnosing brain microhemorrhages in a patient comprising obtaining a biological sample from the patient and evaluating the sample for the abundance of at least one biomarker selected from the group consisting of a peptide having the amino acid sequence of SEQ ID NOs:441-452, wherein the abundance of said at least one biomarker is indicative of brain microhemorrhages.

In some embodiments, the inventive methods comprise, prior to the evaluation step, harvesting low molecular weight peptides from the biological sample to generate at least one fraction comprising the peptides. The size of the low molecular weight peptides can be, for example, less than 50 KDa, less than 25 KDa, or less than 15 KDa. The methods also can comprise digesting the low molecular weight peptides. Such digestion can be accomplished using enzymatic or chemical means. In one example, trypsin can be used to digest the peptides.

In other aspects, antibodies are provided that are specific for biomarkers for a neurological condition, as well as kits for detecting a neurological condition in a patient, comprising at least one such antibody. The antibody can be, for example, a monoclonal or polyclonal antibody, and also be a chimeric, humanized or human antibody.

Other objects, features and advantages will become apparent from the following detailed description. The detailed description and specific examples are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### DETAILED DESCRIPTION

Low molecular weight (LMW) peptides have been discovered from the repertoire of proteins bound to carrier proteins such as albumin that are indicative of a neurological condition. Evaluating patient samples for the presence of such LMW peptides is an effective means of detecting a neurological condition and monitoring the progression of the disease, for example during treatment. The LMW peptides are particularly useful in detecting a neurological condition during its early stages. The LMW peptides are particularly useful for detecting AD, MCI and brain microhemorrhages.

The LMW peptides, which are biomarkers, can be detected using a variety of methods known in the art. For example, antibodies can be utilized in immunoassays to detect the presence of a biomarker. Exemplary immunoassays include, e.g., ELISA, radioimmunoassay, immunofluorescent assay, “sandwich” immunoassay, western blot, immunoprecipitation assay and immunoelectrophoresis assays. In other aspects, microbeads, arrays, microarrays, etc. can be used in detecting the LMW peptides. Exemplary assays include, but are not limited to, a suspension bead assay (Schwenk et al., “Determination of binding specificities in highly multiplexed bead-based assays for antibody proteomics,” Mol. Cell Proteomics, 6(1): 125-132 (2007)), an antibody microarray (Borrebaeck et al., “High-throughput proteomics using antibody microarrays: an update,” Expert Rev. Mol. Diagn. 7(5): 673-686 (2007)), an aptamer array (Walter et al., “High-throughput protein arrays: prospects for molecular diagnostics,” Trends Mol. Med. 8(6): 250-253 (2002)), an affybody array (Renberg et al., “Affibody molecules in protein capture microarrays: evaluation of multidomain ligands and different detection formats,” J. Proteome Res. 6(1): 171-179 (2007)), and a reverse phase array (VanMeter et al., “Reverse-phase protein microarrays: application to biomarker discovery and translational medicine,” Expert Rev. Mol. Diagn. 7(5): 625-633 (2007)). All of these publications are incorporated herein by reference.

In another example, the inventive biomarkers can be detected using mass spectrometry (MS). One example of this approach is tandem mass spectrometry (MS/MS), which involves multiple steps of mass selection or analysis, usually separated by some form of fragmentation. Most such assays use electrospray ionization followed by two stages of mass selection: a first stage (MS1) selecting the mass of the intact analyte (parent ion) and, after fragmentation of the parent by collision with gas atoms, a second stage (MS2) selecting a specific fragment of the parent, collectively generating a selected reaction monitoring assay. In one embodiment, collision-induced dissociation is used to generate a set of fragments from a specific peptide ion. The fragmentation process primarily gives rise to cleavage products that break along peptide bonds. Because of the simplicity in fragmentation, the observed fragment masses can be compared to a database of predicted masses for known peptide sequences. A number of different algorithmic approaches have been described to identify peptides and proteins from tandem mass spectrometry (MS/MS) data, including peptide fragment fingerprinting (SEQUEST, MASCOT, OMSSA and X!Tandem), peptide de novo sequencing (PEAKS, LuteFisk and Sherenga) and sequence tag based searching (SPIDER, GutenTAG).

Likewise, multiple reaction monitoring (MRM) can be used to identify the inventive biomarkers in patient samples. This technique applies the MS/MS approach to, for example, tryptic digests of the input sample, followed by selected ion partitioning and sampling using MS to make the analyte selection more objective and discrete by following the exact m/z ion of the tryptic fragment that represents the analyte. Such an approach can be performed in multiplex so that multiple ions can be measured at once, providing an antibody-free method for analyte measurement. See, e.g. Andersen et al., Molecular & Cellular Proteomics, 5.4: 573-588 (2006); Whiteaker et al., J. Proteome Res. 6(10): 3962-75 (2007). Both publications are incorporated herein by reference.

In another example, the inventive biomarkers can be detected using nanoflow reverse-phase liquid chromatography-tandem mass spectrometry. See, e.g., Domon B, Aebersold R. Science, 312(5771):212-7(2006), which is incorporated herein by reference. Using this approach, practitioners obtain peptide fragments, usually by trypsin digest, and generate mass spectrograms of the fragments, which are then compared to a database, such as SEQUEST, for protein identification.

In another aspect, the inventive biomarkers can be detected using immuno-mass spectrometry. See, e.g., Liotta L et al. J Clin Invest.,116(1):26-30 (2006), Nedelkov, Expert Rev. Proteomics, 3(6): 631-640 (2006), which are incorporated herein by reference. Immuno-mass spectrometry provides a means for rapidly determining the exact size and identity of a peptide biomarker isoform present within a patient sample. When developed as a high throughput diagnostic assay, a drop of patient's blood, serum or plasma can be applied to a high density matrix of microcolumns or microwells filled with a composite substratum containing immobilized polyclonal antibodies, directed against the peptide marker. All isoforms of the peptide that contain the epitope are captured. The captured population of analytes including the analyte fragments are eluted and analyzed directly by a mass spectrometer such as MALDI-TOF MS. The presence of the specific peptide biomarker at its exact mass/charge (m/z) location can be used as a diagnostic test result. The analysis can be performed rapidly by simple software that determines if a series of ion peaks are present at defined m/z locations.

In yet another example, the inventive biomarkers can be detected using standard immunoassay-based approaches whereby fragment specific antibodies are used to measure and record the presence of the diagnostic fragments. See, e.g., Naya et al. “Evaluation of precursor prostate-specific antigen isoform ratios in the detection of prostate cancer.” Urol Oncol. 23(1):16-21 (2005). Moreover, additional immunoassays are well known to one skilled in the field, such as ELISA (Maeda et al., “Blood tests for asbestos-related mesothelioma,” Oncology 71: 26-31 (2006)), microfluidic ELISA (Lee et al., “Microfluidic enzyme-linked immunosorbent assay technology,” Adv. Clin. Chem. 42: 255-259 (2006)), nanocantilever immunoassay (Kurosawa et al., “Quartz crystal microbalance immunosensors for environmental monitoring,” Biosens Bioelectron, 22(4): 473-481 (2006)), and plasmon resonance immunoassay (Nedelkov, “Development of surface Plasmon resonance mass spectrometry array platform,” Anal. Chem. 79(15): 5987-5990 (2007)). All publications are incorporated herein by reference.

In a further example, the inventive biomarkers can be detected using electrochemical approaches. See, e.g., Lin et al., Anal. Sci. 23(9): 1059-1063 (2007)).

In one embodiment, the LMW peptides are harvested from a biological sample prior to the evaluation step. For example, 100 μl of serum can be mixed with 2×SDS-PAGE Laemmli Buffer (containing 200 mM DTT), boiled for 10 minutes, and loaded on Prep Cell (Model 491 Prep Cell, Bio-Rad Laboratories, Calif.) comprising a 5 cm length 10% acrylamide gel. Electrophoresis is performed under a constant voltage of 250V. Immediately after the bromophenol blue indicator dye is eluted from the system, LMW peptides and proteins migrate out of the gel and are trapped in a dialysis membrane in the elution chamber. These molecules can be eluted at a flow rate of 400 ml/min by a buffer with the same composition of the Tris-Glycine running buffer and collected for 10 minutes in one fraction.

Alternatively, LMW peptides can be harvested from a sample using a capture-particle that comprises a molecular sieve portion and an analyte binding portion as described in U.S. patent application Ser. No. 11/527,727, filed Sep. 27, 2006, which is incorporated herein by reference. Briefly, either the molecular sieve portion or the analyte binding portion or both comprise a cross-linked region having modified porosity, or pore dimensions sufficient to exclude high molecular weight molecules.

In another embodiment, the LMW peptides are digested prior to detection, so as to reduce the size of the peptides. Such digestion can be carried out using standard methods well known in the field. Exemplary treatments, include but are not limited to, enzymatic and chemical treatments. Such treatments can yield partial as well as complete digestions. One example of an enzymatic treatment is a trypsin digestion.

The inventive biomarkers are particularly useful in detecting a neurological condition during its early stages, such as while the condition is still associated with MCI or PMCI or for detecting brain vasculopathy, such as brain microhemorrhages. For clarification, mild cognitive impairment (MCI) cases fulfill the Mayo Clinic criteria for classification as MCI-multiple domain impairment (MCI-MCDI) with the following characteristics: i) A memory complaint confirmed by either corrected Logical Memory testing or reports of the informant and a CDR=0.5. ii) Normal activities of daily living. iii) Normal general cognitive function. iv) Abnormal memory for age as measured by standard scores and education. v) A global CDR of 0.5 and no dementia. vi) No history of significant vascular problems, insulin-requiring diabetes, or uncontrolled hypertension. Meanwhile, stable mild cognitive impairment (stable MCI) is based on a Sum of boxes=0.5-3.5 on several evaluations, CDR logical memory impairment with logical memory impairment on at least one evaluation, neuropsychological testing in MCI range inconsistently and clinical judgment. Progressive mild cognitive impairment (PMCI) denotes patients with a Sum of Boxes≧3.5 on two occasions, neuropsychological tests congruent with CDR, a Logical Memory raw score low to zero and clinical judgment.

The abundance of the biomarker can be measured by detecting the biomarker as described above and comparing the amount of the biomarker to a control. The abundance of the biomarker is an indicator of the neurological condition. If the biomarker is “less abundant” in the control, then the biomarker is present in the tested sample in a significantly less amount than in the control sample. If the biomarker is “more abundant” than the control, then the biomarker is present in the tested sample in a significantly greater amount than in the control sample. For instance, the difference may be 5%, 10%, 15%, 20%, 25%, 30%, 35%, 40%, 45%, 50%, 55%, 60%, 65%, 70%, 75%, 80%, 85%, 90%, 95%, 100%, 110%, 125%, 150%, 175%, 200%, 250%, 300%, 350%, 400%, 450%, 500%, 550%, 600%, 650%, 700%, 750%, 800%, 850%, 900%, 950%, 1000%, or greater. The control can be a sample or its equivalent from a normal patient or from a patient in a known disease state. For instance, the control can be from a patient with AD, MCI or brain microhemorrhages. The control can also be a standard or known amount of a reference peptide.

The neurological condition being detected can be, for example, Alzheimer's disease (AD), mild cognitive impairment (MCI), stable mild cognitive impairment (stable MCI), progressive mild cognitive impairment (PMCI), vascular dementia (VD), angiopathy black holes, cerebral amyloid angiopathy (CAA) and brain microhemorrhages. Unless otherwise indicated, the conditions and activities noted herein refer to the commonly accepted definitions thereof. For instance, as described in more detail in the Examples, cognitive impairment is defined according to the Mayo Clinic criteria.

In another embodiment, the biomarker is a peptide associated with a metabolic pathway or cellular process. In further embodiments, the biomarker is a peptide associated with inflammation, estrogen activity, pigment epithelium-derived factor (PEDF)vitamin D metabolism and bone mineralization, coagulation and platelet activity, the complement cascade, acyl-peptide hydrolase (APH) activity, vitamin A and thyroxine, phospholipase activity, globin activity, glycosylation or is glycosylated, protease inhibition, keratins and related proteins, heme degradation, pyruvate metabolism, calcium related proteins, defensin, gelsolin, vitronectin, profilin, thrombospondin, peroxiredoxin, alcohol dehydrogenase, apolipoproteins, iron and copper metabolism, or NMDA receptor-related proteins.

In one aspect, more than one biomarker can be evaluated simultaneously. For example, at least two, at least five, at least 10, at least 20, at least 30, at least 50, at least 75, at least 100 biomarkers are evaluated in the methods. Analyzing more than one biomarker can increase accuracy of the diagnosis.

The present methods can be combined with neuroimaging techniques for the detection of neuropathy and brain microvasculopathy associated with a neurological condition. For example, neuroimaging can be used to detect brain microhemorrages associated with cognitive impairment. Using magnetic resonance imaging, focal signal intensity losses secondary to iron-containing hemosiderin residuals can be detected. These spots on the MR image have been termed “signal voids,” “susceptibility artifacts,” “black holes,” “dots,” “microbleeds,” “old microbleeds” (OMBs), “multifocal signal loss lesions” or “microhemorrhages” (MH). Generically, these spots are called small hypointensities (SH) and are associated with AD and MCI (Cordonnier et al. Neurology (2006) 66:1356-1360; Werring et al. Brain (2004) 127:2265-2275). Suitable MR imaging techniques include gradient refocused echo T₂* (GRE-T₂) and susceptibility weighted imaging (SWI).

Neuroimaging methods that detect metabolic changes in the brain also can be used in conjunction with the present biomarkers. MR spectroscopy that detects, for instance, differences in neurotransmitters, such as glutamine, glutamate and gamma-aminobutryic acid (GABA), can be used to analyze changes in these systems associated with a neurological condition. These metabolic changes can be correlated with cognitive decline and biomarker abundance.

Antibodies specific for the inventive biomarkers can be produced readily using well known methods in the art. (See, J. Sambrook, E. F. Fritsch and T. Maniatis, Molecular Cloning, a Laboratory Manual, second edition, Cold Spring Harbor Laboratory Press, pp. 18.7-18.18, 1989) For example, the inventive biomarkers can be prepared readily using an automated peptide synthesizer. Next, injection of an immunogen, such as (peptide)ₙ-KLH (n=1-30) in complete Freund's adjuvant, followed by two subsequent injections of the same immunogen suspended in incomplete Freund's adjuvant into immunocompetent animals, is followed three days after an i.v. boost of antigen, by spleen cell harvesting. Harvested spleen cells are then fused with Sp2/0-Ag14 myeloma cells and culture supernatants of the resulting clones analyzed for anti-peptide reactivity using a direct-binding ELISA. Fine specificity of generated antibodies can be detected by using peptide fragments of the original immunogen.

In certain embodiments, one or more antibodies directed to the inventive biomarkers is provided in a kit, for use in a diagnostic method. Such kits also can comprise reagents, instructions and other products for performing the diagnostic method.

In other aspects, the biomarkers and antibodies of the present invention are useful for discovering novel aspects of neurological conditions, such as those described herein.

The following examples are illustrative only, and should not be construed as limiting. Also, each reference disclosed herein, and throughout the specification, is incorporated by reference in its entirety.

### Examples

### Example 1

### Background and Patient Summary

A community-based cohort of 103 participants (75 MCI and 28 cognitively normal subjects) was recruited for the study. Of the original 75 MCI subjects, 20 have been censored from the study for various reasons not related to dementia, leaving 55 which are currently being followed. Seventeen of these have become demented over a 0.5 to 4.1-year observation period (15% annual conversion rate) based upon on the Clinical Dementia Rating (CDR) Sum of Boxes score≧3.5 as documented by NINCDS-ADRDA criteria.(Schafer et al. Alzheimer Dis Assoc Disord.(2004) 18:219-222; McKhann et al. Neurology.(1984) 34:939-944) Four of 28 cognitively normal subjects have progressed to the MCI category with significant SH detected by SWI in two. Two MCI cases are on the verge of dementia at present, one with significant SH. SWI brain imaging has demonstrated increasing and “significant” numbers (n≧5) of SH in 7 of the 17 demented and progressively cognitively impaired subjects. This progressive increase in SH in a lobar, posteriorly situated cortical-subcortical pattern fits the diagnostic pattern for “probable CAA.”(Knudsen et. al. Neurology. (2001) 56:537-539) This observation is the first prospective evidence for a subset of sporadic late-onset dementia correlating temporally with increasing SH in a pattern typical for CAA.

### Subject Selection:

After screening 1348 community based individuals at publicized memory clinics, 28 elderly “controls” and 75 subjects with MCI qualified for the study using inclusion and exclusion criteria defined by the Mayo Clinic Group (Petersen R C, et al. Arch Neurol. March (1999) 56:303-308.) Subjects have been continuously evaluated with serial cognitive (bi-yearly) and radiologic (yearly) procedures over 4.1 years (range 0.5 to 4.10 years, average total follow-up time 2.3±1.2 years, total person years of follow-up 241.7 years). All subjects gave informed consent and all studies were approved by the Loma Linda University Institutional Review Board. Complete medication, medical and smoking histories were obtained on all subjects, and thyroid function, serum B12 levels, and ApoE genotype were defined in all subjects.

Normal Subjects: (n=28)

All “control subjects” were without objective or subjective memory deficits and within normal limits on neuropsychological testing (Global CDR of 0, CDR memory component of 0 and a sum of CDR boxes of 1 or less at baseline). The sum of CDR boxes is used as a measure of cognitive performance.(107)

MCI Subjects: (n=75)

All MCI cases fulfilled the Mayo Clinic criteria for classification as MCI-multiple domain impairment (MCI-MCDI) with the following characteristics: i) a memory complaint confirmed by either corrected Logical Memory testing or reports of the informant and a CDR=0.5; ii) normal activities of daily living. iii) normal general cognitive function; iv) abnormal memory for age as measured by standard scores and education; v) a global CDR of 0.5 and no dementia; and vi) no history of significant vascular problems, insulin-requiring diabetes, or uncontrolled hypertension. Twenty MCI subjects have now been censored for varying reasons: cancer 2, co-morbidity 1, claustrophobia 2, loss of care support/moved 9, lost interest 5 and pacemaker 1.

### Cognitive Testing:

All cognitive assessments were conducted within 4 weeks of the MR evaluation by the same team of neuropsychologists with re-evaluations at approximately 6 month intervals. A total of 476 cognitive tests have been performed with some subjects having as many as 9 evaluations. The battery of cognitive tests included a videotaped CDR plus the following: Logical Memory I, II, North American Adult Reading Test, Word Fluency:Phonetic and Semantic, Wisconsin Card Sorting Test, Trail Making Test A&B, Boston Naming Test, Draw-A-Clock, Depression Features Battery Version II, and Geriatric Depression Scale.

Results of radiologic and cognitive assessments were reviewed bimonthly. On the rare occasion if cognitive testing and neurologic examination indicates development of a disorder other than AD, e.g. frontotemporal dementia, progressive supranuclear palsy, primary progressive aphasia, the subject was removed from the study. Results of the neuropsychological testing were noted as abnormal if below>1.5 standard deviation (SD) on normative data based on age and education. The diagnosis of dementia is based on a clinical judgment (consensus conference), NINCDS-ADRDA criteria, and a Sum of Boxes (SOB) on the CDR≧3.5.(107)

### The Cognitive Course of Cohorts and Current Neuropsychological (NP) Classification:

The cognitive course of the cohorts has been carefully monitored over the past 4.1 years and a five stage classification has emerged (Table 5). This classification is the matrix on which the MR and proteomic findings are co-analyzed. Special attention has been given to the MCI and control cases that under observation have proceeded to cognitive loss (MCI), “dementia,” or “progressed MCI.”

TABLE 5
Five Stage Cognitive NP Classification

NORMAL: Sum of Boxes = 0 to 0.5. CDR Memory = 0, with deference to clinical
judgment. Some occasional abnormalities in neuropsychological tests. Logical
memory consistently normal
UNSTABLE NORMAL (U-normal): Sum of Boxes ≦1 but variable. Some
indication of CDR memory impairment. Trend upward or downward based on clinical
judgment. Neuropsychological testing shows moderate abnormalities with
improvement or decline. Clinical judgment.
MCI: Sum of boxes = 0.5-3.5 on several evaluations. CDR logical memory
impairment with logical memory impairment on at least one evaluation.
Neuropsychological testing is in MCI range inconsistently. Clinical judgment.
UNSTABLE MCI (U-MCI): Sum of Boxes varies from 0.5 to 3.5.
Neuropsychological tests are congruent with Sum of Boxes. Considerable logical
memory impairment. A downward trend indicated. Clinical judgment.
PROGRESSED MCI (PMCI) or mild AD [please confirm]: Sum of Boxes ≧3.5 on
two occasions. Neuropsychological tests congruent with CDR. Logical Memory raw score
low to zero. Clinical judgment.

The above scoring was derived after examination of results of multiple NP evaluations. Subjects with only one evaluation at baseline are classed as Normal or MCI.

TABLE 6
Table of 476 NP Evaluations

| | Number of Evaluations: | | | | | | | | |
|---|---|---|---|---|---|---|---|---|---|
| | 1 | 2 | 3 | 4 | 5 | 6 | 7 | 8 | 9 |
| Number of Participants: | 9 | 7 | 12 | 17 | 26 | 12 | 15 | 3 | 2 |

Clear fluctuations in cognitive performance were found in both the Unstable Normal and Unstable MCI cohorts. The unstable MCI cohort has a cognitive status on occasion of dementia (CDR=3.5) but can improve to 3.0 with medication. A complete medication history has been obtained on all cohorts.

TABLE 7
Baseline NP (original two categories) and Current NP status per
Table 5

| | | Current NP | | | | | |
|---|---|---|---|---|---|---|---|
| Baseline NP | | Normal | U-Normal | MCI | U-MCI | PMCI | Total |
| n = 28 | Normal | 17 | 7 | 4 | 0 | 0 | 28 |
| n = 75 | MCI | 1 | 9 | 40 | 8 | 17 | 75 |
| Total | | 18 | 16 | 44 | 8 | 17 | 103 |

Table 7 gives the current NP status of the cohorts using the five stage classification as derived from entrance classification (normal or MCI). Note progressive movement of normal to MCI and 10 MCI cases moving to U-Normal and Normal, 25 of the MCI cases have moved to U-MCI (8) and PMCI (17). The human experiment was designed to determine MR and proteomic changes during dementia development.

### Materials and Methods

Low molecular weight protein harvesting by PrepCell

100 μl of serum was mixed with SDS-PAGE loading buffer, boiled for 10 minutes, and loaded to PrepCell (Bio-Rad, CA). After 2 hours of electrophoresis, low molecular weight proteins migrated out of the gel and were eluted to collection tubes.

Nanoflow reversed-phase liquid chromatography-tandem MS (nanoRPLC-MS/MS)

Eluted proteins from PrepCell were further passed through detergent clean-up micro kit ProteoSpin (Norgen, Canada) to remove the SDS in the elution buffer that could interfere with mass spectrometry analysis. The cleaned proteins were reduced by 10 mM DTT, alkylated by 50 mM iodoacetamide, and digested by trypsin (from Promega) at 37° C. overnight. Tryptic peptides were further purified by Sep-Pak cartridges (Waters, Mass.) and analyzed by reversed-phase liquid chromatography nanospray tandem mass spectrometry using a linear ion-trap mass spectrometer (LTQ, ThermoElectron, San Jose, Calif.). Separation column was slurry-packed in-house with 5 μm, 200 Å pore size C18 resin (Michrom BioResources, CA) in 100 μm i.d.×10 cm long fused silica capillary (Polymicro Technologies, Phoenix, Ariz.) with a laser-pulled tip. After sample injection, the column was washed for 5 minutes with mobile phase A (0.4% acetic acid) and peptides were eluted using a linear gradient of 0% mobile phase B (0.4% acetic acid, 80% acetonitrile) to 50% mobile phase B in 30 minutes at 250 nanoliter/min, then to 100% B in an additional 5 minutes. The LTQ mass spectrometer was operated in a data-dependent mode in which each full MS scan was followed by five MS/MS scans where the five most abundant molecular ions were dynamically selected for collision-induced dissociation (CID) using a normalized collision energy of 35%.

The ETD method with Thermo LTQ instrument also can be used. The ETD method (Syka et al. Proc. Natl. Acad. Sci. U.S.A. (2004) 101:9528-9533) accomplishes peptide fragmentation in the MS-MS analysis by electron transfer, in contrast to the traditional collision-induced dissociation (CID). ETD has been demonstrated to be more powerful than CID in providing more easily interpretable MS-MS sequence data from larger, higher-charge state peptides (including intact small proteins), as well as those with post-translational modifications (PTMs). (Coon et al. Proc. Natl. Acad. Sci. U.S.A. (2005) 102:9463-9468). The novel combination of CID and ETD analysis can enhance peptide identification productivity.

### Example 2

### Serum Proteomic Analysis

Fractionating LMW Proteins

In the first serum proteomic study, A, 100-μL aliquots of whole serum samples were prepared for high performance liquid chromatography/mass spectrometry (LC-MS) analysis by reduction and alkylation (DTT, iodoacetamide) followed by digestion of the proteins followed by LTQ mass spectroscopy. For subsequent studies, B and C a proteome subset consisting of low molecular weight (LMW) proteins was prepared from each serum sample to reduce the complexity of the protein mixture. The resulting LMW proteins were fractionated by SDS-PAGE and proteins were visualized by Coomassie staining.

For study B, the samples consisted of pooled serum samples from 14-15 subjects (control, MCI and PMCI). With improved LMW isolation, serum proteins with molecular weights 25 kDa were collected and fractionated by SDS-PAGE.

For study C serum samples from 5 individuals who had progressed from control to MCI (1 sample) and from MCI to PMCI were prepared to yield LMW proteins and the LC-MS analyses performed using a Thermo hybrid LTQ-Orbitrap mass spectrometer. This represents the state of the art in the MS technology and provides several advantages compared with the LTQ, such as superior high mass resolution and mass accuracy in the spectra acquired of the precursor peptide molecular ions.

### Data Analysis and Results

MS-MS spectra were searched against a public human protein database (NCBI) using the SEQUEST search algorithm to obtain matches. Results in study A only identified abundant serum proteins. The results led to a focus on low molecular weight (LMW) serum proteins (study B). The threshold of 50 kDa was insufficient to reduce the complexity of proteins, and TCA protein precipitation resulted in unacceptable protein loss. As a result, a high-quality analysis of study B was conducted using pooled samples of a relatively large number (14) of individual subject serum samples per group. This study compared LMW proteins identified in control vs. MCI vs. PMCI sample/subject groups. This qualitative analysis identified candidate biomarkers (differentially abundant proteins). The objective of study C was to identify LMW serum proteins with differential abundances that correlated with progression from MCI to PMCI (4 individuals; 4 sample pairs) and control to MCI (1 individual; 1 pair of samples) diagnoses. These 10 sample analyses yielded identification of more than 500 proteins. No major differences in apoE genotype between subjects are found in the subject cohorts.

Determination of candidate biomarker proteins was achieved by comparing the number of tandem mass spectra (MS2 scans) that were matched to peptide sequences corresponding to the source proteins in the database against which the data were searched. A higher abundance protein relative to a lower abundance one will yield a greater number of, and more abundant, peptides from the enzyme digest, and these peptides often will result in more matched MS2 spectra. In this way, the number of MS2 spectra, termed “spectral count”, is an approximate measure of the relative abundance of proteins in a mixture (Analytical Chemistry, 76(14), 4193-4201 (2004)). The evaluation of candidate differentially abundant proteins focuses on proteins that yielded a 50% or greater spectral count difference in one sample set versus the other.

The results of the studies are shown in Tables 8-10.

### Example 3

### Detection of Brain Microhemorrhages

SH are counted independently at two sites (Detroit MRI Institute for Biomedical Research (DMRI) and Loma Linda University (LLU)) but currently primarily at LLU by raters who are integral to the project using an identical protocol blinded to clinical status. SWI filtered phase images were reviewed for the presence of SH one 2 mm slice at a time. All magnitude images, high pass (HP) filtered phase images and contrast enhanced SWI magnitude images were used in the data review process. Images were placed side by side for identifying SH and HP filtered phase images are used to mark them with review above and below to check for vascular connections. One slice may contain more than one SH as in FIG. 2., then every SH was highlighted with a different colored boundary. Any slice that showed a SH appearing in a previous slice was not recounted. SH are assigned a slice and serial number, size (1-3, 3-5, >5 mm O.D.) and anatomical location. Differentiating microaneurysms with blood in and/or around vessel walls was uncertain since blood collecting in a microaneurysm produces a significant signal void. Subarachnoid and sulcal vascular voids, symmetrical focal basal ganglia signal losses were not counted.

The biomarkers identified as associated with brain microhemorrages are presented in Table 11.

### Example 4

### Further Evaluation of Biomarkers

The inventive biomarkers can be evaluated further using a variety of methods. In addition to traditional biological validation assays, mass spectrometric methods can be used. One method of validation is Western assays of serum samples using commercially available antibodies specific for the candidate proteins. If antibodies are not available commercially, they can be produced readily using methods well know in the art and disclosed herein.

In addition, triple quadruple mass spectrometry (TQMS) technology can be used to further evaluate the biomarkers. The technique employs multiple reaction monitoring (MRM), which consists of (1) detection and selection of molecular ions with the first quadruple, (2) fragmentation of these ions in the second quadruple, and (3) detection of a small number of known fragment ions in the third quadruple. The analysis yields an analyte's molecular weight and the relative abundances of fragment ions that are characteristic of analyte structure and chromatographic elution time (LC/MS). Modern TQMS instruments provide advanced MRM performance with higher resolution and accuracy mass measurement, fast electronics for switching between a large number of selected analyte and fragmentation masses monitored, and ease of use. Inherent advantages of LC/TQMS include high detection sensitivity, large dynamic range of detection response, and the ability to incorporate stable isotope labeled synthetic analogs of the targeted analytes, which allows superior quantitative analytical performance.(Anderson, Mol. Cell. Proteomics (2006) 5:573-588; Frewen et al. Anal. Chem. (2006) 78:5678-5684)

Such studies can be augmented with spiked internal standards, as in the discovery phase, and with isotopically-labeled synthetic analogs of the biomarkers. In addition, an autosampler and other methods can be used to enhance throughput (e.g., plate-based sample peptide enrichment and cleanup prior to LC/MS).

TABLE 1
Biomarkers associated with neurological conditions.

| | | | | | | | |
|---|---|---|---|---|---|---|---|
| | | Protein | | | | | |
| | Protein | molecular | | | Calculated | Peptide | Peptide |
| | accession | weight | SEQ ID | Peptide | Peptide | start | stop |
| Protein name | numbers | (Da) | NO: | sequence | Mass (AMU) | index | index |

| | | | | | | | |
|---|---|---|---|---|---|---|---|
| (O60602) Toll-like receptor | TLR5_HUMAN | 97713 | 1 | LYFCGLSDAVLKD | 2379.22 | 126 | 145 |
| 5 precursor (Toll/inter- | | | | GYFRNLK | | | |
| leukin-1 receptor-like | | | | | | | |
| protein 3) | | | | | | | |
| (O60687) Sushi-repeat- | O60687 | 52954 | 2 | DSADGTITRVTLR | 1404.74 | 212 | 224 |
| containing protein, X-linked | | | | | | | |
| 2 | | | | | | | |
| (O95793) Double-stranded RNA- | STAU_HUMAN | 63250 | 3 | ALRILQNEPLPERL | 2217.25 | 156 | 174 |
| binding protein Staufen | | | | EVNGR | | | |
| homolog | | | | | | | |
| (P01764) Ig heavy chain V-III | HV3C_HUMAN | 12565 | 4 | AEDTAVYYCAK | 1290.57 | 107 | 117 |
| region VH26 precursor | | | | | | | |
| (P02766) Transthyretin | TTHY_HUMAN | 15869 | 5 | AADDTWEPFASGK | 1394.62 | 56 | 68 |
| precursor (Prealbumin) (TBPA) | | | | | | | |
| (TTR) (ATTR) | | | | | | | |
| (P02766) Transthyretin | TTHY_HUMAN | 15869 | 6 | ALGISPFHEHAEVV | 2607.31 | 101 | 124 |
| precursor (Prealbumin) (TBPA) | | | | FTANDSGPRR | | | |
| (TTR) (ATTR) | | | | | | | |
| (P02766) Transthyretin | TTHY_HUMAN | 15869 | 7 | GSPAINVAVHVFR | 1366.76 | 42 | 54 |
| precursor (Prealbumin) (TBPA) | | | | | | | |
| (TTR) (ATTR) | | | | | | | |
| (P02766) Transthyretin | TTHY_HUMAN | 15869 | 8 | GSPAINVAVHVFR | 1494.85 | 42 | 55 |
| precursor (Prealbumin) (TBPA) | | | | K | | | |
| (TTR) (ATTR) | | | | | | | |
| (P02766) Transthyretin | TTHY_HUMAN | 15869 | 9 | KAADDTWEPFASG | 1522.72 | 55 | 68 |
| precursor (Prealbumin) (TBPA) | | | | K | | | |
| (TTR) (ATTR) | | | | | | | |
| (P02766) Transthyretin | TTHY_HUMAN | 15869 | 10 | RYTIAALLSPYSYS | 2645.38 | 124 | 147 |
| precursor (Prealbumin) (TBPA) | | | | TTAVVTNPKE | | | |
| (TTR) (ATTR) | | | | | | | |
| (P02766) Transthyretin | TTHY_HUMAN | 15869 | 11 | TSESGELHGLTTEE | 3140.52 | 69 | 96 |
| precursor (Prealbumin) (TBPA) | | | | EFVEGIYKVEIDTK | | | |
| (TTR) (ATTR) | | | | | | | |
| (P02766) Transthyretin | TTHY_HUMAN | 15869 | 12 | VLDAVRGSPAINV | 2020.15 | 36 | 54 |
| precursor (Prealbumin) (TBPA) | | | | AVHVFR | | | |
| (TTR) (ATTR) | | | | | | | |
| (P02766) Transthyretin | TTHY_HUMAN | 15869 | 13 | YTIAALLSPYSYST | 2489.28 | 125 | 147 |
| precursor (Prealbumin) (TBPA) | | | | TAVVTNPKE | | | |
| (TTR) (ATTR) | | | | | | | |
| (P09758) Tumor-associated | TACD2_HUMAN | 35692 | 14 | HRPTAGAFNHSDL | 2163.08 | 160 | 178 |
| calcium signal transducer 2 | | | | DAELRR | | | |
| precursor (Pancreatic carci- | | | | | | | |
| noma marker protein GA733-1) | | | | | | | |
| (Cell surface glycoprotein | | | | | | | |
| Trop-2) | | | | | | | |
| (P09874) Poly [ADP-ribose] | PARP1_HUMAN | 112939 | 15 | EDAIEHFMKLYEE | 1781.84 | 607 | 620 |
| polymerase 1 (EC 2.4.2.30) | | | | K | | | |
| (PARP-1) (ADPRT) (NAD(+) ADP- | | | | | | | |
| ribosyltransferase 1) (Poly | | | | | | | |
| [ADP-ribose]synthetase 1) | | | | | | | |
| (P09874) Poly [ADP-ribose] | PARP1_HUMAN | 112939 | 16 | MKLTLKGGAAVD | 2703.41 | 522 | 547 |
| polymerase 1 (EC 2.4.2.30) | | | | PDSGLEHSAHVLE | | | |
| (PARP-1) (ADPRT) (NAD(+) ADP- | | | | K | | | |
| ribosyltransferase 1) (Poly | | | | | | | |
| [ADP-ribose]synthetase 1 | | | | | | | |
| (P10643) Complement component | CO7_HUMAN | 93499 | 17 | AASGTQNNVLR | 1130.59 | 362 | 372 |
| C7 precursor | | | | | | | |
| (P10643) Complement component | CO7_HUMAN | 93499 | 18 | FSSHGCK | 822.36 | 348 | 354 |
| C7 precursor | | | | | | | |
| (P10643) Complement component | CO7_HUMAN | 93499 | 19 | MHVLHCQGR | 1137.54 | 745 | 753 |
| C7 precursor | | | | | | | |
| (P10643) Complement component | CO7_HUMAN | 93499 | 20 | SYTSHTNEIHK | 1316.62 | 234 | 244 |
| C7 precursor | | | | | | | |
| (P13667) Protein disulfide- | PDIA4_HUMAN | 72916 | 21 | LAPEYEKAAKELS | 1576.86 | 212 | 225 |
| isomerase A4 precursor (EC | | | | K | | | |
| 5.3.4.1) (Protein ERp-72) | | | | | | | |
| (ERp72) | | | | | | | |
| (P16591) Proto-oncogene tyro- | FER_HUMAN | 94609 | 22 | FLQEAKILKQYDH | 2184.22 | 605 | 622 |
| sine-protein kinase FER (EC | | | | PNIVK | | | |
| 2.7.1.112) (p94-FER) (c-FER) | | | | | | | |
| (P20701) Integrin alpha-L | ITAL_HUMAN | 128804 | 23 | EGVNITICFQIKS | 2498.29 | 646 | 666 |
| precursor (Leukocyte adhesion | | | | LYPQFQGR | | | |
| glycoprotein LFA-1 alpha | | | | | | | |
| chain) (LFA-1A) (Leukocyte | | | | | | | |
| function associated molecule | | | | | | | |
| 1, alpha chain) (CD11a) | | | | | | | |
| (P22792) Carboxypeptidase N | CPN2_HUMAN | 60599 | 24 | TLNLAQNLLAQLP | 3045.70 | 173 | 199 |
| subunit 2 precursor (Carboxy- | | | | EELFHPLTSLQTLK | | | |
| peptidase N polypeptide 2) | | | | | | | |
| (Carboxypeptidase N 83 kDa | | | | | | | |
| chain) (Carboxypeptidase N | | | | | | | |
| regulatory subunit) (Carboxy- | | | | | | | |
| peptidase N large subunit) | | | | | | | |
| (P35542) Serum amyloid A-4 | SAA4_HUMAN | 14789 | 25 | FRPDGLPK | 929.52 | 121 | 128 |
| protein precursor (Consti- | | | | | | | |
| tutively expressed serum amy- | | | | | | | |
| loid A protein) (C-SAA) | | | | | | | |
| (Q05513) Protein kinase C, | KPCZ_HUMAN | 67644 | 26 | EGLGPGDTTSTFCG | 2666.28 | 400 | 424 |
| zeta type (EC 2.7.1.37) | | | | TPNYIAPEILR | | | |
| (nPKC-zeta) | | | | | | | |
| (Q15166) Serum paraoxonase/ | PON3_HUMAN | 39590 | 27 | ILIGTVFHK | 1027.63 | 340 | 348 |
| lactonase 3 (EC 3.1.1.-) | | | | | | | |
| (Q4V312) Colony stimulating | Q4V312 | 46884 | 28 | DKLNDNHEVEDE | 2025.89 | 364 | 380 |
| factor 2 receptor, alpha, | | | | MGPQR | | | |
| low-affinity (Granulocyte- | | | | | | | |
| macrophage) | | | | | | | |
| (Q6N092) Hypothetical pro- | Q6N092 | 56405 | 29 | SAVQGPPDR | 926.47 | 335 | 343 |
| tein DKFZp686K18196 | | | | | | | |
| (Fragment) | | | | | | | |
| (Q6N092) Hypothetical pro- | Q6N092 | 56405 | 30 | SAVQGPPDR | 926.47 | 335 | 343 |
| tein DKFZp686K18196 | | | | | | | |
| (Fragment) | | | | | | | |
| (Q7Z3Z2) Protein C1orf36 | CA036_HUMAN | 22686 | 31 | PRGSLATFK | 976.56 | 146 | 154 |
| (Q8N7W7) Hypothetical protein | Q8N7W7 | 66111 | 32 | ILIDGTLIIFR | 1273.79 | 124 | 134 |
| FLJ40259 | | | | | | | |
| (Q96MA6) Hypothetical protein | Q96MA6 | 54909 | 33 | KTVPSALLVQLIQE | 2653.46 | 111 | 133 |
| FLJ32704 (Chromosome 9 open | | | | RLAEEDCIK | | | |
| reading frame 98) | | | | | | | |
| (OTTHUMP00000022737) | | | | | | | |
| (Q96S24) Hypothetical pro- | Q96S24 | 3503 | 34 | GMGSDRTALSLQG | 2606.31 | 3 | 26 |
| tein gs30 | | | | AWGIFLSTFYK | | | |
| (Q99996) A-kinase anchor pro- | AKAP9_HUMAN | 453646 | 35 | CEVNAEDKENSGD | 3218.33 | 1232 | 1258 |
| tein 9 (Protein kinase A | | | | YISENEDPELQDYR | | | |
| anchoring protein 9) (PRKA9) | | | | | | | |
| (A-kinase anchor protein 450 | | | | | | | |
| kDa) (AKAP 450) (A-kinase | | | | | | | |
| anchor protein 350 kDa) (AKAP | | | | | | | |
| 350) (hgAKAP 350) (AKAP 120- | | | | | | | |
| like protein) (Hyperion pro- | | | | | | | |
| tein) (Yotiao protein) | | | | | | | |
| (Centrosome- and Golgi- | | | | | | | |
| localized PKN-associated | | | | | | | |
| protein) (CG-NAP) | | | | | | | |
| (Q99996) A-kinase anchor pro- | AKAP9_HUMAN | 453646 | 36 | KAYINTISSLKDLI | 1808.05 | 2989 | 3004 |
| tein 9 (Protein kinase A | | | | TK | | | |
| anchoring protein 9) (PRKA9) | | | | | | | |
| (A-kinase anchor protein 450 | | | | | | | |
| kDa) (AKAP 450) (A-kinase | | | | | | | |
| anchor protein 350 kDa) | | | | | | | |
| (AKAP 350) (hgAKAP 350) | | | | | | | |
| (AKAP 120-like protein) | | | | | | | |
| (Hyperion protein) (Yotiao | | | | | | | |
| protein) (Centrosome- and | | | | | | | |
| Golgi-localized PKN- | | | | | | | |
| associated protein) (CG-NAP) | | | | | | | |
| (Q9BXB9) LIM mineralization | Q9BXB9 | 46492 | 37 | HSQPATPTPLQSR | 1419.73 | 212 | 224 |
| protein 2 | | | | | | | |
| (Q9H2I2) HNRBF-2 | Q9H2I2 | 31791 | 38 | NCYSLLNLAEVRS | 2128.12 | 236 | 253 |
| | | | | KYLGK | | | |
| (Q9H2I2) HNRBF-2 | Q9H2I2 | 31791 | 39 | NCYSLLNLAEVRS | 2128.12 | 236 | 253 |
| | | | | KYLGK | | | |
| (Q9NPP6) Immunoglobulin heavy | Q9NPP6 | 44767 | 40 | VEDTAVYYCAR | 1346.61 | 48 | 58 |
| chain variant (Fragment) | | | | | | | |
| (Q9NYQ6) Cadherin EGF LAG | CELR1_HUMAN | 329464 | 41 | ENLEVGYEVLTIRA | 2944.51 | 364 | 389 |
| seven-pass G-type receptor 1 | | | | SDRDSPINANLR | | | |
| precursor (Flamingo homolog | | | | | | | |
| 2) (hFmi2) | | | | | | | |
| (Q9NYQ6) Cadherin EGF LAG | CELR1_HUMAN | 329464 | 42 | ETKETHVLR | 1112.61 | 314 | 322 |
| seven-pass G-type receptor 1 | | | | | | | |
| precursor (Flamingo homolog | | | | | | | |
| 2) (hFmi2) | | | | | | | |
| (Q9NYQ6) Cadherin EGF LAG | CELR1_HUMAN | 329464 | 43 | QFVGCMRNLSVDG | 1610.78 | 1616 | 1629 |
| seven-pass G-type receptor 1 | | | | K | | | |
| precursor (Flamingo homolog | | | | | | | |
| 2) (hFmi2) | | | | | | | |
| (Q9NYQ6) Cadherin EGF LAG | CELR1_HUMAN | 329464 | 44 | VPGGTRAFALRPG | 2606.31 | 35 | 59 |
| seven-pass G-type receptor 1 | | | | CTYAVGAACTPR | | | |
| precursor (Flamingo homolog | | | | | | | |
| 2) (hFmi2) | | | | | | | |
| adenylosuccinate synthase | gi\|34577063\| | 50080 | 45 | DGVYFLYEALHGP | 1833.95 | 233 | 248 |
| [Homo sapiens] | ref\| | | | PKK | | | |
| | NP_001117.2\| | | | | | | |
| alpha 1B-glycoprotein | gi\|21071030\| | 54235 | 46 | ATWSGAVLAGR | 1088.59 | 407 | 417 |
| [Homo sapiens] | ref\| | | | | | | |
| | NP_570602.2\| | | | | | | |
| alpha 1B-glycoprotein | gi\|21071030\| | 54235 | 47 | SLPAPWLSMAPVS | 2151.17 | 115 | 134 |
| [Homo sapiens] | ref\| | | | WITPGLK | | | |
| | NP_570602.2\| | | | | | | |
| alpha 1B-glycoprotein | gi\|21071030\| | 54235 | 48 | SWVPHTFESELSDP | 2471.20 | 474 | 495 |
| [Homo sapiens] | ref\| | | | VELLVAES | | | |
| | NP_570602.2\| | | | | | | |
| alpha 1B-glycoprotein | gi\|21071030\| | 54235 | 49 | TPGAAANLELIFVG | 2296.18 | 448 | 469 |
| [Homo sapiens] | ref\| | | | PQHAGNYR | | | |
| | NP_570602.2\| | | | | | | |
| Alpha-1-antichymotrypsin | AACT_HUMAN | 47635 | 50 | ADLSGITGAR | 960.51 | 341 | 350 |
| precursor (ACT) [Contains: | | | | | | | |
| Alpha-1-antichymotrypsin His- | | | | | | | |
| Pro-less] | | | | | | | |
| Alpha-1-antichymotrypsin | AACT_HUMAN | 47635 | 51 | AVLDVFEEGTEAS | 1907.96 | 361 | 379 |
| precursor (ACT) [Contains: | | | | AATAVK | | | |
| Alpha-1-antichymotrypsin His- | | | | | | | |
| Pro-less] | | | | | | | |
| Alpha-1-antichymotrypsin | AACT_HUMAN | 47635 | 52 | DYNLNDILLQLGIE | 2296.17 | 321 | 340 |
| precursor (ACT) [Contains: | | | | EAFTSK | | | |
| Alpha-1-antichymotrypsin His- | | | | | | | |
| Pro-less] | | | | | | | |
| Alpha-1-antichymotrypsin | AACT_HUMAN | 47635 | 53 | ITLLSALVETR | 1215.73 | 380 | 390 |
| precursor (ACT) [Contains: | | | | | | | |
| Alpha-1-antichymotrypsin His- | | | | | | | |
| Pro-less] | | | | | | | |
| Alpha-1-antichymotrypsin | AACT_HUMAN | 47635 | 54 | NLAVSQVVHK | 1094.63 | 351 | 360 |
| precursor (ACT) [Contains: | | | | | | | |
| Alpha-1-antichymotrypsin His- | | | | | | | |
| Pro-less] | | | | | | | |
| Alpha-1-antichymotrypsin | AACT_HUMAN | 47635 | 55 | YNLNDILLQLGIEE | 2181.14 | 322 | 340 |
| precursor (ACT) [Contains: | | | | AFTSK | | | |
| Alpha-1-antichymotrypsin His- | | | | | | | |
| Pro-less] | | | | | | | |
| Apolipoprotein C-I precursor | APOC1_HUMAN | 9314 | 56 | EWFSETFQK | 1201.55 | 66 | 74 |
| (Apo-CI) (ApoC-I) | | | | | | | |
| Apolipoprotein C-I precursor | APOC1_HUMAN | 9314 | 57 | IKQSELSAK | 1003.58 | 55 | 63 |
| (Apo-CI) (ApoC-I) | | | | | | | |
| Apolipoprotein C-I precursor | APOC1_HUMAN | 9314 | 58 | LKEFGNTLEDK | 1293.67 | 37 | 47 |
| (Apo-CI) (ApoC-I) | | | | | | | |
| Apolipoprotein C-I precursor | APOC1_HUMAN | 9314 | 59 | TPDVSSALDK | 1032.52 | 27 | 36 |
| (Apo-CI) (ApoC-I) | | | | | | | |
| apolipoprotein C-II precursor | gi\|32130518\| | 11266 | 60 | ESLSSYWESAK | 1286.59 | 42 | 52 |
| [Homo sapiens] | ref\| | | | | | | |
| | NP_000474.2\| | | | | | | |
| apolipoprotein C-II precursor | gi\|32130518\| | 11266 | 61 | LRDLYSK | 894.50 | 71 | 77 |
| [Homo sapiens] | ref\| | | | | | | |
| | NP_000474.2\| | | | | | | |
| apolipoprotein C-II precursor | gi\|32130518\| | 11266 | 62 | STAAMSTYTGIFTD | 2233.14 | 78 | 98 |
| [Homo sapiens] | ref\| | | | QVLSVLK | | | |
| | NP_000474.2\| | | | | | | |
| apolipoprotein C-II precursor | gi\|32130518\| | 11266 | 63 | STAAMSTYTGIFTD | 2548.25 | 78 | 101 |
| [Homo sapiens] | ref\| | | | QVLSVLKGEE | | | |
| | NP_000474.2\| | | | | | | |
| apolipoprotein C-II precursor | gi\|32130518\| | 11266 | 64 | TAAQNLYEK | 1037.53 | 53 | 61 |
| [Homo sapiens] | ref\| | | | | | | |
| | NP_000474.2\| | | | | | | |
| apolipoprotein C-II precursor | gi\|32130518\| | 11266 | 65 | TYLPAVDEK | 1035.54 | 62 | 70 |
| [Homo sapiens] | ref\| | | | | | | |
| | NP_000474.2\| | | | | | | |
| Apolipoprotein C-IV precursor | APOC4_HUMAN | 14536 | 66 | AWFLESK | 880.46 | 99 | 105 |
| (Apo-CIV) (ApoC-IV) | | | | | | | |
| Apolipoprotein C-IV precursor | APOC4_HUMAN | 14536 | 67 | DGWQWFWSPSTFR | 1699.77 | 67 | 79 |
| (Apo-CIV) (ApoC-IV) | | | | | | | |
| Apolipoprotein C-IV precursor | APOC4_HUMAN | 14536 | 68 | ELLETVVNR | 1072.60 | 56 | 64 |
| (Apo-CIV) (ApoC-IV) | | | | | | | |
| apolipoprotein L1 isoform a | gi\|21735614\| | 43957 | 69 | ILQADQEL | 929.49 | 391 | 398 |
| precursor [Homo sapiens] | ref\| | | | | | | |
| | NP_003652.2\|, | | | | | | |
| | gi\|217353616\| | | | | | | |
| | ref\| | | | | | | |
| | NP_663318.1\| | | | | | | |
| apolipoprotein L1 isoform a | gi\|21735614\| | 43957 | 70 | LNILNNNYK | 1105.60 | 382 | 390 |
| precursor [Homo sapiens] | ref\| | | | | | | |
| | NP_003652.2\|, | | | | | | |
| | gi\|21735616\| | | | | | | |
| | ref\| | | | | | | |
| | NP_663318.1\| | | | | | | |
| apolipoprotein M | gi\|22091452\| | 21236 | 71 | EELATFDPVDNIVF | 3086.51 | 58 | 85 |
| [Homo sapiens] | ref\| | | | NMAAGSAPMQLH | | | |
| | NP_061974.2\| | | | LR | | | |
| apolipoprotein M | gi\|22091452\| | 21236 | 72 | EFPEVHLGQWYFI | 2261.14 | 38 | 57 |
| [Homo sapiens] | ref\| | | | AGAAPTK | | | |
| | NP_061974.2\| | | | | | | |
| apolipoprotein M | gi\|22091452\| | 21236 | 73 | KWIYHLTEGSTDL | 1718.89 | 99 | 112 |
| [Homo sapiens] | ref\| | | | R | | | |
| | NP_061974.2\| | | | | | | |
| apolipoprotein M | gi\|22091452\| | 21236 | 74 | MKDGLCVPR | 1091.53 | 90 | 98 |
| [Homo sapiens] | ref\| | | | | | | |
| | NP_061974.2\| | | | | | | |
| apolipoprotein M | gi\|22091452\| | 21236 | 75 | SLTSCLDSK | 1010.48 | 163 | 171 |
| [Homo sapiens] | ref\| | | | | | | |
| | NP_061974.2\| | | | | | | |
| beta globin [Homo sapiens] | gi\|4504349\| | 15980 | 76 | EFTPPVQAAYQK | 1378.70 | 122 | 133 |
| | ref\| | | | | | | |
| | NP_000509.1\| | | | | | | |
| beta globin [Homo sapiens] | gi\|4504349\| | 15980 | 77 | FFESFGDLSTPDAV | 2058.95 | 42 | 60 |
| | ref\| | | | MGNPK | | | |
| | NP_000509.1\| | | | | | | |
| beta globin [Homo sapiens] | gi\|4504349\| | 15980 | 78 | FFESFGDLSTPDAV | 2286.11 | 42 | 62 |
| | ref\| | | | MGNPKVK | | | |
| | NP_000509.1\| | | | | | | |
| beta globin [Homo sapiens] | gi\|4504349\| | 15980 | 79 | GTFATLSELHCDK | 1478.70 | 84 | 96 |
| | ref\| | | | | | | |
| | NP_000509.1\| | | | | | | |
| beta globin [Homo sapiens] | gi\|4504349\| | 15980 | 80 | GTFATLSELHCDKL | 2586.24 | 84 | 105 |
| | ref\| | | | HVDPENFR | | | |
| | NP_000509.1 | | | | | | |
| beta globin [Homo sapiens] | gi\|4504349\| | 15980 | 81 | KVLGAFSDGLAHL | 1797.99 | 67 | 83 |
| | ref\| | | | DNLK | | | |
| | NP_000509.1\| | | | | | | |
| beta globin [Homo sapiens] | gi\|4504349\| | 15980 | 82 | LHVDPENFR | 1126.56 | 97 | 105 |
| | ref\| | | | | | | |
| | NP_000509.1\| | | | | | | |
| beta globin [Homo sapiens] | gi\|4504349\| | 15980 | 83 | LLGNVLVCVLAHH | 1776.99 | 106 | 121 |
| | ref\| | | | FGK | | | |
| | NP_000509.1\| | | | | | | |
| beta globin [Homo sapiens] | gi\|4504349\| | 15980 | 84 | LLVVYPWTQR | 1274.73 | 32 | 41 |
| | ref\| | | | | | | |
| | NP_000509.1\| | | | | | | |
| beta globin [Homo sapiens] | gi\|4504349\| | 15980 | 85 | LLVVYPWTQRFFE | 3314.66 | 32 | 60 |
| | ref\| | | | SFGDLSTPDAVMG | | | |
| | NP_000509.1\| | | | NPK | | | |
| beta globin [Homo sapiens] | gi\|4504349\| | 15980 | 86 | SAVTALWGK | 932.52 | 10 | 18 |
| | ref\| | | | | | | |
| | NP_000509.1\| | | | | | | |
| beta globin [Homo sapiens] | gi\|4504349\| | 15980 | 87 | SAVTALWGKVNV | 2228.17 | 10 | 31 |
| | ref\| | | | DEVGGEALGR | | | |
| | NP_000509.1\| | | | | | | |
| beta globin [Homo sapiens] | gi\|4504349\| | 15980 | 88 | SAVTALWGKVNV | 3483.87 | 10 | 41 |
| | ref\| | | | DEVGGEALGRLLV | | | |
| | NP_000509.1\| | | | VYPWTQR | | | |
| beta globin [Homo sapiens] | gi\|4504349\| | 15980 | 89 | VLGAFSDGLAHLD | 1669.89 | 68 | 83 |
| | ref\| | | | NLK | | | |
| | NP_000509.1\| | | | | | | |
| beta globin [Homo sapiens] | gi\|4504349\| | 15980 | 90 | VNVDEVGGEALGR | 1314.67 | 19 | 31 |
| | ref\| | | | | | | |
| | NP_000509.1\| | | | | | | |
| beta globin [Homo sapiens] | gi\|4504349\| | 15980 | 91 | VVAGVANALAHK | 1149.67 | 134 | 145 |
| | ref\| | | | | | | |
| | NP_000509.1\| | | | | | | |
| beta globin [Homo sapiens] | gi\|4504349\| | 15980 | 92 | VVAGVANALAHK | 1449.80 | 134 | 147 |
| | ref\| | | | YH | | | |
| | NP_000509.1\| | | | | | | |
| biliverdin reductase B | gi\|4502419\| | 22101 | 93 | LPSEGPRPAHVVV | 2469.31 | 40 | 63 |
| (flavin reductase (NADPH)) | ref\| | | | GDVLQAADVDK | | | |
| [Homo sapiens] | NP_000704.1\| | | | | | | |
| biliverdin reductase B | gi\|4502419\| | 22101 | 94 | NIVAAMK | 746.42 | 93 | 99 |
| (flavin reductase (NADPH)) | ref\| | | | | | | |
| [Homo sapiens] | NP_000704.1\| | | | | | | |
| biliverdin reductase B | gi\|4502419\| | 22101 | 95 | PAHVVVGDVLQA | 1732.92 | 47 | 63 |
| (flavin reductase (NADPH)) | ref\| | | | ADVDK | | | |
| [Homo sapiens] | NP_000704.1\| | | | | | | |
| biliverdin reductase B | gi\|4502419\| | 22101 | 96 | TVAGQDAVIVLLG | 1512.88 | 64 | 78 |
| (flavin reductase (NADPH)) | ref\| | | | TR | | | |
| [Homo sapiens] | NP_000704.1\| | | | | | | |
| biliverdin reductase B | gi\|4502419\| | 22101 | 97 | VVACTSAFLLWDP | 1707.88 | 106 | 120 |
| (flavin reductase (NADPH)) | ref\| | | | TK | | | |
| [Homo sapiens] | NP_000704.1\| | | | | | | |
| biliverdin reductase B | gi\|4502419\| | 22101 | 98 | YVAVMPPHIGDQP | 2671.36 | 146 | 170 |
| (flavin reductase (NADPH)) | ref\| | | | LTGAYTVTLDGR | | | |
| [Homo sapiens] | NP_000704.1\| | | | | | | |
| chromosome 1 open reading | gi\|21361470\| | 32112 | 99 | EISEAMK | 823.39 | 183 | 189 |
| frame 48 [Homo sapiens] | ref\| | | | | | | |
| | NP_056286.2\| | | | | | | |
| chromosome 9 open reading | gi\|11641247\| | 17200 | 100 | EAQQYSEALASTR | 1453.69 | 38 | 50 |
| frame 19 [Homo sapiens] | ref\| | | | | | | |
| | NP_071738.1\| | | | | | | |
| citron [Homo sapiens] | gi\|32698688\| | 231418 | 101 | QQKFYLETQAGK | 1440.75 | 814 | 825 |
| | ref\| | | | | | | |
| | NP_009105.1\| | | | | | | |
| coagulation factor II pre- | gi\|4503635\| | 70019 | 102 | ELLESYIDGR | 1194.60 | 354 | 363 |
| cursor [Homo sapiens] | ref\| | | | | | | |
| | NP_000497.1\| | | | | | | |
| coagulation factor II pre- | gi\|4503636\| | 70019 | 103 | HQDFNSAVQLVEN | 1963.91 | 248 | 263 |
| cursor [Homo sapiens] | ref\| | | | FCR | | | |
| | NP_000497.1\| | | | | | | |
| coagulation factor II pre- | gi\|4503635\| | 70019 | 104 | IVEGSDAEIGMSPW | 2265.11 | 364 | 383 |
| cursor [Homo sapiens] | ref\| | | | QVMLFR | | | |
| | NP_000497.1\| | | | | | | |
| coagulation factor II pre- | gi\|4503635\| | 70019 | 105 | LAVTTHGLPCLAW | 1995.05 | 225 | 243 |
| cursor [Homo sapiens] | ref\| | | | ASAQAK | | | |
| | NP_000497.1\| | | | | | | |
| coagulation factor II pre- | gi\|4503635\| | 70019 | 106 | SEGSSVNLSPPLEQ | 2070.98 | 199 | 217 |
| cursor [Homo sapiens] | ref\| | | | CVPDR | | | |
| | NP_000497.1\| | | | | | | |
| coagulation factor II pre- | gi\|4503635\| | 70019 | 107 | SLEDKTERELLESY | 2153.07 | 346 | 363 |
| cursor [Homo sapiens] | ref\| | | | IDGR | | | |
| | NP_000497.1\| | | | | | | |
| coagulation factor II pre- | gi\|4503635\| | 70019 | 108 | TATSEYQTFFNPR | 1561.73 | 315 | 327 |
| cursor [Homo sapiens] | ref\| | | | | | | |
| | NP_000497.1\| | | | | | | |
| coagulation factor II pre- | gi\|4503635\| | 70019 | 109 | TFGSGEADCGLRP | 1883.90 | 328 | 344 |
| cursor [Homo sapiens] | ref\| | | | LFEK | | | |
| | NP_000497.1\| | | | | | | |
| coagulation factor II pre- | gi\|4503635\| | 70019 | 110 | YGFYTHVFR | 1189.58 | 600 | 608 |
| cursor [Homo sapiens] | ref\| | | | | | | |
| | NP_000497.1\| | | | | | | |
| complement component 4B pre- | gi\|50345296\| | 192735 | 111 | DFALLSLQVPLK | 1343.79 | 81 | 92 |
| proprotein [Homo sapiens] | ref\|NP_ | | | | | | |
| | 001002029.1\| | | | | | | |
| complement component 4B pre- | gi\|50345296\| | 192735 | 112 | DFALLSLQVPLKD | 1657.95 | 81 | 95 |
| proprotein [Homo sapiens] | ref\|NP_ | | | AK | | | |
| | 001002029.1\|, | | | | | | |
| | gi\|67190748\| | | | | | | |
| | ref\| | | | | | | |
| | NP_009224.2\| | | | | | | |
| complement component 4B pre- | gi\|50345296\| | 192735 | 113 | DHAVDLIQK | 1038.56 | 1043 | 1051 |
| proprotein [Homo sapiens] | ref\|NP_ | | | | | | |
| | 001002029.1\| | | | | | | |
| complement component 4B pre- | gi\|50345296\| | 192735 | 114 | EPFLSCCQFAESLR | 1743.78 | 730 | 743 |
| proprotein [Homo sapiens] | ref\|NP_ | | | | | | |
| | 001002029.1\|, | | | | | | |
| | gi\|67190748\| | | | | | | |
| | ref\| | | | | | | |
| | NP_009224.2\| | | | | | | |
| complement component 4B pre- | gi\|50345296\| | 192735 | 115 | GLEEELQFSLGSK | 1436.73 | 1353 | 1365 |
| proprotein [Homo sapiens] | ref\|NP_ | | | | | | |
| | 001002029.1\|, | | | | | | |
| | gi\|67190748\| | | | | | | |
| | ref\| | | | | | | |
| | NP_009224.2\| | | | | | | |
| complement component 4B pre- | gi\|50345296\| | 192735 | 116 | GPEVQLVAHSPWLK | 1560.85 | 105 | 118 |
| proprotein [Homo sapiens] | ref\|NP_ | | | | | | |
| | 001002029.1\| | | | | | | |
| complement component 4B pre- | gi\|50345296\| | 192735 | 117 | KADGSYAAWLSR | 1324.66 | 1061 | 1072 |
| proprotein [Homo sapiens] | ref\|NP_ | | | | | | |
| | 001002029.1\| | | | | | | |
| complement component 4B pre- | gi\|50345296\| | 192735 | 118 | LHLETDSLALVAL | 2613.41 | 589 | 614 |
| proprotein [Homo sapiens] | ref\|NP_ | | | GALDTALYAAGSK | | | |
| | 001002029.1\| | | | | | | |
| complement component 4B pre- | gi\|50345296\| | 192735 | 119 | SCGLHQLLR | 1083.57 | 96 | 104 |
| proprotein [Homo sapiens] | ref\|NP_ | | | | | | |
| | 001002029.1\|, | | | | | | |
| | gi\|67190748\| | | | | | | |
| | ref\| | | | | | | |
| | NP_009224.2\| | | | | | | |
| complement component 4B pre- | gi\|50345296\| | 192735 | 120 | TYNVLDMK | 983.49 | 1383 | 1390 |
| proprotein [Homo sapiens] | ref\|NP_ | | | | | | |
| | 001002029.1\|, | | | | | | |
| | gi\|67190748\| | | | | | | |
| | ref\| | | | | | | |
| | NP_009224.2\| | | | | | | |
| complement component 4B pre- | gi\|50345296\| | 192735 | 121 | VDFTLSSER | 1053.52 | 72 | 80 |
| proprotein [Homo sapiens] | ref\|NP_ | | | | | | |
| | 001002029.1\| | | | | | | |
| complement component 4B pre- | gi\|50345296\| | 192735 | 122 | VGLSGMAIADVTL | 2128.16 | 1478 | 1498 |
| proprotein [Homo sapiens] | ref\|NP_ | | | LSGFHALR | | | |
| | 001002029.1\| | | | | | | |
| complement component 4B pre- | gi\|50345296\| | 192735 | 123 | VLSLAQEQVGGSP | 1541.82 | 1085 | 1099 |
| proprotein [Homo sapiens] | ref\|NP_ | | | EK | | | |
| | 001002029.1\| | | | | | | |
| complement component 4B pre- | gi\|50345296\| | 192735 | 124 | YVSHFETEGPHVL | 2680.30 | 1511 | 1533 |
| proprotein [Homo sapiens] | ref\|NP_ | | | LYFDSVPTSR | | | |
| | 001002029.1\| | | | | | | |
| complement component 5 | gi\|38016947\| | 188291 | 125 | FQNSAILTIQPK | 1359.76 | 79 | 90 |
| [Homo sapiens] | ref\| | | | | | | |
| | NP_001726.2\| | | | | | | |
| complement component 5 | gi\|38016947\| | 188291 | 126 | MVETTAYALLTSL | 1783.95 | 1247 | 1262 |
| [Homo sapiens] | ref\| | | | NLK | | | |
| | NP_001726.2\| | | | | | | |
| complement component 5 | gi\|38016947\| | 188291 | 127 | VFQFLEK | 910.50 | 623 | 629 |
| [Homo sapiens] | ref\| | | | | | | |
| | NP_001726.2\| | | | | | | |
| complement component 5 | gi\|38016947\| | 188291 | 128 | YIYPLDSLTWIEYW | 2115.06 | 1635 | 1650 |
| [Homo sapiens] | ref\| | | | PR | | | |
| | NP_001726.2\| | | | | | | |
| complement component 8, gamma | gi\|4557393\| | 22201 | 129 | AEATTLHVAPQGT | 2159.09 | 70 | 90 |
| polypeptide [Homo sapiens] | ref\| | | | AMAVSTFR | | | |
| | NP_000597.1\| | | | | | | |
| complement component 8, gamma | gi\|4557393\| | 22201 | 130 | ANFDAQQFAGTW | 2382.17 | 40 | 61 |
| polypeptide [Homo sapiens] | ref\| | | | LLVAVGSACR | | | |
| | NP_000597.1\| | | | | | | |
| complement component 8, gamma | gi\|4557393\| | 22201 | 131 | GAVHVVVAETDY | 2466.27 | 121 | 142 |
| polypeptide [Homo sapiens] | ref\| | | | QSFAVLYLER | | | |
| | NP_000597.1\| | | | | | | |
| complement component 8, gamma | gi\|4557393\| | 22201 | 132 | KLDGICWQVR | 1274.67 | 91 | 100 |
| polypeptide [Homo sapiens] | ref\| | | | | | | |
| | NP_000597.1\| | | | | | | |
| complement component 8, gamma | gi\|4557393\| | 22201 | 133 | LDGICWQVR | 1146.57 | 92 | 100 |
| polypeptide [Homo sapiens] | ref\| | | | | | | |
| | NP_000597.1\| | | | | | | |
| complement component 8, gamma | gi\|4557393\| | 22201 | 134 | QLYGDTGVLGR | 1178.62 | 101 | 111 |
| polypeptide [Homo sapiens] | ref\| | | | | | | |
| | NP_000597.1\| | | | | | | |
| complement component 8, gamma | gi\|4557393\| | 22201 | 135 | RPASPISTIQPK | 1294.75 | 28 | 39 |
| polypeptide [Homo sapiens] | ref\| | | | | | | |
| | NP_000597.1\| | | | | | | |
| complement component 8, gamma | gi\|4557393\| | 22201 | 136 | SLPVSDSVLSGFEQ | 1620.82 | 154 | 168 |
| polypeptide [Homo sapiens] | ref\| | | | R | | | |
| | NP_000597.1\| | | | | | | |
| complement component 8, gamma | gi\|4557393\| | 22201 | 137 | VQEAHLTEDQIFYF | 1964.98 | 169 | 184 |
| polypeptide [Homo sapiens] | ref\| | | | PK | | | |
| | NP_000597.1\| | | | | | | |
| complement component 8, gamma | gi\|4557393\| | 22201 | 138 | YGFCEAADQFHVL | 2055.92 | 185 | 201 |
| polypeptide [Homo sapiens] | ref\| | | | DEVR | | | |
| | NP_000597.1\| | | | | | | |
| complement factor H isoform a | gi\|62739186\| | 139052 | 139 | EIMENYNIALR | 1365.68 | 1172 | 1182 |
| precursor [Homo sapiens] | ref\| | | | | | | |
| | NP_000177.2\| | | | | | | |
| complement factor H isoform a | gi\|62739186\| | 139052 | 140 | KGEWVALNPLR | 1282.73 | 68 | 78 |
| precursor [Homo sapiens] | ref\| | | | | | | |
| | NP_000177.2\| | | | | | | |
| complement factor H isoform a | gi\|62739186\| | 139052 | 141 | NTEILTGSWSDQT | 2602.23 | 29 | 51 |
| precursor [Homo sapiens] | ref\| | | | YPEGTQAIYK | | | |
| | NP_000177.2\|, | | | | | | |
| | gi\|62739188\| | | | | | | |
| | ref\|NP_ | | | | | | |
| | 001014975.1\| | | | | | | |
| complement factor H isoform a | gi\|62739186\| | 139052 | 142 | SLGNVIMVCR | 1148.59 | 58 | 67 |
| precursor [Homo sapiens] | ref\| | | | | | | |
| | NP_000177.2\| | | | | | | |
| core-binding factor, runt | gi\|4826663\| | 67115 | 143 | MAKESGISLKEIQV | 1873.06 | 1 | 17 |
| domain, alpha subunit 2; | ref\| | | | LAR | | | |
| translocated to, 2 isoform | NP_005084.1\| | | | | | | |
| MTGR1b [Homo sapiens] | | | | | | | |
| delta globin [Homo sapiens] | gi\|4504351\| | 16037 | 144 | EFTPQMQAAYQK | 1441.68 | 122 | 133 |
| | ref\| | | | | | | |
| | NP_000510.1\| | | | | | | |
| delta globin [Homo sapiens] | gi\|4504351\| | 16037 | 145 | GTFSQLSELHCDK | 1521.70 | 84 | 96 |
| | ref\| | | | | | | |
| | NP_000510.1\| | | | | | | |
| delta globin [Homo sapiens] | gi\|4504351\| | 16037 | 146 | GTFSQLSELHCDKL | 2629.25 | 84 | 105 |
| | ref\| | | | HVDPENFR | | | |
| | NP_000510.1\| | | | | | | |
| delta globin [Homo sapiens] | gi\|4504351\| | 16037 | 147 | LLGNVLVCVLAR | 1326.79 | 106 | 117 |
| | ref\| | | | | | | |
| | NP_000510.1\| | | | | | | |
| delta globin [Homo sapiens] | gi\|4504351\| | 16037 | 148 | LLVVYPWTQR | 1274.73 | 32 | 41 |
| | ref\| | | | | | | |
| | NP_000510.1\| | | | | | | |
| delta globin [Homo sapiens] | gi\|4504351\| | 16037 | 149 | TAVNALWGK | 959.53 | 10 | 18 |
| | ref\| | | | | | | |
| | NP_000510.1\| | | | | | | |
| delta globin [Homo sapiens] | gi\|4504351\| | 16037 | 150 | VLGAFSDGLAHLD | 1669.89 | 68 | 83 |
| | ref\| | | | NLK | | | |
| | NP_000510.1\| | | | | | | |
| delta globin [Homo sapiens] | gi\|4504351\| | 16037 | 151 | VNVDAVGGEALGR | 1256.66 | 19 | 31 |
| | ref\| | | | | | | |
| | NP_000510.1\| | | | | | | |
| delta globin [Homo sapiens] | gi\|4504351\| | 16037 | 152 | VVAGVANALAHK | 1149.67 | 134 | 145 |
| | ref\| | | | | | | |
| | NP_000510.1\| | | | | | | |
| delta globin [Homo sapiens] | gi\|4504351\| | 16037 | 153 | VVAGVANALAHK | 1449.80 | 134 | 147 |
| | ref\| | | | YH | | | |
| | NP_000510.1\| | | | | | | |
| Desmoplakin (DP) (250/210 kDa | DESP_HUMAN | 331765 | 154 | ANSSATETINK | 1135.56 | 1517 | 1527 |
| paraneoplastic pemphigus | | | | | | | |
| antigen) | | | | | | | |
| Desmoplakin (DP) (250/210 kDa | DESP_HUMAN | 331765 | 155 | GFFDPNTEENLTYL | 2028.99 | 2414 | 2430 |
| paraneoplastic pemphigus | | | | QLK | | | |
| antigen) | | | | | | | |
| diacylglycerol kinase, theta | gi\|40806175\| | 101135 | 156 | PGSPACSPVLGSGG | 1625.82 | 20 | 36 |
| [Homo sapiens] | ref\| | | | RAR | | | |
| | NP_001338.2\| | | | | | | |
| Fibrinogen alpha chain pre- | FIBA_HUMAN | 94955 | 157 | TFPGFFSPMLGEFV | 2265.05 | 528 | 547 |
| cursor [Contains: Fibrinopep- | | | | SETESR | | | |
| tide A] | | | | | | | |
| Fibrinogen alpha chain pre- | FIBA_HUMAN | 94955 | 158 | VTSGSTTTTR | 1010.51 | 449 | 458 |
| cursor [Contains: Fibrinopep- | | | | | | | |
| tide A] | | | | | | | |
| fibrinogen, alpha polypeptide | gi\|11761629\| | 69739 | 159 | ESSSHHPGIAEFPS | 1637.77 | 559 | 573 |
| isoform alpha preproprotein | ref\| | | | R | | | |
| [Homo sapiens] | NP_068657.1\|, | | | | | | |
| | gi\|4503689\| | | | | | | |
| | ref\| | | | | | | |
| | NP_000499.1\| | | | | | | |
| fibrinogen, alpha polypeptide | gi\|11761629\| | 69739 | 160 | EVDLKDYEDQQK | 1509.71 | 191 | 202 |
| isoform alpha preproprotein | ref\| | | | | | | |
| [Homo sapiens] | NP_068657.1\|, | | | | | | |
| | gi\|4503689\| | | | | | | |
| | ref\| | | | | | | |
| | NP_000499.1\| | | | | | | |
| fibrinogen, alpha polypeptide | gi\|11761629\| | 69739 | 161 | GLIDEVNQDFTNR | 1520.73 | 72 | 84 |
| isoform alpha preproprotein | ref\| | | | | | | |
| [Homo sapiens] | NP_068657.1\|, | | | | | | |
| | gi\|4503689\| | | | | | | |
| | ref\| | | | | | | |
| | NP_000499.1\| | | | | | | |
| fibrinogen, alpha polypeptide | gi\|11761629\| | 69739 | 162 | GSESGIFTNTK | 1140.55 | 548 | 558 |
| isoform alpha preproprotein | ref\| | | | | | | |
| [Homo sapiens] | NP_068657.1\|, | | | | | | |
| | gi\|4503689\| | | | | | | |
| | ref\| | | | | | | |
| | NP_000499.1\| | | | | | | |
| fibrinogen, alpha polypeptide | gi\|11761629\| | 69739 | 163 | HPDEAAFFDTAST | 1593.72 | 513 | 527 |
| isoform alpha preproprotein | ref\| | | | GK | | | |
| [Homo sapiens] | NP_068657.1\|, | | | | | | |
| | gi\|4503689\| | | | | | | |
| | ref\| | | | | | | |
| | NP_000499.1\| | | | | | | |
| fibrinogen, alpha polypeptide | gi\|11761629\| | 69379 | 164 | HRHPDEAAFFDTA | 1886.88 | 511 | 527 |
| isoform alpha preproprotein | ref\| | | | STGK | | | |
| [Homo sapiens] | NP_068657.1\|, | | | | | | |
| | gi\|4503689\| | | | | | | |
| | ref\| | | | | | | |
| | NP_000499.1\| | | | | | | |
| fibrinogen, alpha polypeptide | gi\|11761629\| | 69739 | 165 | TFPGFFSPMLGEFV | 2265.05 | 528 | 547 |
| isoform alpha preproprotein | ref\| | | | SETESR | | | |
| [Homo sapiens] | NP_068357.1\|, | | | | | | |
| | gi\|4503689\| | | | | | | |
| | ref\| | | | | | | |
| | NP_000499.1\| | | | | | | |
| gelsolin isoform a precursor | gi\|4504165\| | 85680 | 166 | DSQEEEKTEALTSA | 1665.78 | 714 | 728 |
| [Homo sapiens] | ref\| | | | K | | | |
| | NP_000168.1\| | | | | | | |
| gelsolin isoform a precursor | gi\|4504165\| | 85680 | 167 | EVQGFESATFLGYF | 1722.84 | 148 | 162 |
| [Homo sapiens] | ref\| | | | K | | | |
| | NP_000168.1\| | | | | | | |
| gelsolin isoform a precursor | gi\|4504165\| | 85680 | 168 | FDLVPVPTNLYGD | 2704.39 | 76 | 99 |
| [Homo sapiens] | ref\| | | | FFTGDAYVILK | | | |
| | NP_000168.1\| | | | | | | |
| gelsolin isoform a precursor | gi\|4504165\| | 85680 | 169 | HVVPNEVVVQR | 1275.72 | 178 | 188 |
| [Homo sapiens] | ref\| | | | | | | |
| | NP_000168.1\| | | | | | | |
| gelsolin isoform a precursor | gi\|4504165\| | 85680 | 170 | PNSMVVEHPEFLK | 1526.77 | 49 | 61 |
| [Homo sapiens] | ref\| | | | | | | |
| | NP_000168.1\| | | | | | | |
| hect domain and RLD 5 | gi\|7705931\| | 116834 | 171 | RTTEMMPVYLDLN | 1726.85 | 434 | 447 |
| [Homo sapiens] | ref\| | | | K | | | |
| | NP_057407.1\| | | | | | | |
| Hepatitis B virus receptor | Q6PYx1 | 38143 | 172 | GQGTTVTVSSAST | 1323.68 | 9 | 22 |
| binding protein (Fragment) | | | | K | | | |
| Hepatitis B virus receptor | Q6PYX1 | 38143 | 173 | GTTVTVSSASTK | 1138.60 | 11 | 22 |
| binding protein (Fragment) | | | | | | | |
| histidine-rich glycoprotein | gi\|4504489\| | 59559 | 174 | ALDLINKR | 942.57 | 34 | 41 |
| precursor [Homo sapiens] | ref\| | | | | | | |
| | NP_000403.1\| | | | | | | |
| histidine-rich glycoprotein | gi\|4504489\| | 59559 | 175 | DGYLFQLLR | 1124.61 | 44 | 52 |
| precursor [Homo sapiens] | ref\| | | | | | | |
| | NP_000403.1\| | | | | | | |
| histidine-rich glycoprotein | gi\|4504489\| | 59559 | 176 | DSPVLIDFFEDTER | 1682.79 | 140 | 153 |
| precursor [Homo sapiens] | ref\| | | | | | | |
| | NP_000403.1\| | | | | | | |
| histidine-rich glycoprotein | gi\|4504489\| | 59559 | 177 | GEVLPLPEANFPSF | 2226.17 | 467 | 486 |
| precursor [Homo sapiens] | ref\| | | | PLPHHK | | | |
| | NP_000403.1\| | | | | | | |
| histidine-rich glycoprotein | gi\|4504489\| | 59559 | 178 | HPLKPDNQPFPQSV | 2349.13 | 487 | 507 |
| precursor [Homo sapiens] | ref\| | | | SESCPGK | | | |
| | NP_000403.1\| | | | | | | |
| histidine-rich glycoprotein | gi\|4504489\| | 59559 | 179 | KGEVLPLPEANFPS | 2354.27 | 466 | 486 |
| precursor [Homo sapiens] | ref\| | | | FPLPHHK | | | |
| | NP_000403.1\| | | | | | | |
| histidine-rich glycoprotein | gi\|4504489\| | 59559 | 180 | LPPLR | 595.39 | 461 | 465 |
| precursor [Homo sapiens] | ref\| | | | | | | |
| | NP_000403.1\| | | | | | | |
| histidine-rich glycoprotein | gi\|4504489\| | 59559 | 181 | RDGYLFQLLR | 1280.71 | 43 | 52 |
| precursor [Homo sapiens] | ref\| | | | | | | |
| | NP_000403.1\| | | | | | | |
| histidine-rich glycoprotein | gi\|4504489\| | 59559 | 182 | RPSEIVIGQCK | 1286.69 | 96 | 106 |
| precursor [Homo sapiens] | ref\| | | | | | | |
| | NP_000403.1\| | | | | | | |
| histidine-rich glycoprotein | gi\|4504489\| | 59559 | 183 | SGFPQVSMFFTHTF | 1857.90 | 510 | 525 |
| precursor [Homo sapiens] | ref\| | | | PK | | | |
| | NP_000403.1\| | | | | | | |
| Hypothetical protein | Q6MZW0 | 54441 | 184 | GTTTFAVTSILR | 1266.71 | 441 | 452 |
| DKFZp686J11235 (Fragment) | | | | | | | |
| Hypothetical protein | Q6MZW0 | 54441 | 185 | TPLTATLSK | 931.55 | 366 | 374 |
| DKFZp686J11235 (Fragment) | | | | | | | |
| Hypothetical protein | Q6MZW0 | 54441 | 186 | VTVSSASPTSPK | 1160.62 | 149 | 160 |
| DKFZp686J11235 (Fragment) | | | | | | | |
| Hypothetical protein | Q5CZ94 | 24984 | 187 | AGVETTKPSK | 1017.56 | 179 | 188 |
| DKFZp781M0386 | | | | | | | |
| Hypothetical protein | Q5CZ94 | 24984 | 188 | ANPTVTLFPPSSEE | 2043.04 | 133 | 151 |
| DKFZp781M0386 | | | | LQANK | | | |
| Hypothetical protein | Q5CZ94 | 24984 | 189 | VTVLGQPK | 841.51 | 125 | 132 |
| DKFZp781M0386 | | | | | | | |
| Ig heavy chain V-II region | HV2F_HUMAN | 14099 | 190 | TGYYWGWIR | 1201.58 | 32 | 40 |
| WAH | | | | | | | |
| Ig heavy chain V-III region | HV3T_HUMAN | 12708 | 191 | GLEWVANIK | 1029.57 | 44 | 52 |
| GAL | | | | | | | |
| Ig kappa chain V-II region | KV2A_HUMAN | 12659 | 192 | FSGSGSGTDFTLK | 1303.62 | 69 | 81 |
| Cum | | | | | | | |
| inter-alpha (globulin) | gi\|31542984\| | 103340 | 193 | AEAQAQYSAAVAK | 1307.66 | 99 | 111 |
| inhibitor H4 [Homo sapiens] | ref\| | | | | | | |
| | NP_002209.2\| | | | | | | |
| inter-alpha (globulin) | gi\|31542984\| | 103340 | 194 | AGFSWIEVTFK | 1284.66 | 778 | 788 |
| inhibitor H4 [Homo sapiens] | ref\| | | | | | | |
| | NP_002209.2\| | | | | | | |
| inter-alpha (globulin) | gi\|31542984\| | 103340 | 195 | ANTVQEATFQMEL | 1706.84 | 61 | 75 |
| inhibitor H4 [Homo sapiens] | ref\| | | | PK | | | |
| | NP_002209.2\| | | | | | | |
| inter-alpha (globulin) | gi\|31542984\| | 103340 | 196 | FSSHVGGTLGQFY | 2968.39 | 867 | 894 |
| inhibitor H4 [Homo sapiens] | ref\| | | | QEVLWGSPAASDD | | | |
| | NP_002209.2\| | | | GR | | | |
| inter-alpha (globulin) | gi\|31542984\| | 103340 | 197 | GPDVLTATVSGK | 1144.62 | 501 | 512 |
| inhibitor H4 [Homo sapiens] | ref\| | | | | | | |
| | NP_002209.2\| | | | | | | |
| inter-alpha (globulin) | gi\|31542984\| | 103340 | 198 | LWAYLTIQQLLEQ | 2961.53 | 547 | 572 |
| inhibitor H4 [Homo sapiens] | ref\| | | | TVSASDADQQALR | | | |
| | NP_002209.2\| | | | | | | |
| inter-alpha (globulin) | gi\|31542984\| | 103340 | 199 | MNFRPGVLSSR | 1263.66 | 658 | 668 |
| inhibitor H4 [Homo sapiens] | ref\| | | | | | | |
| | NP_002209.2\| | | | | | | |
| inter-alpha (globulin) | gi\|31542984\| | 103340 | 200 | NVHSGSTFFK | 1123.55 | 617 | 626 |
| inhibitor H4 [Homo sapiens] | ref\| | | | | | | |
| | NP_002209.2\| | | | | | | |
| inter-alpha (globulin) | gi\|31542984\| | 103340 | 201 | VTIGLLFWDGR | 1276.71 | 843 | 853 |
| inhibitor H4 [Homo sapiens] | ref\| | | | | | | |
| | NP_002209.2\| | | | | | | |
| keratin 1 [Homo sapiens] | gi\|17318569\| | 66050 | 202 | AEAESLYQSK | 1125.54 | 367 | 376 |
| | ref\| | | | | | | |
| | NP_006112.2\| | | | | | | |
| keratin 1 [Homo sapiens] | gi\|17318569\| | 66050 | 203 | AQYEDIAQK | 1065.52 | 356 | 364 |
| | ref\| | | | | | | |
| | NP_006112.2\| | | | | | | |
| keratin 1 [Homo sapiens] | gi\|17318569\| | 66050 | 204 | DYQELMNTK | 1141.52 | 464 | 472 |
| | ref\| | | | | | | |
| | NP_006112.2\| | | | | | | |
| keratin 1 [Homo sapiens] | gi\|17318569\| | 66050 | 205 | EQIKSLNNQFASFI | 1881.97 | 182 | 197 |
| | ref\| | | | DK | | | |
| | NP_006112.2\| | | | | | | |
| keratin 1 [Homo sapiens] | gi\|17318569\| | 66050 | 206 | FLEQQNQVLQTK | 1475.79 | 200 | 211 |
| | ref\| | | | | | | |
| | NP_006112.2\| | | | | | | |
| keratin 1 [Homo sapiens] | gi\|17318569\| | 66050 | 207 | FSSCGGGGGSFGA | 1765.74 | 46 | 65 |
| | ref\| | | | GGGFGSR | | | |
| | NP_006112.2\| | | | | | | |
| keratin 1 [Homo sapiens] | gi\|17318569\| | 66050 | 208 | GGGGGGYGSGGSS | 2383.95 | 519 | 549 |
| | ref\| | | | YGSGGGSYGSGGG | | | |
| | NP_006112.2\| | | | GGGGR | | | |
| keratin 1 [Homo sapiens] | gi\|17318569\| | 66050 | 209 | IEISELNR | 973.53 | 396 | 403 |
| | ref\| | | | | | | |
| | NP_006112.2\| | | | | | | |
| keratin 1 [Homo sapiens] | gi\|17318569\| | 66050 | 210 | LALDLEIATYR | 1277.71 | 473 | 483 |
| | ref\| | | | | | | |
| | NP_006112.2\| | | | | | | |
| keratin 1 [Homo sapiens] | gi\|17318569\| | 66050 | 211 | LNDLEDALQQAK | 1357.70 | 444 | 455 |
| | ref\| | | | | | | |
| | NP_006112.2\| | | | | | | |
| keratin 1 [Homo sapiens] | gi\|17318569\| | 66050 | 212 | MSGECAPNVSVSV | 2565.17 | 493 | 518 |
| | ref\| | | | STSHTTISGGGSR | | | |
| | NP_006112.2\| | | | | | | |
| keratin 1 [Homo sapiens] | gi\|17318569\| | 66050 | 213 | NKLNDLEDALQQAK | 1599.83 | 442 | 455 |
| | ref\| | | | | | | |
| | NP_006112.2\| | | | | | | |
| keratin 1 [Homo sapiens] | gi\|17318569\| | 66050 | 214 | NKYEDEINKR | 1308.65 | 268 | 277 |
| | ref\| | | | | | | |
| | NP_006112.2\| | | | | | | |
| keratin 1 [Homo sapiens] | gi\|17318569\| | 66050 | 215 | NMQDMVEDYR | 1300.53 | 258 | 267 |
| | ref\| | | | | | | |
| | NP_006112.2\| | | | | | | |
| keratin 1 [Homo sapiens] | gi\|17318569\| | 66050 | 216 | NMQDMVEDYR | 1300.53 | 258 | 267 |
| | ref\| | | | | | | |
| | NP_006112.2\| | | | | | | |
| keratin 1 [Homo sapiens] | gi\|17318569\| | 66050 | 217 | QISNLQQSISDAEQ | 1716.85 | 418 | 432 |
| | ref\| | | | R | | | |
| | NP_006112.2\| | | | | | | |
| keratin 1 [Homo sapiens] | gi\|17318569\| | 66050 | 218 | SKAEAESLYQSK | 1340.67 | 365 | 376 |
| | ref\| | | | | | | |
| | NP_006112.2\| | | | | | | |
| keratin 1 [Homo sapiens] | gi\|17318569\| | 66050 | 219 | SLDLDSIIAEVK | 1302.72 | 344 | 355 |
| | ref\| | | | | | | |
| | NP_006112.2\| | | | | | | |
| keratin 1 [Homo sapiens] | gi\|17318569\| | 66050 | 220 | THNLEPYFESFINN | 1993.98 | 224 | 239 |
| | ref\| | | | LR | | | |
| | NP_006112.2\| | | | | | | |
| keratin 1 [Homo sapiens] | gi\|17318569\| | 66050 | 221 | TLLEGEESR | 1033.52 | 484 | 492 |
| | ref\| | | | | | | |
| | NP_006112.2\| | | | | | | |
| keratin 1 [Homo sapiens] | gi\|17318569\| | 66050 | 223 | TNAENEFVTIK | 1265.64 | 278 | 288 |
| | ref\| | | | | | | |
| | NP_006112.2\| | | | | | | |
| keratin 1 [Homo sapiens] | gi\|17318569\| | 66050 | 224 | WELLQQVDTSTR | 1475.75 | 212 | 223 |
| | ref\| | | | | | | |
| | NP_006112.2\| | | | | | | |
| keratin 1 [Homo sapiens] | gi\|17318569\| | 66050 | 225 | YEELQITAGR | 1179.60 | 377 | 386 |
| | ref\| | | | | | | |
| | NP_006112.2\| | | | | | | |
| Keratin 10 | Q14664 | 57231 | 226 | GSSGGGCFGGSSG | 2342.99 | 53 | 79 |
| | | | | GYGGLGGFGGGSF | | | |
| | | | | R | | | |
| keratin 10 [Homo sapiens] | gi\|40354192\| | 58811 | 227 | ADLEMQIESLTEEL | 2112.04 | 267 | 284 |
| | ref\| | | | AYLK | | | |
| | NP_000412.2\| | | | | | | |
| keratin 10 [Homo sapiens] | gi\|40354192\| | 58811 | 228 | AETECQNTEYQQL | 2082.97 | 423 | 439 |
| | ref\| | | | LDIK | | | |
| | NP_000412.2\| | | | | | | |
| keratin 10 [Homo sapiens] | gi\|40354192\| | 58811 | 229 | ALEESNYELEGK | 1381.65 | 166 | 177 |
| | ref\| | | | | | | |
| | NP_000412.2\| | | | | | | |
| keratin 10 [Homo sapiens] | gi\|40354192\| | 58811 | 230 | DAEAWFNEK | 1109.49 | 335 | 343 |
| | ref\| | | | | | | |
| | NP_000412.2\| | | | | | | |
| keratin 10 [Homo sapiens] | gi\|40354192\| | 58811 | 231 | ELTTEIDNNIEQIS | 1996.97 | 346 | 362 |
| | ref\| | | | SYK | | | |
| | NP_000412.2\| | | | | | | |
| keratin 10 [Homo sapiens] | gi\|40354192\| | 58811 | 232 | GSLGGGFSSGGFSG | 1707.77 | 41 | 59 |
| | ref\| | | | GSFSR | | | |
| | NP_000412.2\| | | | | | | |
| keratin 10 [Homo sapiens] | gi\|40354192\| | 58811 | 233 | GSSGGGCFGGSSG | 2342.99 | 60 | 86 |
| | ref\| | | | GYGGLGGFGGGSF | | | |
| | NP_000412.2\| | | | R | | | |
| keratin 10 [Homo sapiens] | gi\|40354192\| | 58811 | 234 | IKEWYEK | 995.52 | 178 | 184 |
| | ref\| | | | | | | |
| | NP_000412.2\| | | | | | | |
| keratin 10 [Homo sapiens] | gi\|40354192\| | 58811 | 235 | LASYLDK | 809.44 | 157 | 163 |
| | ref\| | | | | | | |
| | NP_000412.2\| | | | | | | |
| keratin 10 [Homo sapiens] | gi\|40354192\| | 58811 | 236 | LENEIQTYR | 1165.59 | 442 | 450 |
| | ref\| | | | | | | |
| | NP_000412.2\| | | | | | | |
| keratin 10 [Homo sapiens] | gi\|40354192\| | 58811 | 237 | LKYENEVALR | 1234.68 | 236 | 245 |
| | ref\| | | | | | | |
| | NP_000412.2\| | | | | | | |
| keratin 10 [Homo sapiens] | gi\|40354192\| | 58811 | 238 | NVSTGDVNVEMN | 2872.39 | 296 | 322 |
| | ref\| | | | AAPGVDLTQLLNN | | | |
| | NP_000412.2\| | | | MR | | | |
| keratin 10 [Homo sapiens] | gi\|40354192\| | 58811 | 239 | SKELTTEIDNNIEQ | 2212.10 | 344 | 362 |
| | ref\| | | | ISSYK | | | |
| | NP_000412.2\| | | | | | | |
| keratin 10 [Homo sapiens] | gi\|40354192\| | 58811 | 240 | SLLEGEGSSGGGGR | 1262.60 | 451 | 464 |
| | ref\| | | | | | | |
| | NP_000412.2\| | | | | | | |
| keratin 10 [Homo sapiens] | gi\|40354192\| | 58811 | 241 | SQYEQLAEQNR | 1365.64 | 323 | 333 |
| | ref\| | | | | | | |
| | NP_000412.2\| | | | | | | |
| keratin 10 [Homo sapiens] | gi\|40354192\| | 58811 | 242 | SQYEQLAEQNRK | 1493.73 | 323 | 334 |
| | ref\| | | | | | | |
| | NP_000412.2\| | | | | | | |
| keratin 10 [Homo sapiens] | gi\|40354192\| | 58811 | 243 | TIDDLKNQILNLTT | 3052.63 | 202 | 228 |
| | ref\| | | | DNANILLQIDNAR | | | |
| | NP_000412.2\| | | | | | | |
| keratin 10 [Homo sapiens] | gi\|40354192\| | 58811 | 244 | VLDELTLTK | 1031.60 | 258 | 266 |
| | ref\| | | | | | | |
| | NP_000412.2\| | | | | | | |
| keratin 10 [Homo sapiens] | gi\|40354192\| | 58811 | 245 | VTMQNLNDR | 1090.53 | 148 | 156 |
| | ref\| | | | | | | |
| | NP_000412.2\| | | | | | | |
| keratin 10 [Homo sapiens] | gi\|40354192\| | 58811 | 246 | YCVQLSQIQAQISA | 2746.42 | 400 | 422 |
| | ref\| | | | LEEQLQQIR | | | |
| | NP_000412.2\| | | | | | | |
| keratin 10 [Homo sapiens] | gi\|40354192\| | 58811 | 247 | YENEVALR | 993.50 | 238 | 245 |
| | ref\| | | | | | | |
| | NP_000412.2\| | | | | | | |
| keratin 9 [Homo sapiens] | gi\|55956899\| | 62048 | 248 | DIENQYETQITQIE | 3264.51 | 340 | 368 |
| | ref\| | | | HEVSSSGQEVQSS | | | |
| | NP_000217.2\| | | | AK | | | |
| keratin 9 [Homo sapiens] | gi\|55956899\| | 62048 | 249 | EIETYHNLLEGGQE | 2510.13 | 450 | 472 |
| | ref\| | | | DFESSGAGK | | | |
| | NP_000217.2\| | | | | | | |
| keratin 9 [Homo sapiens] | gi\|55956899\| | 62048 | 250 | FSSSSGYGGGSSR | 1235.53 | 47 | 59 |
| | ref\| | | | | | | |
| | NP_000217.2\| | | | | | | |
| keratin 9 [Homo sapiens] | gi\|55956899\| | 62048 | 251 | GGGGSFGYSYGGG | 2705.16 | 64 | 95 |
| | ref\| | | | SGGGFSASSLGGGF | | | |
| | NP_000217.2\| | | | GGGSR | | | |
| keratin 9 [Homo sapiens] | gi\|55956899\| | 62048 | 252 | HGVQELEIELQSQL | 1837.97 | 375 | 390 |
| | ref\| | | | SK | | | |
| | NP_000217.2\| | | | | | | |
| keratin 9 [Homo sapiens] | gi\|55956899\| | 62048 | 253 | IKFEMEQNLR | 1307.68 | 241 | 250 |
| | ref\| | | | | | | |
| | NP_000217.2\| | | | | | | |
| keratin 9 [Homo sapiens] | gi\|55956899\| | 62048 | 254 | IQDWYDKK | 1095.55 | 185 | 192 |
| | ref\| | | | | | | |
| | NP_000217.2\| | | | | | | |
| keratin 9 [Homo sapiens] | gi\|55956899\| | 62048 | 255 | LASYLDK | 809.44 | 164 | 170 |
| | ref\| | | | | | | |
| | NP_000217.2\| | | | | | | |
| keratin 9 [Homo sapiens] | gi\|55956899\| | 62048 | 256 | NYSPYYNTIDDLK | 2902.41 | 200 | 224 |
| | ref\| | | | DQIVDLTVGNNK | | | |
| | NP_000217.2\| | | | | | | |
| keratin 9 [Homo sapiens] | gi\|55956899\| | 62048 | 257 | SDLEMQYETLQEE | 2171.03 | 272 | 289 |
| | ref\| | | | LMALK | | | |
| | NP_000217.2\| | | | | | | |
| keratin 9 [Homo sapiens] | gi\|55956899\| | 62048 | 258 | SGGGGGGGLGSGG | 1232.60 | 14 | 29 |
| | ref\| | | | SIR | | | |
| | NP_000217.2\| | | | | | | |
| keratin 9 [Homo sapiens] | gi\|55956899\| | 62048 | 259 | STMQELNSR | 1065.50 | 155 | 163 |
| | ref\| | | | | | | |
| | NP_000217.2\| | | | | | | |
| keratin 9 [Homo sapiens] | gi\|55956899\| | 62048 | 260 | TLLDIDNTR | 1060.56 | 225 | 233 |
| | ref\| | | | | | | |
| | NP_000217.2\| | | | | | | |
| keratin 9 [Homo sapiens] | gi\|55956899\| | 62048 | 261 | VQALEEANNDLEN | 1586.77 | 171 | 184 |
| | ref\| | | | K | | | |
| | NP_000217.2\| | | | | | | |
| keratin 9 [Homo sapiens] | gi\|55956899\| | 62048 | 262 | YCGQLQMIQEQIS | 2753.32 | 405 | 427 |
| | ref\| | | | NLEAQITDVR | | | |
| | NP_000217.2\| | | | | | | |
| Keratin, type I cytoskeletal | K1C10_HUMAN | 59502 | 263 | AETECQNTEYQQL | 2352.15 | 423 | 441 |
| 10 (Cytokeratin-10) (CK 10) | | | | LDIKIR | | | |
| (Keratin-10) (K10) | | | | | | | |
| Keratin, type I cytoskeletal | K1C10_HUMAN | 59502 | 264 | GSLGGGFSSGGFSG | 1464.64 | 41 | 57 |
| 10 (Cytokeratin-10) (CK 10) | | | | GSF | | | |
| (Keratin-10) (K10) | | | | | | | |
| Keratin, type I cytoskeletal | K1C10_HUMAN | 59502 | 265 | ILLQIDNAR | 1055.62 | 220 | 228 |
| 10 (Cytokeratin-10) | | | | | | | |
| (CK 10) (Keratin-10) (K10) | | | | | | | |
| Keratin, type I cytoskeletal | K1C10_HUMAN | 59502 | 266 | IRLENEIQTYR | 1434.77 | 440 | 450 |
| 10 (Cytokeratin-10) | | | | | | | |
| (CK 10) (Keratin-10) (K10) | | | | | | | |
| Keratin, type I cytoskeletal | K1C10_HUMAN | 59502 | 267 | ISALEEQLQQIR | 1427.79 | 411 | 422 |
| 10 (Cytokeratin-10) | | | | | | | |
| (CK 10) (Keratin-10) (K10) | | | | | | | |
| Keratin, type I cytoskeletal | K1C10_HUMAN | 59502 | 268 | LAADDFR | 807.40 | 229 | 235 |
| 10 (Cytokeratin-10) | | | | | | | |
| (CK 10) (Keratin-10) (K10) | | | | | | | |
| Keratin, type I cytoskeletal | K1C10_HUMAN | 59502 | 269 | LASYLDK | 809.44 | 157 | 163 |
| 10 (Cytokeratin-10) | | | | | | | |
| (CK 10) (Keratin-10) (K10) | | | | | | | |
| Keratin, type I cytoskeletal | K1C10_HUMAN | 59502 | 270 | LENEIQTYR | 1165.59 | 442 | 450 |
| 10 (Cytokeratin-10) | | | | | | | |
| (CK 10) (Keratin-10) (K10) | | | | | | | |
| Keratin, type I cytoskeletal | K1C10_HUMAN | 59502 | 271 | LKYENEVALR | 1234.68 | 236 | 245 |
| 10 (Cytokeratin-10) | | | | | | | |
| (CK 10) (Keratin-10) (K10) | | | | | | | |
| Keratin, type I cytoskeletal | K1C10_HUMAN | 59502 | 272 | NQILNLTTDNAN | 1330.66 | 208 | 219 |
| 10 (Cytokeratin-10) | | | | | | | |
| (CK 10 Keratin-10) (K10) | | | | | | | |
| Keratin, type I cytoskeletal | K1C10_HUMAN | 59502 | 273 | NQILNLTTDNANIL | 2367.26 | 208 | 228 |
| 10 (Cytokeratin-10) | | | | LQIDNAR | | | |
| (CK 10) (Keratin-10) (K10) | | | | | | | |
| Keratin, type I cytoskeletal | K1C10_HUMAN | 59502 | 274 | NVQALEIELQSQLA | 1797.01 | 371 | 386 |
| 10 (Cytokeratin-10) | | | | LK | | | |
| (CK 10) (Keratin-10) (K10) | | | | | | | |
| Keratin, type I cytoskeletal | K1C10_HUMAN | 59502 | 275 | RVLDELTLTK | 1187.70 | 257 | 266 |
| 10 (Cytokeratin-10) | | | | | | | |
| (CK 10) (Keratin-10) (K10) | | | | | | | |
| Keratin, type I cytoskeletal | K1C10_HUMAN | 59502 | 276 | SGGGGGGGGCGG | 1549.68 | 16 | 35 |
| 10 (Cytokeratin-10) | | | | GGGVSSLR | | | |
| (CK 10) (Keratin-10) (K10) | | | | | | | |
| Keratin, type I cytoskeletal | K1C10_HUMAN | 59502 | 277 | SKELTTEIDNNIEQ | 2212.10 | 344 | 362 |
| 10 (Cytokeratin-10) | | | | ISSYK | | | |
| (CK 10) (Keratin-10) (K10) | | | | | | | |
| Keratin, type I cytoskeletal | K1C10_HUMAN | 59502 | 278 | SLLEGEGSSGGGG | 1262.60 | 451 | 464 |
| 10 (Cytokeratin-10) | | | | R | | | |
| (CK 10) (Keratin-10) (K10) | | | | | | | |
| Keratin, type I cytoskeletal | K1C10_HUMAN | 59502 | 279 | VQALEIELQSQLAL | 1682.97 | 372 | 386 |
| 10 (Cytokeratin-10) | | | | K | | | |
| (CK 10) (Keratin-10) (K10) | | | | | | | |
| Keratin, type I cytoskeletal | K1C13_HUMAN | 49569 | 280 | LEQEIATYR | 1122.58 | 398 | 406 |
| 13 (Cytokeratin-13) | | | | | | | |
| (CK 13) (Keratin-13) (K13) | | | | | | | |
| Keratin, type I cytoskeletal | K1C14_HUMAN | 51473 | 281 | EVATNSELVQSGK | 1361.69 | 315 | 327 |
| 14 (Cytokeratin-14) | | | | | | | |
| (CK 14) (Keratin-14) (K14) | | | | | | | |
| Keratin, type I cytoskeletal | K1C14_HUMAN | 51473 | 282 | GSCGIGGGIGGGSS | 1278.59 | 15 | 29 |
| 14 (Cytokeratin-14) | | | | R | | | |
| (CK 14) (Keratin-14) (K14) | | | | | | | |
| Keratin, type I cytoskeletal | K1C14_HUMAN | 51473 | 283 | ISSVLAGGSCR | 1106.56 | 30 | 40 |
| 14 (Cytokeratin-14) | | | | | | | |
| (CK 14) (Keratin-14) (K14) | | | | | | | |
| Keratin, type I cytoskeletal | K1C14_HUMAN | 51473 | 284 | VLDELTLAR | 1029.59 | 223 | 231 |
| 14 (Cytokeratin-14) | | | | | | | |
| (CK 14) (Keratin-14) (K14) | | | | | | | |
| Keratin, type II cytoskeletal | K2C1_HUMAN | 65870 | 285 | AEAESLYQSK | 1125.54 | 366 | 375 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 286 | ELLQQVDTSTR | 1289.67 | 212 | 222 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 287 | GGSGGGGGGSSGG | 2079.92 | 588 | 615 |
| 1 (Cytokeratin-1) (CK-1) | | | | RGSGGGSSGGSIG | | | |
| (Keratin-1) (K1) (67 kDa | | | | GR | | | |
| cytokeratin) (Hair alpha | | | | | | | |
| protein) | | | | | | | |
| Keratin, type II cytoskeletal | K2C1_HUMAN | 65870 | 288 | GSGGGSSGGSIGG | 1092.50 | 602 | 615 |
| 1 (Cytokeratin-1) (CK-1) | | | | R | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 289 | KQISNLQQSISDAE | 1844.95 | 416 | 431 |
| 1 (Cytokeratin-1) (CK-1) | | | | QR | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 290 | LALDLEIATYR | 1277.71 | 472 | 482 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 291 | NKLNDLEDALQQAK | 1599.83 | 441 | 454 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 292 | NVEIDPEIQK | 1184.62 | 165 | 174 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 293 | SEIDNVKK | 932.51 | 409 | 416 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 294 | SGGGFSSGSAGIIN | 1657.79 | 12 | 28 |
| 1 (Cytokeratin-1) (CK-1) | | | | YQR | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 295 | SISDAEQR | 905.43 | 424 | 431 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 296 | SISISVARGGGR | 1159.65 | 74 | 85 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 297 | SKAEAESLYQSK | 1340.67 | 364 | 375 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 658780 | 298 | SLDLDSIIAEVK | 1302.72 | 343 | 354 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 299 | SLNNQFASFIDK | 1383.69 | 185 | 196 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 300 | SLNNQFASFIDKVR | 1638.86 | 185 | 198 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 301 | SLVNLGGSK | 874.50 | 65 | 73 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 302 | THNLEPYFESF | 1383.62 | 223 | 233 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 303 | THNLEPYFESFINN | 1993.98 | 223 | 238 |
| 1 (Cytokeratin-1) (CK-1) | | | | LR | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 304 | TLLEGEESR | 1033.52 | 483 | 491 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 305 | TNAENEFVTIK | 1265.64 | 277 | 287 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 306 | VDQLKSDQSR | 1175.60 | 241 | 250 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 307 | VEIDPEIQK | 1070.57 | 166 | 174 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 308 | WELLQQVDTSTR | 1475.75 | 211 | 222 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K2C1_HUMAN | 65870 | 309 | YEELQITAGR | 1179.60 | 376 | 385 |
| 1 (Cytokeratin-1) (CK-1) | | | | | | | |
| (Keratin-1) (K1) (67 kDa | | | | | | | |
| cytokeratin) (Hair alpha | | | | | | | |
| protein) | | | | | | | |
| Keratin, type II cytoskeletal | K22E_HUMAN | 65848 | 310 | GFSSGSAVVSGGS | 1254.61 | 21 | 34 |
| 2 epidermal (Cytokeratin-2e) | | | | R | | | |
| (K2e) (CK 2e) | | | | | | | |
| Keratin, type II cytoskeletal | K22E_HUMAN | 65848 | 311 | IEISELNR | 973.53 | 400 | 407 |
| 2 epidermal (Cytokeratin-2e) | | | | | | | |
| (K2e) (CK 2e) | | | | | | | |
| Keratin, type II cytoskeletal | K22E_HUMAN | 65848 | 312 | LALDVEIATYR | 1263.69 | 477 | 487 |
| 2 epidermal (Cytokeratin-2e) | | | | | | | |
| (K2e) (CK 2e) | | | | | | | |
| Keratin, type II cytoskeletal | K22E_HUMAN | 65848 | 313 | LNDLEEALQQAK | 1371.71 | 448 | 459 |
| 2 epidermal (Cytokeratin-2e) | | | | | | | |
| (K2e) (CK 2e) | | | | | | | |
| Keratin, type II cytoskeletal | K22E_HUMAN | 65848 | 314 | NLDLDSIIAEVK | 1329.73 | 348 | 359 |
| 2 epidermal (Cytokeratin-2e) | | | | | | | |
| (K2e) (CK 2e) | | | | | | | |
| Keratin, type II cytoskeletal | K22E_HUMAN | 65848 | 315 | NVQDAIADAEQR | 1329.64 | 425 | 436 |
| 2 epidermal (Cytokeratin-2e) | | | | | | | |
| (K2e) (CK 2e) | | | | | | | |
| Keratin, type II cytoskeletal | K22E_HUMAN | 65848 | 316 | VDLLNQEIEFLK | 1460.80 | 309 | 320 |
| 2 epidermal (Cytokeratin-2e) | | | | | | | |
| (K2e) (CK 2e) | | | | | | | |
| Keratin, type II cytoskeletal | K22E_HUMAN | 65848 | 317 | YEDEINKR | 1066.52 | 274 | 281 |
| 2 epidermal (Cytokeratin-2e) | | | | | | | |
| (K2e) (CK 2e) | | | | | | | |
| Keratin, type II cytoskeletal | K22E_HUMAN | 65848 | 318 | YEELQVTVGR | 1193.62 | 381 | 390 |
| 2 epidermal (Cytokeratin-2e) | | | | | | | |
| (K2e) (CK 2e) | | | | | | | |
| Keratin, type II cytoskeletal | K22E_HUMAN | 65848 | 319 | YLDGLTAER | 1037.53 | 245 | 253 |
| 2 epidermal (Cytokeratin-2e) | | | | | | | |
| (K2e) (CK 2e) | | | | | | | |
| KIAA1199 [Homo sapiens] | gi\|38638698\| | 152983 | 320 | PFLSIISARYSPHQ | 2255.18 | 755 | 774 |
| | ref\| | | | DADPLK | | | |
| | NP_061159.1\| | | | | | | |
| lipocalin 2 (oncogene 24p3) | gi\|38455402\| | 22571 | 321 | TFVPGCQPGEFTLG | 1864.93 | 102 | 118 |
| [Homo sapiens] | ref\| | | | NIK | | | |
| | NP_005555.2\| | | | | | | |
| lipocalin 2 (oncogene 24p3) | gi\|38455402\| | 22571 | 322 | VPLQQNFQDNQFQ | 1790.88 | 36 | 50 |
| [Homo sapiens] | ref\| | | | GK | | | |
| | NP_005555.2\| | | | | | | |
| lysozyme precursor | gi\|4557894\| | 16519 | 323 | GISLANWMCLAK | 1363.69 | 40 | 51 |
| [Homo sapiens] | ref\| | | | | | | |
| | NP_000230.1\| | | | | | | |
| lysozyme precursor | gi\|4557894\| | 16519 | 324 | LGMDGYR | 811.38 | 33 | 39 |
| [Homo sapiens] | ref\| | | | | | | |
| | NP_000230.1\| | | | | | | |
| lysozyme precursor | gi\|4557894\| | 16519 | 325 | QYVQGCGV | 910.41 | 141 | 148 |
| [Homo sapiens] | ref\| | | | | | | |
| | NP_000230.1\| | | | | | | |
| lysozyme precursor | gi\|4557894\| | 16519 | 326 | RLGMDGYR | 967.48 | 32 | 39 |
| [Homo sapiens] | ref\| | | | | | | |
| | NP_000230.1\| | | | | | | |
| lysozyme precursor | gi\|4557894\| | 16519 | 327 | STDYGIFQINSR | 1400.68 | 69 | 80 |
| [Homo sapiens] | ref\| | | | | | | |
| | NP_000230.1\| | | | | | | |
| lysozyme precursor | gi\|4557894\| | 16519 | 328 | TPGAVNACHLSCS | 2927.38 | 88 | 115 |
| [Homo sapiens] | ref\| | | | ALLQDNIADAVAC | | | |
| | NP_000230.1\| | | | AK | | | |
| lysozyme precursor | gi\|4557894\| | 16519 | 329 | WESGYNTR | 1012.45 | 52 | 59 |
| [Homo sapiens] | ref\| | | | | | | |
| | NP_000230.1\| | | | | | | |
| mannan-binding lectin serine | gi\|21264363\| | 75685 | 330 | VLATLCGQESTDT | 1679.79 | 85 | 99 |
| protease 2 isoform 1 pre- | ref\| | | | ER | | | |
| cursor [Homo sapiens] | NP_006601.2\| | | | | | | |
| matrix Gla protein | gi\|49574514\| | 12336 | 331 | NANTFISPQQR | 1275.64 | 39 | 49 |
| [Homo sapiens] | ref\| | | | | | | |
| | NP_000891.2\| | | | | | | |
| membrane associated guanylate | gi\|66346708\| | 140360 | 332 | NPSELKGKFIHTK | 1498.84 | 462 | 474 |
| kinase, WW and PDZ domain | ref\| | | | | | | |
| containing 1 isoform b | NP_004733.2\|, | | | | | | |
| [Homo sapiens] | gi\|74272282\| | | | | | | |
| | ref\| | | | | | | |
| | NP_056335.1\|, | | | | | | |
| | gi\|74272284\| | | | | | | |
| | ref\|NP_ | | | | | | |
| | 001028229.1\| | | | | | | |
| Mov10, Moloney leukemia virus | gi\|14211540\| | 133658 | 333 | ISFGTPAPGFSSML | 1906.91 | 45 | 62 |
| 10, homolog [Homo sapiens] | ref\| | | | YGMK | | | |
| | NP_066014.1\| | | | | | | |
| myosin IIIA [Homo sapiens] | gi\|24586657\| | 186070 | 334 | DKVNGDK | 775.40 | 89 | 95 |
| | ref\| | | | | | | |
| | NP_059129.2\| | | | | | | |
| myotubularin-related protein | gi\|19923424\| | 63446 | 335 | AGKRGYIIDTR | 1249.70 | 227 | 237 |
| 9 [Homo sapiens] | ref\| | | | | | | |
| | NP_056273.2\| | | | | | | |
| Novel protein (Keratinocyte | Q5T749 | 64115 | 336 | GRPAVCQPQGR | 1225.62 | 167 | 177 |
| proline-rich protein) | | | | | | | |
| Novel protein (Keratinocyte | Q5T749 | 64115 | 337 | IEISSPCCPR | 1218.56 | 327 | 336 |
| proline-rich protein) | | | | | | | |
| Novel protein (Keratinocyte | Q5T749 | 64115 | 338 | RPISSCSQR | 1090.54 | 312 | 320 |
| proline-rich protein) | | | | | | | |
| pericentriolar material 1 | gi\|34878902\| | 228284 | 339 | LPEMEPLVPRVKE | 1780.00 | 1906 | 1920 |
| [Homo sapiens] | ref\| | | | VK | | | |
| | NP_006188.2\| | | | | | | |
| peroxiredoxin 2 isoform a | gi\|32189392\| | 21874 | 340 | ATAVVDGAFK | 978.53 | 17 | 26 |
| [Homo sapiens] | ref\| | | | | | | |
| | NP_005800.3\|, | | | | | | |
| | gi\|33188452\| | | | | | | |
| | ref\| | | | | | | |
| | NP_859427.1\| | | | | | | |
| peroxiredoxin 2 isoform a | gi\|32189392\| | 21874 | 341 | EGGLGPLNIPLLAD | 1734.98 | 93 | 109 |
| [Homo sapiens] | ref\| | | | VTR | | | |
| | NP_005800.3\|, | | | | | | |
| | gi\|33188452\| | | | | | | |
| | ref\| | | | | | | |
| | NP_859427.1\| | | | | | | |
| peroxiredoxin 2 isoform a | gi\|32189392\| | 21874 | 342 | EGGLGPLNIPLLAD | 1734.98 | 93 | 109 |
| [Homo sapiens] | ref\| | | | VTR | | | |
| | NP_005800.3\|, | | | | | | |
| | gi\|33188452\| | | | | | | |
| | ref\| | | | | | | |
| | NP_859427.1\| | | | | | | |
| peroxiredoxin 2 isoform a | gi\|32189392\| | 21874 | 343 | GLFIIDGK | 862.50 | 128 | 135 |
| [Homo sapiens] | ref\| | | | | | | |
| | NP_005800.3\|, | | | | | | |
| | gi\|33188452\| | | | | | | |
| | ref\| | | | | | | |
| | NP_859427.1\| | | | | | | |
| peroxiredoxin 2 isoform a | gi\|32189392\| | 21874 | 344 | KEGGLGPLNIPLLA | 1863.07 | 92 | 109 |
| [Homo sapiens] | ref\| | | | DVTR | | | |
| | NP_005800.3\|, | | | | | | |
| | gi\|33188452\| | | | | | | |
| | ref\| | | | | | | |
| | NP_859427.1\| | | | | | | |
| peroxiredoxin 2 isoform a | gi\|32189392\| | 21874 | 345 | LSEDYGVLK | 1023.54 | 111 | 119 |
| [Homo sapiens] | ref\| | | | | | | |
| | NP_005800.3\|, | | | | | | |
| | gi\|33188452\| | | | | | | |
| | ref\| | | | | | | |
| | NP_859427.1\| | | | | | | |
| peroxiredoxin 2 isoform a | gi\|32189392\| | 21874 | 346 | RLSEDYGVLK | 1179.64 | 110 | 119 |
| [Homo sapiens] | ref\| | | | | | | |
| | NP_005800.3\|, | | | | | | |
| | gi\|33188452\| | | | | | | |
| | ref\| | | | | | | |
| | NP_859427.1\| | | | | | | |
| peroxiredoxin 2 isoform a | gi\|32189392\| | 21874 | 347 | TDEGIAYR | 924.44 | 120 | 127 |
| [Homo sapiens] | ref\| | | | | | | |
| | NP_005800.3\|, | | | | | | |
| | gi\|33188452\| | | | | | | |
| | ref\| | | | | | | |
| | NP_859427.1\| | | | | | | |
| phospholipase D1, | gi\|4505873\| | 124170 | 348 | IAADMSNIIENLDT | 1691.83 | 17 | 31 |
| phophatidylcholine-specific | ref\| | | | R | | | |
| [Homo sapiens] | NP_002653.1\| | | | | | | |
| plasma carboxypeptidase B2 | gi\|4503005\| | 48425 | 349 | DTGTYGFLLPER | 1368.68 | 388 | 399 |
| isoform a preproprotein | ref\| | | | | | | |
| [Homo sapiens] | NP_001863.1\| | | | | | | |
| platelet factor 4 (chemokine | gi\|4505733\| | 10828 | 350 | AGPHCPTAQLIATL | 1577.85 | 63 | 77 |
| (C-X-C motif) ligand 4) | ref\| | | | K | | | |
| [Homo sapiens] | NP_002610.1\| | | | | | | |
| platelet factor 4 (chemokine | gi\|4505733\| | 10828 | 351 | HITSLEVIK | 1039.62 | 54 | 62 |
| (C-X-C motif) ligand 4) | ref\| | | | | | | |
| [Homo sapiens] | NP_002610.1\| | | | | | | |
| platelet factor 4 (chemokine | gi\|4505733\| | 10828 | 352 | ICLDLQAPLYK | 1333.72 | 82 | 92 |
| (C-X-C motif) ligand 4) | ref\| | | | | | | |
| [Homo sapiens] | NP_002610.1\| | | | | | | |
| platelet factor 4 (chemokine | gi\|4505733\| | 10828 | 353 | ICLDLQAPLYKK | 1461.81 | 82 | 93 |
| (C-X-C motif) ligand 4) | ref\| | | | | | | |
| [Homo sapiens] | NP_002610.1\| | | | | | | |
| platelet factor 4 (chemokine | gi\|4505733\| | 10828 | 354 | KICLDLQAPLYK | 1461.81 | 81 | 92 |
| (C-X-C motif) ligand 4) | ref\| | | | | | | |
| [Homo sapiens] | NP_002610.1\| | | | | | | |
| PREDICTED: similar to Neutro- | gi\|113419903\| | 10183 | 355 | ADEVAAAPEQIAA | 3204.63 | 26 | 56 |
| phil defensin 1 precursor | ref\|XP_ | | | DIPEVVVSLAWDE | | | |
| (HNP-1) (HP-1) (HP1) | 001127929.1\|, | | | SLAPK | | | |
| (Defensin, alpha 1) | gi\|4758146\| | | | | | | |
| [Homo sapiens] | ref\| | | | | | | |
| | NP_004075.1\|, | | | | | | |
| | gi\|4885179\| | | | | | | |
| | ref\| | | | | | | |
| | NP_005208.1\| | | | | | | |
| PREDICTED: similar to Neutro- | gi\|113419903\| | 10183 | 356 | IPACIAGER | 986.51 | 70 | 78 |
| phil defensin 1 precursor | ref\|XP_ | | | | | | |
| (HNP-1) (HP-1) (HP1) | 001127929.1\|, | | | | | | |
| (Defensin, alpha 1) | gi\|4758146\| | | | | | | |
| [Homo sapiens] | ref\| | | | | | | |
| | NP_004075.1\|, | | | | | | |
| | gi\|4885179\| | | | | | | |
| | ref\| | | | | | | |
| | NP_005208.1\| | | | | | | |
| PREDICTED: similar to Neutro- | gi\|113419903\| | 10183 | 357 | RYGTCIYQGR | 1273.61 | 79 | 88 |
| phil defensin 1 precursor | ref\|XP_ | | | | | | |
| (HNP-1) (HP-1) (HP1) | 001127929.1\|, | | | | | | |
| (Defensin, alpha 1) | gi\|4758146\| | | | | | | |
| [Homo sapiens] | ref\| | | | | | | |
| | NP_004075.1\|, | | | | | | |
| | gi\|4885179\| | | | | | | |
| | ref\| | | | | | | |
| | NP_005208.1\| | | | | | | |
| PREDICTED: similar to Neutro- | gi\|113419903\| | 10183 | 358 | YGTYCIYQGR | 1117.51 | 80 | 88 |
| phil defensin 1 precursor | ref\|XP_ | | | | | | |
| (HNP-1) (HP-1) (HP1) | 001127929.1\|, | | | | | | |
| (Defensin, alpha 1) | gi\|4758146\| | | | | | | |
| [Homo sapiens] | ref\| | | | | | | |
| | NP_004075.1\|, | | | | | | |
| | gi\|4885179\| | | | | | | |
| | ref\| | | | | | | |
| | NP_005208.1\| | | | | | | |
| PREDICTED: similar to ribo- | gi\|113426784\| | 27009 | 359 | HMKIKSLEEMYVF | 2259.12 | 12 | 29 |
| somal protein S2 | ref\|XP_ | | | SLPMK | | | |
| [Homo sapiens] | 001134158.1\|, | | | | | | |
| | gi\|113427383\| | | | | | | |
| | ref\|XP_ | | | | | | |
| | 001126232.1\| | | | | | | |
| profilin family, member 4 | gi\|40786418\| | 14302 | 360 | CVRADEYSLYAKN | 2514.27 | 73 | 94 |
| [Homo sapiens] | ref\| | | | ENTGVVVVK | | | |
| | NP_955378.1\| | | | | | | |
| propionyl-Coenzyme A carboxy- | gi\|65506442\| | 80041 | 361 | YSSAGTVEFLVDS | 1502.74 | 329 | 342 |
| lase, alpha polypeptide pre- | ref\| | | | K | | | |
| cursor [Homo sapiens] | NP_000273.2\| | | | | | | |
| prosaposin [Homo sapiens] | gi\|11386147\| | 58094 | 362 | LGPGMADICK | 1061.51 | 233 | 242 |
| | ref\| | | | | | | |
| | NP_002769.1\| | | | | | | |
| pyruvate kinase 3 isoform 1 | gi\|33286418\| | 57920 | 363 | IYVDDGLISLQVK | 1462.82 | 174 | 186 |
| [Homo sapiens] | ref\| | | | | | | |
| | NP_002645.3\|, | | | | | | |
| | gi\|33286420\| | | | | | | |
| | ref\| | | | | | | |
| | NP_872270.1\|, | | | | | | |
| | gi\|33286422\| | | | | | | |
| | ref\| | | | | | | |
| | NP_872271.1\| | | | | | | |
| regulatory solute carrier | gi\|5730021\| | 66771 | 364 | DLGQGIQNSVTDR | 1885.94 | 523 | 539 |
| protein, family 1, member 1 | ref\| | | | PETR | | | |
| [Homo sapiens] | NP_006502.1\| | | | | | | |
| ribosomal protein S6 kinase, | gi\|4759050\| | 83721 | 365 | LGMPQFLSPEAQSL | 2306.24 | 285 | 304 |
| 90 kDa, polypeptide 3 | ref\| | | | LRMLFK | | | |
| [Homo sapiens] | NP_004577.1\| | | | | | | |
| roundabout, axon guidance | gi\|61888896\| | 151182 | 366 | QTSGLQATSSWQN | 1834.89 | 674 | 690 |
| receptor, homolog 2 | ref\| | | | LDAK | | | |
| [Homo sapiens] | NP_002933.1\| | | | | | | |
| Rsb-66 protein [Homo sapiens] | gi\|33285006\| | 19271 | 367 | PISSIGQVQSYMEH | 2731.22 | 42 | 64 |
| | ref\| | | | YCNSSTDRR | | | |
| | NP_061829.3\| | | | | | | |
| S100 calcium-binding protein | S10A7_HUMAN | 11309 | 368 | ENFPNFLSACDK | 1441.64 | 37 | 48 |
| A7 (Psoriasin) | | | | | | | |
| S100 calcium-binding protein | S10A7_HUMAN | 11309 | 369 | GTNYLADVFEK | 1256.62 | 50 | 60 |
| A7 (Psoriasin) | | | | | | | |
| S100 calcium-binding protein | S10A7_HUMAN | 11309 | 370 | IDFSEFLSLLGDIA | 2184.09 | 69 | 87 |
| A7 (Psoriasin) | | | | TDYHK | | | |
| S100 calcium-binding protein | S10A7_HUMAN | 11309 | 371 | KIDFSEFLSLLGDI | 2312.18 | 68 | 87 |
| A7 (Psoriasin) | | | | ATDYHK | | | |
| S100 calcium-binding protein | gi\|4506769\| | 11440 | 372 | IDFSEFLSLLGDIA | 2184.09 | 70 | 88 |
| A7 [Homo sapiens] | ref\| | | | TDYHK | | | |
| | NP_002954.1\| | | | | | | |
| S100 calcium-binding protein | gi\|4506769\| | 11440 | 373 | KIDFSEFLSLLGDI | 2312.18 | 69 | 88 |
| A7 [Homo sapiens] | ref\| | | | ATDYHK | | | |
| | NP_002954.1\| | | | | | | |
| secretoglobin, family 3A, | gi\|50363226\| | 10082 | 374 | LLLSSLGIPVNHLI | 2018.17 | 59 | 77 |
| member 1 [Homo sapiens] | ref\| | | | EGSQK | | | |
| | NP_443095.2\| | | | | | | |
| secretoglobin, family 3A, | gi\|50363226\| | 10082 | 375 | PVAQPVAALESAA | 2941.60 | 28 | 58 |
| member 1 [Homo sapiens] | ref\| | | | EAGAGTLANPLGT | | | |
| | NP_443095.2\| | | | LNPLK | | | |
| serine (or cysteine) pro- | gi\|50363217\| | 46720 | 376 | AVLTIDEK | 888.50 | 360 | 367 |
| teinase inhibitor, clade A | ref\| | | | | | | |
| (alpha-1 antiproteinase, | NP_000286.3\|, | | | | | | |
| antitrypsin), member 1 | gi\|50363219\| | | | | | | |
| [Homo sapiens] | ref\|NP_ | | | | | | |
| | 001002236.1\|, | | | | | | |
| | gi\|50363221\| | | | | | | |
| | ref\|NP_ | | | | | | |
| | 001002235.1\| | | | | | | |
| serine (or cysteine) pro- | gi\|50363217\| | 46720 | 377 | FLENEDR | 922.43 | 299 | 305 |
| teinase inhibitor, clade A | ref\| | | | | | | |
| (alpha-1 antiproteinase, | NP_000286.3\|, | | | | | | |
| antitrypsin), member 1 | gi\|50363219\| | | | | | | |
| [Homo sapiens] | ref\|NP_ | | | | | | |
| | 001002236.1\|, | | | | | | |
| | gi\|50363221\| | | | | | | |
| | ref\|NP_ | | | | | | |
| | 001002235.1\| | | | | | | |
| serine (or cysteine) pro- | gi\|50363217\| | 46720 | 378 | FNKPFVFLMIEQNT | 1855.98 | 390 | 404 |
| teinase inhibitor, clade A | ref\| | | | K | | | |
| (alpha-1 antiproteinase, | NP_000286.3\|, | | | | | | |
| antitrypsin), member 1 | gi\|50363219\| | | | | | | |
| [Homo sapiens] | ref\|NP_ | | | | | | |
| | 001002236.1\|, | | | | | | |
| | gi\|50363221\| | | | | | | |
| | ref\|NP_ | | | | | | |
| | 001002235.1\| | | | | | | |
| serine (or cysteine) pro- | gi\|50363217\| | 46720 | 379 | GTEAAGAMFLEAI | 2259.14 | 368 | 389 |
| teinase inhibitor, clade A | ref\| | | | PMSIPPEVK | | | |
| (alpha-1 antiproteinase, | NP_000286.3\|, | | | | | | |
| antitrypsin), member 1 | gi\|50363219\| | | | | | | |
| [Homo sapiens] | ref\|NP_ | | | | | | |
| | 001002236.1\|, | | | | | | |
| | gi\|50363221\| | | | | | | |
| | ref\|NP_ | | | | | | |
| | 001002235.1\| | | | | | | |
| serine (or cysteine) pro- | gi\|50363217\| | 46720 | 380 | ITPNLAEFAFSLYR | 1641.86 | 50 | 63 |
| teinase inhibitor, clade A | ref\| | | | | | | |
| (alpha-1 antiproteinase, | NP_000286.3\|, | | | | | | |
| antitrypsin), member 1 | gi\|50363219\| | | | | | | |
| [Homo sapiens] | ref\|NP_ | | | | | | |
| | 001002236.1\|, | | | | | | |
| | gi\|50363221\| | | | | | | |
| | ref\|NP_ | | | | | | |
| | 001002235.1\| | | | | | | |
| serine (or cysteine) pro- | gi\|50363217\| | 46720 | 381 | KLSSWVLLMK | 1204.71 | 258 | 267 |
| teinase inhibitor, clade A | ref\| | | | | | | |
| (alpha-1 antiproteinase, | NP_000286.3\|, | | | | | | |
| antitrypsin), member 1 | gi\|50363219\| | | | | | | |
| [Homo sapiens] | ref\|NP_ | | | | | | |
| | 001002236.1\|, | | | | | | |
| | gi\|50363221\| | | | | | | |
| | ref\|NP_ | | | | | | |
| | 001002235.1\| | | | | | | |
| serine (or cysteine) pro- | gi\|50363217\| | 46720 | 382 | LQHLENELTHDIIT | 1803.96 | 284 | 298 |
| teinase inhibitor, clade A | ref\| | | | K | | | |
| (alpha-1 antiproteinase, | NP_000286.3\|, | | | | | | |
| antitrypsin), member 1 | gi\|50363219\| | | | | | | |
| [Homo sapiens] | ref\|NP_ | | | | | | |
| | 001002236.1\|, | | | | | | |
| | gi\|50363221\| | | | | | | |
| | ref\|NP_ | | | | | | |
| | 001002235.1\| | | | | | | |
| serine (or cysteine) pro- | gi\|50363217\| | 46720 | 383 | LSITGTYDLK | 1110.61 | 315 | 324 |
| teinase inhibitor, clade A | ref\| | | | | | | |
| (alpha-1 antiproteinase, | NP_000286.3\|, | | | | | | |
| antitrypsin), member 1 | gi\|50363219\| | | | | | | |
| [Homo sapiens] | ref\|NP_ | | | | | | |
| | 001002236.1\|, | | | | | | |
| | gi\|50363221\| | | | | | | |
| | ref\|NP_ | | | | | | |
| | 001002235.1\| | | | | | | |
| serine (or cysteine) pro- | gi\|50363217\| | 46720 | 384 | LSSWVLLMK | 1076.62 | 259 | 267 |
| teinase inhibitor, clade A | ref\| | | | | | | |
| (alpha-1 antiproteinase, | NP_000286.3\|, | | | | | | |
| antitrypsin), member 1 | gi\|50363219\| | | | | | | |
| [Homo sapiens] | ref\|NP_ | | | | | | |
| | 001002236.1\|, | | | | | | |
| | gi\|50363221\| | | | | | | |
| | ref\|NP_ | | | | | | |
| | 001002235.1\| | | | | | | |
| serine (or cysteine) pro- | gi\|50363217\| | 46720 | 385 | SASLHLPK | 852.49 | 307 | 314 |
| teinase inhibitor, clade A | ref\| | | | | | | |
| (alpha-1 antiproteinase, | NP_000286.3\|, | | | | | | |
| antitrypsin), member 1 | gi\|50363219\| | | | | | | |
| [Homo sapiens] | ref\|NP_ | | | | | | |
| | 001002236.1\|, | | | | | | |
| | gi\|50363221\| | | | | | | |
| | ref\|NP_ | | | | | | |
| | 001002235.1\| | | | | | | |
| serine (or cysteine) pro- | gi\|50363217\| | 46720 | 386 | SPLFMGK | 779.41 | 405 | 411 |
| teinase inhibitor, clade A | ref\| | | | | | | |
| (alpha-1 antiproteinase, | NP_000286.3\|, | | | | | | |
| antitrypsin), member 1 | gi\|50363219\| | | | | | | |
| [Homo sapiens] | ref\|NP_ | | | | | | |
| | 001002236.1\|, | | | | | | |
| | gi\|50363221\| | | | | | | |
| | ref\|NP_ | | | | | | |
| | 001002235.1\| | | | | | | |
| serine (or cysteine) pro- | gi\|50363217\| | 46720 | 387 | SVLGQLGITK | 1015.62 | 325 | 334 |
| teinase inhibitor, clade A | ref\| | | | | | | |
| (alpha-1 antiproteinase, | NP_000286.3\|, | | | | | | |
| antitrypsin), member 1 | gi\|50363219\| | | | | | | |
| [Homo sapiens] | ref\|NP_ | | | | | | |
| | 001002236.1\|, | | | | | | |
| | gi\|50363221\| | | | | | | |
| | ref\|NP_ | | | | | | |
| | 001002235.1\| | | | | | | |
| serine (or cysteine) pro- | gi\|50363217\| | 46720 | 388 | VFSNGADLSGVTE | 1833.92 | 335 | 352 |
| teinase inhibitor, clade A | ref\| | | | EAPLK | | | |
| (alpha-1 antiproteinase, | NP_000286.3\|, | | | | | | |
| antitrypsin), member 1 | gi\|50363219\| | | | | | | |
| [Homo sapiens] | ref\|NP_ | | | | | | |
| | 001002236.1\|, | | | | | | |
| | gi\|50363221\| | | | | | | |
| | ref\|NP_ | | | | | | |
| | 001002235.1\| | | | | | | |
| serine (or cysteine) pro- | gi\|28076869\| | 44837 | 389 | FMFDLFQQFR | 1378.66 | 11 | 20 |
| teinase inhibitor, clade B | ref\| | | | | | | |
| (ovalbumin), member 4 | NP_002965.1\|, | | | | | | |
| [Homo sapiens] | gi\|5902072\| | | | | | | |
| | ref\| | | | | | | |
| | NP_008850.1\| | | | | | | |
| serum amyloid A2 | gi\|13540475\| | 13491 | 390 | DPNHFRPAGLPEK | 1640.82 | 109 | 122 |
| [Homo sapiens] | ref\| | | | Y | | | |
| | NP_110381.1\| | | | | | | |
| serum amyloid A2 | gi\|13540475\| | 13491 | 391 | EANYIGSDKYFHA | 1670.79 | 44 | 57 |
| [Homo sapiens] | ref\| | | | R | | | |
| | NP_110381.1\| | | | | | | |
| serum amyloid A2 | gi\|13540475\| | 13491 | 392 | LTGHGAEDSLADQ | 1697.81 | 86 | 102 |
| [Homo sapiens] | ref\| | | | AANK | | | |
| | NP_110381.1\| | | | | | | |
| serum amyloid A2 | gi\|13540475\| | 13491 | 393 | RGPGGAWAAEVIS | 1611.84 | 65 | 80 |
| [Homo sapiens] | ref\| | | | NAR | | | |
| | NP_110381.1\| | | | | | | |
| serum amyloid A2 | gi\|13540475\| | 13491 | 394 | SFFSFLGEAFDGAR | 1550.73 | 20 | 33 |
| [Homo sapiens] | ref\| | | | | | | |
| | NP_110381.1\| | | | | | | |
| serum amyloid A4, constitu- | gi\|10835095\| | 14789 | 395 | AYWDIMISNHQNS | 1848.84 | 38 | 52 |
| tive [Homo sapiens] | ref\| | | | NR | | | |
| | NP_006503.1\| | | | | | | |
| serum amyloid A4, constitu- | gi\|10835095\| | 14789 | 396 | EALQGVGDMGR | 1132.54 | 27 | 37 |
| tive [Homo sapiens] | ref\| | | | | | | |
| | NP_006503.1\| | | | | | | |
| serum amyloid A4, constitu- | gi\|10835095\| | 14789 | 397 | FRPDGLPK | 929.52 | 121 | 128 |
| tive [Homo sapiens] | ref\| | | | | | | |
| | NP_006503.1\| | | | | | | |
| serum amyloid A4, constitu- | gi\|10835095\| | 14789 | 398 | GPGGVWAAK | 842.45 | 66 | 74 |
| tive [Homo sapiens] | ref\| | | | | | | |
| | NP_006503.1\| | | | | | | |
| serum amyloid A4, constitu- | gi\|10835095\| | 14789 | 399 | SFFKEALQGVGDM | 1641.81 | 23 | 37 |
| tive [Homo sapiens] | ref\| | | | GR | | | |
| | NP_006503.1\| | | | | | | |
| serum amyloid A4, constitu- | gi\|10835095\| | 14789 | 400 | SNEKAEEWGR | 1205.56 | 104 | 113 |
| tive [Homo sapiens] | ref\| | | | | | | |
| | NP_006503.1\| | | | | | | |
| serum amyloid A4, constitu- | gi\|10835095\| | 14789 | 401 | VYLQGLIDYYLFG | 2624.31 | 81 | 103 |
| tive [Homo sapiens] | ref\| | | | NSSTVLEDSK | | | |
| | NP_006503.1\| | | | | | | |
| stanniocalcin 2 precursor | gi\|4507267\| | 33230 | 402 | EMVSQLQRECYLK | 1699.81 | 130 | 142 |
| [Homo sapiens] | ref\| | | | | | | |
| | NP_003705.1\| | | | | | | |
| Tetranectin precursor (TN) | TETN_HUMAN | 22549 | 403 | GGTLSTPQTGSEN | 2272.07 | 102 | 122 |
| (Plasminogen-kringle 4 | | | | DALYEYLR | | | |
| binding protein) | | | | | | | |
| Tetranectin precursor (TN) | TETN_HUMAN | 22549 | 404 | LDTLAQEVALLK | 1313.77 | 51 | 62 |
| (Plasminogen-kringle 4 | | | | | | | |
| binding protein) | | | | | | | |
| thrombospondin 4 precursor | gi\|31543806\| | 105851 | 405 | LNPGALLPVLTDP | 2684.49 | 41 | 65 |
| [Homo sapiens] | ref\| | | | ALNDLYVISTFK | | | |
| | NP_003239.2\| | | | | | | |
| transferrin [Homo sapiens] | gi\|4557871\| | 77032 | 406 | AIAANEADAVTLD | 3954.02 | 70 | 107 |
| | ref\| | | | AGLVYDAYLAPN | | | |
| | NP_001054.1\| | | | NLKPVVAEFYGSK | | | |
| transferrin [Homo sapiens] | gi\|4557871\| | 77032 | 407 | ASYLDCIR | 997.48 | 62 | 69 |
| | ref\| | | | | | | |
| | NP_001054.1\| | | | | | | |
| transferrin [Homo sapiens] | gi\|4557871\| | 77032 | 408 | CSTSSLLEAGTFR | 1531.69 | 684 | 696 |
| | ref\| | | | | | | |
| | NP_001054.1\| | | | | | | |
| transferrin [Homo sapiens] | gi\|4557871\| | 77032 | 409 | DDTVCLAK | 921.44 | 652 | 659 |
| | ref\| | | | | | | |
| | NP_001054.1\| | | | | | | |
| transferrin [Homo sapiens] | gi\|4557871\| | 77032 | 410 | DGAGDVAFVK | 978.49 | 216 | 225 |
| | ref\| | | | | | | |
| | NP_001054.1\| | | | | | | |
| transferrin [Homo sapiens] | gi\|4557871\| | 77032 | 411 | DLLFRDDTVCLAK | 1565.80 | 647 | 659 |
| | ref\| | | | | | | |
| | NP_001054.1\| | | | | | | |
| transferrin [Homo sapiens] | gi\|4557871\| | 77032 | 412 | DSGFQMNQLR | 1195.55 | 123 | 132 |
| | ref\| | | | | | | |
| | NP_001054.1\| | | | | | | |
| transferrin [Homo sapiens] | gi\|4557871\| | 77032 | 413 | DYELLCLDGTR | 1354.63 | 577 | 587 |
| | ref\| | | | | | | |
| | NP_001054.1\| | | | | | | |
| transferrin [Homo sapiens] | gi\|4557871\| | 77032 | 414 | EDPQTFYYAVAVV | 1629.82 | 108 | 121 |
| | ref\| | | | K | | | |
| | NP_001054.1\| | | | | | | |
| transferrin [Homo sapiens] | gi\|4557871\| | 77032 | 415 | HSTIFENLANK | 1273.65 | 226 | 236 |
| | ref\| | | | | | | |
| | NP_001054.1\| | | | | | | |
| transferrin [Homo sapiens] | gi\|4557871\| | 77032 | 416 | KASYLDCIR | 1125.57 | 61 | 69 |
| | ref\| | | | | | | |
| | NP_001054.1\| | | | | | | |
| transferrin [Homo sapiens] | gi\|4557871\| | 77032 | 417 | KCSTSSLLEACTFR | 1659.78 | 683 | 696 |
| | ref\| | | | | | | |
| | NP_001054.1\| | | | | | | |
| transferrin [Homo sapiens] | gi\|4557871\| | 77032 | 418 | KDSGFQMNQLR | 1323.65 | 122 | 132 |
| | ref\| | | | | | | |
| | NP_001054.1\| | | | | | | |
| transferrin [Homo sapiens] | gi\|4557871\| | 77032 | 419 | KPVEEYANCHLAR | 1586.77 | 588 | 600 |
| | ref\| | | | | | | |
| | NP_001054.1\| | | | | | | |
| transferrin [Homo sapiens] | gi\|4557871\| | 77032 | 420 | NLNEKDYELLCLD | 1952.94 | 572 | 587 |
| | ref\| | | | GTR | | | |
| | NP_001054.1\| | | | | | | |
| transferrin [Homo sapiens] | gi\|4557871\| | 77032 | 421 | NPDPWAK | 827.41 | 565 | 571 |
| | ref\| | | | | | | |
| | NP_001054.1\| | | | | | | |
| transferrin [Homo sapiens] | gi\|4557871\| | 77032 | 422 | SAGWNIPIGLLYCD | 2171.10 | 144 | 162 |
| | ref\| | | | LPEPR | | | |
| | NP_001054.1\| | | | | | | |
| transferrin [Homo sapiens] | gi\|4557871\| | 77032 | 423 | SVIPSDGPSVACVK | 1415.72 | 47 | 60 |
| | ref\| | | | | | | |
| | NP_001054.1\| | | | | | | |
| transferrin [Homo sapiens] | gi\|4557871\| | 77032 | 424 | WCAVSEHEATK | 1317.59 | 27 | 37 |
| | ref\| | | | | | | |
| | NP_001054.1\| | | | | | | |
| transferrin [Homo sapiens] | gi\|4557871\| | 77032 | 425 | YLGEEYVK | 1000.50 | 669 | 676 |
| | ref\| | | | | | | |
| | NP_001054.1\| | | | | | | |
| triggering receptor expressed | gi\|30794266\| | 32661 | 426 | FLPEGCQPLVSSAV | 1774.88 | 54 | 69 |
| on myeloid cells-like 1 | ref\| | | | DR | | | |
| [Homo sapiens] | NP_835468.1\| | | | | | | |
| vitamin D-binding protein | gi\|32483410\| | 52900 | 427 | EDFTSLSLVLYSR | 1529.79 | 38 | 50 |
| precursor [Homo sapiens] | ref\| | | | | | | |
| | NP_000574.2\| | | | | | | |
| vitamin D-binding protein | gi\|32483410\| | 52900 | 428 | EFSHLGKEDFTSLS | 2328.19 | 31 | 50 |
| precursor [Homo sapiens] | ref\| | | | LVLYSR | | | |
| | NP_000574.2\| | | | | | | |
| vitamin D-binding protein | gi\|32483410\| | 52900 | 429 | ELSSFIDK | 938.48 | 395 | 402 |
| precursor [Homo sapiens] | ref\| | | | | | | |
| | NP_000574.2\| | | | | | | |
| vitamin D-binding protein | gi\|32483410\| | 52900 | 430 | FPSGTFEQVSQLVK | 1566.82 | 52 | 65 |
| precursor [Homo sapiens] | ref\| | | | | | | |
| | NP_000574.2\| | | | | | | |
| vitamin D-binding protein | gi\|32483410\| | 52900 | 431 | KFPSGTFEQVSQLV | 1694.91 | 51 | 65 |
| precursor [Homo sapiens] | ref\| | | | K | | | |
| | NP_000574.2\| | | | | | | |
| vitamin D-binding protein | gi\|32483410\| | 52900 | 432 | LPDATPTELAK | 1155.63 | 430 | 440 |
| precursor [Homo sapiens] | ref\| | | | | | | |
| | NP_000574.2\| | | | | | | |
| vitamin D-binding protein | gi\|32483410\| | 52900 | 433 | RTHLPEVFLSK | 1326.75 | 353 | 363 |
| precursor [Homo sapiens] | ref\| | | | | | | |
| | NP_000574.2\| | | | | | | |
| vitamin D-binding protein | gi\|32483410\| | 52900 | 434 | VPTADLEDVLPLA | 2366.27 | 243 | 264 |
| precursor [Homo sapiens] | ref\| | | | EDITNILSK | | | |
| | NP_000574.2\| | | | | | | |
| vitamin D-binding protein | gi\|32483410\| | 52900 | 435 | YTFELSR | 915.46 | 346 | 352 |
| precursor [Homo sapiens] | ref\| | | | | | | |
| | NP_000574.2\| | | | | | | |
| vitronectin precursor | gi\|88853069\| | 54288 | 436 | IYISGMAPR | 1007.53 | 354 | 362 |
| [Homo sapiens] | ref\| | | | | | | |
| | NP_000629.3\| | | | | | | |
| vitronectin precursor | gi\|88853069\| | 54288 | 437 | MDWLVPATCEPIQ | 2574.21 | 422 | 443 |
| [Homo sapiens] | ref\| | | | SVFFFSGDK | | | |
| | NP_000629.3\| | | | | | | |
| vitronectin precursor | gi\|88853069\| | 54288 | 438 | RVDTVDPPYPR | 1314.68 | 453 | 463 |
| [Homo sapiens] | ref\| | | | | | | |
| | NP_000629.3\| | | | | | | |
| vitronectin precursor | gi\|88853069\| | 54288 | 439 | SIAQYWLGCPAPG | 1669.82 | 464 | 478 |
| [Homo sapiens] | ref\| | | | HL | | | |
| | NP_000629.3\| | | | | | | |
| vitronectin precursor | gi\|88853069\| | 54288 | 440 | VDTVDPPYPR | 1158.58 | 454 | 463 |
| [Homo sapiens] | ref\| | | | | | | |
| | NP_000629.3\| | | | | | | |
| (P21817) Ryanodine receptor 1 | RYR1_HUMAN | 565156 | 441 | EEQNFVVQNEINN | 2584.20 | 3454 | 3475 |
| (Skeletal muscle-type | | | | MSFLTADNK | | | |
| ryanodine receptor) (RyR1) | | | | | | | |
| (RYR-1) (Skeletal muscle | | | | | | | |
| calcium release channel) | | | | | | | |
| (Q9NVE5) Ubiquitin carboxyl- | Q9NVE5 | 140130 | 442 | IGISWNKK | 945.55 | 372 | 379 |
| terminal hydrolase 40 (EC | | | | | | | |
| 3.1.2.15) (U | | | | | | | |
| (Q5NV79) V5-4 protein | QSNV79 | 10661 | 443 | FSGSSSGADR | 970.42 | 66 | 75 |
| (Fragment) | | | | | | | |
| (P51843) Nuclear receptor 0B1 | DAX1_HUMAN | 51700 | 444 | YLPCFQVLPLDQQ | 2201.22 | 271 | 288 |
| (Nuclear receptor DAX-1) | | | | LVLVR | | | |
| (DSS-AHC critical region on | | | | | | | |
| the X chromosome protein 1) | | | | | | | |
| (Q96SU4) Oxysterol binding | Q96SU4 | 83185 | 445 | HCIVLLQIAKDQSN | 1967.04 | 172 | 188 |
| protein-related protein 9 | | | | AEK | | | |
| (OSBP-related | | | | | | | |
| (P17706) Tyrosine-protein | PTN2_HUMAN | 48511 | 446 | VKLQNAENDYINA | 2632.32 | 59 | 81 |
| phosphatase, non-receptor | | | | SLVDIEEAQR | | | |
| type 2 (EC 3.1.3.48) (T-cell | | | | | | | |
| protein-tyrosine phospha- | | | | | | | |
| tase) (TCPTP) | | | | | | | |
| (O15013) Rho guanine nucleo- | ARHGA_HUMAN | 127102 | 447 | YILGSVVDSEKNY | 2169.16 | 194 | 212 |
| tide exchange factor 10 | | | | VDALKR | | | |
| (O75129) KIAA0634 protein | O75129 | 145410 | 448 | EAFKSALMSSYWC | 1851.84 | 1006 | 1021 |
| (Fragment) | | | | SGK | | | |
| (Q8N543) Hypothetical pro- | Q8N543 | 63230 | 449 | EPHISTLRKILFED | 2001.09 | 109 | 124 |
| tein FLJ10826 | | | | FR | | | |
| (Q96AE7) Tetratricopeptide | TTC17_HUMAN | 129543 | 450 | IHIEENEDRDTGLE | 2333.12 | 91 | 109 |
| repeat protein 17 (TPR | | | | QRHNK | | | |
| repeat protein 17) | | | | | | | |
| (Q96AE7) Tetratricopeptide | TTC17_HUMAN | 129543 | 451 | GLRIHELSSDDYST | 3371.50 | 530 | 558 |
| repeat protein 17 (TPR | | | | EEEAQTPDCSITDF | | | |
| repeat protein 17) | | | | R | | | |
| (Q96AE7) Tetratricopeptide | TTC17_HUMAN | 129543 | 452 | IQQQVDSPMNLK | 1400.72 | 50 | 61 |
| repeat protein 17 (TPR | | | | | | | |
| repeat protein 17) | | | | | | | |

TABLE 2
Biomarkers associated with Alzheimer's Disease

| | | | | | Calculated | | | |
|---|---|---|---|---|---|---|---|---|
| | | | | | Peptide | Peptide | | |
| | Protein | Protein molecular | SEQ ID | | Mass | start | Peptide | |
| Protein name | accession numbers | weight (Da) | NO: | Peptide sequence | (AMU) | index | stop index | |
| (O60602) Toll-like receptor 5 | TLR5_HUMAN | 97713 | 1 | LYFCGLSDAVLKDGYF | 2379.22 | 126 | 145 | |
| precursor (Toll/interleukin-1 receptor- | | | | RNLK | | | | |
| like protein 3) | | | | | | | | |
| (O95793) Double-stranded RNA- | STAU_HUMAN | 63250 | 3 | ALRILQNEPLPERLEVN | 2217.25 | 156 | 174 | |
| binding protein Staufen homolog | | | | GR | | | | |
| (P01764) Ig heavy chain V-III region | HV3C_HUMAN | 12565 | 4 | AEDTAVYYCAK | 1290.57 | 107 | 117 | |
| VH26 precursor | | | | | | | | |
| (P02766) Transthyretin precursor | TTHY_HUMAN | 15869 | 5 | AADDTWEPFASGK | 1394.62 | 56 | 68 | |
| (Prealbumin) (TBPA) (TTR) (ATTR) | | | | | | | | |
| (P02766) Transthyretin precursor | TTHY_HUMAN | 15869 | 6 | ALGISPFHEHAEVVFT | 2607.31 | 101 | 124 | |
| (Prealbumin) (TBPA) (TTR) (ATTR) | | | | ANDSGPRR | | | | |
| (P02766) Transthyretin precursor | TTHY_HUMAN | 15869 | 7 | GSPAINVAVHVFR | 1366.76 | 42 | 54 | |
| (Prealbumin) (TBPA) (TTR) (ATTR) | | | | | | | | |
| (P02766) Transthyretin precursor | TTHY_HUMAN | 15869 | 8 | GSPAINVAVHVFRK | 1494.85 | 42 | 55 | |
| (Prealbumin) (TBPA) (TTR) (ATTR) | | | | | | | | |
| (P02766) Transthyretin precursor | TTHY_HUMAN | 15869 | 9 | KAADDTWEPFASGK | 1522.72 | 55 | 68 | |
| (Prealbumin) (TBPA) (TTR) (ATTR) | | | | | | | | |
| (P02766) Transthyretin precursor | TTHY_HUMAN | 15869 | 10 | RYTIAALLSPYSYSTTA | 2645.38 | 124 | 147 | |
| (Prealbumin) (TBPA) (TTR) (ATTR) | | | | | | | | |
| (P02766) Transthyretin precursor | TTHY_HUMAN | 15869 | 11 | TSESGELHGLTTEEEFV | 3140.52 | 69 | 96 | |
| (Prealbumin) (TBPA) (TTR) (ATTR) | | | | EGIYKVEIDTK | | | | |
| (P02766) Transthyretin precursor | TTHY_HUMAN | 15869 | 12 | VLDAVRGSPAINVAV | 2020.15 | 36 | 54 | |
| (Prealbumin) (TBPA) (TTR) (ATTR) | | | | HVFR | | | | |
| (P02766) Transthyretin precursor | ITHY_HUMAN | 15869 | 13 | YTIAALLSPYSYSTTAV | 2489.28 | 125 | 147 | |
| (Prealbumin) (TBPA) (TTR) (ATTR) | | | | VTNPKE | | | | |
| (P09874) Poly [ADP-ribose] | PARP1_HUMAN | 112939 | 15 | EDAIEHFMKIYEEK | 1781.84 | 607 | 620 | |
| polymerase 1 (EC 2.4.2.30) (PARP | | | | | | | | |
| 1) (ADPRT) (NAD(+) ADP- | | | | | | | | |
| ribosyltransferase 1) (Poly[ADP | | | | | | | | |
| ribose]synthetase 1) | | | | | | | | |
| (P09874) Poly [ADP-ribose] | PARP1_HUMAN | 112939 | 16 | MKLTLKGGAAVDPDS | 2703.41 | 522 | 547 | |
| polymerase 1 (EC 2.4.2.30) (PARP | | | | GLEHSAHVLEK | | | | |
| 1) (ADPRT) (NAD(+) ADP- | | | | | | | | |
| ribosyltransferase 1) (Poly[ADP- | | | | | | | | |
| ribose]synthetase 1) | | | | | | | | |
| (P13667) Protein disulfide-isomerase | PDIA4_HUMAN | 72916 | 21 | LAPEYEKAAKELSK | 1576.86 | 212 | 225 | |
| A4precursor(EC5.3.4.1)(Protein | | | | | | | | |
| ERp-72) (ERp72) | | | | | | | | |
| (P16591) Proto-oncogene tyrosine- | FER_HUMAN | 94609 | 22 | FLQEAKILKQYDHPNI | 2184.22 | 605 | 622 | |
| protein kinase FER (EC 2.7.1.112) | | | | VK | | | | |
| (p94-FER) (c-FER) | | | | | | | | |
| (P22792) Carboxypeptidase N | CPN2_HUMAN | 60599 | 24 | TLNLAQNLLAQLPEEL | 3045.70 | 173 | 199 | |
| subunit 2 precursor | | | | FHPLTSLQTLK | | | | |
| (Carboxypeptidase N polypeptide 2) | | | | | | | | |
| (Carboxypeptidase N 83 kDa chain) | | | | | | | | |
| (Carboxypeptidase N regulatory | | | | | | | | |
| subunit) (Carboxypeptidase N large | | | | | | | | |
| subunit) | | | | | | | | |
| (P35542) Serum amyloid A-4 protein | SAA4_HUMAN | 14789 | 25 | FRPDGLPK | 929.52121 | 128 | | |
| precursor (Constitutively expressed | | | | | | | | |
| serum amyloid A protein) (C-SAA) | | | | | | | | |
| (Q05513) Protein kinase C, zeta type | KPCZ_HUMAN | 67644 | 26 | EGLGPGDTTSTFCGTP | 2666.28 | 400 | 424 | |
| EC 2.7.137) (nPKC-zeta) | | | | NYIAPEILR | | | | |
| (Q15166) Serum | PON3_HUMAN | 39590 | 27 | ILIGTVFHK | 1027.63 | 340 | 348 | |
| paraoxonase/Iactonase 3 (EC 3.1.1.-) | | | | | | | | |
| (Q4V312) Colony stimulating factor | Q4V312 | 46884 | 28 | DKLNDNHEVEDEMGP | 2025.89 | 364 | 380 | |
| 2 receptor, alpha, low-affinity | | | | | | | | |
| (Granulocyte-macrophage) | QR | | | | | | | |
| (Q7Z3Z2) Protein Clorf36 | CA036_HUMAN | 22686 | 31 | PRGSLATFK | 976.56 | 146 | 154 | |
| (Q8N7W7) Hypothetical protein | Q8N7W7 | 66111 | 32 | ILIDGTLIIFR | 1273.79 | 124 | 134 | |
| FLJ40259 | | | | | | | | |
| (Q96MA6) Hypothetical protein | Q96MA6 | 54909 | 33 | KTVPSALLVQLIQERL | 2653.46 | 111 | 133 | |
| FLJ32704 (Chromosome 9 open | | | | AEEDCIK | | | | |
| reading frame 98) | | | | | | | | |
| (OTTHUMP00000022737) | | | | | | | | |
| (Q9BXB9) LIM mineralization | Q9BXB9 | 46492 | 37 | HSQPATPTPLQSR | 1419.73 | 212 224 | | |
| protein 2 | | | | | | | | |
| (Q9H212) HNRBF-2 | Q9H212 | 31791 | 38 | NCYSLLNLAEVRSKYL | 2128.12 | 236 | 253 | |
| | | | | GK | | | | |
| (Q9H212) HNRBF-2 | Q9H212 | 31791 | 39 | NCYSLLNLAEVRSKYL | 2128.12 | 236 | 253 | |
| | | | | GK | | | | |
| (Q9NPP6) Immunoglobulin heavy | Q9NPP6 | 44767 | 40 | VEDTAVYYCAR | 1346.61 | 48 | 58 | |
| chain variant (Fragment) | | | | | | | | |
| (Q9NYQ6) Cadherin EGF LAG | CELR1_HUMAN | 329464 | 41 | ENLEVGYEVLTIRASD | 2944.51 | 364 389 | | |
| seven-pass G-type receptor 1 | | | | RDSPINANLR | | | | |
| precursor (Flamingo homolog 2) | | | | | | | | |
| (hFmi2) | | | | | | | | |
| (Q9NYQ6) Cadherin EGF LAG | CELR1_HUMAN | 329464 | 42 | ETKETHVLR | 1112.61 314 | 322 | | |
| seven-pass G-type receptor 1 | | | | | | | | |
| precursor (Flamingo homolog 2) | | | | | | | | |
| (hFmi2) | | | | | | | | |
| (Q9NYQ6) Cadherin EGF LAG | CELR1_HUMAN | 329464 | 43 | QFVGCMRNLSVDGK | 1610.78 | 1616 | 1629 | |
| seven-pass G-type receptor 1 | | | | | | | | |
| precursor (Flamingo homolog 2) | | | | | | | | |
| (hFmi2) | | | | | | | | |
| (Q9NYQ6) Cadherin EGF LAG | CELR1_HUMAN | 329464 | 44 | VPGGTRAFALRPGCTY | 2606.31 | 35 | 59 | |
| seven-pass G-precursor (Flamingo homolog 2) | | | | AVGAACTPR | | | | |
| (hFmi2) | | | | | | | | |
| Apolipoprotein C-I precursor (Apo- | APOC1_HUMAN | 9314 | 56 | EWFSETFQK | 1201.55 | 66 | 74 | |
| CI) (ApoC-I) | | | | | | | | |
| Apolipoprotein C-I precursor (Apo- | APOC1_HUMAN | 9314 | 57 | IKQSELSAK | 1003.58 | 55 | 63 | |
| CI) (ApoC-I) | | | | | | | | |
| Apolipoprotein C-I precursor (Apo- | APOC1_HUMAN | 9314 | 58 | LKEFGNTLEDK | 1293.67 | 37 | 47 | |
| CI) (ApoC-I) | | | | | | | | |
| Apolipoprotein C-I precursor (Apo- | APOC1_HUMAN | 9314 | 59 | TPDVSSALDK | 1032.52 | 27 | 36 | |
| CI) (ApoC-I) | | | | | | | | |
| Apolipoprotein C-IV precursor (Apo- | APOC4_HUMAN | 14536 | 66 | AWFLESK | 880.46 | 99 | 105 | |
| CIV) (ApoC-IV) | | | | | | | | |
| Apolipoprotein C-IV precursor (Apo- | APOC4_HUMAN | 14536 | 67 | DGWQWFWSPSTFR | 1699.77 | 67 | 79 | |
| CIV) (ApoC-IV) | | | | | | | | |
| Apolipoprotein C-IV precursor (Apo- | APOC4_HUMAN | 14536 | 68 | ELLETVVNR | 1072.60 | 56 | 64 | |
| CIV) (ApoC-IV) | | | | | | | | |
| biliverdin reductase B (flavin | gi\|4502419\|ref\|NP_000704.1\| | 22101 | 93 | LPSEGPRPAHVVVGDV | 2469.31 | 40 | 63 | |
| reductase (NADPH)) [Homosapiens] | | | | | | | LQAADVDK | |
| biliverdin reducrase B (flavin | gi\|4502419\|ref\|NP_000704.1\| | 22101 | 94 | NIVAAMK | 746.42 | 93 | 99 | |
| reductase (NADPH)) [Homo sapiens] | | | | | | | | |
| biliverdinreductase B (flavin | gi\|4502419\|ref\|NP_000704.1\| | 22101 | 95 | PAHVVVGDVLQAADV | 1732.92 | 47 | 63 | |
| reductase (NADPH)) [Homo sapiens] | | | | DK | | | | |
| biliverdin reductase B (flavin | giF4502419\|ref\|NP_000704.1\| | 22101 | 96 | TVAGQDAVIVLLGTR | 1512.88 | 64 | 78 | |
| reductase (NADPH)) [Homo sapiens] | | | | | | | | |
| biliverdin reductase B (flavin | gi\|4502419fref\|NP_000704.1\| | 22101 | 97 | VVACTSAFLLWDPTK | 1707.88 | 106 | 120 | |
| reductase (NADPH)) [Homo sapiens] | | | | | | | | |
| biliverdin reductase B (flavin | gi\|4502419\|ref\|NP_000704.1\| | 22101 | 98 | YVAVMPPHIGDQPLTG | 2671.36 | 146 | 170 | |
| reductase (NADPH)) [Homo sapiens] | | | | AYTVTLDGR | | | | |
| chromosome 1 open reading frame 48 | gi\|21361470\|ref\|NP_056286.21 | 32112 | 99 | EISEAMK | 823.39 | 183 | 189 | |
| [Homo sapiens] | | | | | | | | |
| chromosome 9 open reading frame 19 | gi\|11641247\|ref\|NP_071738.11 | 17200 | 100 | EAQQYSEALASTR | 1453.69 | 38 | 50 | |
| [Homo sapiens] | | | | | | | | |
| citron [Homo sapiens] | gi\|32698688\|ref\|NP_009105.1\| | 231418 | 101 | QQKFYLETQAGK | 1440.75 | 814 | 825 | |
| complement component 4B | gi\|50345296\|ref\|NP_00100202 | 192735 | 111 | DFALLSLQVPLK | 1343.79 | 81 | 92 | |
| preproprotein [Homo sapiens] | 9.1\| | | | | | | | |
| complement component 4B | gi\|50345296\|ref\|NP_00100202 | 192735 | 112 | DFALLSLQVPLKDAK | 1657.95 | 81 | 95 | |
| preproprotein [Homo sapiens] | 9.1\|,gi167190748\|ref\|NP_00922 | | | | | | | |
| | 4.2\| | | | | | | | |
| complement component 4B | gi\|50345296\|ref\|NP_00100202 | 192735 | 113 | DHAVDLIQK | 1038.56 | 1043 1051 | | |
| preproprotein [Homo sapiens] | 9.1\| | | | | | | | |
| complement component 4B | gi\|50345296\|ref\|NP_00100202 | 192735 | 114 | EPFLSCCQFAESLR | 1743.78 | 730 | 743 | |
| preproprotein [Homo sapiens] | 9.1\|,gi167190748\|ref\|NP_00922 | | | | | | | |
| | 4.21 | | | | | | | |
| complement component 4B | gi\|50345296\|ref\|NP_00100202 | 192735 | 115 | GLEEELQFSLGSK | 1436.73 | 1353 1365 | | |
| preproprotein [Homo sapiens] | 9.1\|,gi\|67190748\|ref\|NP_00922 | | | | | | | |
| | 4.21 | | | | | | | |
| complement component 4B | gi\|50345296fref\|NP_00100202 | 192735 | 116 | GPEVQLVAHSPWLK | 1560.85 | 105 | 118 | |
| preproprotein [Homo sapiens] | 9.1\| | | | | | | | |
| complement component 4B | gi\|50345296\|ref\|NP_00100202 | 192735 | 117 | KADGSYAAWLSR | 1324.66 | 1061 | 1072 | |
| preproprotein [Homo sapiens] | 9.1\| | | | | | | | |
| complement component 4B | gi\|50345296\|ref\|NP_00100202 | 192735 | 118 | LHLETDSLALVALGAL | 2613.41 | 589 | 614 | |
| preproprotein [Homo sapiens] | 9.1\| | | | DTALYAAGSK | | | | |
| complement component 4B | gi1503452961ref\|NP_00100202 | 192735 | 119 | SCGLHQLLR | 1083.57 | 96 | 104 | |
| preproprotein [Homo sapiens] | 9.1\|,gi\|67190748\|ref\|NP_00922 | | | | | | | |
| | 4.2\| | | | | | | | |
| complement component 4B | gi\|50345296\|ref\|NP00100202 | 192735 | 120 | TYNVLDMK | 983.49 | 1383 1390 | | |
| preproprotein [Homo sapiens] | 9.1\|,gi\|67190748\|ref\|NP_00922 | | | | | | | |
| 4.2\| | | | | | | | | |
| complement component 4B | gi\|50345296\|ref\|NP_00100202 | 192735 | 121 | VDFTLSSER | 1053.52 | 72 | 80 | |
| preproprotein [Homo sapiens] | 9.1\| | | | | | | | |
| complement component 4B | gi\|50345296\|ref\|NP_00100202 | 192735 | 122 | VGLSGMAIADVTLLSG | 2128.16 | 1478 1498 | | |
| preproprotein [Homo sapiens] | 9.1\| | | | FHALR | | | | |
| complement component 4B | gi\|50345296\|ref\|NP_00100202 | 192735 | 123 | VLSLAQEQVGGSPEK | 1541.82 | 1085 | 1099 | |
| preproprotein [Homo sapiens] | 9.1\| | | | | | | | |
| complement component 4B | gi\|50345296\|ref\|NP_00100202 | 192735 | 124 | YVSHFETEGPHVLLYF | 2680.30 | 1511 | 1533 | |
| preproprotein [Homo sapiens] | 9.1\| | | | DSVPTSR | | | | |
| complement component 5 [Homo | gi\|38016947\|ref\|NP_001726.2\| | 188291 | 125 | FQNSAILTIQPK | 1359.76 | 79 | 90 | |
| sapiens] | | | | | | | | |
| complement component 5 [Homo | gi\|38016947\|ref\|NP_001726.2\| | 188291 | 126 | MVETTAYALLTSLNLK | 1783.95 | 1247 | 1262 | |
| sapiens] | | | | | | | | |
| complement component 5 [Homo | gi\|38016947\|ref\|NP_001726.2\| | 188291 | 127 | VFQFLEK | 910.50 | 623 | 629 | |
| sapiens] | | | | | | | | |
| complement components [Homo | gi\|38016947\|ref\|NP_001726.2\| | 188291 | 128 | YIYPLDSLTWIEYWPR | 2115.06 | 1635 | 1650 | |
| sapiens] | | | | | | | | |
| core-binding factor, runt domain, | gi\|4826663\|ref\|NP_005084.11 | 67115 | 143 | MAKESGISLKEIQVLA | 1873.06 | 1 | 17 | |
| alpha subunit 2; translocated to, 2 | | | | R | | | | |
| isoform MTGR1b [Homo sapiens] | | | | | | | | |
| delta globin [Homo sapiens] | gi\|4504351\|ref1NP_000510.1\| | 16037 | 144 | EFTPQMQAAYQK | 1441.68 | 122 | 133 | |
| delta globin [Homo sapiens] | gi\|4504351\|ref\|NP_000510.1\| | 16037 | 145 | GTFSQLSELHCDK | 1521.70 | 84 | 96 | |
| delta globin [Homo sapiens] | gi\|4504351\|ref\|NP_000510.1\| | 16037 | 146 | GTFSQLSELHCDKLHV | 2629.25 | 84 | 105 | |
| | | | | DPENFR | | | | |
| delta globin [Homo sapiens] | gi\|4504351\|ref\|NP_000510.1\| | 16037 | 147 | LLGNVLVCVLAR | 1326.79 | 106 | 117 | |
| delta globin [Homo sapiens] | gi\|4504351\|ref\|NP_000510.1\| | 16037 | 148 | LLVVYPWTQR | 1274.73 | 32 | 41 | |
| delta globin [Homo sapiens] | gi\|4504351\|ref\|NP_000510.1\| | 16037 | 149 | TAVNALWGK | 959.53 | 10 | 18 | |
| delta globin [Homo sapiens] | gi\|4504351\|ref\|NP_000510.1\| | 16037 | 150 | VLGAFSDGLAHLDNL | 1669.89 | 68 | 83 | |
| | | | | K | | | | |
| delta globin [Homo sapiens] | gi\|4504351\|ref\|NP_000510.1\| | 16037 | 151 | VNVDAVGGEALGR | 1256.66 | 19 | 31 | |
| delta globin [Homo sapiens] | gi\|4504351\|ref\|NP_000510.1\| | 16037 | 152 | VVAGVANALAHK | 1149.67 | 134 | 145 | |
| delta globin [Homo sapiens] | gi\|4504351\|ref\|NP_000510.1\| | 16037 | 153 | VVAGVANALAHKYH | 1449.80 | 134 | 147 | |
| diacyiglycerol kinase, theta [Homo | gi\|40806175\|ref1NP_001338.21 | 101135 | 156 | PGSPACSPVLGSGGRA | 1625.82 | 20 | 36 | |
| sapiens] | | | | R | | | | |
| Fibrinogen alpha chain precursor | FIBA_HUMAN | 94955 | 157 | TFPGFFSPMLGEFVSET | 2265.0 | 5528 | 547 | |
| [Contains: Fibrinopeptide A] | | | | ESR | | | | |
| Fibrinogen alpha chain precursor | FIBA_HUMAN | 94955 | 158 | VTSGSTTTTR | 1010.51 | 449 | 458 | |
| [Contains: Fibrinopeptide A] | | | | | | | | |
| fibrinogen, alpha polypeptide isoform | gi\|11761629\|ref1NP_068657.1\|, | 69739 | 159 | ESSSHHPGIAEFPSR | 1637.77 | 559 | 573 | |
| alpha preproprotein [Homo sapiens] | gi\|4503689\|ref\|NP_000499.1\| | | | | | | | |
| fibrinogen, alpha polypeptide isoform | gi\|11761629\|ref\|NP_068657.1\|, | 69739 | 160 | EVDLKDYEDQQK | 1509.71 | 191 | 202 | |
| alpba preproprotein [Homo sapiens] | gi\|45O3689\|ref\|NP_000499.1\| | | | | | | | |
| fibrinogen, alpha polypeptide isoform | gi\|11761629\|ref\|NP_068657.1\|, | 69739 | 161 | GLIDEVNQDFTNR | 1520.73 | 72 | 84 | |
| alpha preproprotein [Homo sapiens] | gi\|4SO3689\|ref\|NP_000499.1\| | | | | | | | |
| fibrinogen, alpha polypeptide isoform | gi\|11761629\|ref\|NP_068657.1\|, | 69739 | 162 | GSESGIFTNTK | 1140.55 | 548 | 558 | |
| alpha preproprotein [Homo sapiens] | gi\|4503689\|ref\|NP_000499.1\| | | | | | | | |
| fibrinogen, alpha polypeptide isoform | gi\|11761629\|ref\|NP_068657.1\|, | 69739 | 163 | HPDEAAFFDTASTGK | 1593.72 | 513 | 527 | |
| alpha preproprotein [Homo sapiens] | gi\|4SO3689\|ref\|NP_000499.1\| | | | | | | | |
| fibrinogen, alpha polypeptide isoform | gi\|11761629\|ref\|NP_068657.1\|, | 69739 | 164 | HRHIPDEAAFFDTASTG | | 1886.88 | 511 | 527 |
| alpha preproprotein [Homo sapiens] | gi\|4503689\|ref\|NP_000499.1\| | | | K | | | | |
| fibrinogen, alpha polypeptide isoform | gi\|11761629\|ref\|NP_068657.1\|, | 69739 | 165 | TFPGFFSPMLGEFVSET | 2265.05 | 528 | 547 | |
| alpha preproprotein [Homo sapiens] | gi\|4503689\|ref\|NP_000499.1\| | | | ESR | | | | |
| gelsolin isoform a precursor [Homo | gi\|4504165\|ref\|NP_000168.1\| | 85680 | 166 | DSQEEEKTEALTSAK | 1665.78 | 714 | 728 | |
| sapiens] | | | | | | | | |
| gelsolin isoform a precursor [Homo | gi\|4504165\|refaNP_000168.1\| | 85680 | 167 | EVQGFESATFLGYFK | 1722.84 | 148 | 162 | |
| sapiens] | | | | | | | | |
| gelsolin isoform a precursor [Homo | gi\|4504165\|ref\|NP_000168.1\| | 85680 | 168 | FDLVPVPTNLYGDFFT | 2704.39 | 76 | 99 | |
| sapiens] | | | | GDAYVILK | | | | |
| gelsolin isoform a precursor [Homo | gi\|4504165\|ref\|NP_000168.1\| | 85680 | 169 | HVVPNEVVVQR | 1275.72 | 178 | 188 | |
| sapiens] | | | | | | | | |
| gelsolin isoform a precursor [Homo | gi\|4504165\|ref\|NP_000168.1\| | 85680 | 170 | PNSMVVEHPEFLK | 1526.77 | 49 | 61 | |
| sapiens] | | | | | | | | |
| Hepatitis B virus receptor binding | Q6PYX1 | 38143 | 172 | GQGTTVTVSSASTK | 1323.68 | 9 | 22 | |
| protein (Fragment) | | | | | | | | |
| Hepatitis B virus receptor binding | Q6PYX1 | 38143 | 173 | GTTVTVSSASTK | 1138.60 | 11 | 22 | |
| protein (Fragment) | | | | | | | | |
| histidine-rich glycoprotein precursor | gi\|4504489\|ref\|NP_000403.1\| | 59559 | 174 | ALDLINKR | 942.57 | 34 | 41 | |
| [Homo sapiens] | | | | | | | | |
| histidine-rich glycoprotein precursor | gi\|4504489\|refINP_000403.1\| | 59559 | 175 | DGYLFQLLR | 1124.61 | 44 | 52 | |
| [Homo sapiens] | | | | | | | | |
| histidine-rich glycoprotein precursor | gi\|4504489\|ref\|NP_000403.1\| | 59559 | 176 | DSPVLIDFFEDTER | 1682.79 | 140 | 153 | |
| [Homo sapiens] | | | | | | | | |
| histidine-rich glycoprotein precursor | gi\|4504489\|ref\|NP_000403.11 | 59559 | 177 | GEVLPLPEANFPSFPLP | 2226.17 | 467 | 486 | |
| [Homo sapiens] | | | | HHK | | | | |
| histidine-rich glycoprotein precursor | gi\|4504489\|refINP_000403.1\| | 159559 | 178 | HPLKPDNQPFPQSVSE | 2349.13 | 487 | 507 | |
| [Homo sapiens] | | | | SCPGK | | | | |
| histidine-rich glycoprotein precursor | gi\|4504489\|ref\|NP_000403.1\| | 59559 | 179 | KGEVLPLPEANFPSFPL | 2354.27 | 466 | 486 | |
| [Homo sapiens] | | | | PHHK | | | | |
| histidine-rich glycoprotein precursor | gi\|4504489\|ref\|NP_000403.1\| | 59559 | 180 | LPPLR | 595.39 | 461 | 465 | |
| [Homo sapiens] | | | | | | | | |
| histidine-rinch glycoprotein precursor | gi\|4504489\|ref\|NP_000403.11 | 59559 | 181 | RDGYLFQLLR | 1280.71 | 43 | 52 | |
| [Homo sapiens] | | | | | | | | |
| histidine-rich glycoprotein precursor | gi\|4504489\|ref\|NP_000403.1\| | 59559 | 182 | RPSEIVIGQCK | 1286.69 | 96 | 106 | |
| [Homo sapiens] | | | | | | | | |
| histidine-rich glycoprotein precursor | gi\|4504489\|ref\|NP_000403.1\| | 59559 | 183 | SGFPQVSMFFTHTFPK | 1857.90 | 510 | 525 | |
| [Homo sapiens] | | | | | | | | |
| Keratin, type I cytoskeletal 10 | KIC10_HUMAN | 59502 | 263 | AETECQNTEYQQLLDI | 2352.15 | 423 | 441 | |
| (Cytokeratin-10) (CK-10) (Keratin- | | | | KIR | | | | |
| 10) (K10) | | | | | | | | |
| Keratin, type I cytoskeletal 10 | KIC10_HUMAN | 59502 | 264 | GSLGGGFSSGGFSGGS | 1464.6 | 441 | 57 | |
| (Cytokeratin-10) (CK-10) (Keratin- | | | | F | | | | |
| 10) (K10) | | | | | | | | |
| Keratin, type I cytoskeletal 10 | KIC10_HUMAN | 59502 | 265 | ILLQIDNAR | 1055.62 | 220 | 228 | |
| (Cytokeratin-10) (CK-10) (Keratin- | | | | | | | | |
| 10) (K10) | | | | | | | | |
| Keratin, type I cytoskeletal 10 | KIC10_HUMAN | 59502 | 266 | IRLENEIQTYR | 1434.77 | 440 | 450 | |
| (Cytokeratin-10) (CK-10) (Keratin- | | | | | | | | |
| 10) (K10) | | | | | | | | |
| Keratin, type I cytoskeletal 10 | KIC10_HUMAN | 59502 | 267 | ISALEEQLQQIR | 1427.79 | 411 | 422 | |
| (Cytokeratin-10) (CK-10) (Keratin- | | | | | | | | |
| 10) (K10) | | | | | | | | |
| Keratin, type I cytoskeletal 10 | KIC10_HUMAN | 59502 | 268 | LAADDFR | 807.40 | 229 | 235 | |
| (Cytokeratin-10) (CK-10) (Keratin- | | | | | | | | |
| 10) (K10) | | | | | | | | |
| Keratin, type I cytoskeletal 10 | KIC10_HUMAN | 59502 | 269 | LASYLDK | 809.44 | 157 | 163 | |
| (Cytokeratin-10) (CK-10) (Keratin- | | | | | | | | |
| 10) (K10) | | | | | | | | |
| Keratin, type I cytoskeletal 10 | KIC10_HUMAN | 59502 | 270 | LENEIQTYR | 1165.59 | 442 | 450 | |
| (Cytokeratin-10) (CK-10) (Keratin- | | | | | | | | |
| 10) (K10) | | | | | | | | |
| Keratin, type I cytoskeletal 10 | KIC10_HUMAN | 59502 | 271 | LKYENEVALR | 1234.68 | 236 | 245 | |
| (Cytokeratin-10) (CK-10) (Keratin- | | | | | | | | |
| 10) (K10) | | | | | | | | |
| Keratin, type I cytoskeletal 10 | KIC10_HUMAN | 59502 | 272 | NQILNLTTDNAN | 1330.66 | 208 | 219 | |
| (Cytokeratin-10) (CK-10) (Keratin- | | | | | | | | |
| 10) (K10) | | | | | | | | |
| Keratin, type I cytoskeletal 10 | KIC10_HUMAN | 59502 | 273 | NQILNLTTDNANILLQI | 2367.26 | 208 | 228 | |
| (Cytokeratin-10) (CK-10) (Keratin- | | | | DNAR | | | | |
| 10) (K10) | | | | | | | | |
| Keratin, type I cytoskeletal 10 | KIC10_HUMAN | 59502 | 274 | NVQALEIELQSQLALK | 1797.01 | 371 | 386 | |
| (Cytokeratin-10) (CK-10) (Keratin- | | | | | | | | |
| 10) (K10) | | | | | | | | |
| Keratin, type I cytoskeletal 10 | KIC10_HUMAN | 59502 | 275 | RVLDELTLTK | 1187.70 | 257 | 266 | |
| (Cytokeratin-10)(CK-10)(Keratin- | | | | | | | | |
| 10) (K10) | | | | | | | | |
| Keratin, type I cytoskeletal 10 | KIC10_HUMAN | 59502 | 276 | SGGGGGGGGCGGGGG | 1549.68 | 16 | 35 | |
| (Cytokeratin-10) (CK-10) (Keratin- | | | | VSSLR | | | | |
| 10) (K10) | | | | | | | | |
| Keratin, type I cytoskeletal 10 | KIC10_HUMAN | 59502 | 277 | SKELTTEIDNNIEQISSY | 2212.10 | 334 | 362 | |
| (Cytokeratin-10) (CK-10) (Keratin- | | | | K | | | | |
| 10) (K10) | | | | | | | | |
| Keratin, type I cytoskeletal 10 | KIC10_HUMAN | 59502 | 278 | SLLEGEGSSGGGGR | 1262.60 | 451 | 464 | |
| (Cytokeratin-10) (CK-10) (Keratin- | | | | | | | | |
| 10) (K10) | | | | | | | | |
| Keratin, type I cytoskeletal 10 | KIC10_HUMAN | 59502 | 279 | VQALEIELQSQLALK | 1682.97 | 372 | 386 | |
| (Cytokeratin-10) (CK-10) (Keratin- | | | | | | | | |
| 10) (K10) | | | | | | | | |
| Keratin, type II cytoskeletal 2 | K22E_HUMAN | 65848 | 310 | GFSSGSAVVSGGSR | 1254.61 | 21 | 34 | |
| epidermal (Cytokeratin-2e) (K2e) | | | | | | | | |
| (CK 2e) | | | | | | | | |
| Keratin, type II cytoskeletal 2 | K22E_HUMAN | 65848 | 311 | IEISELNR | 973.53 | 400 | 407 | |
| epidermal (Cytokeratin-2e) (K2e) | | | | | | | | |
| (CK 2e) | | | | | | | | |
| Keratin, type II cytoskeletal 2 | K22E_HUMAN | 65848 | 312 | LALDVEIATYR | 1263.69 | 477 | 487 | |
| epidermal (Cytokeratin-2e) (K2e) | | | | | | | | |
| (CK 2e) | | | | | | | | |
| Keratin, type II cytoskeletal 2 | K22E_HUMAN | 65848 | 313 | LNDLEEALQQAK | 1371.71 | 448 | 459 | |
| epidermal (Cytokeratin-2e) (K2e) | | | | | | | | |
| (CK 2e) | | | | | | | | |
| Keratin, type II cytoskeletal 2 | K22E_HUMAN | 65848 | 314 | NLDLDSIIAEVK | 1329.73 | 348 | 359 | |
| epidermal (Cytokeratin-2e) (K2e) | | | | | | | | |
| (CK 2e) | | | | | | | | |
| Keratin, type II cytoskeletal 2 | K22E_HUMAN | 65848 | 315 | NVQDAIADAEQR | 1329.64 | 425 | 436 | |
| epidermal (Cytokeratin-2e) (K2e) | | | | | | | | |
| (CK 2e) | | | | | | | | |
| Keratin, type II cytoskeletal 2 | K22E_HUMAN | 65848 | 316 | VDLLNQEIEFLK | 1460.80 | 309 | 320 | |
| epidermal (Cytokeratin-2e) (K2e) | | | | | | | | |
| (CK 2e) | | | | | | | | |
| Keratin, type II cytoskeletal 2 | K22E_HUMAN | 65848 | 317 | YEDEINKR | 1066.52 | 274 | 281 | |
| epidermal (Cytokeratin-2e) (K2e) | | | | | | | | |
| (CK2e) | | | | | | | | |
| Keratin, type II cytoskeletal 2 | K22E_HUMAN | 65848 | 318 | YEELQVTVGR | 1193.62 | 381 | 390 | |
| epidermal (Cytokeratin-2e) (K2e) | | | | | | | | |
| (CK 2e) | | | | | | | | |
| Keratin, type II cytoskeletal 2 | K22E_HUMAN | 65848 | 319 | YLDGLTAER | 1037.53 | 245 | 253 | |
| epidermal (Cytokeratin-2e) (K2e) | | | | | | | | |
| (CK2e) | | | | | | | | |
| KIAA1199 [Homo sapiens] | gi\|38638698\|ref\|NP_061159.1\| | 152983 | 320 | PFLSIISARYSPHQDAD | 2255.18 | 755 | 774 | |
| | | | | PLK | | | | |
| lipocalin 2 (oncogene 24p3) [Homo | gi\|38455402\|ref\|NP_005555.21 | 22571 | 321 | TFVPGCQPGEFTLGNI | 1864.93 | 102 | 118 | |
| sapiens] | | | | K | | | | |
| lipocalin 2 (oncogene 24p3) [Homo | gi\|38455402\|ref\|NP_005555.21 | 22571 | 322 | VPLQQNFQDNQFQGK | 1790.88 | 36 | 50 | |
| sapiens] | | | | | | | | |
| lysozyme precursor [Homo sapiens] | gi\|45S7894\|ref\|NP_000230.1\| | 16519 | 323 | GISLANWMCLAK | 1363.69 | 40 | 51 | |
| lysozyme precursor [Homo sapiens] | gi\|4S57894\|ref\|NP_000230.1\| | 16519 | 324 | LGMDGYR | 811.38 | 33 | 39 | |
| lysozyme precursor [Homo sapiens] | gi\|4557894\|ref\|NP_000230.1\| | 16519 | 325 | QYVQGCGV | 910.41 | 141 | 148 | |
| lysozyme precursor [Homo sapiens] | gi\|4557894\|ref\|NP_000230.1\| | 16519 | 326 | RLGMDGYR | 967.48 | 32 | 39 | |
| lysozyme precursor [Homo sapiens] | gi\|45S7894\|ref\|NP_000230.1\| | 16519 | 327 | STDYGIFQINSR | 1400.68 | 69 | 80 | |
| lysozyme precursor [Homo sapiens] | gi\|4557894\|ref\|NP_000230.1\| | 16519 | 328 | TPGAVNACHLSCSALL | 2927.38 | 88 | 115 | |
| | | | | QDNIADAVACAK | | | | |
| lysozyme precursor [Homo sapiens] | gi\|4557894fref\|NP_000230.11\| | 16519 | 329 | WESGYNTR | 1012.45 | 52 | 59 | |
| mannan-binding lectin serine | gi\|21264363\|ref\|NP_006601.2\| | 75685 | 330 | VLATLCGQESTDTER | 1679.79 | 85 | 99 | |
| protease 2 isoform 1 precursor | | | | | | | | |
| [Homo sapiens] | | | | | | | | |
| matrix Gla protein [Homo sapiens] | gi\|49574514\|ref\|NP_000891.2\| | 12336 | 331 | NANTFISPQQR | 1275.64 | 39 | 49 | |
| membrane associated guanylate | gi\|66346708\|ref\|NP_004733.2\|, | 140360 | 332 | NPSELKGKFIHTK | 1498.84 | 462 | 474 | |
| kinase, WW and PDZ domain | gi\|74272282\|ref\|NP_056335.1\|, | | | | | | | |
| containing 1 isoform b [Homo | gi\|74272284\|ref\|NP_00102822 | | | | | | | |
| sapiens] | 9.1\| | | | | | | | |
| myosin IIIA [Homo sapiens] | gi\|24586657\|ref\|NP_059129.21 | 186070 | 334 | DKVNGDK | 775.40 | 89 | 95 | |
| myotubularin-related protein 9 | gi\|19923424\|ref\|NP_056273.21 | 63446 | 335 | AGKRGYIIDTR | 1249.70 | 227 | 237 | |
| [Homo sapiens] | | | | | | | | |
| phospholipase D1, | gi\|4505873\|ref\|NP_002653.1\| | 124170 | 348 | IAADMSNIIENLDTR | 1691.83 | 17 | 31 | |
| phophatidylcholine-specific [Homo | | | | | | | | |
| sapiens] | | | | | | | | |
| PREDICTED: similar to Neutrophil | gi\|13419903\|ref\|XP_0011279 | 10183 | 349 | ADEVAAAPEQIAADIP | 3204.63 | 26 | 56 | |
| defensin I precursor (KNP-I) (HP-1) | 29.1\|,gi\|4758146\|ref\|NP_00407 | | | EVVVSLAWDESLAPK | | | | |
| (HP1) (Defensin, alpha 1) [Homo | 5.1\|,gi\|4885179\|ref\|NP_00520 | | | | | | | |
| sapiens] | 8.1\| | | | | | | | |
| PREDICTED: similar to Neutrophil | gi\|\|13419903\|ref\|XP_0011279 | 10183 | 356 | IPACIAGER | 986.51 | 70 | 78 | |
| defensin 1 precursor (HNP-1) (HP-1) | 29.1\|,gi\|4758146\|ref\|NP_00407 | | | | | | | |
| (HP1) (Defensin, alpha 1) [Homo | 5.1\|,gi\|4885179\|ref\|NP_00520 | | | | | | | |
| sapiens] | 8.1\| | | | | | | | |
| PREDICTED: similar to Neutrophil | gi\|113419903\|ref1XP_0011279 | 10183 | 357 | RYGTCIYQGR | 1273.61 | 79 | 88 | |
| defensin I precursor (HNP-1) (HP-1) | 29.11,gi\|47581461ref\|NP_00407 | | | | | | | |
| (HP1) (Defensin, alpha 1) [Homo | 5.11,gi\|48851791ref\|NP_00520 | | | | | | | |
| sapiens] | 8.1\| | | | | | | | |
| PREDICTED: similar to Neutrophil | gi\|113419903\|ref1XP_0011279 | 10183 | 358 | YGTCIYQGR | 1117.51 | 80 | 88 | |
| defensin I precursor (HNP-1) (HP-1) | 29,1\|,gi\|4758146\|ref\|NP_00407 | | | | | | | |
| (HP1) (Defensin, alpha 1) [Homo | 5.1\|,gi\|4885179\|ref\|NP_00520 | | | | | | | |
| sapiens] | 8.1\| | | | | | | | |
| PREDICTED: similar to ribosomal | gi\|113426784\|ref\|XP_0011341 | 27009 | 359 | HMKIKSLEEMYVFSLP | 2259.12 | 12 | 29 | |
| protein S2 [Homo sapiens] | 58.1\|,gi\|113427383\|ref\|XP_001 | | | MK | | | | |
| | 126232.1\| | | | | | | | |
| prosaposin [Homo sapiens] | gi\|11386147\|ref\|NP_002769.1\| | 58094 | 362 | LGPGMADICK | 1061.51 | 233 | 242 | |
| pyruvate kinase 3 isoform I [Homo | gi\|33286418\|ref\|NP_002645.31, | 57920 | 363 | IYVDDGLISLQVK | 1462.82 | 174 | 186 | |
| sapiens] | gi\|33286420\|ref\|NP_872270.1\|, | | | | | | | |
| | gi\|33286422\|ref\|NP_872271.1\| | | | | | | | |
| ribosomal protein S6 kinase, 90kDa, | gi\|4759050\|ref\|NP_004577.1\| | 83721 | 365 | LGMPQFLSPEAQSLLR | 2306.24 285 | 304 | | |
| polypeptide 3 [Homo sapiens] | | | | MLFK | | | | |
| S100 calcium-binding protein A7 | gi\|506769\|ref\|NP_002954.1\| | 11440 | 372 | IDFSEFLSLLGDIATDY | 2184.09 | 70 | 88 | |
| [Homo sapiens] | | | | HK | | | | |
| S100 calcium-binding protein A7 | gi\|4506769\|ref\|NP_002954.1\| | 11440 | 373 | KIDFSEFLSLLGDIATD | 2312.18 | 69 | 88 | |
| [Homo sapiens] | | | | YHK | | | | |
| serine (or cysteine) proteinase | gi\|50363217\|ref\|NP_000286.3\|, | 46720 | 376 | AVLTIDEK | 888.50 | 360 | 367 | |
| inhibitor, clade A (alpha-1 | gi\|50363219\|ref\|NP_00100223 | | | | | | | |
| antiproteinase, antitrypsin), member 1 | 6.1\|,gi\|50363221\|ref\|NP_00100 | | | | | | | |
| [Homo sapiens] | 2235.1\| | | | | | | | |
| serine (or cysteine) proteinase | gi\|50363217\|ref1NP_000286.3\|, | 46720 | 377 | FLENEDR | 922.43 | 299 | 305 | |
| inhibitor, clade A (alpha-1 | gi\|50363219\|ref\|NP_00100223 | | | | | | | |
| antiproteinase, antitrypsin), member 1 | 6.1\|,gi\|50363221\|ref\|NP_00100 | | | | | | | |
| [Homo sapiens] | 2235.1\| | | | | | | | |
| serine (or cysteine) proteinase | gi\|50363217\|ref\|NP_000286.3\|, | 46720 | 378 | FNKPFVFLMIEQNTK | 1855.98 | 390 | 404 | |
| inhibitor, clade A (alpha-1 | gi\|50363219\|ref\|NP_00100223 | | | | | | | |
| antiproteinase, antitrypsin), member 1 | 6.1\|,gi\|50363221\|ref\|NP_00100 | | | | | | | |
| [Homo sapiens] | 2235.1\| | | | | | | | |
| serine (or cysteine) proteinase | gi\|50363217\|ref\|NP_000286.3\|, | 46720 | 379 | GTEAAGAMFLEAIPMS | 2259.14 | 368 | 389 | |
| inhibitor, clade A (alpha-1 | gi\|50363219\|ref\|NP_00100223 | | | IPPEVK | | | | |
| antiproteinase, antitrypsin), member 1 | 6.1\|,gi\|50363221\|ref\|NP_00100 | | | | | | | |
| [Homo sapiens] | 2235.1\| | | | | | | | |
| serine (or cysteine) proteinase | gi\|50363217\|ref\|NP_000286.3\|, | 46720 | 380 | ITPNLAEFAFSLYR | 1641.86 | 50 | 63 | |
| inhibitor, clade A (alpha-1 | gi\|50363219\|ref\|NP_00100223 | | | | | | | |
| antiproteinase, antitrypsin), member 1 | 6.1\|,gi\|50363221\|ref\|NP_00100 | | | | | | | |
| [Homo sapiens] | 2235.1\| | | | | | | | |
| serine (or cysteine) proteinase | gi\|50363217\|ref\|NP_000286.3\|, | 46720 | 381 | KLSSWVLLMK | 1204.71 | 258 | 267 | |
| inhibitor, clade A (alpha-1 | gi\|50363219\|ref\|NP_00100223 | | | | | | | |
| antiproteinase, antitrypsin), member 1 | 6.1\|,gi\|50363221\|ref\|NP_00100 | | | | | | | |
| [Homo sapiens] | 2235.1\| | | | | | | | |
| serine (or cysteine) proteinase | gi\|50363217\|ref\|NP_000286.3\|, | 46720 | 382 | LQHLENELTHDIITK | 1803.96 | 284 | 298 | |
| inhibitor, clade A (alpha-1 | gi\|50363219\|ref\|NP_00100223 | | | | | | | |
| antiproteinase, antitrypsin), member 1 | 6.1\|,gi\|50363221\|ref\|NP_00100 | | | | | | | |
| [Homo sapiens] | 2235.1\| | | | | | | | |
| serine (or cysteine) proteinase | gi\|50363217\|ref\|NP_000286.3\|, | 46720 | 383 | LSITGTYDLK | 1110.61 | 315 | 324 | |
| inhibitor, clade A (alpha-1 | gi\|50363219\|ref\|NP_00100223 | | | | | | | |
| antiproteinase, antitlypsin), member 1 | 6.1\|,gi\|50363221\|ref\|NP_00100 | | | | | | | |
| [Homo sapiens] | 2235.1\| | | | | | | | |
| serine (or cysteine) proteinase | gi\|50363217\|ref\|NP_000286.3\|, | 46720 | 384 | LSSWVLLMK | 1076.62 | 259 | 267 | |
| inhibitor; clade A (alpha-1 | gi\|50363219\|ref\|NP_00100223 | | | | | | | |
| antiproteinase, antitrypsin), member 1 | 6.1\|,gi\|50363221\|ref\|NP_00100 | | | | | | | |
| [Homo sapiens] | 2235.1\| | | | | | | | |
| serine (or cysteine) proteinase | gi\|50363217\|ref\|NP_000286.3\|, | 46720 | 385 | SASLHLPK | 852.49 | 307 | 314 | |
| inhibitor, clade A (alpha-1 | gi\|50363219\|ref\|NP_00100223 | | | | | | | |
| antiproteinase, antitrypsin), member 1 | 6.1\|,gi\|50363221\|ref\|NP_00100 | | | | | | | |
| [Homo sapiens] | 2235.1\| | | | | | | | |
| serine (or cysteine) proteinase | gi\|50363217\|ref\|NP_000286.3\|, | 46720 | 386 | SPLFMGK | 779.41 | 405 | 411 | |
| inhibitor, clade A (alpha-1 | gi\|5O363219\|ref\|NP_00100223 | | | | | | | |
| antiproteinase, antitrypsin), member 1 | 6.1\|,gi\|50363221\|ref\|NP_00100 | | | | | | | |
| [Homo sapiens] | 2235.1\| | | | | | | | |
| serine (or cysteine) proteinase | gi\|50363217\|ref\|NP_000286.3\|, | 46720 | 387 | SVLGQLGITK | 1015.62 | 325 | 334 | |
| inhibitor, clade A (alpha-1 | gi\|50363219\|ref\|NP_00100223 | | | | | | | |
| antiproteinase, antitrypsin), member 1 | 6.1\|,gi\|50363221\|ref\|NP_00100 | | | | | | | |
| [Homo sapiens] | 2235.1\| | | | | | | | |
| serine (or cysteine) proteinase | gi\|50363217\|ref\|NP_000286.3\|, | 46720 | 388 | VFSNGADLSGVTEEAP | 1833.92 | 335 | 352 | |
| inhibitor, clade A (alpha-1 | gi\|50363219fref\|NP_00100223 | | | LK | | | | |
| antiproteinase, antitrypsin), member 1 | 6.1\|,gi\|50363221\|ref\|NP_00100 | | | | | | | |
| [Homo sapiens] | 2235.1\| | | | | | | | |
| serine (or cysteine) proteinase | gi\|28076869\|ref\|NP_002965.1\|, | 44837 | 389 | FMFDLFQQFR | 1378.66 | 11 | 20 | |
| inhibitor, clade B (ovalbumin), | gi\|5902072\|ref\|NP_008850.1\| | | | | | | | |
| member 4 [Homo sapiens] | | | | | | | | |
| serum amyloid A2 [Homo sapiens] | gi\|13540475\|ref\|NP_110381.1\| | 13491 | 390 | DPNHFRPAGLPEKY | 1640.82 | 109 | 122 | |
| serum amyloid A2 [Homo sapiens] | gi\|13540475\|ref\|NP_110381.1\| | 13491 | 391 | EANYIGSDKYFHAR | 1670.79 | 44 | 57 | |
| serum amyloid A2 [Homo sapiens] | gi\|13540475\|ref\|NP_110381.1\| | 13491 | 392 | LTGHGAEDSLADQAA | 1697.81 | 86 | 102 | |
| | | | | NK | | | | |
| serum amyloid A2 [Homo sapiens] | gi\|l354O475\|ref\|NP_110381.1\| | 13491 | 393 | RGPGGAWAAEVISNA | 1611.84 | 65 | 80 | |
| | | | | R | | | | |
| serum amyloid A2 [Homo sapiens] | gi\|13540475\|ref\|NP_110381.1\| | 13491 | 394 | SFFSFLGEAFDGAR | 1550.73 | 20 | 33 | |
| serum amyloid A4, constitutive | gi\|10835095\|ref\|NP_006503.1\| | 14789 | 395 | AYWDIMISNHQNSNR | 1848.84 | 38 | 52 | |
| [Homo sapiens] | | | | | | | | |
| serum amyloid A4, constitutive | gi\|10835095\|ref\|NP_006503.1\| | 14789 | 396 | EALQGVGDMGR | 1132.54 | 27 | 37 | |
| [Homo sapiens] | | | | | | | | |
| serum amyloid A4, constitutive | gi\|10835095\|ref\|NP_006503.1\| | 14789 | 397 | FRPDGLPK | 929.52 | 121 | 128 | |
| [Homo sapiens] | | | | | | | | |
| serum amyloid A4, constitutive | gi\|10835095\|ref\|NP_006503.1\| | 14789 | 398 | GPGGVWAAK | 842.45 66 | 74 | | |
| [Homo sapiens] | | | | | | | | |
| serum amyloid A4, constitutive | gi\|10835095\|ref\|NP_006503.1\| | 14789 | 399 | SFFKEALQGVGDMGR | 1641.81 | 23 | 37 | |
| [Homo sapiens] | | | | | | | | |
| serum amyloid A4, constitutive | gi\|10835095\|ref\|NP_006503.1\| | 14789 | 400 | SNEKAEEWGR | 1205.56 | 104 | 113 | |
| [Homo sapiens] | | | | | | | | |
| serum amyloid A4, constitutive | gi\|10835095\|ref\|NP_006503.1\| | 14789 | 401 | VYLQGLIDYYLFGNSS | 2624.31 | 81 | 103 | |
| [Homo sapiens] | | | | TVLEDSK | | | | |
| stanniocalcin 2 precursor [Homo | gi\|4507267\|ref\|NP_003705.1\| | 33230 | 402 | EMVSQLQRECYLK | 1699.81 | 130 | 142 | |
| sapiens] | | | | | | | | |
| transferrin [Homo sapiens] | gi\|4557871\|ref\|NP_001054.11 | 77032 | 406 | AIAANEADAVTLDAG | 3954.02 | 70 | 107 | |
| | | | | LVYDAYLAPNNLKPV | | | | |
| | | | | VAEFYGSK | | | | |
| transferrin [Homo sapiens] | gi\|4557871\|ref\|NP_001054.1\| | 77032 | 407 | ASYLDCIR | 997.48 | 62 | 69 | |
| transferrin [Homo sapiens] | gi\|4557871\|ref\|NP_001054.1\| | 77032 | 408 | CSTSSLLEACTFR | 1531.69 | 684 | 696 | |
| transferrin [Homo sapiens] | gi\|4557871\|ref\|NP_001054.1\| | 77032 | 409 | DDTVCLAK | 921.44 | 652 | 659 | |
| transferrin [Homo sapiens] | gi\|4557871\|ref\|NP_001054.1\| | 77032 | 410 | DGAGDVAFVK | 978.49 | 216 | 225 | |
| transferrin [Homo sapiens] | gi\|4557871\|ref\|NP_001054.1\| | 77032 | 411 | DLLFRDDTVCLAK | 1565.80 | 647 | 659 | |
| transferrin [Homo sapiens] | gi\|4557871\|ref\|NP_001054.1\| | 77032 | 412 | DSGFQMNQLR | 1195.55 | 123 | 132 | |
| transferrin [Homo sapiens] | gi\|4557871\|ref\|NP_001054.1\| | 77032 | 413 | DYELLCLDGTR | 1354.63 | 577 | 587 | |
| transferrin [Homo sapiens] | gi\|4557871\|ref\|NP_001054.1\| | 77032 | 414 | EDPQTFYYAVAVVK | 1629.82 | 108 | 121 | |
| transferrin [Homo sapiens] | gi\|4557871\|ref\|NP_001054.1\| | 77032 | 415 | HSTIFENLANK | 1273.65 | 226 | 236 | |
| transferrin [Homo sapiens] | gi\|4557871\|ref\|NP_001054.1\| | 77032 | 416 | KASYLDCIR | 1125.57 | 61 | 69 | |
| transferrin [Homo sapiens] | gi\|4557871\|ref\|NP_001054.1\| | 77032 | 417 | KCSTSSLLEACTFR | 1659.78 | 683 | 696 | |
| transferrin [Homo sapiens] | gi\|4557871\|ref\|NP_001054.1\| | 77032 | 418 | KDSGFQMNQLR | 1323.65 | 122 | 132 | |
| transferrin [Homo sapiens] | gi\|4557871\|ref\|NP_001054.1\| | 77032 | 419 | KPVEEYANCHLAR | 1586.77 | 588 | 600 | |
| transferrin [Homo sapiens] | gi\|4557871\|ref\|NP_001054.1\| | 77032 | 420 | NLNEKDYELLCLDGTR | 1952.94 | 572 | 587 | |
| transferrin [Homo sapiens] | gi\|4557871\|ref\|NP_001054.1\| | 77032 | 421 | NPDPWAK | 827.41 | 565 | 571 | |
| transferrin [Homo sapiens] | gi\|4557871\|ref\|NP_001054.1\| | 77032 | 422 | SAGWNIPIGLLYCDLPE | 2171.10 | 144 | 162 | |
| | | | | PR | | | | |
| transferrin [Homo sapiens] | gi\|4557871\|ref\|NP_001054.1\| | 77032 | 423 | SVIPSDGPSVACVK | 1415.72 | 47 | 60 | |
| transferrin [Homo sapiens] | gi\|4557871\|ref\|NP_001054.1\| | 77032 | 424 | WCAVSEHEATK | 1317.59 | 27 | 37 | |
| transferrin [Homo sapiens] | gi\|4557871\|ref\|NP_001054.1\| | 77032 | 425 | YLGEEYVK | 1000.50 | 669 | 676 | |
| triggering receptor expressed on | gi\|30794266\|ref\|NP_835468.11 | 32661 | 426 | FLPEGCQPLVSSAVDR | 1774.88 | 54 | 69 | |
| myeloid cells-like 1 [Homo sapiens] | | | | | | | | |
| vitronectin precursor [Homo sapiens] | gi\|88853069\|ref\|NP_000629.3\| | 54288 | 436 | IYISGMAPR | 1007.53 | 354 | 362 | |
| vitronectin precursor [Homo sapiens] | gi\|88853069\|ref\|NP_000629.3\| | 54288 | 437 | MDWLVPATCEPIQSVF | 2574.21 | 422 | 443 | |
| | | | | FFSGDK | | | | |
| vitronectin precursor [Homo sapiens] | gi\|88853069\|ref\|NP_000629.3\| | 54288 | 438 | RVDTVDPPYPR | 1314.68 | 453 | 463 | |
| vitronectin precursor [Homo sapiens] | gi\|88853069\|ref\|NP_000629.3\| | 54288 | 439 | SIAQYWLGCPAPGHL | 1669.82 | 464 | 478 | |
| vitronectin precursor [Homo sapiens] | gi\|88853069\|ref\|NP_000629.3\| | 54288 | 440 | VDTVDPPYPR | 1158.58 | 454 | 463 | |

TABLE 3
Biomarkers associated with Mild Cognitive Impairment (MCI)

| | | | | | | | |
|---|---|---|---|---|---|---|---|
| | | Protein | | | Calculated | | |
| | Protein | molecular | SEQ | | Peptide | Peptide | |
| | accession | weight | ID | | Mass | start | Peptide |
| Protein name | numbers | (Da) | NO: | Peptide sequence | (AMU) | index | stop index |

| | | | | | | | |
|---|---|---|---|---|---|---|---|
| (O60687) Sushi-repeat-containing | O60687 | 52954 | 2 | DSADGTITRVTLR | 1404.74 | 212 | 224 |
| protein, X-linked 2 | | | | | | | |
| (P01764) Ig heavy chain VIII region | HV3C_HUMAN | 12565 | 4 | AEDTAVYYCAK | 1290.57 | 107 | 117 |
| VH26 precursor | | | | | | | |
| (P09758) Tumor-associated calcium | TACD2_HUMAN | 35692 | 14 | HRPTAGAFNHSDLDAELRR | 2163.08 | 160 | 178 |
| signal transducer 2 precursor (Pancreatic | | | | | | | |
| carcinoma marker protein GA733-1) | | | | | | | |
| (Cell surface glycoprotein Trop-2) | | | | | | | |
| (P10643) Complement component C7 | CO7_HUMAN | 93499 | 17 | AASGTQNNVLR | 1130.59 | 362 | 372 |
| precursor | | | | | | | |
| (P20701) Integrin alpha-L precursor | ITAL_HUMAN | 128804 | 23 | EGVNITICFQIKSLYPQFQGR | 2498.29 | 646 | 666 |
| (Leukocyte adhesion glycoprotein LFA- | | | | | | | |
| 1 alpha chain) (LFA-1A) (Leukocyte | | | | | | | |
| function associated molecule 1, alpha | | | | | | | |
| chain CD11a) | | | | | | | |
| (Q6N092) Hypothetical protein | Q6N092 | 56405 | 29 | SAVQGPPDR | 926.47 | 335 | 343 |
| DKFZp686K18196 (Fragment) | | | | | | | |
| (Q96S24) Hypothetical protein gs30 | Q96S24 | 3503 | 34 | GMGSDRTALSLQGAWGIFLST | 2606.31 | 3 | 26 |
| | | | | FYK | | | |
| adenylosuccinate synthase [Homo | gi\|34577063\|ref\|NP_00 | 50080 | 45 | DGVYFLYEALHGPPKK | 1833.95 | 233 | 248 |
| sapiens] | 1117.2\| | | | | | | |
| alpha 1B-glycoprotein [Homo sapiens] | gi\|21071030\|ref\|NP_57 | 54235 | 46 | ATWSGAVLAGR | 1088.59 | 407 | 417 |
| | 0602.2\| | | | | | | |
| alpha 1B-glycoprotein [Homo sapiens] | gi\|21071030\|ref\|NP_57 | 54235 | 47 | SLPAPWLSMAPVSWITPGLK | 2151.17 | 115 | 134 |
| | 0602.2\| | | | | | | |
| alpha 1B-glycoprotein [Homo sapiens] | gi\|21071030\|ref\|NP_57 | 54235 | 48 | SWVPHTFESELSDPVELLVAES | 2471.20 | 474 | 495 |
| | 0602.21 | | | | | | |
| alpha 1B-glycoprotein [Homo sapiens] | gi\|21071030\|ref\|NP_57 | 54235 | 49 | TPGAAANLELIFVGPQHAGNY | 2296.18 | 448 | 469 |
| | 0602.21 | | | R | | | |
| Alpha-1-antichymotrypsin precursor | AACT_HUMAN | 47635 | 50 | ADLSGITGAR | 960.51 | 341 | 350 |
| (ACT) [Contains: Alpha-1- | | | | | | | |
| antichymotrypsin His-Pro-less] | | | | | | | |
| Alpha-1-antichymotrypsin precursor | AACT_HUMAN | 47635 | 51 | AVLDVFEEGTEASAATAVK | 1907.96 | 361 | 379 |
| (ACT) [Contains: Alpha-1- | | | | | | | |
| antichymotrypsin His-Pro-less] | | | | | | | |
| Alpha-1-antichymotrypsin precursor | AACT_HUMAN | 47635 | 52 | DYNLNDILLQLGIEEAFTSK | 2296.17 | 321 | 340 |
| (ACT) [Contains: Alpha-1- | | | | | | | |
| antichymotrypsin His-Pro-less] | | | | | | | |
| Alpha-1-antichymotrypsin precursor | AACT_HUMAN | 47635 | 53 | ITLLSALVETR | 1215.73 | 380 | 390 |
| (ACT) [Contains: Alpha-1- | | | | | | | |
| antichymotrypsin His-Pro-less] | | | | | | | |
| Alpha-1-antichymotrypsin precursor | AACT_HUMAN | 47635 | 54 | NLAVSQVVHK | 1094.63 | 351 | 360 |
| (ACT) [Contains: Alpha-1- | | | | | | | |
| antichymotrypsin His-Pro-less] | | | | | | | |
| Alpha-1-antichymotrypsin precursor | AACT_HUMAN | 47635 | 55 | YNLNDILLQLGIEEAFTSK | 2181.14 | 322 | 340 |
| (ACT) [Contains: Alpha-1- | | | | | | | |
| antichymotrypsin His-Pro-less] | | | | | | | |
| apolipoprotein C-II precursor [Homo | gi\|32130518\|ref\|NP_00 | 11266 | 60 | ESLSSYWESAK | 1286.59 | 42 | 52 |
| sapiens] | 0474.21 | | | | | | |
| apolipoprotein C-II precursor [Homo | gi\|32130348\|ref1NP_00 | 11266 | 61 | LRDLYSK | 894.50 | 71 | 77 |
| sapiens] | 0474.21 | | | | | | |
| apolipoprotein C-II precursor [Homo | gi\|32130518\|ref\|NP_00 | 11266 | 62 | STAAMSTYTGIFTDQVLSVLK | 2233.14 | 78 | 98 |
| sapiens] | 0474.21 | | | | | | |
| apolipoprotein C-II precursor [Homo | gi\|32130518\|ref\|NP_00 | 11266 | 63 | STAAMSTYTGIFTDQVLSVLK | 2548.25 | 78 | 101 |
| sapiens] | 0474.21 | | | GEE | | | |
| apolipoprotein C-II precursor [Homo | gi\|32130518\|ref\|NP_00 | 11266 | 64 | TAAQNLYEK | 1037.53 | 53 | 61 |
| sapiens] | 0474.21 | | | | | | |
| apolipoprotein C-II precursor [Homo | gi\|32130518\|ref\|NP_00 | 11266 | 65 | TYLPAVDEK | 1035.54 | 62 | 70 |
| sapiens] | 0474.21 | | | | | | |
| apolipoprotein L1 isoform a precursor | gi\|21735614\|ref\|NP_00 | 43957 | 69 | ILQADQEL | 929.49 | 391 | 398 |
| [Homo sapiens] | 3652.2\|,gi\|21735616\|re | | | | | | |
| | f\|NP_663318.1\| | | | | | | |
| apolipoprotein L1 isoform a precursor | gi\|21735614\|ref\|NP_00 | 43957 | 70 | LNILNNNYK | 1105.60 | 382 | 390 |
| [Homo sapiens] | 3652.2\|,gi\|21735616\|re | | | | | | |
| | f\|NP_663318.1\| | | | | | | |
| apolipoprotein M [Homo sapiens] | gi\|22091452\|ref\|NP_06 | 21236 | 71 | EELATFDPVDNIVFNMAAGSA | 3086.51 | 58 | 85 |
| | 1974.2\| | | | PMQLHLR | | | |
| apolipoprotein M [Homo sapiens] | gi\|22091452\|ref\|NP_O6 | 21236 | 72 | EFPEVHLGQWYFIAGAAPTK | 2261.14 | 38 | 57 |
| | 1974.2\| | | | | | | |
| apolipoprotein M [Homo sapiens] | gi\|22091452\|ref\|NP_06 | 21236 | 73 | KWIYHLTEGSTDLR | 1718.89 | 99 | 112 |
| | 1974.2\| | | | | | | |
| apolipoprotein M [Homo sapiens] | gi\|22091452\|ref\|NP_06 | 21236 | 74 | MKDGLCVPR | 1091.53 | 90 | 98 |
| | 1974.2\| | | | | | | |
| apolipoprotein M [Homo sapiens] | gi\|22091452\|ref\|NP_06 | 21236 | 75 | SLTSCLDSK | 1010.48 | 163 | 171 |
| | 1974.2\| | | | | | | |
| beta globin [Homo sapiens] | gi\|450349\|ref\|NP_000 | 15980 | 76 | EFTPPVQAAYQK | 1378.70 | 122 | 133 |
| | 509.1\| | | | | | | |
| beta globin [Homo sapiens] | gi\|4504349\|ref\|NP_000 | 15980 | 77 | FFESFGDLSTPDAVMGNPK | 2058.95 | 42 | 60 |
| | 509.1\| | | | | | | |
| beta globin [Homo sapiens] | gi\|4504349\|ref\|NP_000 | 15980 | 78 | FFESFGDLSTPDAVMGNPKVK | 2286.11 | 42 | 62 |
| | 509.1\| | | | | | | |
| beta globin [Homo sapiens] | gi\|4504349\|ref\|NP_000 | 15980 | 79 | GTFATLSELHCDK | 1478.70 | 84 | 96 |
| | 509.1\| | | | | | | |
| beta globin [Homo sapiens] | gi\|4504349\|ref\|NP_000 | 15980 | 80 | GTFATLSELHCDKLHVDPENFR | 2586.24 | 84 | 105 |
| | 509.1\| | | | | | | |
| beta globin [Homo sapiens] | gi\|4504349\|ref\|NP_000 | 15980 | 81 | KVLGAFSDGLAHLDNLK | 1797.99 | 67 | 83 |
| | 509.1\| | | | | | | |
| beta globin [Homo sapiens] | gi\|4504349\|ref\|NP_000 | 15980 | 82 | LHVDPENFR | 1126.56 97 | 105 | |
| | 509.1\| | | | | | | |
| beta globin [Homo sapiens] | gi\|4504349\|ref\|NP_000 | 15980 | 83 | LLGNVLVCVLAHHFGK | 1776.99 | 106 | 121 |
| | 509.1\| | | | | | | |
| beta globin [Homo sapiens] | gi\|4504349\|ref\|NP_000 | 15980 | 84 | LLVVYPWTQR | 1274.73 | 32 | 41 |
| | 509.1\| | | | | | | |
| beta globin [Homo sapiens] | gi\|4504349\|ref\|NP_000 | 15980 | 85 | LLVVYPWTQRFFESFGDLSTP | 3314.66 | 32 | 60 |
| | 509.1\| | | | DAVMGNPK | | | |
| beta globin [Homo sapiens] | gi\|4504349ref\|NP_000 | 15980 | 86 | SAVTALWGK | 932.52 | 10 | 18 |
| | 509.1\| | | | | | | |
| beta globin [Homo sapiens] | gi\|4504349\|ref\|NP_000 | 15980 | 87 | SAVTALWGKVNVDEVGGEAL | 2228.17 | 10 | 31 |
| | 509.1\| | | | GR | | | |
| beta globin [Homo sapiens] | gi\|4504349\|ref\|NP_000 | 15980 | 88 | SAVTALWGKVNVDEVGGEAL | 3483.87 | 10 | 41 |
| | 509.1\| | | | GRLLVVYPWTQR | | | |
| beta globin [Homo sapiens] | gi\|4504349\|ref\|NP_000 | 15980 | 89 | VLGAFSDGLAHLDNLK | 1669.89 | 68 | 83 |
| | 509.1\| | | | | | | |
| beta globin [Homo sapiens] | gi\|4504349\|ref\|NP_000 | 15980 | 90 | VNVDEVGGEALGR | 1314.67 | 19 | 31 |
| | 509.1\| | | | | | | |
| beta globin [Homo sapiens] | gi\|4504349\|ref\|NP_000 | 15980 | 91 | VVAGVANALAHK | 1149.67 | 134 | 145 |
| | 509.1\| | | | | | | |
| beta globin [Homo sapiens] | gi\|4504349\|ref\|NP_000 | 15980 | 92 | VVAGVANALAHKYH | 1449.80 | 134 | 147 |
| 509.1\| | | | | | | | |
| coagulation factor II precursor [Homo | gi\|4503635\|ref\|NP_000 | 70019 | 102 | ELLESYIDGR | 1194.60 | 354 | 363 |
| sapiens] | 497.1\| | | | | | | |
| coagulation factor II precursor [Homo | gi\|4503635\|ref\|NP_000 | 70019 | 103 | HQDFNSAVQLVENFCR | 1963.91 | 248 | 263 |
| sapiens] | 497.1\| | | | | | | |
| coagulation factor II precursor [Homo | gi\|4503635\|ref\|NP_000 | 70019 | 104 | IVEGSDAEIGMSPWQVMLFR | 2265.11 | 364 | 383 |
| sapiens] | 497.11 | | | | | | |
| coagulation factor II precursor [Homo | gi\|4503635ref\|NP_000 | 70019 | 105 | LAVITHGLPGLAWASAQAK | 1995.05 | 225 | 243 |
| sapiens] | 497.1\| | | | | | | |
| coagulation factor II precursor [Homo | gi\|4503635\|ref\|NP_000 | 70019 | 106 | SEGSSVNLSPPLEQCVPDR | 2070.98 | 199 | 217 |
| sapiens] | 497.1\| | | | | | | |
| coagulation factor II precursor [Homo | gi\|4503635\|ref\|NP_000 | 70019 | 107 | SLEDKTERELLESYIDGR | 2153.07 | 346 | 363 |
| sapiens] | 497.1\| | | | | | | |
| coagulation factor II precursor [Homo | gi\|4503635\|ref\|NP_000 | 70019 | 108 | TATSEYQTFFNPR | 1561.73 | 315 | 327 |
| sapiens] | 497.1\| | | | | | | |
| coagulation factor II precursor [Homo | gi\|4503635\|ref\|NP_000 | 70019 | 109 | TFGSGEADCGLRPLFEK | 1883.90 | 328 | 344 |
| sapiens] | 497.1\| | | | | | | |
| coagulation factor II precursor [Homo | gi\|4503635\|ref\|NP_000 | 70019 | 110 | YGFYTHVFR | 1189.58 | 600 | 608 |
| sapiens] | 497.1\| | | | | | | |
| complement component 8, gamma | gi14557393\|ref\|NP_000 | 22201 | 129 | AEATTLHVAPQGTAMAVSTFR | 2159.09 | 70 | 90 |
| polypeptide [Homo sapiens] | 597.1\| | | | | | | |
| complement componeni 8, gamma | gi\|4557393\|ref\|NP_000 | 22201 | 130 | ANFDAQQFAGTWLLVAVGSA | 2382.17 | 40 | 61 |
| polypeptide [Homo sapiens] | 597.1\| | | | CR | | | |
| complement component 8, gamma | gi\|4557393\|ref\|NP_000 | 22201 | 131 | GAVHVVVAETDYQSFAVLYLE | 2466.27 | 121 | 142 |
| polypeptide [Homo sapiens] | 597.1\| | | | R | | | |
| complement component 8, gamma | gi\|4557393\|ref\|NP_000 | 22201 | 132 | KLDGICWQVR | 1274.67 | 91 | 100 |
| nolypeptide [Homo sapiens] | 597.1\| | | | | | | |
| complement component 8, gamma | gi\|4557393\|ref\|NP_000 | 22201 | 133 | LDGICWQVR | 1146.57 | 92 | 100 |
| polypeptide [Homo sapiens] | 597.1\| | | | | | | |
| complement component 8, gamma | gi\|4557393\|ref\|NP_000 | 22201 | 134 | QLYGDTGVLGR | 1178.62 | 101 | 111 |
| polypeptide [Homo sapiens] | 597.1\| | | | | | | |
| complement component 8, gamma | gi\|4557393\|ref\|NP_\|000 | 22201 | 135 | RPASPISTIQPK | 1294.75 | 28 | 39 |
| polypeptide [Homo sapiens] | 597.1\| | | | | | | |
| complement component 8, gamma | gi\|4557393\|ref\|NP_000 | 22201 | 136 | SLPVSDSVLSGFEQR | 1620.82 | 154 | 168 |
| polypeptide [Homo sapiens] | 597.1\| | | | | | | |
| complement component 8, gamma | gi\|4557393\|ref\|NP_000 | 22201 | 137 | VQEAHLTEDQIFYFPK | 1964.98 | 169 | 184 |
| polypeptide [Homo sapiens] | 597.1\| | | | | | | |
| complement component 8, gamma | gi\|4557393˜refjNP_000 | 22201 | 138 | YGFCEAADQFHVLDEVR | 2055.92 | 185 | 201 |
| nolypeptide [Homo sapiens] | 597.1\| | | | | | | |
| complement factor H isoform a | gi\|62739186\|ref\|NP_00 | 139052 | 139 | EIMENYNIALR | 1365.68 | 1172 | 1182 |
| recursor [Homo sapiens] | 0177.2\| | | | | | | |
| complement factor H isoform a | gi\|62739186\|ref\|NP_00 | 139052 | 140 | KGEWVALNPLR | 1282.73 | 68 | 78 |
| precursor [Homo sapiens] | 0177.2\| | | | | | | |
| complement factor H isoform a | gi\|62739186\|ref\|NP_00 | 139052 | 141 | NTEILTGSWSDQTYPEGTQAI | 2602.23 | 29 | 51 |
| precursor [Homo sapiens] | 0177.2\|,gi\|62739188\|re | | | YK | | | |
| | f\|NP_001014975.\| | | | | | | |
| complement factor H isoform a | gi\|62739186\|ref\|NP_00 | 139052 | 142 | SLGNVIMVCR | 1148.59 | 58 | 67 |
| recursor [Homo sapiens] | 0177.2\| | | | | | | |
| Desmoplakin (DP) (250/210 kDa | DESP_HUMAN | 331765 | 154 | ANSSATETINK | 1135.56 | 1517 | 1527 |
| paraneoplastic pemphigus antigen) | | | | | | | |
| Desmoplakin (DP) (250/210 kDa | DESP_HUMAN | 331765 | 155 | GFFDPNTEENLTYLQLK | 2028.99 | 2414 | 2430 |
| paraneoplastic pemphigus antigen) | | | | | | | |
| hect domain and RLDS [Homo sapiens] | gi\|7705931\|ref\|NP_057 | 116834 | 171 | RTTEMMPVYLDLNK | 1726.85 | 434 | 447 |
| | 407.1\| | | | | | | |
| Hypothetical protein DKFZp686J11235 | Q6MZW0 | 54441 | 184 | GTTTFAVTSILR | 1266.71 | 441 | 452 |
| Hypothetical protein DKFZp686J11235 | Q6MZW0 | 54441 | 185 | TPLTATLSK | 931.55 | 366 | 374 |
| Hypothetical protein DKFZp686J11235 | Q6MZW0 | 54441 | 186 | VTVSSASPTSPK | 1160.62 | 149 | 160 |
| Hypothetical protein DKFZp781MO386 | Q5CZ94 | 24984 | 187 | AGVETTKPSK | 1017.56 | 179 | 188 |
| Hypothetical protein DKFZp781MO386 | QSCZ94 | 24984 | 188 | ANPTVTLFPPSSEELQANK | 2043.04 | 133 | 151 |
| Hypothetical protein DKFZp781MO386 | QSCZ94 | 24984 | 189 | VTVLGQPK | 841.51 | 125 | 132 |
| Ig heavy chain V-III region GAL | HV3T_HUMAN | 12708 | 191 | GLEWVANIK | 1029.57 | 44 | 52 |
| inter-alpha (globulin) inhibitor H4 | gi\|31542984\|ref\|NP_00 | 103340 | 193 | AEAQAQYSAAVAK | 1307.66 | 99 | 111 |
| [Homo sapiens] | 2209.2\| | | | | | | |
| inter-alpha (globulin) inhibitor H4 | gi\|315429841ref\|NP_00 | 103340 | 194 | AGFSWIEVTFK | 1284.66 | 778 | 788 |
| [Homo sapiens] | 2209.2\| | | | | | | |
| inter-alpha (globulin) inhibitor H4 | gi\|31542984\|ref\|NP_00 | 103340 | 195 | ANTVQEATFQMELPK | 1706.84 | 61 | 75 |
| [Homo sapiens] | 2209.2\| | | | | | | |
| inter-alpha (globulin) inhibitor H4 | gi\|31542984\|ref\|NP_00 | 103340 | 196 | FSSHVGGTLGQFYQEVLWGSP | 2968.39 | 867 | 894 |
| [Homo sapiens] | 2209.2\| | | | AASDDGR | | | |
| inter-alpha (globulin) inhibitor H4 | gi\|31542984\|ref\|NP_00 | 103340 | 197 | GPDVLTATVSGK | 1144.62 | 501 | 512 |
| [Homo sapiens] | 2209.2\| | | | | | | |
| inter-alpha (globulin) inhibitor H4 | gi\|31542984\|ref\|NP_00 | 103340 | 198 | LWAYLTIQQLLEQTVSASDAD | 2961.53 | 547 | 572 |
| [Homo sapiens] | 2209.2\| | | | QQALR | | | |
| inter-alpha (globulin) inhibitor H4 | gi\|31542984\|ref\|NP_00 | 103340 | 199 | MNFRPGVLSSR | 1263.66 | 658 | 668 |
| [Homo sapiens] | 2209.2\| | | | | | | |
| inter-alpha (globulin) inhibitor H4 | gi\|31542984\|ref\|NP_00 | 103340 | 200 | NVHSGSTFFK | 1123.55 | 617 | 626 |
| [Homo sapiens] | 2209.2\| | | | | | | |
| inter-alpha (globulin) inhibitor H4 | gi\|31542984\|ref\|NP_00 | 103340 | 201 | VTIGLLFWDGR | 1276.71 | 843 | 853 |
| [Homo sapiens] | 2209.2\| | | | | | | |
| keratin 1 [Homo sapiens] | gi\|17318569\|ref\|NP_00 | 66050 | 202 | AEAESLYQSK | 1125.54 | 367 | 376 |
| | 6112.2\| | | | | | | |
| keratin 1 [Homo sapiens] | gi\|17318569\|ref\|NP_00 | 66050 | 203 | AQYEDIAQK | 1065.52 | 356 | 364 |
| | 6112.2\| | | | | | | |
| keratin 1 [Homo sapiens] | gi\|17318569\|ref\|NP_00 | 66050 | 204 | DYQELMNTK | 1141.52 | 464 | 472 |
| | 6112.2\| | | | | | | |
| keratin 1 [Homo sapiens] | gi\|17318569\|ref\|NP_00 | 66050 | 205 | EQIKSLNNQFASFIDK | 1881.97 | 182 | 197 |
| | 6112.2\| | | | | | | |
| keratin 1 [Homo sapiens] | gi\|17318569\|ref\|NP_00 | 66050 | 206 | FLEQQNQVLQTK | 1475.79 | 200 | 211 |
| | 6112.2\| | | | | | | |
| keratin 1 [Homo sapiens] | gi\|17318569\|ref\|NP_00 | 66050 | 207 | FSSCGGGGGSFGAGGGFGSR | 1765.74 | 46 | 65 |
| 6112.2\| | | | | | | | |
| keratin 1 [Homo sapiens] | gi\|17318569\|ref\|NP_00 | 66050 | 208 | GGGGGGYGSGGSSYGSGGGS | 2383.95 | 519 549 | |
| | 6112.2\| | | | YGSGGGGGGGR | | | |
| keratin 1 [Homo sapiens] | gi\|17318569\|ref\|NP_00 | 66050 | 209 | IEISELNR | 973.53 | 396 | 403 |
| | 6112.2\| | | | | | | |
| keratin 1 [Homo sapiens] | gi\|17318569ref\|NP_\|00 | 66050 | 210 | LALDLEIATYR | 1277.71 | 473 | 483 |
| | 6112.2\| | | | | | | |
| keratin 1 [Homo sapiens] | gi\|17318569\|re\|NP_00 | 66050 | 211 | LNDLEDALQQAK | 1357.70 | 444 | 455 |
| | 6112.2\| | | | | | | |
| keratin 1 [Homo sapiens] | gi\|17318569\|ref\|NP_00 | 66050 | 212 | MSGECAPNVSVSVSTSHTTIS | 2565.17 | 493 | 518 |
| | 6112.2\| | GGGSR | | | | | |
| keratan 1 [Homo sapiens] | gi\|17318569\|ref\|NP_00 | 66050 | 213 | NKLNDLEDALQQAK | 1599.83 | 442 | 455 |
| | 6112.2\| | | | | | | |
| keratin 1 [Homo sapiens] | gi\|17318569\|ref\|NP_00 | 66050 | 214 | NKYEDEINKR | 1308.65 | 268 | 277 |
| | 6112.2\| | | | | | | |
| keratin 1 [Homo sapiens] | gi\|17318569\|ref\|NP_00 | 66050 | 215 | NMQDMVEDYR | 1300.53 | 258 | 267 |
| | 6112.2\| | | | | | | |
| keratin 1 [Homo sapiens] | gi\|17318569\|ref\|NP_00 | 66050 | 216 | NMQDMVEDYR | 1300.53 | 258 | 267 |
| | 6112.2\| | | | | | | |
| keratin 1 [Homo sapiens] | gi\|17318569\|ref\|NP_00 | 66050 | 217 | QISNLQQSISDAEQR | 1716.85 | 418 | 432 |
| | 6112.2\| | | | | | | |
| keratin 1 [Homo sapiens] | gi\|17318569\|ref\|NP_00 | 66050 | 218 | SKAEAESLYQSK | 1340.67 | 365 | 376 |
| | 6112.2\| | | | | | | |
| keratin 1 [Homo sapiens] | gi\|17318569\|ref\|NP_00 | 66050 | 219 | SLDLDSIIAEVK | 1302.72 | 344 | 355 |
| | 6112.2\| | | | | | | |
| keratin 1 [Homo sapiens] | gi\|17318569\|ref\|NP_00 | 66050 | 220 | THNLEPYFESFINNLR | 1993.98 | 224 | 239 |
| | 6112.2\| | | | | | | |
| keratin 1 [Homo sapiens] | gi\|17318569\|ref\|NP_00 | 66050 | 221 | TLLEGEESR | 1033.52 | 484 | 492 |
| | 6112.2\| | | | | | | |
| keratin 1 [Homo sapiens] | gi\|17318569\|ref\|NP_00 | 66050 | 223 | TNAENEFVTIK | 1265.64 | 278 | 288 |
| | 6112.2\| | | | | | | |
| keratin 1 [Homo sapiens] | gi\|17318569\|ref\|NP_00 | 66050 | 224 | WELLQQVDTSTR | 1475.75 | 212 | 223 |
| | 6112.2\| | | | | | | |
| keratin 1 [Homo sapiens] | gi\|17318569\|ref\|NP_00 | 66050 | 225 | YEELQITAGR | 1179.60 | 377 | 386 |
| | 6112.2\| | | | | | | |
| Keratin 10 | Q14664 | 57231 | 226 | GSSGGGGFGGSSGGYGGLGGF | 2342.99 | 53 | 79 |
| | | | | GGGSFR | | | |
| keratin 9 [Homo sapiens] | gi\|55956899\|ref\|NP_00 | 62048 | 248 | DIENQYETQITQIEHEVSSSG | 3264.51 | 340 | 368 |
| | 0217.2\| | | | QEVQSSAK | | | |
| keratin 9 [Homo sapiens] | gi\|55956899\|ref\|NP_00 | 62048 | 249 | EIETYHNLLEGGQEDFESSGA | 2510.13 | 450 | 472 |
| | 0217.2\| | | | GK | | | |
| keratin 9 [Homo sapiens] | gi\|55956899\|ref\|NP_00 | 62048 | 250 | FSSSSGYGGGSSR | 1235.53 | 47 | 59 |
| | 0217.2\| | | | | | | |
| keratin 9 [Homo sapiens] | gi\|559568991ref]NP_00 | 62048 | 251 | GGGGSFGYSYGGGSGGGFSAS | 2705.16 | 64 | 95 |
| | 0217.2\| | | | SLGGGFGGGSR | | | |
| keratin 9 [Homo sapiens] | gi\|55956899\|ref\|NP_00 | 62048 | 252 | HGVQELEIELQSQLSK | 1837.97 | 375 | 390 |
| | 0217.2\| | | | | | | |
| keratin 9 [Homo sapiens] | gi\|55956899\|ref\|NP_00 | 62048 | 253 | IKFEMEQNLR | 1307.68 | 241 | 250 |
| | 0217.2\| | | | | | | |
| keratin 9 [Homo sapiens] | gi\|55956899\|ref\|NP_00 | 62048 | 254 | IQDWYDKK | 1095.55 | 185 | 192 |
| | 0217.2\| | | | | | | |
| keratin 9 [Homo sapiens] | gi\|55956899\|ref\|NP_00 | 62048 | 255 | LASYLDK | 809.44 | 164 | 170 |
| | 0217.2\| | | | | | | |
| keratin 9 [Homo sapiens] | gi\|55956899\|ref\|NP_00 | 62048 | 256 | NYSPYYNTIDDLKDQIVDLTV | 2902.41 | 200 | 224 |
| | 0217.2\| | | | GNNK | | | |
| keratin 9 [Homo sapiens] | gi\|55956899\|ref\|NP_00 | 62048 | 257 | SDLEMQYETLQEELMALK | 2171.03 | 272 | 289 |
| | 0217.2\| | | | | | | |
| keratin 9 [Homo sapiens] | gi\|55956899\|ref\|NP_00 | 62048 | 258 | SGGGGGGGLGSGGSIR | 1232.60 | 14 | 29 |
| | 0217.2\| | | | | | | |
| keratin 9 [Homo sapiens] | gi\|55956899\|ref\|NP_00 | 62048 | 259 | STMQELNSR | 1065.50 | 155 | 163 |
| | 0217.2\| | | | | | | |
| keratin 9 [Homo sapiens] | gi\|55956899\|ref\|NP_00 | 62048 | 260 | TLLDIDNTR | 1060.56 | 225 | 233 |
| | 0217.2\| | | | | | | |
| keratin 9 [Homo sapiens] | gi\|55956899\|ref\|NP_00 | 62048 | 261 | VQALEEANNDLENK | 1586.77 | 171 | 184 |
| | 0217.2\| | | | | | | |
| keratin 9 [Homo sapiens] | gi\|55956899\|ref\|NP_00 | 62048 | 262 | YCGQLQMIQEQISNLEAQITD | 2753.32 | 405 | 427 |
| | 0217.2\| | | | VR | | | |
| Keratin, type I cytoskeletal 10 | K1CIO_HUMAN | 59502 | 263 | AETECQNTEYQQLLDIKIR | 2352.15 | 423 | 441 |
| (Cytokeratin-10) (CK-10) (Keratin-10) | | | | | | | |
| (K10) | | | | | | | |
| Keratin, type I cytoskeletal 10 | K1C10_HUMAN | 59502 | 264 | GSLGGGFSSGGFSGGSF | 1464.64 | 41 | 57 |
| (Cytokeratin-10) (CK-10) (Keratin-10) | | | | | | | |
| (K10) | | | | | | | |
| Keratin, type I cytoskeletal 10 | K1C10_HUMAN | 59502 | 265 | ILLQIDNAR | 1055.62 | 220 | 228 |
| (Cytokeratin-10) (CK-10) (Keratin-10) | | | | | | | |
| (K10) | | | | | | | |
| Keratin, type I cytoskeletal 10 | K1CIO_HUMAN | 59502 | 266 | IRLENEIQTYR | 1434.77 | 440 | 450 |
| (Cytokeratin-10) (CK-10) (Keratin-10) | | | | | | | |
| (K10) | | | | | | | |
| Keratin, type I cytoskeletal 10 | K1C10_HUMAN | 59502 | 267 | ISALEEQLQQIR | 1427.79 | 411 | 422 |
| (Cytokeratin-10) (CK-10) (Keratin-10) | | | | | | | |
| (K10) | | | | | | | |
| Keratin, type I cytoskeletal 10 | K1C10_HUMAN | 59502 | 268 | LAADDFR | 807.40 | 229 | 235 |
| (Cytokeratin-10) (CK-10) (Keratin-10) | | | | | | | |
| (K10) | | | | | | | |
| Keratin, type I cytoskeletal 10 | K1C10_HUMAN | 59502 | 269 | LASYLDK | 809.44 | 157 | 163 |
| (Cytokeratin-10) (CK-10) (Keratin-10) | | | | | | | |
| (K10) | | | | | | | |
| Keratin, type I cytoskeletal 10 | K1C10_HUMAN | 59502 | 270 | LENEIQTYR | 1165.59 | 442 | 450 |
| (Cytokeratin-10) (CK-10) (Keratin-10) | | | | | | | |
| (K10) | | | | | | | |
| Keratin, type I cytoskeletal 10 | K1C1O_HUMAN | 59502 | 271 | LKYENEVALR | 1234.68 | 236 | 245 |
| (Gytokeratin-10) (CK-10) (Keratin-10) | | | | | | | |
| (K10) | | | | | | | |
| Keratin, type I cytoskeletal 10 | K1C10_HUMAN | 59502 | 272 | NQILNLTTDNAN | 1330.66 | 208 | 219 |
| (Cytokeratin-10) (CK-10) (Keratin-10) | | | | | | | |
| (K10) | | | | | | | |
| Keratin, type I cytoskeletal 10 | K1C1O_HUMAN | 59502 | 273 | NQILNLTTDNANILLQIDNAR | 2367.26 | 208 | 228 |
| (Cytokeratin-10) (CK-10) (Keratin-10) | | | | | | | |
| (K10) | | | | | | | |
| Keratin, type I cytoskeletal 10 | K1C1O_HUMAN | 59502 | 274 | NVQALEIELQSQLALK | 1797.01 | 371 | 386 |
| (Cytokeratin-10) (CK-10) (Keratin-10) | | | | | | | |
| (K10) | | | | | | | |
| Keratin, type I cytoskeletal 10 | K1C10_HUMAN | 59502 | 275 | RVLDELTLTK | 1187.70 | 257 | 266 |
| (Cytokeratin-10) (CK-10) (Keratin-10) | | | | | | | |
| (K10) | | | | | | | |
| Keratin, type I cytoskeletal 10 | K1C10_HUMAN | 59502 | 276 | SGGGGGGGGCGGGGGVSSLR | 1549.68 | 16 | 35 |
| (Cytokeratin-10) (CK-l0) (Keratin-10) | | | | | | | |
| (K10) | | | | | | | |
| Keratin, type I cytoskeletal 10 | K1C10_HUMAN | 59502 | 277 | SKELTTEIDNNIEQISSYK | 2212.10 | 344 | 362 |
| (Cytokeratin-10) (CK-10) (Keratin-10) | | | | | | | |
| K10) | | | | | | | |
| Keratin, type I cytoskeletal 10 | K1C10_HUMAN | 59502 | 278 | SLLEGEGSSGGGGR | 1262.60 | 451 | 464 |
| (Cytokeratin-10) (CK-l0) (Keratin-10) | | | | | | | |
| (K10) | | | | | | | |
| Keratin, type I cytoskeletal 10 | K1C10_HUMAN | 59502 | 279 | VQALEIELQSQLALK | 1682.97 | 372 | 386 |
| (Cytokeratin-10) (CK-l0) (Keratin-10) | | | | | | | |
| (K10) | | | | | | | |
| Keratin, type I cytoskeletal 14 | K1C14_HUMAN | 51473 | 281 | EVATNSELVQSGK | 1361.69 | 315 | 327 |
| (Cytokeratin-14) (CK-14) (Keratin-14) | | | | | | | |
| (K14) | | | | | | | |
| Keratin, type I cytoskeletal 14 | K1C14_HUMAN | 51473 | 282 | GSCGIGGGIGGGSSR | 1278.59 | 15 | 29 |
| (Cytokeratin-14) (CK-14) (Keratin-14) | | | | | | | |
| (K14) | | | | | | | |
| Keratin, type I cytoskeletal 14 | K1C14_HUMAN | 51473 | 283 | ISSVLAGGSCR1106.56 | 30 | 40 | |
| (Cytokeratin-14) (CK-14) (Keratin-14) | | | | | | | |
| (K14) | | | | | | | |
| Keratin, type I cytoskeletal 14 | K1C14_HUMAN | 51473 | 284 | VLDELTLAR | 1029.59 | 223 | 231 |
| (Cytokeratin-14) (CK-14) (Keratin-14) | | | | | | | |
| (K14) | | | | | | | |
| Keratin, type II cytoskeletal 1 | K2C1_HUMAN | 65870 | 285 | AEAESLYQSK | 1125.54 | 366 | 375 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 286 | ELLQQVDTSTR | 1289.67 | 212 | 222 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 287 | GGSGGGGGGSSGGRGSGGGSS | 2079.92 | 588 | 615 |
| (Cytokeratin-1) (CK-1) (Keratin-1) (K1) | GGSIGGR | | | | | | |
| (67 kDa cytokeratin) (Hair alpha protein) | | | | | | | |
| Keratin, type II cytoskeletal 1 | K2C1_HUMAN | 65870 | 288 | GSGGGSSGGSIGGR | 1092.50 | 602 | 615 |
| (Cytokeratin-I) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 289 | KQISNLQQSISDAEQR | 1844.95 | 416 | 431 |
| (Cytokeratin-1) (CK-1) (Keratini) (K1) | | | | | | | |
| (67 kDa cytokeratin) (Hair alpha protein) | | | | | | | |
| Keratin, type II cytoskeletal 1 | K2C1_HUMAN | 65870 | 290 | LALDLEIATYR | 1277.71 | 472 | 482 |
| (Cytokeratin-l) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 291 | NKLNDLEDALQQAK | 1599.83 | 441 | 454 |
| (Cytokeratin-l) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 292 | NVEIDPEIQK | 1184.62 | 165 | 174 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 293 | SEIDNVKK | 932.51 | 409 | 416 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 294 | SGGGFSSGSAGIINYQR | 1657.79 | 12 | 28 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 295 | SISDAEQR | 905.43 | 424 | 431 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 296 | SISISVARGGGR | 1159.65 | 74 | 85 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 297 | SKAEAESLYQSK | 1340.67 | 364 | 375 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 298 | SLDLDSIIAEVK | 1302.72 | 343 | 354 |
| (Cytokeratin-1) (CK-1) (Keratin-I) (K1) | | | | | | | |
| (67 kDa cytokeratin) (Hair alpha protein) | | | | | | | |
| Keratin, type 11 cytoskeletal 1 | K2C1_HUMAN | 65870 | 299 | SLNNQFASFIDK | 1383.69 | 185 | 196 |
| (Cytokeratin-1) (CK-1) (Keratin1) (K1) | | | | | | | |
| (67 kDa cytokeratin) (Hair alpha protein) | | | | | | | |
| Keratin, type II cytoskeletal 1 | K2C1_HUMAN | 65870 | 300 | SLNNQFASFIDKVR | 1638.86 | 185 | 198 |
| (Cytokeratin-1) (CK-1) (Keratin-1) (K1) | | | | | | | |
| (67 kDa cytokeratin) (Hair alpha protein) | | | | | | | |
| Keratin, type 11 cytoskeletal 1 | K2C1_HUMAN | 65870 | 301 | SLVNLGGSK | 874.50 | 65 | 73 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 302 | THNLEPYFESF | 1383.62 | 223 | 233 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 303 | THNLEPYFESFINNLR | 1993.98 | 223 | 238 |
| (Cytokeratin-1) (GK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 304 | TLLEGEESR | 1033.52 | 483 | 491 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 305 | TNAENEFVTIK | 1265.64 | 277 | 287 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 306 | VDQLKSDQSR | 1175.60 | 241 | 250 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 307 | VEIDPEIQK | 1070.57 | 166 | 174 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 308 | WELLQQVDTSTR | 1475.75 | 211 | 222 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 1 | K2C1_HUMAN | 65870 | 309 | YEELQITAGR | 1179.60 | 376 | 385 |
| (Cytokeratin-1) (CK-1) (Keratin-1) (K1) | | | | | | | |
| (67 kDa cytokeratin) (Hair alpha protein) | | | | | | | |
| Keratin, type II cytoskeletal 2 epidermal | K22E_HUMAN | 65848 | 310 | GFSSGSAVVSGGSR | 1254.61 | 21 | 34 |
| (Cytokeratin-2e) (K2e) (CK 2e) | | | | | | | |
| Keratin, type II cytoskeletal 2 epidermal | K22E_HUMAN | 65848 | 311 | IEISELNR | 973.53 | 400 | 407 |
| (Cytokeratin-2e) (K2e) (CK 2e) | | | | | | | |
| Keratin, type II cytoskeletal 2 epidermal | K22E_HUMAN | 65848 | 312 | LALDVEIATYR | 1263.69 | 477 | 487 |
| (Cytokeratin-2e) (K2e) (CK 2e) | | | | | | | |
| Keratin, type II cytoskeletal 2 epidermal | K22E_HUMAN | 65848 | 313 | LNDLEEALQQAK | 1371.71 | 448 | 459 |
| (Cytokeratin-2e) (K2e) (CK 2e) | | | | | | | |
| Keratin, type II cytoskeletal 2 epidermal | K22E_HUMAN | 65848 | 314 | NLDLDSHAEVK | 1329.73 | 348 | 359 |
| (Cytokeratin-2e) (K2e) (CK 2e) | | | | | | | |
| Keratin, type II cytoskeletal 2 epidermal | K22E_HUMAN | 65848 | 315 | NVQDAIADAEQR | 1329.64 | 425 | 436 |
| (Cytokeratin-2e) (K2e) (CK 2e) | | | | | | | |
| Keratin, type II cytoskeletal 2 epidermal | K22E_HUMAN | 65848 | 316 | VDLLNQEIEFLK | 1460.80 | 309 | 320 |
| (Cytokeratin-2e) (K2e) (CK 2e) | | | | | | | |
| Keratin, type II cytoskeletal 2 epidermal | K22E_HUMAN | 65848 | 317 | YEDEINKR | 1066.52 | 274 | 281 |
| (Cytokeratin-2e) (K2e) (CK 2e) | | | | | | | |
| Keratin, type II cytoskeletal 2 epidermal | K22E_HUMAN | 65848 | 318 | YEELQVTVGR | 1193.62 | 381 | 390 |
| (Cytokeratin-2e) (K2e) (CK 2e) | | | | | | | |
| Keratin, type II cytoskeletal 2 epidermal | K22E_HUMAN | 65848 | 319 | YLDGLTAER | 1037.53 | 245 | 253 |
| (Cytokeratin-2e) (K2e) (CK 2e) | | | | | | | |
| KIAA1199 [Homo sapiens] | gi\|38638698\|re\|NP_06 | 152983 | 320 | PFLSIISARYSPHQDADPLK | 2255.18 | 755 | 774 |
| | 1159.1\| | | | | | | |
| Moy10, Moloney leukemia virus 10, | gi\|14211540\|ref\|NP_O6 | 113658 | 333 | ISFGTPAPGFSSMLYGMK | 1906.91 | 45 | 62 |
| homolog [Homo sapiens] | 6014.1\| | | | | | | |
| Novel protein (Keratinocyte proline-rich | Q5T749 | 64115 | 336 | GRPAVCQPQGR | 1225.62 | 167 | 177 |
| protein) | | | | | | | |
| Novel protein (Keratinocyte proline-rich | Q5T749 | 64115 | 337 | IEISSPCCPR | 1218.56 | 327 | 336 |
| protein) | | | | | | | |
| Novel protein (Keratinocyte praline-rich | Q5T749 | 64115 | 338 | RPISSCSQR | 1090.54 | 312 | 320 |
| protein) | | | | | | | |
| pericentriolar material 1 [Homo sapiens] | gi\|34878902\|ref\|NP_00 | 228284 | 339 | LPEMEPLVPRVKEVK | 1780.00 | 1906 | 1920 |
| | 6188.2\| | | | | | | |
| peroxiredoxin 2 isoform a [Homo | gi\|32189392\|ref\|NP_00 | 21874 | 340 | ATAVVDGAFK | 978.53 | 17 | 26 |
| sapiens] | 5800.31,gi\|33188452\|re | | | | | | |
| | f\|NP_859427.1\| | | | | | | |
| peroxiredoxin 2 isoform a [Homo | gi\|32189392\|ref\|NP_00 | 21874 | 341 | EGGLGPLNIPLLADVTR | 1734.98 | 93 | 109 |
| sapiens] | 5800.3\|,gi\|33188452\|re | | | | | | |
| | f\|NP_859427.11 | | | | | | |
| peroxiredoxin 2 isoform a [Homo | gi\|32189392\|ref\|NP_00 | 21874 | 342 | EGGLGPLNIPLLADVTR | 1734.98 | 93 | 109 |
| sapiens] | 5800.3\|,gi\|33188452\|re | | | | | | |
| | f\|NP_859427.1\| | | | | | | |
| peroxiredoxin 2 isoform a [Homo | gi\|32189392\|ref\|NP_00 | 21874 | 343 | GLFIIDGK | 862.50 | 128 | 135 |
| sapiens] | 5800.3\|,gi\|33188452\|re | | | | | | |
| | f\|NP_859427.1\| | | | | | | |
| peroxiredoxin 2 isoform a [Homo | gi\|32189392\|ref\|NP_00 | 21874 | 344 | KEGGLGPLNIPLLADVTR | 1863.07 | 92 | 109 |
| sapiens] | 5800.3\|,gi\|33188452\|re | | | | | | |
| | f\|NP_859427.1\| | | | | | | |
| peroxiredoxin 2 isoform a [Homo | gi\|32189392\|ref\|NP_00 | 21874 | 345 | LSEDYGVLK | 1023.54 | 111 | 119 |
| sapiens] | 5800.3\|,gi\|331884521re | | | | | | |
| | f\|NP_859427.1\| | | | | | | |
| peroxiredoxin 2 isoform a [Homo | gi\|32189392\|ref\|NP_00 | 21874 | 346 | RLSEDYGVLK | 1179.64 | 110 | 119 |
| sapiens] | 5800.3\|,gi\|33188452\|re | | | | | | |
| | f\|NP_859427.1\| | | | | | | |
| peroxiredoxin 2 isoform a [Homo | gi\|32189392\|ref\|NP_00 | 21874 | 347 | TDEGIAYR | 924.44 | 120 | 127 |
| sapiens] | 5800.3\|,gi\|33188452\|re | | | | | | |
| | f\|NP_859427.1\| | | | | | | |
| plasma carboxypeptidase B2 isoform a | gi\|4503005\|ref\|NP_001 | 48425 | 349 | DTGTYGFLLPER | 1368.68 | 388 | 399 |
| preproprotein [Homo sapiens] | 863.1\| | | | | | | |
| platelet factor 4 (chemokine (C-X-C | gi\|45O5733\|ref\|NP_002 | 10828 | 350 | AGPHCPTAQLIATLK | 1577.85 | 63 | 77 |
| motif) ligand 4) [Homo sapiens] | 610.1\| | | | | | | |
| platelet factor 4 (chemokine (C-X-C | gi\|4505733\|ref\|NP_002 | 10828 | 351 | HTSLEVIK | 1039.62 | 54 | 62 |
| motif) ligand 4) [Homo sapiens] | 610.1\| | | | | | | |
| platelet factor 4 (chemokine (C-X-C | gi\|4505733\|ref\|NP_002 | 10828 | 352 | ICLDLQAPLYK | 1333.72 | 82 | 92 |
| motif) ligand 4) [Homo sapiens] | 610.1\| | | | | | | |
| platelet factor 4 (chemokine (C-X-C | gi\|4505733\|ref\|NP_002 | 10828 | 353 | ICLDLQAPLYKK | 1461.81 | 82 | 93 |
| motif) ligand 4) [Homo sapiens] | 610.1\| | | | | | | |
| platelet factor 4 (chemokine (C-X-C | gi\|4505733\|ref\|NP_002 | 10828 | 354 | KICLDLQAPLYK | 1461.81 | 81 | 92 |
| motif) ligand 4) [Homo sapiens]610.1\| | | | | | | | |
| profilin family, member 4 [Homo | gi\|40786418\|ref\|NP_95 | 14302 | 360 | CVRADEYSLYAKNENTGVVV | 2514.27 | 73 | 94 |
| sapiens] | 5378.1\| | | | VK | | | |
| propionyl-Coenzyme A carboxylase, | gi\|655O6442\|ref\|NP_00 | 80041 | 361 | YSSAGTVEFLVDSK | 1502.74 | 329 | 342 |
| alpha polypeptide precursor [Homo | 0273.2\| | | | | | | |
| sapiens] | | | | | | | |
| regulatory solute carrier protein, family | gi\|5730021\|ref\|NP_006 | 66771 | 364 | DLGQGIQNSVTDRPETR | 1885.94 | 523 | 539 |
| 1, member 1 [Homo sapiens] | 502.1\| | | | | | | |
| roundabout, axon guidance receptor, | gi\|61888896\|ref\|NP_00 | 151182 | 366 | QTSGLQATSSWQNLDAK | 1834.89 | 674 | 690 |
| homolog 2 [Homo sapiens] | 2933.1\| | | | | | | |
| Rsb-66 protein [Homo sapiens] | gi\|33285006\|ref\|NP_06 | 19271 | 367 | PISSIGQVQSYMEHYCNSSTD | 2731.22 | 42 | 64 |
| | 1829.3\| | | | RR | | | |
| S100 calcium-binding protein A7 | S10A7_HUMAN | 11309 | 368 | ENFPNFLSACDK | 1441.64 | 37 | 48 |
| (Psoriasin) | | | | | | | |
| S100 calcium-binding protein A7 | S10A7_HUMAN | 11309 | 369 | GTNYLADVFEK | 1256.62 | 50 | 60 |
| (Psoriasin) | | | | | | | |
| S100 calcium-binding protein A7 | S10A7_HUMAN | 11309 | 370 | IDFSEFLSLLGDIATDYHK | 2184.09 | 69 | 87 |
| (Psoriasin) | | | | | | | |
| S100 calcium-binding protein A7 | S10A7_HUMAN | 11309 | 371 | KIDFSEFLSLLGDIATDYHK | 2312.18 | 68 | 87 |
| (Psoriasin) | | | | | | | |
| secretoglobin, family 3A, member 1 | gi\|50363226\|ref\|NP_44 | 10082 | 374 | LLLSSLGIPVNHLIEGSQK | 2018.17 | 59 | 77 |
| [Homo sapiens] | 3095.2\| | | | | | | |
| secretoglobin, family 3A, member 1 | gi\|50363226\|ref\|NP_44 | 10082 | 375 | PVAQPVAALESAAEAGAGTLA | 2941.60 | 28 | 58 |
| [Homo sapiens] | 3095.21 | | | NPLGTLNPLK | | | |
| Tetranectin precursor (TN) | TETN_HUMAN | 22549 | 403 | GGTLSTPQTGSENDALYEYLR | 2272.07 | 102 | 122 |
| (Plasminogen-kringle 4 binding protein) | | | | | | | |
| Tetranectin precursor (TN) | TETN_HUMAN | 22549 | 404 | LDTLAQEVALLK | 1313.77 | 51 | 62 |
| (Plasminogen-kringle 4 binding protein) | | | | | | | |
| thrombospondin 4 precursor [Homo | gi\|31543806\|ref\|NP_00 | 105851 | 405 | LNPGALLPVLTDPALNDLYVI | 2684.49 | 41 | 65 |
| sapiens] | 3239.2\| | | | STFK | | | |
| vitamin D-binding protein precursor | gi\|32483410\|ref\|NP_00 | 52900 | 427 | EDFTSLSLVLYSR | 1529.79 | 38 | 50 |
| [Homo sapiens] | 0574.2\| | | | | | | |
| vitamin D-binding protein precursor | gi\|32483410\|ref\|NP_00 | 52900 | 428 | EFSHLGKEDFTSLSLVLYSR | 2328.19 | 31 | 50 |
| [Homo sapiens] | 0574.2\| | | | | | | |
| vitamin D-binding protein precursor | gi\|32483410\|ref\|NP_00 | 52900 | 429 | ELSSFIDK | 938.48 | 395 | 402 |
| [Homo sapiens] | 0574.2\| | | | | | | |
| vitamin D-binding protein precursor | gi\|32483410\|ref\|NP_00 | 52900 | 430 | FPSGTFEQVSQLVK | 1566.82 | 52 | 65 |
| Homo sapiens] | 0574.2\| | | | | | | |
| vitamin D-binding protein precursor | gi\|32483410\|ref\|NP_00 | 52900 | 431 | KFPSGTFEQVSQLVK | 1694.91 | 51 | 65 |
| [Homo sapiens] | 0574.2\| | | | | | | |
| vitamin D-binding protein precursor | gi\|32483410\|ref\|NP_00 | 52900 | 432 | LPDATPTELAK | 1155.63 | 430 | 440 |
| [Homo sapiens] | 0574.2\| | | | | | | |
| vitamin D-binding protein precursor | gi\|32483410\|ref\|NP_00 | 52900 | 433 | RTHLPEVFLSK | 1326.75 | 353 | 363 |
| [Homo sapiens] | 0574.2\| | | | | | | |
| vitamin D-binding protein precursor | gi\|32483410\|ref\|NP_00 | 52900 | 434 | VPTADLEDVLPLAEDITNILS | 2366.27 | 243 | 264 |
| [Homo sapiens] | 0574.2\| | | | K | | | |
| vitamin D-binding protein precursor | gi\|32483410\|ref\|NP_00 | 52900 | 435 | YTFELSR | 915.46 | 346 | 352 |
| [Homo sapiens] | 0574.2\| | | | | | | |

TABLE 4
Biomarkers associated with brain microhemorrhages

| | | | | | | |
|---|---|---|---|---|---|---|
| | | Protein | | | | |
| | Protein | molecular | | | Calculated | Peptide |
| | accession | weight | SEQ ID | | Peptide | start |
| Protein name | numbers | (Da) | NO: | Peptide sequence | Mass (AMU) | index |

| | | | | | | |
|---|---|---|---|---|---|---|
| (P21817) Ryanodine receptor 1 | RYR1_HUMAN | 565156 | 441 | EEQNFVVQNEINNM | 2584.20 | 3454 |
| (Skeletal muscle-type ryanodine | | | | SFLTADNK | | |
| receptor) (RyR1) (RYR-1) (Skeletal | | | | | | |
| muscle calcium release channel) | | | | | | |
| (Q9NVES) Ubi\|uitin carboxyl- | Q9NVE5 | 140130 | 442 | IGISWNKK | 945.55 | 372 |
| terminal hydrolase 40 (EC 3.1.2.15) | | | | | | |
| (U | | | | | | |
| (Q5NV79) V5-4 protein (Fragment) | Q5NV79 | 10661 | 443 | FSGSSSGADR | 970.42 | 66 |
| (P51843) Nuclear receptor 0B1 | DAX1_HUMAN | 51700 | 444 | YLPCFQVLPLDQQL | 2201.22 | 271 |
| (Nuclear receptor DAX-1) (DSS- | | | | VLVR | | |
| AHC critical region on the X | | | | | | |
| chromosome protein 1) | | | | | | |
| (Q96SU4) Oxysterol binding protein- | Q96SU4 | 83185 | 445 | HCIVLLQIAKDQSNA | 1967.04 | 172 |
| related protein 9 (OSBP-related | | | | EK | | |
| (P17706) Tyrosine-protein | PTN2_HUMAN | 48511 | 446 | VKLQNAENDYINAS | 2632.32 | 59 |
| phosphatase, non-receptor type 2 (EC | | | | LVDIEEAQR | | |
| 3.1.3.48) (T-cell protein-tyrosine | | | | | | |
| phosphatase) (TCPTP) | | | | | | |
| (O15013) Rho guanine nucleotide | ARHGA_HUMAN | 127102 | 447 | YILGSVVDSEKNYV | 2169.16 | 194 |
| exchange factor 10 | | | | DALKR | | |
| (O75129) KJAA0634 protein | O75129 | 145410 | 448 | EAFKSALMSSYWCS | 1851.84 | 1006 |
| (Fragment) | | | | GK | | |
| (Q8N543) Hypothetical protein | Q8N543 | 63230 | 449 | EPHISTLRKILFEDFR | 2001.09 | 109 |
| FLJ10826 | | | | | | |
| (Q96AE7) Tetratricopeptide repeat | TTC17_HUMAN | 129543 | 450 | IHIEENEDRDTGLEQ | 2333.12 | 91 |
| protein 17 (TPR repeat protein 17) | | | | RHNK | | |
| (Q96AE7) Tetratricopeptide repeat | TTC17_HUMAN | 129543 | 451 | GLRIHELSSDDYSTE | 3371.50 | 530 |
| protein 17 (TPR repeat protein 17) | | | | EEAQTPDCSITDFR | | |
| (Q96AE7) Tetratricopeptide repeat | TTC17_HUMAN | 129543 | 452 | IQQQVDSPMNLK | 1400.72 | 50 |
| protein 17 (TPR repeat protein 17) | | | | | | |

TABLE 8
Analysis of candidate biomarkers in normal and mild AD patients.

| | | | | | | Normal | Normal | MCI vs |
|---|---|---|---|---|---|---|---|---|
| | | | | | | vs | vs | mild |
| Accession | Protein name | MW | Normal | MCI | mild AD | mild AD | MCI | AD |
| P13645 | (P13645) Keratin, type I cytoskeletal 10 (Cytokeratin-10) (CK- | 59502.3 | 0 | 57 | 4 | 100% | 100% | 87% |
| | 10) (Keratin-10) (K10) | | | | | | | |
| P35908 | (P35908) Keratin, type II cytoskeletal 2 epidermal | 65848.4 | 0 | 16 | 4 | 100% | 100% | 60% |
| | (Cytokeratin-2e) (K2e) (CK 2e) | | | | | | | |
| P02671 | (P02671) Fibrinogen alpha chain precursor [Contains: | 94955.4 | 1 | 5 | 7 | 75% | 67% | 17% |
| | Fibrinopeptide A] | | | | | | | |
| Q6PYX1 | (Q6PYX1) Hepatitis B virus receptor binding protein | 38143 | 1 | 8 | 5 | 67% | 78% | 23% |
| | (Fragment) | | | | | | | |
| O75179 | (O75179) KIAA0697 protein (Fragment) | 263225.4 | 1 | 2 | 5 | 67% | 33% | 43% |
| P55056 | (P55056) Apolipoprotein C-IV precursor (Apo-CIV) (ApoC-IV) | 14535.5 | 2 | 2 | 7 | 56% | 0% | 56% |
| Q6GTG1 | (Q6GTG1) Vitamin D-binding protein, | 52919.5 | 6 | 6 | 0 | 100% | 0% | 100% |
| O95978 | (O95978) VH1 protein precursor (Fragment) | 17285 | 6 | 1 | 0 | 100% | 71% | 100% |
| P01781 | (P01781) Ig heavy chain V-III region GAL | 12708.1 | 5 | 4 | 0 | 100% | 11% | 100% |
| P01614 | (P01614) Ig kappa chain V-II region Cum | 12658.6 | 4 | 2 | 0 | 100% | 33% | 100% |
| P13798 | (P13798) Acylamino-acid-releasing enzyme (EC 3.4.19.1) | 81206.1 | 4 | 0 | 0 | 100% | 100% | |
| | (AARE) (Acyl-peptide hydrolase) (APH) (Acylaminoacyl- | | | | | | | |
| | peptidase) (Oxidized protein hydrolase) (OPH) (DNF15S2 | | | | | | | |
| | protein) | | | | | | | |
| P55073 | (P55073) Type III iodothyronine deiodinase (EC 1.97.1.11) (Type- | 31386.2 | 4 | 0 | 0 | 100% | 100% | |
| | III 5′deiodinase) (DIOIII) (Type 3 DI) (5DIII) | | | | | | | |
| Q6MZW0 | (Q6MZW0) Hypothetical protein DKFZp686J11235 (Fragment) | 54440.5 | 6 | 5 | 1 | 71% | 9% | 67% |
| Q5CZ94 | (Q5CZ94) Hypothetical protein DKFZp781M0386 | 24983.5 | 8 | 6 | 2 | 60% | 14% | 50% |
| P01011 | (P01011) Alpha-1-antichymotrypsin precursor (ACT) [Contains: | 47635 | 12 | 13 | 4 | 50% | 4% | 53% |
| | Alpha-1-antichymotrypsin His-Pro-less] | | | | | | | |

| | |
|---|---|
| Biomarkers for: | Normal versus mild AD |
| Experiment: | LMW_b (pooled samples of Normal/MCI/mild AD) |
| Filters: | SEQUEST => XC +1 > 1.9, +2 > 2.2, +3 > 3.5 |
| | at least 4 spectra per protein in one group |
| | over 50% difference between normal and mild AD |
| Key: | more abundant in mild AD |
| | more abundant in Normals |

| | | | | | | Normal | | | | | | | | | |
|---|---|---|---|---|---|---|---|---|---|---|---|---|---|---|---|
| | | | | Stable | Unstable | (stable + | | | | | Stable | | Stable | | Stable |
| | | | | Normal | Normal | unstable) | Stable | mild AD | | Unstable | Normal | Unstable | MCI | Unstable | Normal |
| | | | | [av | [av | [av | MCI [av | [av | mild | Normal | vs | Normal | vs | Normal | vs |
| | | | | samples] | samples] | samples] | samples] | samples] | AD vs | vs Stable | Stable | vs Stable | mild | vs mild | mild |
| Accession | Name | Probability | MW | (n = 3) | (n = 4) | (n = 7) | (n = 5) | (n = 12) | Normal | Normal | MCI | MCI | AD | AD | AD |
| P16591 | FER_HUMAN | 1.25E−04 | 94563.8 | 0.00 | 0.00 | 0.00 | 0.40 | 0.67 | 100% | 0% | 100% | 100% | 25% | 100% | 100% |
| | (P16591) Proto- | | | | | | | | | | | | | | |
| | oncogene | | | | | | | | | | | | | | |
| | tyrosine-protein | | | | | | | | | | | | | | |
| | kinase FER (EC | | | | | | | | | | | | | | |
| | 2.7.1.112) (p94- | | | | | | | | | | | | | | |
| | FER) (c-FER) | | | | | | | | | | | | | | |
| P22792 | CPN2_HUMAN | 2.00E−07 | 60576.3 | 0.00 | 0.00 | 0.00 | 0.60 | 0.58 | 100% | 0% | 100% | 100% | 1% | 100% | 100% |
| | (P22792) | | | | | | | | | | | | | | |
| | Carboxypeptidase | | | | | | | | | | | | | | |
| | N subunit 2 | | | | | | | | | | | | | | |
| | precursor | | | | | | | | | | | | | | |
| | (Carboxypeptidase | | | | | | | | | | | | | | |
| | N polypeptide | | | | | | | | | | | | | | |
| | 2) (Carb | | | | | | | | | | | | | | |
| Q6ZVQ3 | Q6ZVQ3 | 9.19E−04 | 17595.2 | 0.00 | 0.00 | 0.00 | 0.80 | 0.50 | 100% | 0% | 100% | 100% | 23% | 100% | 100% |
| | (Q6ZVQ3) | | | | | | | | | | | | | | |
| | Hypothetical | | | | | | | | | | | | | | |
| | protein FLJ42220 | | | | | | | | | | | | | | |
| Q9H2I2 | Q9H2I2 (Q9H2I2) | 2.53E−04 | 31787.4 | 0.00 | 0.00 | 0.00 | 0.20 | 0.33 | 100% | 0% | 100% | 100% | 25% | 100% | 100% |
| | HNRBF-2 | | | | | | | | | | | | | | |
| P13667 | PDIA4_HUMAN | 2.62E−04 | 72887.1 | 0.00 | 0.00 | 0.00 | 0.20 | 0.33 | 100% | 0% | 100% | 100% | 25% | 100% | 100% |
| | (P13667) Protein | | | | | | | | | | | | | | |
| | disulfide- | | | | | | | | | | | | | | |
| | isomerase A4 | | | | | | | | | | | | | | |
| | precursor (EC | | | | | | | | | | | | | | |
| | 5.3.4.1) (Protein | | | | | | | | | | | | | | |
| | ERp-72) (ERp72) | | | | | | | | | | | | | | |
| O60602 | TLR5_HUMAN | 2.76E−04 | 97663.4 | 0.00 | 0.00 | 0.00 | 0.00 | 0.33 | 100% | 0% | 0% | 0% | 100% | 100% | 100% |
| | (O60602) Toll- | | | | | | | | | | | | | | |
| | like receptor 5 | | | | | | | | | | | | | | |
| | precursor | | | | | | | | | | | | | | |
| | (Toll/interleukin- | | | | | | | | | | | | | | |
| | 1 recepto | | | | | | | | | | | | | | |
| O95793 | STAU_HUMAN | 2.83E−04 | 63227.9 | 0.00 | 0.00 | 0.00 | 0.40 | 0.33 | 100% | 0% | 100% | 100% | 9% | 100% | 100% |
| | (O95793) | | | | | | | | | | | | | | |
| | Double- | | | | | | | | | | | | | | |
| | stranded | | | | | | | | | | | | | | |
| | RNA-binding | | | | | | | | | | | | | | |
| | protein | | | | | | | | | | | | | | |
| | Staufen | | | | | | | | | | | | | | |
| | homolog | | | | | | | | | | | | | | |
| Q7Z3Z2 | CA036_HUMAN | 8.02E−05 | 22689.6 | 0.00 | 0.00 | 0.00 | 0.20 | 0.33 | 100% | 0% | 100% | 100% | 25% | 100% | 100% |
| | (Q7Z3Z2) | | | | | | | | | | | | | | |
| | Protein | | | | | | | | | | | | | | |
| | C1orf36 | | | | | | | | | | | | | | |
| Q05513 | KPCZ_HUMAN | 1.14E−04 | 67616.8 | 0.00 | 0.00 | 0.00 | 0.40 | 0.33 | 100% | 0% | 100% | 100% | 9% | 100% | 100% |
| | (Q05513) | | | | | | | | | | | | | | |
| | Protein kinase | | | | | | | | | | | | | | |
| | C, zeta type | | | | | | | | | | | | | | |
| | (EC 2.7.1.37) | | | | | | | | | | | | | | |
| | (nPKC-zeta) | | | | | | | | | | | | | | |
| Q4V312 | Q4V312 | 5.36E−05 | 46871.6 | 0.00 | 0.00 | 0.00 | 0.60 | 0.33 | 100% | 0% | 100% | 100% | 29% | 100% | 100% |
| | (Q4V312) | | | | | | | | | | | | | | |
| | Colony | | | | | | | | | | | | | | |
| | stimulating | | | | | | | | | | | | | | |
| | factor 2 | | | | | | | | | | | | | | |
| | receptor, | | | | | | | | | | | | | | |
| | alpha, low- | | | | | | | | | | | | | | |
| | affinity | | | | | | | | | | | | | | |
| | (Granulocyte- | | | | | | | | | | | | | | |
| | macrophage) | | | | | | | | | | | | | | |
| Q93088 | BHMT_HUMAN | 6.52E−04 | 44941.8 | 0.00 | 0.25 | 0.14 | 0.60 | 0.75 | 68% | 100% | 100% | 41% | 11% | 50% | 100% |
| | (Q93088) | | | | | | | | | | | | | | |
| | Betaine-- | | | | | | | | | | | | | | |
| | homocysteine | | | | | | | | | | | | | | |
| | S- | | | | | | | | | | | | | | |
| | methyltransferase | | | | | | | | | | | | | | |
| | (EC | | | | | | | | | | | | | | |
| | 2.1.1.5) | | | | | | | | | | | | | | |
| Q8N7W7 | Q8N7W7 | 5.41E−04 | 66086.9 | 0.00 | 0.25 | 0.14 | 0.40 | 0.58 | 61% | 100% | 100% | 23% | 19% | 40% | 100% |
| | (Q8N7W7) | | | | | | | | | | | | | | |
| | Hypothetical | | | | | | | | | | | | | | |
| | protein | | | | | | | | | | | | | | |
| | FLJ40259 | | | | | | | | | | | | | | |
| P35542 | SAA4_HUMAN | 9.09E−04 | 14797.3 | 0.00 | 0.25 | 0.14 | 0.00 | 0.58 | 61% | 100% | 0% | 100% | 100% | 40% | 100% |
| | (P35542) | | | | | | | | | | | | | | |
| | Serum | | | | | | | | | | | | | | |
| | amyloid A-4 | | | | | | | | | | | | | | |
| | protein | | | | | | | | | | | | | | |
| | precursor | | | | | | | | | | | | | | |
| | (Constitutively | | | | | | | | | | | | | | |
| | expres | | | | | | | | | | | | | | |
| Q9BXB9 | Q9BXB9 | 2.79E−04 | 46480 | 0.00 | 0.25 | 0.14 | 0.60 | 0.50 | 56% | 100% | 100% | 41% | 9% | 33% | 100% |
| | (Q9BXB9) LIM | | | | | | | | | | | | | | |
| | mineralization | | | | | | | | | | | | | | |
| | protein 2 | | | | | | | | | | | | | | |
| Q99996 | AKAP9_HUMAN | 1.98E−04 | 453386.1 | 0.33 | 0.50 | 0.43 | 0.00 | 0.00 | 100% | 20% | 100% | 100% | 0% | 100% | 100% |
| | (Q99996) A- | | | | | | | | | | | | | | |
| | kinase anchor | | | | | | | | | | | | | | |
| | protein 9 | | | | | | | | | | | | | | |
| | (Protein kinase | | | | | | | | | | | | | | |
| | A anchoring | | | | | | | | | | | | | | |
| | pro | | | | | | | | | | | | | | |
| P09758 | TACD2_HUMAN | 3.03E−04 | 35686.6 | 0.67 | 0.25 | 0.43 | 0.40 | 0.08 | 67% | 45% | 25% | 23% | 66% | 50% | 78% |
| | (P09758) | | | | | | | | | | | | | | |
| | Tumor- | | | | | | | | | | | | | | |
| | associated | | | | | | | | | | | | | | |
| | calcium signal | | | | | | | | | | | | | | |
| | transducer 2 | | | | | | | | | | | | | | |
| | precursor (P | | | | | | | | | | | | | | |
| Q5VVM6 | Q5VVM6 | 3.55E−04 | 91277.5 | 0.67 | 0.25 | 0.43 | 0.20 | 0.08 | 67% | 45% | 54% | 11% | 41% | 50% | 78% |
| | (Q5VVM6) | | | | | | | | | | | | | | |
| | Novel protein | | | | | | | | | | | | | | |
| P12814 | ACTN1_HUMAN | 4.70E−04 | 102992.7 | 1.00 | 0.00 | 0.43 | 0.00 | 0.08 | 67% | 100% | 100% | 0% | 100% | 100% | 85% |
| | (P12814) | | | | | | | | | | | | | | |
| | Alpha-actinin 1 | | | | | | | | | | | | | | |
| | (Alpha-actinin | | | | | | | | | | | | | | |
| | cytoskeletal | | | | | | | | | | | | | | |
| | isoform) (Non | | | | | | | | | | | | | | |

| | |
|---|---|
| Biomarkers for: | Normal versus mild AD |
| Experiment: | whole serum (individual samples of |
| | Normal/MCI/mild AD) |
| Filters: | SEQUEST |
| | found in more than 33% of either normals or mild |
| | AD patients |
| | over 50% difference between normal and mild |
| | AD (in average samples with positive |
| | identification) |
| | probability score of less than 1.00E−03 |
| Key: | more abundant in mild AD |
| | more abundant in Normals |

TABLE 9
Analysis of candidate biomarkers in MCI and mild AD patients

| | | | | | | Normal vs | Normal | MCI vs |
|---|---|---|---|---|---|---|---|---|
| Accession | Protein name | MW | Normal | MCI | mild AD | mild AD | vs MCI | mild AD |
| P02654 | (P02654) Apolipoprotein C-I precursor (Apo-CI) (ApoC-I) | 9314.4 | 6 | 2 | 9 | 20% | 50% | 64% |
| P55056 | (P55056) Apolipoprotein C-IV precursor (Apo-CIV) (ApoC-IV) | 14535.5 | 2 | 2 | 7 | 56% | 0% | 56% |
| Q6GTG1 | (Q6GTG1) Vitamin D-binding protein, | 52919.5 | 6 | 6 | 0 | 100% | 0% | 100% |
| P01824 | (P01824) Ig heavy chain V-II region WAH | 14099.1 | 0 | 5 | 0 | | 100% | 100% |
| P02533 | (P02533) Keratin, type I cytoskeletal 14 (Cytokeratin-14) (CK-14) | 51473.4 | 0 | 5 | 0 | | 100% | 100% |
| | (Keratin-14) (K14) | | | | | | | |
| P15924 | (P15924) Desmoplakin (DP) (250/210 kDa paraneoplastic | 331765.3 | 0 | 5 | 0 | | 100% | 100% |
| | pemphigus antigen) | | | | | | | |
| Q5KSL6 | (Q5KSL6) Diacylglycerol kinase kappa | 141814.6 | 0 | 5 | 0 | | 100% | 100% |
| P31151 | (P31151) S100 calcium-binding protein A7 (Psoriasin) | 11308.5 | 0 | 4 | 0 | | 100% | 100% |
| Q5T749 | (Q5T749) Novel protein (Keratinocyte proline-rich protein) | 64114.6 | 0 | 4 | 0 | | 100% | 100% |
| Q14664 | (Q14664) Keratin 10 | 57231.3 | 1 | 4 | 0 | 100% | 60% | 100% |
| P01781 | (P01781) Ig heavy chain V-III region GAL | 12708.1 | 5 | 4 | 0 | 100% | 11% | 100% |
| P13645 | (P13645) Keratin, type I cytoskeletal 10 (Cytokeratin-10) (CK-10) | 59502.3 | 0 | 57 | 4 | 100% | 100% | 87% |
| | (Keratin-10) (K10) | | | | | | | |
| Q6MZW0 | (Q6MZW0) Hypothetical protein DKFZp686J11235 (Fragment) | 54440.5 | 6 | 5 | 1 | 71% | 9% | 67% |
| P04264 | (P04264) Keratin, type II cytoskeletal 1 (Cytokeratin-1) (CK-1) | 65870 | 6 | 78 | 17 | 48% | 86% | 64% |
| | (Keratin-1) (K1) (67 kDa cytokeratin) (Hair alpha protein) | | | | | | | |
| P35908 | (P35908) Keratin, type II cytoskeletal 2 epidermal (Cytokeratin-2e) | 65848.4 | 0 | 16 | 4 | 100% | 100% | 60% |
| | (K2e) (CK 2e) | | | | | | | |
| P05452 | (P05452) Tetranectin precursor (TN) (Plasminogen-kringle 4 | 22549.1 | 0 | 4 | 1 | 100% | 100% | 60% |
| | binding protein) | | | | | | | |
| P23945 | (P23945) Follicle-stimulating hormone receptor precursor (FSH-R) | 78280.1 | 1 | 7 | 2 | 33% | 75% | 56% |
| | (Follitropin receptor) | | | | | | | |
| P01011 | (P01011) Alpha-1-antichymotrypsin precursor (ACT) [Contains: | 47635 | 12 | 13 | 4 | 50% | 4% | 53% |
| | Alpha-1-antichymotrypsin His-Pro-less] | | | | | | | |
| Q5CZ94 | (Q5CZ94) Hypothetical protein DKFZp781M0386 | 24983.5 | 8 | 6 | 2 | 60% | 14% | 50% |

| | |
|---|---|
| Biomarkers for: | MCI versus mild AD |
| Experiment: | LMW_b (pooled samples of Normal/MCI/mild AD) |
| Filters: | SEQUEST => XC +1 > 1.9, +2 > 2.2, +3 > 3.5 |
| | at least 4 spectra per protein in one group |
| | over 50% difference between MCI and mild AD |
| Key: | more abundant in mild AD |
| | more abundant in MCI |

| | | | | | | PMCI/unst. | | | | | | | |
|---|---|---|---|---|---|---|---|---|---|---|---|---|---|
| Accession | | | MCI | | | MCI | | | | | | MCI − | % |
| (GI_Number) | Protein name | MW | 397 | 531 | 667 | 397 | 531 | 667 | MCI + PMCI | MCI | PMCI | PMCI | Diff. |
| gi\|11386147 | prosaposin [symbol] | 58094 | 0 | 0 | 0 | 1 | 2 | 1 | 4 | 0 | 4 | −4 | 100% |
| gi\|4505873 | phospholipase D1, | 124170 | 0 | 0 | 1 | 2 | 1 | 2 | 6 | 1 | 5 | −4 | 67% |
| | phophatidylcholine-specific [symbol] | | | | | | | | | | | | |
| | [symbol] | | | | | | | | | | | | |
| gi\|13540475 | serum amyloid A2 [symbol] | 13491 | 0 | 0 | 1 | 1 | 1 | 2 | 5 | 1 | 4 | −3 | 60% |
| gi\|4504351 | delta globin [symbol] | 16037 | 22 | 9 | 12 | 50 | 14 | 13 | 120 | 43 | 77 | −34 | 28% |
| gi\|4759050 | ribosomal protein S6 kinase, 90 kDa, | 83721 | 4 | 4 | 3 | 6 | 5 | 4 | 26 | 11 | 15 | −4 | 15% |
| | polypeptide 3 [symbol] | | | | | | | | | | | | |
| gi\|4504489 | histidine-rich glycoprotein | 59559 | 5 | 10 | 13 | 8 | 11 | 19 | 66 | 28 | 38 | −10 | 15% |
| | precursor [symbol] | | | | | | | | | | | | |
| gi\|11761629 | fibrinogen, alpha polypeptide | 69739 | 25 | 44 | 18 | 46 | 54 | 19 | 206 | 87 | 119 | −32 | 16% |
| | isoform alpha preproprotein [symbol] | | | | | | | | | | | | |
| | [symbol] | | | | | | | | | | | | |
| gi\|4557871 | transferrin [symbol] | 77032 | 61 | 78 | 81 | 68 | 104 | 101 | 493 | 220 | 273 | −53 | 11% |
| gi\|10835095 | serum amyloid A4, constitutive | 14789 | 60 | 134 | 120 | 70 | 139 | 128 | 651 | 314 | 337 | −23 | 4% |
| | [symbol] | | | | | | | | | | | | |
| gi\|49574514 | matrix Gla protein [symbol] | 12336 | 0 | 0 | 0 | 3 | 0 | 1 | 4 | 0 | 4 | −4 | 100% |
| gi\|4506769 | S100 calcium-binding protein A7 | 11440 | 0 | 1 | 0 | 1 | 1 | 4 | 7 | 1 | 6 | −5 | 71% |
| | [symbol] | | | | | | | | | | | | |
| gi\|4502419 | biliverdin reductase B (flavin | 22101 | 0 | 0 | 0 | 1 | 0 | 3 | 4 | 0 | 4 | −4 | 100% |
| | reductase (NADPH)) [symbol] | | | | | | | | | | | | |
| | [symbol] | | | | | | | | | | | | |
| gi\|30794266 | triggering receptor expressed on | 32661 | 1 | 0 | 0 | 1 | 1 | 1 | 4 | 1 | 3 | −2 | 50% |
| | myeloid cells-like 1 [symbol] | | | | | | | | | | | | |
| gi\|32698688 | citron [symbol] | 231418 | 0 | 0 | 0 | 2 | 2 | 0 | 4 | 0 | 4 | −4 | 100% |
| gi\|28076869 | serine (or cysteine) proteinase | 44837 | 0 | 0 | 0 | 1 | 0 | 3 | 4 | 0 | 4 | −4 | 100% |
| | inhibitor, clade B (ovalbumin), | | | | | | | | | | | | |
| | member 4 [symbol] | | | | | | | | | | | | |
| gi\|40806175 | diacylglycerol kinase, theta [symbol] | 101135 | 0 | 0 | 0 | 2 | 0 | 1 | 3 | 0 | 3 | −3 | 100% |
| | [symbol] | | | | | | | | | | | | |
| gi\|38455402 | lipocalin 2 (oncogene 24p3) [symbol] | 22571 | 0 | 0 | 0 | 0 | 1 | 1 | 2 | 0 | 2 | −2 | 100% |
| | [symbol] | | | | | | | | | | | | |
| gi\|113417691 | PREDICTED: hypothetical protein | 24550 | 0 | 0 | 0 | 0 | 1 | 1 | 2 | 0 | 2 | −2 | 100% |
| | [symbol] | | | | | | | | | | | | |
| gi\|21361470 | chromosome 1 open reading frame | 32112 | 0 | 0 | 0 | 1 | 1 | 0 | 2 | 0 | 2 | −2 | 100% |
| | 48 [symbol] | | | | | | | | | | | | |
| gi\|33286418 | pyruvate kinase 3 isoform 1 [symbol] | 57920 | 0 | 0 | 0 | 1 | 1 | 0 | 2 | 0 | 2 | −2 | 100% |
| | [symbol] | | | | | | | | | | | | |
| gi\|66346708 | membrane associated guanylate | 136902 | 0 | 0 | 0 | 0 | 1 | 1 | 2 | 0 | 2 | −2 | 100% |
| | kinase, WW and PDZ domain | | | | | | | | | | | | |
| | containing 1 isoform b [symbol] | | | | | | | | | | | | |
| | [symbol] | | | | | | | | | | | | |
| gi\|24586657 | myosin IIIA [symbol] | 186070 | 0 | 0 | 0 | 0 | 1 | 1 | 2 | 0 | 2 | −2 | 100% |
| gi\|11641247 | chromosome 9 open reading frame | 17200 | 0 | 0 | 0 | 1 | 1 | 0 | 2 | 0 | 2 | −2 | 100% |
| | 19 [symbol] | | | | | | | | | | | | |
| gi\|4507267 | stanniocalcin 2 precursor [symbol] | 33230 | 0 | 0 | 0 | 1 | 0 | 1 | 2 | 0 | 2 | −2 | 100% |
| | [symbol] | | | | | | | | | | | | |
| gi\|113426784 | PREDICTED: similar to ribosomal | 27009 | 0 | 0 | 0 | 0 | 1 | 1 | 2 | 0 | 2 | −2 | 100% |
| | protein S2 [symbol] | | | | | | | | | | | | |
| gi\|19923424 | myotubularin-related protein 9 | 63446 | 0 | 0 | 0 | 1 | 1 | 0 | 2 | 0 | 2 | −2 | 100% |
| | [symbol] | | | | | | | | | | | | |
| gi\|4826663 | core-binding factor, runt domain, | 67115 | 0 | 0 | 0 | 1 | 0 | 1 | 2 | 0 | 2 | −2 | 100% |
| | alpha subunit 2; translocated to, 2 | | | | | | | | | | | | |
| | isoform MTGR1b [symbol] | | | | | | | | | | | | |
| gi\|21264361 | mannan-binding lectin serine | 75685 | 0 | 0 | 1 | 1 | 0 | 2 | 4 | 1 | 3 | −2 | 50% |
| | protease 2 isoform 1 precursor | | | | | | | | | | | | |
| | [symbol] | | | | | | | | | | | | |
| gi\|113419903 | PREDICTED: similar to Neutrophil | 10183 | 0 | 2 | 1 | 0 | 4 | 18 | 25 | 3 | 22 | −19 | 76% |
| | defensin 1 precursor (HNP-1) (HP-1) | | | | | | | | | | | | |
| | (HP1) (Defensin, alpha 1) [symbol] | | | | | | | | | | | | |
| | [symbol] | | | | | | | | | | | | |
| gi\|4557894 | lysozyme precursor [symbol] | 16519 | 1 | 0 | 3 | 1 | 2 | 5 | 12 | 4 | 8 | −4 | 33% |
| gi\|38016947 | complement component 5 [symbol] | 188291 | 4 | 3 | 2 | 4 | 5 | 4 | 22 | 9 | 13 | −4 | 18% |
| | [symbol] | | | | | | | | | | | | |
| gi\|38044288 | gelsolin isoform a precursor [symbol] | 85680 | 2 | 5 | 2 | 5 | 7 | 2 | 23 | 9 | 14 | −5 | 22% |
| | [symbol] | | | | | | | | | | | | |
| gi\|50363217 | serine (or cysteine) proteinase | 46720 | 21 | 25 | 17 | 30 | 25 | 31 | 149 | 63 | 86 | −23 | 15% |
| | inhibitor, clade A (alpha-1 | | | | | | | | | | | | |
| | antiproteinase, antitrypsin), member | | | | | | | | | | | | |
| | 1 [symbol] | | | | | | | | | | | | |
| gi\|51339291 | sterile alpha motif domain | 184523 | 3 | 2 | 4 | 4 | 3 | 4 | 20 | 9 | 11 | −2 | 10% |
| | containing 9-like [symbol] | | | | | | | | | | | | |
| gi\|88853069 | vitronectin precursor [symbol] | 54288 | 7 | 5 | 12 | 9 | 7 | 12 | 52 | 24 | 28 | −4 | 8% |
| | [symbol] | | | | | | | | | | | | |
| gi\|50345296 | complement component 4B | 192735 | 12 | 12 | 10 | 13 | 16 | 10 | 73 | 34 | 39 | −5 | 7% |
| | preproprotein [symbol] | | | | | | | | | | | | |
| gi\|31542984 | inter-alpha (globulin) inhibitor H4 | 103340 | 2 | 4 | 4 | 0 | 0 | 0 | 10 | 10 | 0 | 10 | 100% |
| | [Homo sapiens] | | | | | | | | | | | | |
| gi\|55956899 | keratin 9 [Homo sapiens] | 62048 | 6 | 1 | 7 | 0 | 0 | 0 | 14 | 14 | 0 | 14 | 100% |
| gi\|62739186 | complement factor H isoform a | 139052 | 4 | 2 | 4 | 1 | 0 | 0 | 11 | 10 | 1 | 9 | 82% |
| | precursor [Homo sapiens] | | | | | | | | | | | | |
| gi\|21735614 | apolipoprotein L1 isoform a precursor | 43957 | 3 | 3 | 1 | 0 | 1 | 0 | 8 | 7 | 1 | 6 | 75% |
| | [Homo sapiens] | | | | | | | | | | | | |
| gi\|22091452 | apolipoprotein M [Homo sapiens] | 21236 | 4 | 4 | 2 | 2 | 1 | 0 | 13 | 10 | 3 | 7 | 54% |
| gi\|24234699 | keratin 10 [Homo sapiens] | 58811 | 8 | 7 | 11 | 2 | 3 | 3 | 34 | 26 | 8 | 18 | 53% |
| gi\|7705931 | hect domain and RLD 5 [Homo | 116834 | 3 | 3 | 2 | 1 | 1 | 1 | 11 | 8 | 3 | 5 | 45% |
| | sapiens] | | | | | | | | | | | | |
| gi\|17318569 | keratin 1 [Homo sapiens] | 66050 | 36 | 5 | 7 | 3 | 4 | 3 | 58 | 48 | 10 | 38 | 66% |
| gi\|21071030 | alpha 1B-glycoprotein [Homo sapiens] | 54235 | 3 | 5 | 5 | 1 | 3 | 2 | 19 | 13 | 6 | 7 | 37% |
| gi\|4505733 | platelet factor 4 (chemokine (C—X—C | 10828 | 13 | 13 | 10 | 7 | 7 | 7 | 57 | 36 | 21 | 15 | 26% |
| | motif) ligand 4) [Homo sapiens] | | | | | | | | | | | | |
| gi\|4557393 | complement component 8, gamma | 22201 | 25 | 36 | 64 | 23 | 24 | 16 | 188 | 125 | 63 | 62 | 33% |
| | polypeptide [Homo sapiens] | | | | | | | | | | | | |
| gi\|4504349 | beta globin [Homo sapiens] | 15980 | 289 | 100 | 110 | 109 | 61 | 95 | 764 | 499 | 265 | 234 | 31% |
| gi\|32130518 | apolipoprotein C-II precursor [Homo | 11266 | 41 | 96 | 268 | 38 | 52 | 154 | 649 | 405 | 244 | 161 | 25% |
| | sapiens] | | | | | | | | | | | | |
| gi\|4503635 | coagulation factor II precursor [Homo | 70019 | 13 | 9 | 14 | 11 | 5 | 11 | 63 | 36 | 27 | 9 | 14% |
| | sapiens] | | | | | | | | | | | | |
| gi\|32483410 | vitamin D-binding protein precursor | 52900 | 9 | 10 | 12 | 7 | 7 | 10 | 55 | 31 | 24 | 7 | 13% |
| | [Homo sapiens] | | | | | | | | | | | | |
| gi\|34878902 | pericentriolar material 1 [Homo | 228284 | 1 | 1 | 1 | 1 | 0 | 0 | 4 | 3 | 1 | 2 | 50% |
| | sapiens] | | | | | | | | | | | | |
| gi\|5730021 | regulatory solute carrier protein, family | 66771 | 0 | 2 | 1 | 0 | 0 | 0 | 3 | 3 | 0 | 3 | 100% |
| | 1, member 1 [Homo sapiens] | | | | | | | | | | | | |
| gi\|38045901 | leader-binding protein 32 isoform 1 | 70096 | 0 | 1 | 2 | 0 | 0 | 0 | 3 | 3 | 0 | 3 | 100% |
| | [Homo sapiens] | | | | | | | | | | | | |
| gi\|40786418 | profilin family, member 4 [Homo | 14302 | 1 | 2 | 0 | 0 | 0 | 0 | 3 | 3 | 0 | 3 | 100% |
| | sapiens] | | | | | | | | | | | | |
| gi\|34577063 | adenylosuccinate synthase [Homo | 50080 | 1 | 0 | 1 | 0 | 0 | 0 | 2 | 2 | 0 | 2 | 100% |
| | sapiens] | | | | | | | | | | | | |
| gi\|88973313 | PREDICTED: similar to Zinc finger | 58993 | 1 | 1 | 0 | 0 | 0 | 0 | 2 | 2 | 0 | 2 | 100% |
| | CCHC domain-containing protein 4 | | | | | | | | | | | | |
| | isoform 1 [Homo sapiens] | | | | | | | | | | | | |
| gi\|4503005 | plasma carboxypeptidase B2 isoform a | 48425 | 1 | 1 | 0 | 0 | 0 | 0 | 2 | 2 | 0 | 2 | 100% |
| | preproprotein [Homo sapiens] | | | | | | | | | | | | |
| gi\|50363226 | secretoglobin, family 3A, member 1 | 10082 | 1 | 1 | 0 | 0 | 0 | 0 | 2 | 2 | 0 | 2 | 100% |
| | [Homo sapiens] | | | | | | | | | | | | |
| gi\|38638698 | KIAA1199 [Homo sapiens] | 152983 | 1 | 1 | 0 | 0 | 0 | 0 | 2 | 2 | 0 | 2 | 100% |
| gi\|31742536 | fidgetin-like 1 [Homo sapiens] | 74061 | 1 | 1 | 0 | 0 | 0 | 0 | 2 | 2 | 0 | 2 | 100% |
| gi\|65506442 | propionyl-Coenzyme A carboxylase, | 80041 | 10 | 7 | 5 | 3 | 0 | 5 | 30 | 22 | 8 | 14 | 47% |
| | alpha polypeptide precursor [Homo | | | | | | | | | | | | |
| | sapiens] | | | | | | | | | | | | |
| gi\|61888896 | roundabout, axon guidance receptor, | 151182 | 2 | 2 | 0 | 0 | 1 | 0 | 5 | 4 | 1 | 3 | 60% |
| | homolog 2 [Homo sapiens] | | | | | | | | | | | | |
| gi\|33285006 | Rsb-66 protein [Homo sapiens] | 19271 | 2 | 0 | 2 | 0 | 0 | 1 | 5 | 4 | 1 | 3 | 60% |
| gi\|31543806 | thrombospondin 4 precursor [Homo | 105851 | 2 | 1 | 1 | 1 | 1 | 0 | 6 | 4 | 2 | 2 | 33% |
| | sapiens] | | | | | | | | | | | | |
| gi\|32189392 | peroxiredoxin 2 isoform a [Homo | 21874 | 3 | 2 | 3 | 3 | 0 | 2 | 13 | 8 | 5 | 3 | 23% |
| | sapiens] | | | | | | | | | | | | |
| gi\|14211540 | Mov10, Moloney leukemia virus 10, | 113658 | 3 | 2 | 3 | 2 | 2 | 2 | 14 | 8 | 6 | 2 | 14% |
| | homolog [Homo sapiens] | | | | | | | | | | | | |

| | |
|---|---|
| Biomarkers for: | MCI versus mild AD |
| Experiment: | LMW_c (same patients before and after cognitive |
| | decline) |
| Filters: | SEQUEST |
| | increases in at least two of three samples OR |
| | decreases in at least two of three samples |
| | third sample can not decrease OR third sample can |
| | not increase (can only be equal) |
| Key: | more abundant in mild AD |
| | more abundant in MCI |

| | | | | | | Normal | | | | | | | | | | |
|---|---|---|---|---|---|---|---|---|---|---|---|---|---|---|---|---|
| | | | | Stable | Unstable | (stable + | Stable | | | | | Stable | | | | |
| | | | | Normal | Normal | unstable) | MCI | mild AD | | mild | Unstable | Normal | | | Unstable | Stable |
| | | | | [av | [av | [av | [av | [av | Stable | AD | Normal vs | vs | Unstable | | Normal | Normal |
| | | | | samples] | samples] | samples] | samples] | samples] | MCI vs | vs | Stable | Stable | Normal vs | Stable MCI | vs | vs |
| Accession | Name | Probability | MW | (n = 3) | (n = 4) | (n = 7) | (n = 5) | (n = 12) | Normal | Normal | Normal | MCI | Stable MCI | vs mild AD | mild AD | mild AD |
| P35542 | SAA4_HUMAN | 9.09E−04 | 14797.3 | 0.00 | 0.25 | 0.14 | 0.00 | 0.58 | 100% | 61% | 100% | 0% | 100% | 100% | 40% | 100% |
| | (P35542) | | | | | | | | | | | | | | | |
| | Serum | | | | | | | | | | | | | | | |
| | amyloid A-4 | | | | | | | | | | | | | | | |
| | protein | | | | | | | | | | | | | | | |
| | precursor | | | | | | | | | | | | | | | |
| | (Constitutively | | | | | | | | | | | | | | | |
| | expres | | | | | | | | | | | | | | | |
| P02766 | TTHY_HUMAN | 2.78E−13 | 15877.1 | 1.00 | 0.50 | 0.71 | 0.00 | 0.50 | 100% | 18% | 33% | 100% | 100% | 100% | 0% | 33% |
| | (P02766) | | | | | | | | | | | | | | | |
| | Transthyretin | | | | | | | | | | | | | | | |
| | precursor | | | | | | | | | | | | | | | |
| | (Prealbumin) | | | | | | | | | | | | | | | |
| | (TBPA) (TTR) | | | | | | | | | | | | | | | |
| | (ATTR) | | | | | | | | | | | | | | | |
| P09874 | PARP1_HUMAN | 4.55E−04 | 112881.4 | 0.00 | 0.50 | 0.29 | 0.00 | 0.42 | 100% | 19% | 100% | 0% | 100% | 100% | 9% | 100% |
| | (P09874) | | | | | | | | | | | | | | | |
| | Poly [ADP- | | | | | | | | | | | | | | | |
| | ribose] | | | | | | | | | | | | | | | |
| | polymerase | | | | | | | | | | | | | | | |
| | 1 (EC | | | | | | | | | | | | | | | |
| | 2.4.2.30) | | | | | | | | | | | | | | | |
| | (PARP-1) | | | | | | | | | | | | | | | |
| | (AD | | | | | | | | | | | | | | | |
| P01764 | HV3C_HUMAN | 4.40E−05 | 12574.2 | 0.00 | 0.75 | 0.43 | 0.00 | 0.42 | 100% | 1% | 100% | 0% | 100% | 100% | 29% | 100% |
| | (P01764) Ig | | | | | | | | | | | | | | | |
| | heavy chain | | | | | | | | | | | | | | | |
| | V-III region | | | | | | | | | | | | | | | |
| | VH26 | | | | | | | | | | | | | | | |
| | precursor | | | | | | | | | | | | | | | |
| Q9NPP6 | Q9NPP6 | 7.65E−04 | 44758.1 | 0.33 | 0.50 | 0.43 | 0.00 | 0.33 | 100% | 13% | 20% | 100% | 100% | 100% | 20% | 0% |
| | (Q9NPP6) | | | | | | | | | | | | | | | |
| | Immunoglobulin | | | | | | | | | | | | | | | |
| | heavy | | | | | | | | | | | | | | | |
| | chain variant | | | | | | | | | | | | | | | |
| | (Fragment) | | | | | | | | | | | | | | | |
| Q96MA6 | Q96MA6 | 5.15E−04 | 54890.7 | 0.33 | 0.25 | 0.29 | 0.00 | 0.33 | 100% | 8% | 14% | 100% | 100% | 100% | 14% | 0% |
| | (Q96MA6) | | | | | | | | | | | | | | | |
| | Hypothetical | | | | | | | | | | | | | | | |
| | protein | | | | | | | | | | | | | | | |
| | FLJ32704 | | | | | | | | | | | | | | | |
| | (Chromosome | | | | | | | | | | | | | | | |
| | 9 open | | | | | | | | | | | | | | | |
| | reading | | | | | | | | | | | | | | | |
| | frame | | | | | | | | | | | | | | | |
| Q9NYQ6 | CELR1_HUMAN | 1.30E−04 | 329276.7 | 0.33 | 0.50 | 0.43 | 0.00 | 0.33 | 100% | 13% | 20% | 100% | 100% | 100% | 20% | 0% |
| | (Q9NYQ6) | | | | | | | | | | | | | | | |
| | Cadherin | | | | | | | | | | | | | | | |
| | EGF LAG | | | | | | | | | | | | | | | |
| | seven-pass | | | | | | | | | | | | | | | |
| | G-type | | | | | | | | | | | | | | | |
| | receptor 1 | | | | | | | | | | | | | | | |
| | precursor ( | | | | | | | | | | | | | | | |
| O60602 | TLR5_HUMAN | 2.76E−04 | 97663.4 | 0.00 | 0.00 | 0.00 | 0.00 | 0.33 | | 100% | 0% | 0% | 0% | 100% | 100% | 100% |
| | (O60602) | | | | | | | | | | | | | | | |
| | Toll-like | | | | | | | | | | | | | | | |
| | receptor 5 | | | | | | | | | | | | | | | |
| | precursor | | | | | | | | | | | | | | | |
| | (Toll/interleukin- | | | | | | | | | | | | | | | |
| | 1 recepto | | | | | | | | | | | | | | | |
| Q15166 | PON3_HUMAN | 1.52E−05 | 39582.4 | 0.33 | 0.00 | 0.14 | 0.00 | 0.33 | 100% | 40% | 100% | 100% | 0% | 100% | 100% | 0% |
| | (Q15166) | | | | | | | | | | | | | | | |
| | Serum | | | | | | | | | | | | | | | |
| | paraoxonase/ | | | | | | | | | | | | | | | |
| | lactonase 3 | | | | | | | | | | | | | | | |
| | (EC 3.1.1.—) | | | | | | | | | | | | | | | |
| O60687 | O60687 | 2.32E−04 | 52937.8 | 0.00 | 0.00 | 0.00 | 0.40 | 0.00 | 100% | | 0% | 100% | 100% | 100% | 0% | 0% |
| | (O60687) | | | | | | | | | | | | | | | |
| | Sushi-repeat- | | | | | | | | | | | | | | | |
| | containing | | | | | | | | | | | | | | | |
| | protein, X- | | | | | | | | | | | | | | | |
| | linked 2 | | | | | | | | | | | | | | | |
| Q6N092 | Q6N092 | 9.38E−04 | 56387.9 | 0.00 | 0.25 | 0.14 | 0.40 | 0.00 | 47% | 100% | 100% | 100% | 23% | 100% | 100% | 0% |
| | (Q6N092) | | | | | | | | | | | | | | | |
| | Hypothetical | | | | | | | | | | | | | | | |
| | protein | | | | | | | | | | | | | | | |
| | DKFZp686K18196 | | | | | | | | | | | | | | | |
| | (Fragment) | | | | | | | | | | | | | | | |
| Q96S24 | Q96S24 | 9.13E−05 | 3518.7 | 0.00 | 0.00 | 0.00 | 0.40 | 0.00 | 100% | | | 100% | 100% | 100% | 0% | 0% |
| | (Q96S24) | | | | | | | | | | | | | | | |
| | Hypothetical | | | | | | | | | | | | | | | |
| | protein gs30 | | | | | | | | | | | | | | | |
| P20701 | ITAL_HUMAN | 2.34E−04 | 128738 | 0.00 | 0.00 | 0.00 | 0.60 | 0.08 | 100% | 100% | 0% | 100% | 100% | 76% | 100% | 100% |
| | (P20701) | | | | | | | | | | | | | | | |
| | Integrin | | | | | | | | | | | | | | | |
| | alpha-L | | | | | | | | | | | | | | | |
| | precursor | | | | | | | | | | | | | | | |
| | (Leukocyte | | | | | | | | | | | | | | | |
| | adhesion | | | | | | | | | | | | | | | |
| | glycoprotein | | | | | | | | | | | | | | | |
| | LFA-1 alpha | | | | | | | | | | | | | | | |
| | chain) | | | | | | | | | | | | | | | |
| Q8N549 | Q8N549 | 9.74E−05 | 27915 | 0.00 | 0.25 | 0.14 | 0.40 | 0.08 | 47% | 26% | 100% | 100% | 23% | 66% | 50% | 100% |
| | (Q8N549) | | | | | | | | | | | | | | | |
| | Hypothetical | | | | | | | | | | | | | | | |
| | protein | | | | | | | | | | | | | | | |
| | C8orf36 | | | | | | | | | | | | | | | |
| P09758 | TACD2_HUMAN | 3.03E−04 | 35686.6 | 0.67 | 0.25 | 0.43 | 0.40 | 0.08 | 3% | 67% | 45% | 25% | 23% | 66% | 50% | 78% |
| | (P09758) | | | | | | | | | | | | | | | |
| | Tumor- | | | | | | | | | | | | | | | |
| | associated | | | | | | | | | | | | | | | |
| | calcium signal | | | | | | | | | | | | | | | |
| | transducer 2 | | | | | | | | | | | | | | | |
| | precursor (P | | | | | | | | | | | | | | | |
| P10643 | CO7_HUMAN | 1.10E−04 | 93457.3 | 0.00 | 0.50 | 0.29 | 0.80 | 0.25 | 47% | 7% | 100% | 100% | 23% | 52% | 33% | 100% |
| | (P10643) | | | | | | | | | | | | | | | |
| | Complemet | | | | | | | | | | | | | | | |
| | component | | | | | | | | | | | | | | | |
| | C7 precursor | | | | | | | | | | | | | | | |

| | |
|---|---|
| Biomarkers for: | MCI versus mild AD |
| Experiment: | whole serum (individual samples of Normal/MCI/mild |
| | AD) |
| Filters: | SEQUEST |
| | found in more than 33% of either normals or mild AD |
| | patients |
| | over 50% difference between normal and mild AD (in |
| | average samples with positive identification) |
| | probability score of less than 1.00E−03 |
| Key: | more abundant in mild AD |
| | more abundant in MCI |

TABLE 10
Analysis of biomarkers in different patient populations.

| whole | Accession | Name | StudySet | Identified spectra/Number of samples | | | | Samples with hits/Number of samples | | | | | |
|---|---|---|---|---|---|---|---|---|---|---|---|---|---|
| serum: | | | | 0 (n = 3) | 1 (n = 4) | 2 (n = 5) | 4 | 0 | 1 | 0 + 1 | 2 | 4 | |
| | | | | | | | (n = 12) | (n = 3) | (n = 4) | (n = 7) | (n = 5) | (n = 12) | |
| LMW_b: | Accession | Name | StudySet | Identified spectra | | | | | | | | | |
| | | | | Norm | MCI | PMCI | | | | | | | |
| | | | | (n = 14) | (n = 14) | (n = 14) | | | | | | | |
| LMW_c: | GI_Number | Protein name | StudySet | Norm | Unst | MCI | MCI | MCI | | | PMCI/unst MCI | | |
| | | | | 50 | Norm | 1305 | 1305 | 397 | 531 | 667 | 397 | 531 | 667 |
| | | | | | 50 | | | | | | | | |
| homocysteine | Q13867 | Bleomycin hydrolase | whole | 0.67 | 0.25 | 0.00 | 0.00 | 0.67 | 0.25 | 0.43 | 0.00 | 0.00 | |
| metabolism | | | serum | | | | | | | | | | |
| | Q13867 | Bleomycin hydrolase | LMW_b | 0 | 1 | 0 | | | | | | | |
| | Q93088 | Betaine-homocysteine S- | whole | 0.00 | 1.25 | 2.80 | 2.33 | 0.00 | 0.25 | 0.14 | 0.60 | 0.75 | |
| | | methyltransferase | serum | | | | | | | | | | |
| | P27169 | Serum paraoxonase 1 | LMW_b | 3 | 2 | 1 | | | | | | | |
| | Q6B0J6 | Serum paraoxonase 1 | whole | 0.00 | 0.00 | 0.00 | 0.08 | 0.00 | 0.00 | 0.00 | 0.00 | 0.08 | |
| | | | serum | | | | | | | | | | |
| | P27169 | Serum paraoxonase 1 | whole | 7.33 | 4.00 | 3.20 | 2.00 | 1.00 | 0.75 | 0.86 | 0.80 | 0.67 | |
| | | | serum | | | | | | | | | | |
| | gi\|19923106 | Serum paraoxonase 1 | LMW_c | 3 | 0 | 2 | 0 | 4 | 2 | 2 | 3 | 2 | 4 |
| | Q15166 | Serum paraoxonase 3 | whole | 0.33 | 0.00 | 0.00 | 0.58 | 0.33 | 0.00 | 0.14 | 0.00 | 0.33 | |
| | | | serum | | | | | | | | | | |

Bleomycin hydrolase protects against homocysteine toxicity (Zimny2006)
Elevated serum homocysteine is a risk factor for Alzheimer's disease
Polymorphisms in bleomycin hydrolase have been found to be correlated with the incidence of AD - however replication of the finding shows mixed results
Bleomycin hydrolase regulates the secretion of soluble APP and Abeta (Lefterov2001)
Betaine--homocysteine S-methyltransferase is one of two key enzymes involved in the removal of homocysteine (Brosnan2004)
Betaine is considered as a therapeutic in AD to lower plasma homocysteine (Knopman2001)
Methionyl tRNA synthetase edits homocysteine out if it gets inserted into proteins instead of methionine - this results in homocysteine-thiolacetone (Zimny2006)
Homocysteine-thiolacetone gets cleared by bleomycin hydrolase inside cells (Zimny2006)
Homocysteine-thiolacetone gets cleared by serum paraoxonase 1 in the serum (Himbergen2006)
Homocysteine-thiolacetone can impair protein synthesis and lead to endothelial dysfunction and vascular damage (Himbergen2006)

| | | | | | | | | | | | | | |
|---|---|---|---|---|---|---|---|---|---|---|---|---|---|
| inflammation | Q4V312 | Colony stimulating factor 2 receptor (GM- | whole | 0.00 | 0.00 | 2.40 | 1.50 | 0.00 | 0.00 | 0.00 | 0.60 | 0.33 | |
| | | CSF) | serum | | | | | | | | | | |
| | gi\|4506765 | S100 calcium-binding protein A4 | LMW_c | 0 | 0 | 1 | 0 | 0 | 0 | 0 | 0 | 0 | 0 |
| | gi\|7657532 | S100 calcium-binding protein A6 | LMW_c | 0 | 0 | 0 | 1 | 0 | 0 | 0 | 0 | 0 | 0 |
| | P31151 | S100 calcium-binding protein A7 | LMW_b | 0 | 4 | 0 | | | | | | | |
| | gi\|4506769 | S100 calcium-binding protein A7 | LMW_c | 0 | 6 | 1 | 0 | 0 | 1 | 0 | 1 | 1 | 4 |
| | P05109 | S100 calcium-binding protein A8 | LMW_b | 2 | 6 | 3 | | | | | | | |
| | gi\|21614544 | S100 calcium-binding protein A8 | LMW_c | 3 | 11 | 15 | 3 | 5 | 4 | 4 | 2 | 14 | 5 |
| | P06702 | S100 calcium-binding protein A9 | LMW_b | 0 | 1 | 0 | | | | | | | |
| | gi\|4506773 | S100 calcium-binding protein A9 | LMW_c | 2 | 4 | 3 | 4 | 3 | 3 | 6 | 3 | 5 | 5 |
| | P02735 | Serum amyloid A protein precursor (SAA) | LMW_b | 3 | 6 | 5 | | | | | | | |
| | P02735 | Serum amyloid A protein precursor (SAA) | whole | 0.00 | 0.00 | 0.00 | 0.25 | 0.00 | 0.00 | 0.00 | 0.00 | 0.17 | |
| | | | serum | | | | | | | | | | |
| | gi\|40316910 | Serum amyloid A1 isoform 2 (SAA1) | LMW_c | 11 | 7 | 5 | 8 | 15 | 7 | 2 | 10 | 13 | 0 |
| | gi\|13540475 | Serum amyloid A2 (SAA2) | LMW_c | 0 | 0 | 4 | 1 | 0 | 0 | 1 | 1 | 1 | 2 |
| | P22614 | Putative serum amyloid A3 (SAA3) | whole | 0.00 | 0.00 | 0.00 | 0.08 | 0.00 | 0.00 | 0.00 | 0.00 | 0.08 | |
| | | | serum | | | | | | | | | | |
| | P22614 | Putative serum amyloid A3 (SAA3) | LMW_b | 0 | 1 | 1 | | | | | | | |
| | P35542 | Serum amyloid A4 protein precursor | whole | 0.00 | 0.25 | 0.00 | 1.25 | 0.00 | 0.25 | 0.14 | 0.00 | 0.58 | |
| | | (SAA4) | serum | | | | | | | | | | |
| | P35542 | Serum amyloid A4 protein precursor (SAA4) | LMW_b | 41 | 53 | 28 | | | | | | | |
| | gi\|10835095 | Serum amyloid A4 protein precursor (SAA4) | LMW_c | 98 | 87 | 82 | 82 | 60 | 134 | 120 | 70 | 139 | 128 |

GM-CSF receptor alpha is upregulated in ischemic brain + increased GM-CSF helps to reduce neuronal injury (Schaebitz2007)
GM-CSF levels are highly increased in AD and vascular dementia serum and CSF (Tarkowski2001)
Abeta activated microglia upregulate S100A8 expression (Walker2006) - however, it is not associated with AD brain lesions (Shepherd2006)
SAA2 is an acute phase protein that is induced by the inflammatory cytokines IL-1 and IL-6 (Longley1999)

| | | | | | | | | | | | | | |
|---|---|---|---|---|---|---|---|---|---|---|---|---|---|
| estrogen | P03372 | Estrogen receptor alpha | whole | 0.00 | 0.00 | 1.00 | 0.25 | 0.00 | 0.00 | 0.00 | 0.60 | 0.25 | |
| | | | serum | | | | | | | | | | |
| | Q92731 | Estrogen receptor beta | whole | 0.00 | 0.25 | 0.00 | 0.00 | 0.00 | 0.25 | 0.14 | 0.00 | 0.00 | |
| | | | serum | | | | | | | | | | |
| | O95718 | Estrogen-related receptor (Steroid hormone | whole | 0.33 | 0.00 | 0.00 | 0.17 | 0.33 | 0.00 | 0.14 | 0.00 | 0.17 | |
| | | receptor ERR2) | serum | | | | | | | | | | |
| | Q5TF95 | Estrogen nuclear receptor coactivator 1 | whole | 0.33 | 0.50 | 0.00 | 0.08 | 0.33 | 0.25 | 0.29 | 0.00 | 0.08 | |
| | | | serum | | | | | | | | | | |
| | P49888 | Estrogen sulfotransferase (EC 2.8.2.4) | whole | 0.33 | 0.00 | 0.40 | 0.17 | 0.33 | 0.00 | 0.14 | 0.20 | 0.08 | |
| | | | serum | | | | | | | | | | |
| | P50224 | Placental estrogen sulfotransferase (EC | LMW_b | 1 | 0 | 0 | | | | | | | |
| | | 2.8.2.1) | | | | | | | | | | | |
| | P11511 | Estrogen synthetase (Cytochrome P450 | whole | 0.00 | 0.00 | 0.00 | 0.08 | 0.00 | 0.00 | 0.00 | 0.00 | 0.08 | |
| | | 19A1) | serum | | | | | | | | | | |
| | gi\|62244004 | Modulator of estrogen induced transcription | LMW_c | 0 | 0 | 0 | 0 | 1 | 0 | 0 | 0 | 0 | 0 |
| | P23945 | Follicle-stimulating hormone receptor | LMW_b | 78280.1 | 1 | 7 | 2 | | | | | | |
| | | precursor (Follitropin receptor) | | | | | | | | | | | |

Estrogen has a neuroprotective effect - possibly via mitochondria (Singh2006)
Brain estrogen is reduced in AD women (Combarros2007)
Estrogen receptor alpha and butylylcholinesterase genes seem to interact with regard to AD (Combarros2007)
Estrogen receptor alpha gene variants are associated with AD (Luckhaus2007)
FSH regulates estrogen production
FSH-R knockout mice have reduced estrogen production and neurodegenerative symptoms (Danilovich2004)

| | | | | | | | | | | | | | |
|---|---|---|---|---|---|---|---|---|---|---|---|---|---|
| PEDF | P36955 | Pigment epithelium-derived factor | whole | 0.00 | 0.25 | 1.20 | 0.33 | 0.00 | 0.25 | 0.14 | 0.80 | 0.33 | |
| | | precursor | serum | | | | | | | | | | |
| | gi\|39725934 | Pigment epithelium-derived factor precursor | LMW_c | 0 | 0 | 0 | 0 | 0 | 0 | 0 | 0 | 0 | 1 |
| | Q9HD28 | Retinitis pigmentosa GTPase regulator | whole | 0.00 | 0.25 | 0.00 | 0.00 | 0.00 | 0.25 | 0.14 | 0.00 | 0.00 | |
| | | | serum | | | | | | | | | | |
| | Q8IWN7 | Retinitis pigmentosa 1-like 1 protein | whole | 0.00 | 0.00 | 0.00 | 0.17 | 0.00 | 0.00 | 0.00 | 0.00 | 0.17 | |
| | | | serum | | | | | | | | | | |

PEDF is upregulated in AD brain (Yamagishi2004)
PEDF has been identified in CSF proteomics to be increased in AD (Castano2006)

| | | | | | | | | | | | | | |
|---|---|---|---|---|---|---|---|---|---|---|---|---|---|
| vitamin D | Q6GTG1 | Vitamin D-binding protein | LMW_b | 6 | 6 | 2 | | | | | | | |
| metabolism | Q6GTG1 | Vitamin D-binding protein | whole | 33.00 | 40.25 | 46.00 | 40.50 | 1.00 | 1.00 | 1.00 | 1.00 | 1.00 | |
| and | | | serum | | | | | | | | | | |
| bone | | Vitamin D-binding protein precursor | LMW_c | 8 | 4 | 11 | 4 | 9 | 10 | 12 | 7 | 7 | 10 |
| mineralization | O95425 | Supervillin | whole | 1.00 | 0.00 | 2.00 | 1.17 | 0.33 | 0.00 | 0.14 | 0.80 | 0.67 | |
| | | | serum | | | | | | | | | | |
| | gi\|49574514 | Matrix Gla protein | LMW_c | 2 | 0 | 1 | 1 | 0 | 0 | 0 | 3 | 0 | 1 |

Vitamin D-binding protein shows higher carbonylation in AD men (identified in CSF) (Korolainen2007)
Vitamin D-binding protein is found in AD CSF (Finehout2007)
Vitamin D receptor expression is downregulated in AD hippocampal CA1 cells (Sutherland1992)
Vitamin D is neuroprotective (regulates neuronal calcium homeostasis) and vitamin D receptor polymorphisms are associated with AD (Gezen-Ak2007)
Supervillin enhances the transactivation of vitamin D receptor (Ting2005)
Elderly women with AD show reduced bone density, vitamin D deficiency and higher Gla concentration (Sato1998)
Vitamin D is involved in bone mineralization (Demay2007)
Gla is cleaved by HtrA1, which is implicated in AD (Canfield2007)
HtrA1 degrades APP fragments, colocalizes with Abeta deposits in brain and causes accumulation of Abeta if inhibited (Grau2005)
Gla is a potent matrix mineralization inhibitor (Demay2007)

| | | | | | | | | | | | | | |
|---|---|---|---|---|---|---|---|---|---|---|---|---|---|
| coagulation | P02671 | Fibrinogen alpha chain precursor | LMW_b | 0 | 4 | 4 | | | | | | | |
| and | gi\|11761629 | Fibrinogen, alpha polypeptide isoform | LMW_c | 31 | 28 | 52 | 20 | 25 | 44 | 18 | 46 | 54 | 19 |
| platelets | | alpha preproprotein | | | | | | | | | | | |
| | P02671 | Fibrinogen alpha chain precursor | whole | 0.00 | 0.25 | 0.40 | 0.75 | 0.00 | 0.25 | 0.14 | 0.40 | 0.33 | |
| | | | serum | | | | | | | | | | |
| | gi\|70906437 | fibrinogen, gamma chain isoform gamma-A | LMW_c | 0 | 0 | 0 | 0 | 1 | 0 | 0 | 0 | 0 | 0 |
| | | precursor | | | | | | | | | | | |
| | gi\|4503635 | Coagulation factor II precursor | LMW_c | 11 | 8 | 10 | 4 | 13 | 9 | 14 | 11 | 5 | 11 |
| | | (Prothrombin precursor) | | | | | | | | | | | |
| | P00734 | Coagulation factor II precursor | LMW_b | 2 | 0 | 0 | | | | | | | |
| | | (Prothrombin precursor) | | | | | | | | | | | |
| | P00734 | Coagulation factor II precursor (Prothrombin | whole | 9.67 | 8.75 | 10.40 | 10.25 | 1.00 | 1.00 | 1.00 | 1.00 | 1.00 | |
| | | precursor) | serum | | | | | | | | | | |
| | P12259 | Coagulation factor V precursor | whole | 3.00 | 0.00 | 0.40 | 0.83 | 1.00 | 0.00 | 0.43 | 0.20 | 0.42 | |
| | | | serum | | | | | | | | | | |
| | gi\|4503643 | Coagulation factor V precursor | LMW_c | 2 | 0 | 0 | 0 | 0 | 0 | 0 | 0 | 1 | 0 |
| | P00451 | Coagulation factor VIII precursor | LMW_b | 1 | 0 | 0 | | | | | | | |
| | P00451 | Coagulation factor VIII precursor | whole | 0.00 | 0.25 | 0.00 | 0.00 | 0.00 | 0.25 | 0.14 | 0.00 | 0.00 | |
| | | | serum | | | | | | | | | | |
| | gi\|4503649 | Coagulation factor IX | LMW_c | 0 | 1 | 0 | 0 | 0 | 0 | 0 | 0 | 0 | 0 |
| | gi\|4503625 | Coagulation factor X preproprotein | LMW_c | 0 | 0 | 4 | 0 | 0 | 1 | 0 | 0 | 1 | 1 |
| | P00742 | Coagulation factor X precursor | whole | 0.00 | 0.00 | 0.20 | 0.00 | 0.00 | 0.00 | 0.00 | 0.20 | 0.00 | |
| | | | serum | | | | | | | | | | |
| | P03951 | Coagulation factor XI precursor | whole | 0.00 | 0.00 | 0.00 | 0.25 | 0.00 | 0.00 | 0.00 | 0.00 | 0.25 | |
| | | | serum | | | | | | | | | | |
| | P00748 | Coagulation factor XII precursor | whole | 0.67 | 0.75 | 0.60 | 0.50 | 0.33 | 0.50 | 0.43 | 0.40 | 0.42 | |
| | | | serum | | | | | | | | | | |
| | P00488 | Coagulation factor XIII A chain precursor | whole | 0.00 | 0.00 | 0.00 | 0.08 | 0.00 | 0.00 | 0.00 | 0.00 | 0.08 | |
| | | | serum | | | | | | | | | | |
| | P05160 | Coagulation factor XIII B chain precursor | whole | 0.33 | 0.00 | 0.00 | 0.00 | 0.33 | 0.00 | 0.14 | 0.00 | 0.00 | |
| | | | serum | | | | | | | | | | |
| | Q7KZ97 | Antithrombin III variant | LMW_b | 0 | 1 | 1 | | | | | | | |
| | Q7KZ97 | Antithrombin III variant | whole | 2.00 | 1.75 | 1.20 | 2.00 | 1.00 | 0.75 | 0.86 | 0.80 | 0.83 | |
| | | | serum | | | | | | | | | | |
| | gi\|4502261 | Antithrombin (serine (or cysteine) proteinase | LMW_c | 14 | 8 | 12 | 4 | 5 | 11 | 12 | 7 | 7 | 8 |
| | | inhibitor, clade C member 1) | | | | | | | | | | | |
| | Q96P15 | SERPINB11 | whole | 0.00 | 0.00 | 0.00 | 0.08 | 0.00 | 0.00 | 0.00 | 0.00 | 0.08 | |
| | | | serum | | | | | | | | | | |
| | Q8TCE1 | SERPINC1 | LMW_b | 2 | 1 | 1 | | | | | | | |
| | Q8TCE1 | SERPINC1 | whole | 1.00 | 1.25 | 3.20 | 2.50 | 0.33 | 0.75 | 0.57 | 0.80 | 0.83 | |
| | | | serum | | | | | | | | | | |
| | P05546 | SERPIND1 percursor (Heparin cofactor II | LMW_b | 2 | 1 | 0 | | | | | | | |
| | | precursor) | | | | | | | | | | | |
| | gi\|50659080 | Serpin peptidase inhibitor, clade A, member | LMW_c | 9 | 4 | 5 | 6 | 5 | 4 | 3 | 7 | 10 | 1 |
| | | 3 precursor | | | | | | | | | | | |
| | gi\|4507377 | Serine (or cysteine) proteinase inhibitor, | LMW_c | 0 | 1 | 0 | 0 | 1 | 0 | 0 | 0 | 0 | 1 |
| | | clade A, member 7 | | | | | | | | | | | |
| | gi\|39725934 | Serine (or cysteine) proteinase inhibitor, | LMW_c | 0 | 0 | 0 | 0 | 0 | 0 | 0 | 0 | 0 | 1 |
| | | clade F member 1 (PEDF) | | | | | | | | | | | |
| | gi\|50363217 | Alpha-1-antitrypsin (serine (or cysteine) | LMW_c | 27 | 21 | 24 | 19 | 21 | 25 | 17 | 30 | 25 | 31 |
| | | proteinase inhibitor, clade A member 1) | | | | | | | | | | | |
| | gi\|28076869 | Ovalbumin (serine (or cysteine) | LMW_c | 0 | 3 | 0 | 0 | 0 | 0 | 0 | 1 | 0 | 3 |
| | | proteinase inhibitor, clade B member 4) | | | | | | | | | | | |
| | gi\|4505733 | Platelet factor 4 | LMW_c | 13 | 8 | 10 | 7 | 13 | 13 | 10 | 7 | 7 | 7 |
| | gi\|4505735 | Platelet factor 4 variant 1 | LMW_c | 0 | 0 | 0 | 1 | 0 | 1 | 0 | 0 | 1 | 1 |
| | P10720 | Platelet factor 4 variant precursor | whole | 2.33 | 0.75 | 0.20 | 1.00 | 1.00 | 0.75 | 0.86 | 0.20 | 0.58 | |
| | | | serum | | | | | | | | | | |
| | P10720 | Platelet factor 4 variant precursor | LMW_b | 4 | 4 | 6 | | | | | | | |
| | P02776 | Platelet factor 4 precursor | whole | 1.67 | 1.00 | 0.40 | 0.67 | 0.67 | 0.75 | 0.71 | 0.20 | 0.33 | |
| | | | serum | | | | | | | | | | |
| | P02776 | Platelet factor 4 precursor | LMW_b | 4 | 3 | 5 | | | | | | | |
| | P02775 | Platelet basic protein precursor | LMW_b | 17 | 9 | 18 | | | | | | | |
| | P02775 | Platelet basic protein precursor | whole | 0.33 | 0.75 | 0.40 | 1.58 | 0.33 | 0.25 | 0.29 | 0.40 | 0.67 | |
| | | | serum | | | | | | | | | | |
| | gi\|4505981 | Pro-platelet basic protein precursor | LMW_c | 33 | 20 | 23 | 16 | 20 | 14 | 12 | 19 | 11 | 15 |
| | gi\|4503445 | Endothelial cell growth factor 1 (platelet- | LMW_c | 0 | 0 | 0 | 0 | 0 | 0 | 0 | 0 | 0 | 1 |
| | | derived) | | | | | | | | | | | |
| | gi\|30794266 | Triggering receptor expressed on myeloid | LMW_c | 1 | 0 | 1 | 0 | 1 | 0 | 0 | 1 | 1 | 1 |
| | | cells-like 1 | | | | | | | | | | | |
| | O76036 | Natural cytotoxicity triggering receptor 1 | whole | 0.67 | 3.50 | 0.80 | 1.33 | 0.33 | 0.75 | 0.57 | 0.40 | 0.58 | |
| | | precursor | serum | | | | | | | | | | |
| | gi\|4503005 | Plasma carboxypeptidase B2 isoform a | LMW_c | 1 | 1 | 0 | 0 | 1 | 1 | 0 | 0 | 0 | 0 |
| | | preproprotein | | | | | | | | | | | |
| | Q96IY4 | Carboxypeptidase B2 precursor | whole | 0.00 | 0.00 | 0.00 | 0.08 | 0.00 | 0.00 | 0.00 | 0.00 | 0.08 | |
| | | | serum | | | | | | | | | | |
| | P14384 | Carboxypeptidase M precursor | whole | 0.00 | 0.00 | 0.00 | 0.08 | 0.00 | 0.00 | 0.00 | 0.00 | 0.08 | |
| | | | serum | | | | | | | | | | |
| | P22792 | Carboxypeptidase N subunit 2 precursor | whole | 0.00 | 0.00 | 2.00 | 1.50 | 0.00 | 0.00 | 0.00 | 0.60 | 0.58 | |
| | | | serum | | | | | | | | | | |
| | Q96SM3 | Probable carboxypeptidase X precursor | LMW_b | 0 | 0 | 1 | | | | | | | |
| | Q04609 | Glutamate carboxypeptidase II | whole | 0.00 | 0.00 | 0.00 | 0.08 | 0.00 | 0.00 | 0.00 | 0.00 | 0.08 | |
| | | | serum | | | | | | | | | | |
| | Q96KN2 | Glutamate carboxypeptidase-like protein 2 | whole | 0.00 | 0.00 | 0.40 | 0.08 | 0.00 | 0.00 | 0.00 | 0.20 | 0.08 | |
| | | precursor | serum | | | | | | | | | | |
| | O75636 | Ficolin-3 precursor | whole | 0.33 | 0.00 | 0.00 | 0.00 | 0.33 | 0.00 | 0.14 | 0.00 | 0.00 | |
| | | | serum | | | | | | | | | | |

Serum fibrinogen is elevated in AD (Oijen2005)
Platelets are one of the major sources for APP and Abeta outside of the brain
Platelets are activated in elderly and depressed individuals (Whyte2001)
Platelet factor 4 can be used as coagulant (Schulman2007)
Platelet factor 4 acts in immune response and affects every cell in the blood - with different results (depending on receptor) (Hundelshausen2007)
Triggering receptor TLT-1 is expressed in platelets and exists as platelet-membrane bound version and soluble version (in serum) (Gattis2006
The soluble version of TLT-1 is released from platelets in response to activation by thrombin (Gattis2006)
Triggering receptors (of other cell lines) are known to play a role in immune response and polymorphisms are studied in AD (with negative results so far) (Fenoglio2007)
Abeta fibrils cause aggregation of platelets (Kowalska1994)

| | | | | | | | | | | | | | |
|---|---|---|---|---|---|---|---|---|---|---|---|---|---|
| complement | gi\|21264361 | Mannan-binding lectin serine protease 2 | LMW_c | 1 | 2 | 0 | 0 | 0 | 0 | 1 | 1 | 0 | 2 |
| cascade | | isoform 1 precursor | | | | | | | | | | | |
| | O00187 | Mannan-binding lectin serine protease 2 | LMW_b | 0 | 1 | 1 | | | | | | | |
| | | precursor | | | | | | | | | | | |
| | Q96RS4 | Complement factor MASP-3 | whole | 0.00 | 0.00 | 0.00 | 0.08 | 0.00 | 0.00 | 0.00 | 0.00 | 0.08 | |
| | | | serum | | | | | | | | | | |
| | P00751 | Complement factor B precursor (C3/C5 | LMW_b | 5 | 5 | 5 | | | | | | | |
| | | convertase) | | | | | | | | | | | |
| | P00751 | Complement factor B precursor | whole | 21.33 | 19.75 | 24.20 | 16.92 | 1.00 | 1.00 | 1.00 | 1.00 | 1.00 | |
| | | | serum | | | | | | | | | | |
| | gi\|67782358 | Complement factor B preproprotein | LMW_c | 2 | 2 | 23 | 3 | 2 | 2 | 2 | 0 | 15 | 1 |
| | P02747 | Complement C1q subcomponent, C chain | LMW_b | 0 | 0 | 1 | | | | | | | |
| | | precursor | | | | | | | | | | | |
| | P02745 | Complement C1q subcomponent, A chain | whole | 0.00 | 0.00 | 0.00 | 0.17 | 0.00 | 0.00 | 0.00 | 0.00 | 0.17 | |
| | | precursor | serum | | | | | | | | | | |
| | P02746 | Complement C1q subcomponent, B chain | whole | 0.00 | 0.00 | 0.00 | 0.17 | 0.00 | 0.00 | 0.00 | 0.00 | 0.08 | |
| | | precursor | serum | | | | | | | | | | |
| | P02746 | Complement C1q subcomponent, B chain | LMW_b | 0 | 0 | 1 | | | | | | | |
| | | precursor | | | | | | | | | | | |
| | P02746 | Complement C1q subcomponent, B chain | LMW_b | 0 | 0 | 1 | | | | | | | |
| | | precursor | | | | | | | | | | | |
| | gi\|87298828 | Complement component 1, q subcomponent, | LMW_c | 0 | 1 | 0 | 0 | 0 | 0 | 1 | 1 | 0 | 0 |
| | | B chain precursor | | | | | | | | | | | |
| | P02747 | Complement C1q subcomponent, C chain | LMW_b | 0 | 0 | 1 | | | | | | | |
| | | precursor | | | | | | | | | | | |
| | P02747 | Complement C1q subcomponent, C chain | whole | 3.33 | 0.50 | 1.80 | 1.50 | 1.00 | 0.25 | 0.57 | 0.80 | 0.67 | |
| | | precursor | serum | | | | | | | | | | |
| | gi\|56786155 | Complement component 1, q subcomponent, | LMW_c | 2 | 3 | 2 | 5 | 2 | 1 | 1 | 2 | 2 | 1 |
| | | gamma polypeptide | | | | | | | | | | | |
| | P00736 | Complement C1r subcomponent precursor | LMW_b | 1 | 1 | 0 | | | | | | | |
| | P00736 | Complement C1r subcomponent precursor | whole | 2.67 | 0.50 | 0.40 | 0.67 | 0.67 | 0.50 | 0.57 | 0.20 | 0.58 | |
| | | | serum | | | | | | | | | | |
| | gi\|66347875 | Complement component 1, r subcomponent | LMW_c | 0 | 0 | 0 | 0 | 0 | 1 | 0 | 0 | 1 | 0 |
| | Q53GX9 | Complement component 1, r subcomponent- | whole | 0.00 | 0.00 | 0.00 | 0.08 | 0.00 | 0.00 | 0.00 | 0.00 | 0.08 | |
| | | like variant | serum | | | | | | | | | | |
| | P09871 | Complement C1s subcomponent precursor | whole | 0.33 | 0.25 | 0.20 | 0.08 | 0.33 | 0.25 | 0.29 | 0.20 | 0.08 | |
| | | | serum | | | | | | | | | | |
| | P06681 | Complement C2 precursor (C3/C5 convertase) | whole | 0.33 | 0.25 | 0.00 | 0.08 | 0.33 | 0.25 | 0.29 | 0.00 | 0.08 | |
| | | | serum | | | | | | | | | | |
| | P01024 | Complement C3 precursor | LMW_b | 32 | 21 | 19 | | | | | | | |
| | P01024 | Complement C3 precursor | whole | 143.67 | 146.00 | 190.80 | 169.42 | 1.00 | 1.00 | 1.00 | 1.00 | 1.00 | |
| | | | serum | | | | | | | | | | |
| | gi\|4557385 | Complement component 3 precursor | LMW_c | 20 | 30 | 19 | 16 | 10 | 17 | 16 | 11 | 20 | 15 |
| | P0C0L4 | Complement C4-A precursor | LMW_b | 14 | 17 | 8 | | | | | | | |
| | P0C0L4 | Complement C4-A precursor | whole | 45.33 | 44.00 | 59.80 | 65.92 | 1.00 | 1.00 | 1.00 | 1.00 | 1.00 | |
| | | | serum | | | | | | | | | | |
| | Q5JQM8 | Complement component 4A | whole | 0.67 | 0.25 | 0.80 | 0.83 | 0.33 | 0.25 | 0.29 | 0.40 | 0.33 | |
| | | | serum | | | | | | | | | | |
| | P0C0L5 | Complement C4-B precursor | whole | 1.67 | 1.25 | 0.80 | 0.33 | 0.67 | 0.50 | 0.57 | 0.40 | 0.33 | |
| | | | serum | | | | | | | | | | |
| | Q6U2E9 | Complement component C4B | whole | 0.00 | 0.00 | 0.20 | 0.33 | 0.00 | 0.00 | 0.00 | 0.20 | 0.08 | |
| | | | serum | | | | | | | | | | |
| | gi\|50345296 | Complement component 4B preproprotein | LMW_c | 24 | 15 | 17 | 18 | 12 | 12 | 10 | 13 | 16 | 10 |
| | P01031 | Complement C5 precursor | LMW_b | 3 | 1 | 2 | | | | | | | |
| | P01031 | Complement C5 precursor | whole | 0.67 | 4.25 | 5.60 | 4.17 | 0.67 | 1.00 | 0.86 | 1.00 | 0.83 | |
| | | | serum | | | | | | | | | | |
| | gi\|38016947 | Complement component 5 | LMW_c | 3 | 2 | 2 | 1 | 4 | 3 | 2 | 4 | 5 | 4 |
| | P13671 | Complement component C6 precursor | whole | 1.00 | 1.00 | 0.80 | 1.42 | 0.67 | 0.75 | 0.71 | 0.60 | 0.75 | |
| | | | serum | | | | | | | | | | |
| | P10643 | Complement component C7 precursor | whole | 0.00 | 0.50 | 1.00 | 0.42 | 0.00 | 0.50 | 0.29 | 0.80 | 0.25 | |
| | | | serum | | | | | | | | | | |
| | P07357 | Complement component C8 alpha chain | whole | 0.33 | 0.25 | 0.00 | 0.00 | 0.33 | 0.25 | 0.29 | 0.00 | 0.00 | |
| | | precursor | serum | | | | | | | | | | |
| | P07358 | Complement component C8 beta chain | whole | 0.00 | 0.00 | 0.20 | 0.00 | 0.00 | 0.00 | 0.00 | 0.20 | 0.00 | |
| | | precursor | serum | | | | | | | | | | |
| | P07360 | Complement component C8 gamma chain | whole | 0.33 | 1.25 | 2.60 | 1.83 | 0.33 | 0.50 | 0.43 | 0.60 | 0.58 | |
| | | precursor | serum | | | | | | | | | | |
| | P07360 | Complement component C8 gamma chain | LMW_b | 14 | 11 | 9 | | | | | | | |
| | | precursor | | | | | | | | | | | |
| | gi\|4557393 | Complement component 8, gamma | LMW_c | 18 | 14 | 33 | 50 | 25 | 36 | 64 | 23 | 24 | 16 |
| | | polypeptide | | | | | | | | | | | |
| | P02748 | Complement component C9 precursor | LMW_b | 1 | 1 | 0 | | | | | | | |
| | P02748 | Complement component C9 precursor | whole | 3.33 | 2.25 | 1.40 | 1.50 | 1.00 | 0.75 | 0.86 | 0.60 | 0.75 | |
| | | | serum | | | | | | | | | | |
| | gi\|4502511 | Complement component 9 | LMW_c | 2 | 1 | 1 | 0 | 0 | 3 | 0 | 1 | 2 | 0 |
| | P00746 | Complement factor D precursor | whole | 0.00 | 0.00 | 0.00 | 0.08 | 0.00 | 0.00 | 0.00 | 0.00 | 0.08 | |
| | | | serum | | | | | | | | | | |
| | gi\|42544239 | Complement factor D preproprotein | LMW_c | 0 | 0 | 0 | 3 | 0 | 0 | 0 | 0 | 0 | 0 |
| | P08603 | Complement factor H precursor | LMW_b | 1 | 1 | 0 | | | | | | | |
| | gi\|62739186 | Complement factor H isoform a precursor | LMW_c | 2 | 1 | 4 | 0 | 4 | 2 | 4 | 1 | 0 | 0 |
| | P08603 | Complement factor H precursor | whole | 18.33 | 14.75 | 16.60 | 20.42 | 1.00 | 1.00 | 1.00 | 1.00 | 1.00 | |
| | | | serum | | | | | | | | | | |
| | Q03591 | Complement factor H-related protein 1 | whole | 0.00 | 0.00 | 0.00 | 0.50 | 0.00 | 0.00 | 0.00 | 0.00 | 0.25 | |
| | | precuror | serum | | | | | | | | | | |
| | gi\|11321587 | Complement factor H isoform a precursor | LMW_c | 0 | 0 | 0 | 0 | 0 | 0 | 0 | 0 | 0 | 3 |
| | P05156 | Complement factor I precursor | whole | 0.67 | 0.75 | 2.20 | 1.67 | 0.33 | 0.50 | 0.43 | 0.80 | 0.75 | |
| | | | serum | | | | | | | | | | |
| | P05156 | Complement factor I precursor (C3B/C4B | LMW_b | 2 | 2 | 0 | | | | | | | |
| | | inactivator) | | | | | | | | | | | |
| | P10909 | Clusterin precursor (Complement- | whole | 6.33 | 5.00 | 3.20 | 4.83 | 1.00 | 1.00 | 1.00 | 0.80 | 0.92 | |
| | | associated protein SP-40,4) | serum | | | | | | | | | | |
| | Q5VWT3 | Complement component (3b/4b) receptor 1- | whole | 0.00 | 0.00 | 0.20 | 0.17 | 0.00 | 0.00 | 0.00 | 0.20 | 0.17 | |
| | | like | serum | | | | | | | | | | |
| | gi\|4502503 | Complement component 4 binding protein, | LMW_c | 1 | 0 | 0 | 0 | 1 | 0 | 0 | 0 | 1 | 0 |
| | | alpha chain precursor | | | | | | | | | | | |
| | gi\|4502505 | Complement component 4 binding protein, | LMW_c | 0 | 0 | 0 | 0 | 1 | 0 | 0 | 0 | 0 | 0 |
| | | beta chain isoform 1 precursor | | | | | | | | | | | |
| | gi\|4504579 | I factor (complement) | LMW_c | 3 | 1 | 1 | 2 | 0 | 2 | 0 | 2 | 1 | 3 |
| APH | P13798 | Acylamino-acid-releasing enzyme | LMW_b | 81206.1 | 4 | 0 | 0 | | | | | | |
| | | (Acyl-peptide hydrolase) (APH) | | | | | | | | | | | |
| | P49748 | Acyl-CoA dehydrogenase | whole | 0.33 | 0.25 | 0.20 | 0.50 | 0.33 | 0.25 | 0.29 | 0.20 | 0.17 | |
| | | | serum | | | | | | | | | | |
| | P28330 | Acyl-CoA dehydrogenase | whole | 0.00 | 0.00 | 0.00 | 0.08 | 0.00 | 0.00 | 0.00 | 0.00 | 0.08 | |
| | | | serum | | | | | | | | | | |
| | Q5SQN7 | Acyl-Coenzyme A dehydrogenase | whole | 0.00 | 0.00 | 0.00 | 0.08 | 0.00 | 0.00 | 0.00 | 0.00 | 0.08 | |
| | | | serum | | | | | | | | | | |
| | Q6IEE5 | Acyl CoA:monoacylglycerol | whole | 0.00 | 0.25 | 0.00 | 0.00 | 0.00 | 0.25 | 0.14 | 0.00 | 0.00 | |
| | | acyltransferase 2 | serum | | | | | | | | | | |
| | gi\|81295404 | Acyl-Coenzyme A thioesterase 2, | LMW_c | 0 | 0 | 0 | 1 | 0 | 0 | 0 | 0 | 0 | 0 |
| | | mitochondrial isoform b | | | | | | | | | | | |
| | gi\|4557233 | Acyl-Coenzyme A dehydrogenase, C-2 to | LMW_c | 0 | 0 | 0 | 0 | 0 | 0 | 0 | 1 | 0 | 0 |
| | | C-3 short chain precursor | | | | | | | | | | | |
| | Q59FX8 | Hydroxyacylglutathione hydrolase-like | whole | 0.00 | 0.00 | 0.00 | 0.17 | 0.00 | 0.00 | 000 | 0.00 | 0.17 | |
| | | isoform 1 variant | serum | | | | | | | | | | |
| | P49753 | Peroxisomal acyl-coenzyme A thioester | whole | 0.00 | 0.00 | 0.00 | 0.08 | 0.00 | 0.00 | 0.00 | 0.00 | 0.08 | |
| | | hydrolase 2a | serum | | | | | | | | | | |
| | gi\|23510451 | N-acylaminoacyl-peptide hydrolase | LMW_c | 0 | 0 | 0 | 0 | 1 | 0 | 0 | 0 | 0 | 0 |

APH is a serine protease that degrades Abeta (Yamin2007)
APH expression in AD brain is reduced (Yamin2007)
APH can be a blood marker for exposure to organophosphate toxicants (Quistad2005)
APH may be the target for the cognitive-enhancing effects of certain organophosphorus compounds (Richards2000)
APH clears preferentially oxidatively damaged proteins (Fujino2000)

| | | | | | | | | | | | | | |
|---|---|---|---|---|---|---|---|---|---|---|---|---|---|
| vitamin A/ | P02753 | Plasma retinol-binding protein | LMW_b | 22992.3 | 4 | 14 | 7 | | | | | | |
| thyroxine | | precursor | | | | | | | | | | | |
| | P02753 | Plasma retinol-binding protein precursor | whole | 1.67 | 3.00 | 6.40 | 4.58 | 0.67 | 1.00 | 0.86 | 1.00 | 1.00 | |
| | | | serum | | | | | | | | | | |
| | gi\|55743122 | Retinol-binding protein 4, plasma | LMW_c | 298 | 409 | 435 | 349 | 351 | 435 | 454 | 410 | 336 | 446 |
| | | precursor | | | | | | | | | | | |
| | Q9HBH5 | Retinol dehydrogenase 14 | whole | 0.00 | 0.25 | 0.00 | 0.00 | 0.00 | 0.25 | 0.14 | 0.00 | 0.00 | |
| | | | serum | | | | | | | | | | |
| | P02766 | Transthyretin precursor (Prealbumin) | whole | 2.00 | 2.00 | 0.00 | 7.50 | 1.00 | 0.50 | 0.71 | 0.00 | 0.50 | |
| | | | serum | | | | | | | | | | |
| | P02766 | Transthyretin precursor | LMW_b | 126 | 155 | 173 | | | | | | | |
| | gi\|4507725 | Transthyretin | LMW_c | 486 | 517 | 567 | 347 | 429 | 451 | 694 | 424 | 527 | 513 |

PRBP binds transthyretin in the plasma to transport vitamin A (retinol) and thyroxine (thyroid hormone T4) - this also keeps PRBP from being cleared from the plasma (Raghu2004)
PRBP binding prohibits transthyretin misfolding (Raghu2004)
Vitamin A is necessary for good cognitive function and vitamin A deficiency is seen in AD brain (Goodman2006)
Proteins involved in vitamin A transport to the brain (including PRBP) are proposed as therapeutic targets in AD (Goodman2006)
Transthyretin inhibits Abeta aggregation (Liu2006, Choi2007)
Thyroxine inhibits the expression of APP (O'Barr2006)
Free plasma thyroxine (fT4) negatively correlates with MMSE scores: the higher the levels, the more cognitive decline (Stuerenburg2006)

| | | | | | | | | | | | | | |
|---|---|---|---|---|---|---|---|---|---|---|---|---|---|
| phospholipases | O95712 | Cytosolic phospholipase A2 beta | whole | 0.00 | 0.00 | 0.00 | 0.08 | 0.00 | 0.00 | 0.00 | 0.00 | 0.08 | |
| | | | serum | | | | | | | | | | |
| | Q9Y263 | Phospholipase A-2-activating protein | LMW_b | 0 | 1 | 1 | | | | | | | |
| | Q5VWL4 | Phospholipase C, epsilon 1 | LMW_b | 0 | 0 | 1 | | | | | | | |
| | Q4KWH8 | Phospholipase C-eta1b | whole | 0.00 | 1.00 | 1.80 | 1.42 | 0.00 | 0.50 | 0.29 | 0.60 | 0.58 | |
| | | | serum | | | | | | | | | | |
| | gi\|4505873 | Phospholipase D1 | LMW_c | 3 | 2 | 0 | 1 | 0 | 0 | 1 | 2 | 1 | 2 |
| | O14939 | Phospholipase D2 | LMW_b | 0 | 0 | 2 | | | | | | | |
| | O14939 | Phospholipase D2 | whole | 0.00 | 0.25 | 0.00 | 0.00 | 0.00 | 0.25 | 0.14 | 0.00 | 0.00 | |
| | | | serum | | | | | | | | | | |
| | P41247 | Patatin-like phospholipase domain | whole | 0.00 | 0.00 | 0.20 | 0.00 | 0.00 | 0.00 | 0.00 | 0.20 | 0.00 | |
| | | containing protein 4 | serum | | | | | | | | | | |

PLD is upregulated in AD brain mitochondria + interacts with APP/Abeta (Jin2006)
PLD is upregulated in AD reactive astroglia + induced by APP (Jin2007)
PLD changes Abeta trafficking in cell an may be able to rescue cell function (Cai2005)

| | | | | | | | | | | | | | |
|---|---|---|---|---|---|---|---|---|---|---|---|---|---|
| globins | Q53F97 | Alpha 2 globin variant | whole | 4.00 | 6.50 | 5.40 | 2.92 | 1.00 | 0.75 | 0.86 | 0.80 | 0.67 | |
| | | | serum | | | | | | | | | | |
| | Q53F97 | Alpha 2 globin variant | LMW_b | 74 | 90 | 52 | | | | | | | |
| | gi\|4504345 | Alpha 2 globin | LMW_c | 149 | 185 | 194 | 239 | 127 | 80 | 168 | 212 | 72 | 234 |
| | gi\|4504349 | Beta globin | LMW_c | 102 | 112 | 213 | 128 | 289 | 100 | 110 | 109 | 61 | 95 |
| | Q14473 | Beta globin | whole | 7.33 | 10.25 | 8.80 | 6.67 | 1.00 | 1.00 | 1.00 | 1.00 | 0.83 | |
| | | | serum | | | | | | | | | | |
| | Q14473 | Beta globin | LMW_b | 55 | 61 | 51 | | | | | | | |
| | P69891 | Gamma globin | whole | 0.00 | 0.25 | 0.00 | 0.00 | 0.00 | 0.25 | 0.14 | 0.00 | 0.00 | |
| | | | serum | | | | | | | | | | |
| | P69891 | Gamma globin | LMW_b | 1 | 1 | 0 | | | | | | | |
| | gi\|28302131 | G-gamma globin | LMW_c | 2 | 4 | 2 | 1 | 1 | 1 | 1 | 1 | 0 | 4 |
| | gi\|4504351 | Delta globin | LMW_c | 14 | 15 | 25 | 15 | 22 | 9 | 12 | 50 | 14 | 13 |
| | P02042 | Delta globin | whole | 9.00 | 10.50 | 11.40 | 9.50 | 1.00 | 0.75 | 0.86 | 1.00 | 1.00 | |
| | | | serum | | | | | | | | | | |
| | P02042 | Delta globin | LMW_b | 60 | 56 | 66 | | | | | | | |
| | gi\|4826762 | Haptoglobin | LMW_c | 303 | 155 | 256 | 238 | 352 | 205 | 105 | 201 | 285 | 126 |
| | P00738 | Haptoglobin precursor | whole | 34.00 | 42.50 | 45.40 | 46.92 | 1.00 | 1.00 | 1.00 | 1.00 | 1.00 | |
| | | | serum | | | | | | | | | | |
| | P00738 | Haptoglobin precursor | LMW_b | 26 | 34 | 40 | | | | | | | |
| | gi\|45580723 | Haptoglobin-related protein | LMW_c | 3 | 2 | 3 | 3 | 1 | 0 | 0 | 0 | 2 | 3 |
| | P00739 | Haptoglobin-related protein precursor | whole | 43.33 | 60.50 | 60.80 | 74.00 | 1.00 | 1.00 | 1.00 | 1.00 | 1.00 | |
| | | | serum | | | | | | | | | | |
| | P00739 | Haptoglobin-related protein precursor | LMW_b | 54 | 53 | 58 | | | | | | | |
| | gi\|4507809 | Secretoglobin, family 1A, member 1 | LMW_c | 0 | 1 | 0 | 0 | 0 | 0 | 0 | 0 | 1 | 0 |
| | gi\|50363226 | Secretoglobin, family 3A, member 1 | LMW_c | 0 | 0 | 0 | 0 | 1 | 1 | 0 | 0 | 0 | 0 |

An increased number of hemoglobin alpha and beta fragments are found in AD cerebellum (Slemmon1994)
Hemoglobin alpha binds to Abeta (Oyama2000)
Red blood cells take up Abeta fibrils in blood, which leads to increased oxidative damage and hemoglobin degradation (Jayakumar2003)
Hemoglobin alpha (and beta?) fragments are more abundant in AD serum (Zhang2004)
Hemorphins (opioid receptor-binding proteins that originate from hemoglobin beta chain) are increased in AD temporal cortex - indicative of vascular abnormalities resulting from CAA (Poljak2004)
Hemoglobin alpha and beta induce oligomerization of Abeta and are found increasingly in AD brain (leakage from blood + increased expression in brain) (Wu2004)
Female patients with severe cognitive impairment have lower hemoglobin (Riccio2007)
Binding to Abeta is strongest for gamma-globin, followed by oxidized adult hemoglobin (alpha + beta chains) and then oxidized fetal hemoglobin (gamma chains) (Perry2006)

| | | | | | | | | | | | | | | |
|---|---|---|---|---|---|---|---|---|---|---|---|---|---|---|
| glycoproteins | gi\|4504489 | Histidine-rich glycoprotein precursor | LMW_c | 6 | 6 | 9 | 3 | 5 | 10 | 13 | 8 | 11 | 19 | A |
| | P04196 | Histidine-rich glycoprotein precursor | LMW_b | 6 | 10 | 5 | | | | | | | | |
| | P04196 | Histidine-rich glycoprotein precursor | whole | 15.33 | 8.00 | 8.40 | 8.17 | 1.00 | 1.00 | 1.00 | 1.00 | 1.00 | | |
| | | | serum | | | | | | | | | | | |
| | P02790 | Hemopexin precursor (Beta-1B-glycoprotein) | LMW_b | 3 | 5 | 5 | | | | | | | | |
| | P02790 | Hemopexin precursor (Beta-1B-glycoprotein) | whole | 46.33 | 53.75 | 73.40 | 66.33 | 1.00 | 1.00 | 1.00 | 1.00 | 1.00 | | |
| | | | serum | | | | | | | | | | | |
| | gi\|11321561 | Hemopexin | LMW_c | 10 | 8 | 9 | 6 | 6 | 11 | 15 | 7 | 10 | 7 | |
| | P02763 | Alpha-1-acid glycoprotein 1 precursor | whole | 24.00 | 31.00 | 31.60 | 35.67 | 1.00 | 1.00 | 1.00 | 1.00 | 1.00 | | |
| | | | serum | | | | | | | | | | | |
| | P02763 | Alpha-1-acid glycoprotein 1 precursor | LMW_b | 1 | 0 | 0 | | | | | | | | |
| | P04217 | Alpha-1B-glycoprotein precursor | whole | 24.33 | 15.75 | 28.80 | 24.42 | 1.00 | 1.00 | 1.00 | 1.00 | 1.00 | | |
| | | | serum | | | | | | | | | | | |
| | gi\|21071030 | Alpha 1B-glycoprotein | LMW_c | 3 | 2 | 3 | 2 | 3 | 5 | 5 | 1 | 3 | 2 | B |
| | P19652 | Alpha-1-acid glycoprotein 2 precursor | whole | 4.33 | 9.25 | 12.00 | 11.50 | 1.00 | 1.00 | 1.00 | 0.80 | 1.00 | | |
| | | | serum | | | | | | | | | | | |
| | P02765 | Alpha-2-HS-glycoprotein precursor | LMW_b | 7 | 7 | 5 | | | | | | | | |
| | gi\|4502005 | Alpha-2-HS-glycoprotein | LMW_c | 22 | 11 | 6 | 4 | 7 | 31 | 16 | 9 | 11 | 51 | |
| | P02765 | Alpha-2-HS-glycoprotein precursor | whole | 13.33 | 6.50 | 8.80 | 8.75 | 1.00 | 1.00 | 1.00 | 1.00 | 1.00 | | |
| | | | serum | | | | | | | | | | | |
| | gi\|4502337 | Alpha-2-glycoprotein 1, zinc | LMW_c | 1 | 1 | 1 | 0 | 1 | 0 | 2 | 5 | 1 | 0 | |
| | P02749 | Beta-2-glycoprotein I precursor (Apolipoprotein | whole | 26.67 | 22.25 | 24.00 | 23.33 | 1.00 | 1.00 | 1.00 | 1.00 | 1.00 | | |
| | | H) | serum | | | | | | | | | | | |
| | Q6X3Y4 | Rhesus-associated C glycoprotein | whole | 0.00 | 0.00 | 0.20 | 0.00 | 0.00 | 0.00 | 0.00 | 0.20 | 0.00 | | |
| | | | serum | | | | | | | | | | | |
| | Q5SZW4 | Rhesus blood group, B glycoprotein | whole | 0.00 | 0.00 | 0.00 | 0.08 | 0.00 | 0.00 | 0.00 | 0.00 | 0.08 | | |
| | | | serum | | | | | | | | | | | |
| | P25311 | Zinc-alpha-2-glycoprotein precursor | whole | 0.00 | 1.00 | 1.00 | 2.00 | 0.00 | 0.50 | 0.29 | 0.40 | 0.50 | | |
| | | | serum | | | | | | | | | | | |
| | P25311 | Zinc-alpha-2-glycoprotein precursor | LMW_b | 1 | 2 | 0 | | | | | | | | |
| | P00751 | Complement factor B precursor (Glycine-rich | LMW_b | 5 | 5 | 5 | | | | | | | | |
| | | beta glycoprotein) | | | | | | | | | | | | |
| | Q14624 | Inter-alpha-trypsin inhibitor heavy chain H4 | LMW_b | 3 | 5 | 3 | | | | | | | | |
| | | precursor (ITIH4) (Plasma kallikrein sensitive | | | | | | | | | | | | |
| | | glycoprotein 120) | | | | | | | | | | | | |
| | P21439 | Multidrug resistance protein 3 (P-glycoprotein | LMW_b | 0 | 1 | 0 | | | | | | | | |
| | | 3) | | | | | | | | | | | | |
| | Q9NYU2 | UDP-glucose:glycoprotein glucosyltransferase | whole | 0.00 | 0.00 | 0.00 | 0.25 | 0.00 | 0.00 | 0.00 | 0.00 | 0.25 | | |
| | | 1 precursor | serum | | | | | | | | | | | |
| | P20701 | Integrin alpha-L precursor (Leukocyte | whole | 0.00 | 0.00 | 0.60 | 0.08 | 0.00 | 0.00 | 0.00 | 0.60 | 0.08 | | B |
| | | adhesion glycoprotein LFA-1 alpha chain) | serum | | | | | | | | | | | |
| | Q5VU65 | Nuclear pore membrane glycoprotein 210-like | whole | 0.33 | 0.00 | 0.00 | 0.00 | 0.33 | 0.00 | 0.14 | 0.00 | 0.00 | | |
| | | | serum | | | | | | | | | | | |
| | P02750 | Leucine-rich alpha-2-glycoprotein | whole | 5.67 | 1.75 | 1.00 | 1.08 | 1.00 | 0.50 | 0.71 | 0.40 | 0.58 | | |
| | | precursor | serum | | | | | | | | | | | |
| | P02760 | AMBP protein precursor [Contains: Alpha-1- | LMW_b | 1 | 2 | 0 | | | | | | | | |
| | | microglobulin (Protein HC) (Complex-forming | | | | | | | | | | | | |
| | | glycoprotein heterogeneous in charge) (Alpha- | | | | | | | | | | | | |
| | | 1 microglycoprotein); Inter-alpha-trypsin | | | | | | | | | | | | |
| | | inhibitor light chain (ITI-LC) (Bikunin) (HI-30)] | | | | | | | | | | | | |
| | gi\|4504489 | Aistidine-rich glycoprotein precursor | LMW_c | 6 | 6 | 9 | 3 | 5 | 10 | 13 | 8 | 11 | 19 | B |
| | gi\|10863987 | Zona pellucida glycoprotein 4 preproprotein | LMW_c | 2 | 0 | 0 | 1 | 1 | 0 | 1 | 0 | 1 | 0 | |

HRG is more abundant in AD serum (Zhang2004)
HRG can inhibit coagulation (Jones2005)
HRG binds heme (Jones2005)
HRG binds metals (Jones2005)
HRG inhibits plasminogen activation by fibrin (Mosesson2005)
HRG binds to cells (platelets, lymphocytes, monocytes) much more strongly in the presence of zinc and copper (Horne2001)
HRG is involved in the disposal of immune complexes and apoptotic cells (Gorgani2007)

| | | | | | | | | | | | | | | |
|---|---|---|---|---|---|---|---|---|---|---|---|---|---|---|
| protease | gi\|50363217 | Alpha-1-antitrypsin (serine (or cysteine) | LMW_c | 27 | 21 | 24 | 19 | 21 | 25 | 17 | 30 | 25 | 31 | B |
| inhibitors | | proteinase inhibitor, clade A member 1) | | | | | | | | | | | | |
| | P01009 | Alpha-1-antitrypsin precursor | whole | 98.33 | 115.00 | 124.60 | 115.50 | 1.00 | 1.00 | 1.00 | 1.00 | 1.00 | | |
| | | | serum | | | | | | | | | | | |
| | P01009 | Alpha-1-antitrypsin precursor | LMW_b | 14 | 15 | 8 | | | | | | | | |
| | P20848 | Alpha-1-antitrypsin-related protein precursor | LMW_b | 1 | 2 | 0 | | | | | | | | |
| | P01011 | Alpha-1-antichymotrypsin precursor | LMW_b | 12 | 13 | 4 | | | | | | | | A |
| | P01011 | Alpha-1-antichymotrypsin precursor | whole | 11.33 | 12.00 | 12.40 | 16.17 | 1.00 | 1.00 | 1.00 | 1.00 | 1.00 | | |
| | | | serum | | | | | | | | | | | |
| | P08697 | Alpha-2-antiplasmin precursor | whole | 2.00 | 0.75 | 2.20 | 1.75 | 1.00 | 0.50 | 0.71 | 0.60 | 0.92 | | |
| | | | serum | | | | | | | | | | | |
| | gi\|39725934 | Alpha-2-antiplasmin (PEDF/serine (or | LMW_c | 0 | 0 | 0 | 0 | 0 | 0 | 0 | 0 | 0 | 1 | |
| | | cysteine) proteinase inhibitor, clade F, member 1 | | | | | | | | | | | | |
| | P01042 | Alpha-2-thiol proteinase inhbitor (Kininogen-1 | whole | 15.00 | 13.00 | 24.20 | 17.50 | 1.00 | 1.00 | 1.00 | 1.00 | 1.00 | | |
| | | precursor) | serum | | | | | | | | | | | |
| | P01042 | Alpha-2-thiol proteinase inhbitor | LMW_b | 6 | 3 | 3 | | | | | | | | B |
| | | (Kininogen-1 precursor) | | | | | | | | | | | | |
| | gi\|4502261 | Antithrombin (serine (or cysteine) proteinase | LMW_c | 14 | 8 | 12 | 4 | 5 | 11 | 12 | 7 | 7 | 8 | |
| | | inhibitor, clade C member 1) | | | | | | | | | | | | |
| | gi\|28076869 | Ovalbumin (serine (or cysteine) proteinase | LMW_c | 0 | 3 | 0 | 0 | 0 | 0 | 0 | 1 | 0 | 3 | |
| | | inhibitor, clade B member 4) | | | | | | | | | | | | |
| | gi\|4504781 | Inter-alpha (globulin) inhibitor H1 | LMW_c | 1 | 1 | 2 | 0 | 0 | 2 | 0 | 2 | 1 | 3 | |
| | gi\|70778918 | Inter-alpha (globulin) inhibitor H2 polypeptide | LMW_c | 0 | 0 | 1 | 0 | 0 | 2 | 2 | 1 | 2 | 1 | |
| | gi\|31542984 | Inter-alpha (Globulin) inhibitor H4 | LMW_c | 6 | 2 | 4 | 1 | 2 | 4 | 4 | 0 | 0 | 0 | B |
| | Q59FS1 | Inter-alpha (Globulin) inhibitor H4 | whole | 0.33 | 0.00 | 0.60 | 1.08 | 0.33 | 0.00 | 0.14 | 0.40 | 0.42 | | |
| | | | serum | | | | | | | | | | | |
| | P19827 | Inter-alpha-trypsin inhibitor heavy chain H1 | LMW_b | 3 | 2 | 0 | | | | | | | | B |
| | | precursor (ITIH1) | | | | | | | | | | | | |
| | P19827 | Inter-alpha-trypsin inhibitor heavy chain H1 | whole | 4.33 | 3.50 | 4.60 | 5.92 | 1.00 | 1.00 | 1.00 | 1.00 | 0.92 | | |
| | | precursor (ITIH1) | serum | | | | | | | | | | | |
| | P19823 | Inter-alpha-trypsin inhibitor heavy chain H2 | whole | 12.00 | 13.00 | 21.00 | 20.00 | 1.00 | 1.00 | 1.00 | 1.00 | 1.00 | | B |
| | | precursor (ITIH2) | serum | | | | | | | | | | | |
| | P19823 | Inter-alpha-trypsin inhibitor heavy chain H2 | LMW_b | 1 | 0 | 0 | | | | | | | | |
| | | precursor (ITIH2) | | | | | | | | | | | | |
| | Q14624 | Inter-alpha-trypsin inhibitor heavy chain H4 | whole | 7.00 | 8.00 | 11.80 | 11.00 | 1.00 | 1.00 | 1.00 | 1.00 | 1.00 | | |
| | | precursor (ITIH4) | serum | | | | | | | | | | | |
| | Q14624 | Inter-alpha-trypsin inhibitor heavy chain H4 | LMW_b | 3 | 5 | 3 | | | | | | | | |
| | | precursor (ITIH4) | | | | | | | | | | | | |
| | P05546 | Heparin cofactor II precursor (Protease | LMW_b | 2 | 1 | 0 | | | | | | | | |
| | | inhibitor leuserpin 2) | | | | | | | | | | | | |
| | P05546 | Heparin cofactor II precursor (Protease | whole | 0.67 | 2.25 | 0.80 | 1.00 | 0.67 | 1.00 | 0.86 | 0.40 | 0.58 | | |
| | | inhibitor leuserpin 2) | serum | | | | | | | | | | | |
| | gi\|4507377 | Serine (or cysteine) proteinase inhibitor, clade | LMW_c | 0 | 1 | 0 | 0 | 1 | 0 | 0 | 0 | 0 | 1 | |
| | | A member 7 | | | | | | | | | | | | |
| | gi\|50659080 | Serpin peptidase inhibitor, clade A member 3 | LMW_c | 9 | 4 | 5 | 6 | 5 | 4 | 3 | 7 | 10 | 1 | |
| | | precursor | | | | | | | | | | | | |
| | Q9NQ38 | Serine protease inhibitor Kazal-type 5 | whole | 0.00 | 0.00 | 0.00 | 0.08 | 0.00 | 0.00 | 0.00 | 0.00 | 0.08 | | |
| | | precursor | serum | | | | | | | | | | | |
| | P05155 | Plasma protease C1 inhibitor precursor | whole | 9.00 | 8.00 | 8.80 | 11.00 | 1.00 | 1.00 | 1.00 | 1.00 | 1.00 | | |
| | | | serum | | | | | | | | | | | |
| | P02760 | AMBP protein precursor [Contains: Alpha-1- | LMW_b | 1 | 2 | 0 | | | | | | | | |
| | | microglobulin (Protein HC) (Complex-forming | | | | | | | | | | | | |
| | | glycoprotein heterogeneous in charge) (Alpha- | | | | | | | | | | | | |
| | | 1 microglycoprotein); Inter-alpha-trypsin | | | | | | | | | | | | |
| | | inhibitor light chain (ITI-LC) (Bikunin) (HI-30)] | | | | | | | | | | | | |

Alpha 1 antichymotrypsin (ACT) is found to be upregulated in AD brain and serum (Licastro1998 & Licastro2000, DeKosky2003)
ITI is the most similar inhibitor to the Kunitz-type serine protease inhibitor domain found in APP (Yoshida1991)
ITI has been investigated as serum marker for AD (together with ACT) (Furby1991)
Alpha 1 antitrypsin is the inhibitor of HO-1 activity in AD plasma (HO-1 activity and expression is reduced in AD) (Maes2006)
Alpha 1 antitrypsin shows greater abundance and oxidation indices in AD blood (Yu2003)

| | | | | | | | | | | | | | | |
|---|---|---|---|---|---|---|---|---|---|---|---|---|---|---|
| keratins | gi\|17318569 | Keratin 1 | LMW_c | 45 | 4 | 3 | 22 | 36 | 5 | 7 | 3 | 4 | 3 | |
| and | P04264 | Keratin 1 | LMW_b | 6 | 65 | 15 | | | | | | | | A |
| related | gi\|47132620 | Keratin 2 | LMW_c | 23 | 7 | 2 | 4 | 2 | 7 | 4 | 2 | 1 | 6 | |
| proteins | Q4VAQ2 | Keratin 2a | LMW_b | 0 | 1 | 0 | | | | | | | | |
| | P35908 | Keratin 2e | LMW_b | 0 | 14 | 4 | | | | | | | | B |
| | P19013 | Keratin 4 | whole | 0.00 | 0.00 | 0.00 | 0.08 | 0.00 | 0.00 | 0.00 | 0.00 | 0.08 | | |
| | | | serum | | | | | | | | | | | |
| | P13647 | Keratin 5 | LMW_b | 1 | 3 | 0 | | | | | | | | |
| | gi\|4557890 | keratin 5 [Homo sapiens] | LMW_c | 1 | 1 | 0 | 2 | 0 | 0 | 0 | 0 | 0 | 0 | |
| | gi\|5031839 | keratin 6A [Homo sapiens] | LMW_c | 2 | 0 | 0 | 1 | 0 | 0 | 0 | 0 | 0 | 0 | |
| | gi\|55956899 | Keratin 9 | LMW_c | 29 | 4 | 0 | 17 | 6 | 1 | 7 | 0 | 0 | 0 | |
| | P35527 | Keratin 9 | LMW_b | 1 | 3 | 3 | | | | | | | | |
| | P13645 | Keratin 10 | LMW_b | 0 | 48 | 2 | | | | | | | | B |
| | P13646 | Keratin 13 | whole | 0.00 | 0.00 | 0.00 | 0.08 | 0.00 | 0.00 | 0.00 | 0.00 | 0.08 | | |
| | | | serum | | | | | | | | | | | |
| | P13646 | Keratin 13 | LMW_b | 0 | 2 | 0 | | | | | | | | |
| | P02533 | Keratin 14 | LMW_b | 0 | 5 | 0 | | | | | | | | B |
| | gi\|15431310 | Keratin 14 | LMW_c | 0 | 0 | 0 | 3 | 0 | 0 | 0 | 0 | 0 | 0 | |
| | Q04695 | Keratin 17 | LMW_b | 0 | 1 | 0 | | | | | | | | |
| | gi\|24234699 | Keratin 19 | LMW_c | 49 | 6 | 0 | 12 | 8 | 7 | 11 | 2 | 3 | 3 | |
| | Q9C075 | Keratin 23 | LMW_b | 0 | 0 | 1 | | | | | | | | |
| | Q7Z3Y9 | Keratin 25 | LMW_b | 0 | 1 | 0 | | | | | | | | |
| | Q14664 | Keratin ?? | LMW_b | 1 | 3 | 0 | | | | | | | | B |
| | Q92764 | Hair keratin, type I Ha5 | whole | 0.00 | 0.00 | 0.00 | 0.08 | 0.00 | 0.00 | 0.00 | 0.00 | 0.08 | | |
| | | | serum | | | | | | | | | | | |
| | O76013 | Hair keratin, type I Ha6 | whole | 0.00 | 0.00 | 0.00 | 0.08 | 0.00 | 0.00 | 0.00 | 0.00 | 0.08 | | |
| | | | serum | | | | | | | | | | | |
| | Q14533 | Hair keratin, type II Hb1 | whole | 0.00 | 1.00 | 2.60 | 3.08 | 0.00 | 0.75 | 0.43 | 0.80 | 0.67 | | |
| | | | serum | | | | | | | | | | | |
| | Q9NSB4 | Hair keratin, type II Hb2 | whole | 0.00 | 0.00 | 0.00 | 0.08 | 0.00 | 0.00 | 0.00 | 0.00 | 0.08 | | |
| | | | serum | | | | | | | | | | | |
| | gi\|10337581 | Hair keratin, type IIIb | LMW_c | 4 | 1 | 0 | 0 | 0 | 0 | 0 | 0 | 0 | 0 | |
| | Q7Z794 | Keratin, type II cytoskeletal 1b | LMW_b | 0 | 2 | 0 | | | | | | | | |
| | Q01546 | Keratin, type II cytoskeletal 2 oral | LMW_b | 0 | 2 | 0 | | | | | | | | |
| | O95678 | Cytokeratin type II | whole | 0.00 | 0.00 | 0.20 | 0.00 | 0.00 | 0.00 | 0.00 | 0.20 | 0.00 | | |
| | | | serum | | | | | | | | | | | |
| | Q9BYQ6 | Keratin-associated protein 4-14 | whole | 0.00 | 0.00 | 0.00 | 0.08 | 0.00 | 0.00 | 0.00 | 0.00 | 0.08 | | |
| | | | serum | | | | | | | | | | | |
| | gi\|38490587 | Keratin associated protein 10-12 | LMW_c | 1 | 1 | 0 | 0 | 0 | 0 | 0 | 0 | 0 | 0 | |
| | Q5T749 | Novel protein (Keratinocyte proline-rich | LMW_b | 0 | 4 | 0 | | | | | | | | B |
| | | protein) | | | | | | | | | | | | |
| | P15924 | Desmoplakin | LMW_b | 0 | 4 | 0 | | | | | | | | B |

Keratin antibodies have been found in serum of neurodegenerative disease patients - however, in AD alone this was not significant (Schott1996)
Presinilin-1 (responsible for early onset AD) is involved in development of skin barrier formation - however, keratin 1 did not change with PS1−/− (Nakajima2006)
Cytokeratin 1 is involved in bradykinin generation - which has been implicated in AD
Chronic oxidative stress can lead to mallory bodies in the liver - which consist of among other ubiquinated keratins (Janig2005)

| | | | | | | | | | | | | | | |
|---|---|---|---|---|---|---|---|---|---|---|---|---|---|---|
| heme | gi\|4502419 | Biliverdin reductase B | LMW_c | 0 | 4 | 3 | 1 | 0 | 0 | 0 | 1 | 0 | 3 | A |
| degradation | P53004 | Biliverdin reductase A precursor | whole | 0.00 | 0.00 | 0.00 | 0.08 | 0.00 | 0.00 | 0.00 | 0.00 | 0.08 | | |
| | | | serum | | | | | | | | | | | |

Biliverdin reductase and heme oxygenase degrade heme to bilirubin
heme oxygenase activity is upregulated in AD - and so are CSF bilirubin derivatives (Kimpara2000)

| | | | | | | | | | | | | | | |
|---|---|---|---|---|---|---|---|---|---|---|---|---|---|---|
| pyruvate | gi\|33286418 | Pyruvate kinase 3 isoform 1 | LMW_c | 0 | 0 | 0 | 0 | 0 | 0 | 0 | 1 | 1 | 0 | B |
| metabolism | P08559 | Pyruvate dehydrogenase E1 component alpha | whole | 0.00 | 0.00 | 0.20 | 0.00 | 0.00 | 0.00 | 0.00 | 0.20 | 0.00 | | |
| | | subunit | serum | | | | | | | | | | | |
| | Q5JPU1 | Pyruvate dehydrogenase alpha 1 | whole | 0.00 | 0.00 | 0.00 | 0.17 | 0.00 | 0.00 | 0.00 | 0.00 | 0.17 | | |
| | | | serum | | | | | | | | | | | |
| | O00330 | Pyruvate dehydrogenase protein X component | whole | 0.00 | 0.00 | 0.00 | 0.08 | 0.00 | 0.00 | 0.00 | 0.00 | 0.08 | | |
| | | | serum | | | | | | | | | | | |

PK activity is upregulated in frontal and temporal cortex in AD brain (BigI1999) - however, different PK isoform (?)
PK is one of the glycolytic enzymes
PK is upregulated in mitochondria of neurons stimulated with Abeta (LoveII2005) - however, different PK isoform
PK is oxidatively modified in MCI hippocampus (Butterfield2006) - however, different PK isoform

| | | | | | | | | | | | | | | |
|---|---|---|---|---|---|---|---|---|---|---|---|---|---|---|
| calcium | gi\|4507267 | Stanniocalcin 2 precursor | LMW_c | 0 | 0 | 0 | 0 | 0 | 0 | 0 | 1 | 0 | 1 | A |
| related | Q9Y6J0 | Calcineurin-binding protein (Cabin 1) | whole | 0.00 | 0.25 | 0.00 | 0.00 | 0.00 | 0.25 | 0.14 | 0.00 | 0.00 | | |
| proteins | | | serum | | | | | | | | | | | |
| | Q9Y6J0 | Calcineurin-binding protein (Cabin 1) | LMW_b | 1 | 0 | 1 | | | | | | | | |
| | Q16602 | Calcitonin gene-related peptide type 1 receptor | whole | 0.00 | 0.25 | 0.00 | 0.00 | 0.00 | 0.25 | 0.14 | 0.00 | 0.00 | | |
| | | precursor | serum | | | | | | | | | | | |
| | Q9NZU7 | Calcium-binding protein 1 (Calbrain) | whole | 0.00 | 0.00 | 0.00 | 0.17 | 0.00 | 0.00 | 0.00 | 0.00 | 0.17 | | |
| | | | serum | | | | | | | | | | | |
| | P09758 | Tumor-associated calcium signal | whole | 1.00 | 0.50 | 0.40 | 0.08 | 0.67 | 0.25 | 0.43 | 0.40 | 0.08 | | B |
| | | transducer 2 precursor | serum | | | | | | | | | | | |
| | gi\|32129214 | Neuronal calcium-binding protein 2 | LMW_c | 0 | 0 | 1 | 0 | 0 | 0 | 0 | 0 | 0 | 0 | |
| | P84074 | Neuron-specific calcium-binding protein | whole | 0.00 | 0.00 | 0.00 | 0.17 | 0.00 | 0.00 | 0.00 | 0.00 | 0.17 | | |
| | | (Hippocalcin) | serum | | | | | | | | | | | |
| | P37235 | Hippocalcin-like protein 1 | whole | 0.00 | 0.00 | 0.00 | 0.08 | 0.00 | 0.00 | 0.00 | 0.00 | 0.08 | | |
| | | | serum | | | | | | | | | | | |
| | P02458 | Collagen alpha 1(II) chain precursor [Contains: | whole | 0.00 | 0.00 | 0.00 | 0.08 | 0.00 | 0.00 | 0.00 | 0.00 | 0.08 | | |
| | | Chondrocalcin] | serum | | | | | | | | | | | |
| | Q9H4F8 | SPARC related modular calcium-binding | whole | 0.00 | 0.00 | 0.00 | 0.08 | 0.00 | 0.00 | 0.00 | 0.00 | 0.08 | | |
| | | protein 1 precursor | serum | | | | | | | | | | | |
| | Q16566 | Calcium/calmodulin-dependent protein kinase | whole | 0.00 | 0.00 | 0.00 | 0.08 | 0.00 | 0.00 | 0.00 | 0.00 | 0.08 | | |
| | | type IV | serum | | | | | | | | | | | |
| | Q9HAR2 | Latrophilin-3 precursor (Calcium- | whole | 0.00 | 0.00 | 0.60 | 0.08 | 0.00 | 0.00 | 0.00 | 0.40 | 0.08 | | B |
| | | independent alpha-latrotoxin receptor 3) | serum | | | | | | | | | | | |
| | Q9H9S4 | Calcium-binding protein 39-like | LMW_b | 0 | 1 | 0 | | | | | | | | |
| | P20020 | Plasma membrane calcium-transporting | whole | 0.00 | 0.25 | 0.00 | 0.00 | 0.00 | 0.25 | 0.14 | 0.00 | 0.00 | | |
| | | ATPase 1 | serum | | | | | | | | | | | |
| | Q01814 | Plasma membrane calcium-transporting | LMW_b | 1 | 0 | 1 | | | | | | | | |
| | | ATPase 2 | | | | | | | | | | | | |
| | gi\|48255953 | Plasma membrane calcium ATPase 3 isoform | LMW_c | 0 | 0 | 0 | 0 | 0 | 1 | 0 | 0 | 0 | 0 | |
| | | 3a | | | | | | | | | | | | |
| | P98194 | Calcium-transporting ATPase type 2C member 1 | whole | 0.00 | 0.00 | 0.20 | 0.00 | 0.00 | 0.00 | 0.00 | 0.20 | 0.00 | | |
| | | | serum | | | | | | | | | | | |
| | P16615 | Sarcoplasmic/endoplasmic reticulum calcium | whole | 0.00 | 0.00 | 0.00 | 0.08 | 0.00 | 0.00 | 0.00 | 0.00 | 0.08 | | |
| | | ATPase 2 | serum | | | | | | | | | | | |
| | Q9UHB1 | Voltage-gated L-type calcium channel alpha-1 | whole | 0.33 | 0.00 | 0.00 | 0.00 | 0.33 | 0.00 | 0.14 | 0.00 | 0.00 | | |
| | | subunit | serum | | | | | | | | | | | |
| | O60721 | Sodium/potassium/calcium exchanger 1 | whole | 0.00 | 0.25 | 0.00 | 0.08 | 0.00 | 0.25 | 0.14 | 0.00 | 0.08 | | |
| | | | serum | | | | | | | | | | | |
| | Q12791 | Calcium-activated potassium channel alpha | whole | 0.00 | 0.00 | 0.00 | 0.08 | 0.00 | 0.00 | 0.00 | 0.00 | 0.08 | | |
| | | subunit 1 | serum | | | | | | | | | | | |
| | O95151 | Calcium-dependent chloride channel-1 | LMW_b | 1 | 0 | 0 | | | | | | | | |
| | gi\|4506765 | S100 calcium-binding protein A4 | LMW_c | 0 | 0 | 1 | 0 | 0 | 0 | 0 | 0 | 0 | 0 | |
| | gi\|7657532 | S100 calcium-binding protein A6 | LMW_c | 0 | 0 | 0 | 1 | 0 | 0 | 0 | 0 | 0 | 0 | |
| | gi\|4506769 | S100 calcium-binding protein A7 | LMW_c | 0 | 6 | 1 | 0 | 0 | 1 | 0 | 1 | 1 | 4 | |
| | P31151 | S100 calcium-binding protein A7 | LMW_b | 0 | 4 | 0 | | | | | | | | |
| | gi\|21614544 | S100 calcium-binding protein A8 | LMW_c | 3 | 11 | 15 | 3 | 5 | 4 | 4 | 2 | 14 | 5 | |
| | P05109 | S100 calcium-binding protein A8 | LMW_b | 2 | 6 | 3 | | | | | | | | |
| | gi\|4506773 | S100 calcium-binding protein A9 | LMW_c | 2 | 4 | 3 | 4 | 3 | 3 | 6 | 3 | 5 | 5 | |
| | P06702 | S100 calcium-binding protein A9 | LMW_b | 0 | 1 | 0 | | | | | | | | |

STC 2 is upregulated in neurons stimulated with Abeta (Kim2003)
STC's are involved in calcium and phosphate regulation

| | | | | | | | | | | | | | | |
|---|---|---|---|---|---|---|---|---|---|---|---|---|---|---|
| defensin | gi\|113419903 | PREDICTED: similar to Neutrophil defensin | LMW_c | 11 | 3 | 2 | 3 | 0 | 2 | 1 | 0 | 4 | 18 | A |
| | | 1 precursor | | | | | | | | | | | | |
| | P59665 | Neutrophil defensin 1 precursor | LMW_b | 1 | 1 | 1 | | | | | | | | |

Defensin expression is changed in peripheral lymphocytes in Ad (Kalman2005)

| | | | | | | | | | | | | | | |
|---|---|---|---|---|---|---|---|---|---|---|---|---|---|---|
| gelsolin | gi\|38044288 | Gelsolin isoform a precursor | LMW_c | 3 | 4 | 5 | 3 | 2 | 5 | 2 | 5 | 7 | 2 | B |
| | P06396 | Gelsolin precursor | whole | 3.67 | 4.75 | 3.60 | 4.00 | 0.67 | 0.75 | 0.71 | 1.00 | 0.92 | | |
| | | | serum | | | | | | | | | | | |
| | P06396 | Gelsolin precursor | LMW_b | 0 | 2 | 0 | | | | | | | | A |

Peripheral injection of plasmid DNA encoding for gelsolin reduces brain Abeta in mice (Hirko2007)
Low intake of n-3 polyunsaturated fatty acids is a risk factor for AD - they cause NMDA receptor subunit loss and increased levels of gelsolin fragments in APP transgenic mice (Calon2005)
Mutations in gelsolin can lead to CAA (Tian2004)
Gelsolin inhibits Abeta induced cell death (Qiao2005)
Gelsolin binds Abeta very well and peripheral application of Gelsolin reduces brain Abeta (Matsuoka2003)

| | | | | | | | | | | | | | | |
|---|---|---|---|---|---|---|---|---|---|---|---|---|---|---|
| vitronectin | gi\|88853069 | Vitronectin precursor | LMW_c | 5 | 6 | 7 | 6 | 7 | 5 | 12 | 9 | 7 | 12 | B |
| | P04004 | Vitronectin precursor | whole | 5.33 | 5.25 | 11.20 | 7.33 | 1.00 | 1.00 | 1.00 | 1.00 | 1.00 | | |
| | | | serum | | | | | | | | | | | |
| | P04004 | Vitronectin precursor | LMW_b | 3 | 1 | 3 | | | | | | | | |

Vitronectin precursor is found upregulated in AD serum (proteomics study) (Zhang2004)
Vitronectin receptor is found in glial cells associated with plaques and vitronectin is found in plaques (Eikelenboom 1994)
Microglial expression of vitronectin receptor is upregulated in AD (McGeer1993)
Vitronectin protects against bystander lysis by the membrane attack complex (complement) (McGeer1993)
Microglia in gray and white matter of AD brain stain for vitronectin receptor - as do control and AD platelets (Akiyama1991)

| | | | | | | | | | | | | | | |
|---|---|---|---|---|---|---|---|---|---|---|---|---|---|---|
| profilin | gi\|40786418 | Profilin 4 | LMW_c | 0 | 0 | 0 | 0 | 1 | 2 | 0 | 0 | 0 | 0 | B |
| | gi\|4826898 | Profilin 1 | LMW_c | 1 | 1 | 0 | 0 | 4 | 1 | 0 | 0 | 2 | 1 | |
| | P07737 | Profilin 1 | LMW_b | 0 | 1 | 1 | | | | | | | | |

Profilin shows specific binding to Abeta (like Gelsolin) (Chauhan1999)
Profilin-2 is less oxidized after reduction of mouse brain Abeta burden (Poon2005)

| | | | | | | | | | | | | | | |
|---|---|---|---|---|---|---|---|---|---|---|---|---|---|---|
| thrombospondin | gi\|40317626 | Thrombospondin 1 precursor | LMW_c | 0 | 0 | 0 | 1 | 0 | 0 | 0 | 0 | 0 | 0 | |
| | P07996 | Thrombospondin 1 precursor | whole | 0.00 | 0.00 | 0.20 | 0.25 | 0.00 | 0.00 | 0.00 | 0.20 | 0.17 | | |
| | | | serum | | | | | | | | | | | |
| | P35442 | Thrombospondin 2 precursor | whole | 0.00 | 0.50 | 0.20 | 0.17 | 0.00 | 0.25 | 0.14 | 0.20 | 0.17 | | |
| | | | serum | | | | | | | | | | | |
| | gi\|31543806 | Thrombospondin 4 precursor | LMW_c | 1 | 0 | 2 | 1 | 2 | 1 | 1 | 1 | 1 | 0 | B |
| | P35443 | Thrombospondin 4 precursor | LMW_b | 2 | 1 | 2 | | | | | | | | |

Thrombospondin immunostaining is increased in a subset of pyramidal neurons that may be affected early in AD (Buee1992)
Thrombospondin immunostaining is found in plaques (Buee1992)

| | | | | | | | | | | | | | | |
|---|---|---|---|---|---|---|---|---|---|---|---|---|---|---|
| peroxiredoxin | gi\|32189392 | Peroxiredoxin 2 isoform a | LMW_c | 4 | 7 | 9 | 4 | 3 | 2 | 3 | 3 | 0 | 2 | B |
| and | P32119 | Peroxiredoxin 2 | LMW_b | 0 | 1 | 0 | | | | | | | | |
| alcohol | gi\|32483377 | Peroxiredoxin 3 isoform b | LMW_c | 0 | 0 | 0 | 0 | 0 | 1 | 0 | 0 | 0 | 1 | |
| dehydrogenase | P11766 | Alcohol dehydrogenase class III chi chain | whole | 0.00 | 0.00 | 0.00 | 0.08 | 0.00 | 0.00 | 0.00 | 0.00 | 0.08 | | |
| | | | serum | | | | | | | | | | | |
| | P07327 | Alcohol dehydrogenase alpha chain | whole | 0.00 | 0.00 | 0.00 | 0.08 | 0.00 | 0.00 | 0.00 | 0.00 | 0.08 | | |
| | | | serum | | | | | | | | | | | |

Peroxiredoxin 2 is more abundant in AD hippocampus (proteomics study) (Sultana2007)
Peroxiredoxin 2 is upregulated in AD peripheral leukocytes after stimulation with divalproex sodium (proteomics study) (Mhyre2007)
Peroxiredoxin 2 is upregulated in the brains of mice having high levels of Abeta42 and alcohol dehydrogenase - Peroxiredoxin 2 levels go down if the interaction between Abeta and alcohol
dehydrogenase is disturbed (Yao2007)
Peroxiredoxins are antioxidant enzymes (Krapfenbauer2003)

| | | | | | | | | | | | | | |
|---|---|---|---|---|---|---|---|---|---|---|---|---|---|
| apolipoproteins | P08519 | Apolipoprotein(a) precursor | whole | 0.67 | 0.25 | 0.00 | 0.67 | 0.33 | 0.25 | 0.29 | 0.00 | 0.17 | |
| | | | serum | | | | | | | | | | |
| | P02647 | Apolipoprotein A-I precursor | LMW_b | 47 | 57 | 52 | | | | | | | |
| | gi\|4557321 | Apolipoprotein A-I preproprotein | LMW_c | 388 | 253 | 274 | 285 | 426 | 327 | 430 | 333 | 374 | 325 |
| | P02647 | Apolipoprotein A-I precursor | whole | 129.67 | 135.75 | 154.80 | 150.00 | 1.00 | 1.00 | 1.00 | 1.00 | 1.00 | |
| | | | serum | | | | | | | | | | |
| | P02652 | Apolipoprotein A-II precursor | LMW_b | 109 | 85 | 114 | | | | | | | |
| | P02652 | Apolipoprotein A-II precursor | whole | 26.00 | 31.75 | 56.80 | 44.83 | 1.00 | 1.00 | 1.00 | 1.00 | 1.00 | |
| | | | serum | | | | | | | | | | |
| | gi\|4502149 | Apolipoprotein A-II preproprotein | LMW_c | 88 | 86 | 245 | 163 | 65 | 185 | 110 | 89 | 95 | 78 |
| | P06727 | Apolipoprotein A-IV precursor | LMW_b | 0 | 0 | 1 | | | | | | | |
| | P06727 | Apolipoprotein A-IV precursor | whole | 16.33 | 18.75 | 18.20 | 24.33 | 1.00 | 1.00 | 1.00 | 1.00 | 1.00 | |
| | | | serum | | | | | | | | | | |
| | gi\|71773110 | Apolipoprotein A-IV precursor | LMW_c | 5 | 7 | 3 | 2 | 4 | 7 | 4 | 4 | 4 | 5 |
| | gi\|4502153 | Apolipoprotein B precursor | LMW_c | 3 | 3 | 10 | 5 | 2 | 6 | 6 | 5 | 8 | 5 |
| | P04114 | Apolipoprotein B-100 precursor | LMW_b | 5 | 6 | 3 | | | | | | | |
| | P04114 | Apolipoprotein B-100 precursor | whole | 41.67 | 25.00 | 35.80 | 41.25 | 1.00 | 1.00 | 1.00 | 1.00 | 1.00 | |
| | | | serum | | | | | | | | | | |
| | P02654 | Apolipoprotein C-I precursor | LMW_b | 6 | 2 | 9 | | | | | | | |
| | P02654 | Apolipoprotein C-I precursor | whole | 5.00 | 1.50 | 0.40 | 0.83 | 1.00 | 0.75 | 0.86 | 0.40 | 0.42 | |
| | | | serum | | | | | | | | | | |
| | gi\|4502157 | Apolipoprotein C-I precursor | LMW_c | 15 | 14 | 11 | 20 | 13 | 20 | 17 | 19 | 19 | 24 |
| | P02655 | Apolipoprotein C-II precursor | LMW_b | 25 | 26 | 26 | | | | | | | |
| | P02655 | Apolipoprotein C-II precursor | whole | 0.67 | 0.50 | 0.60 | 0.67 | 0.33 | 0.25 | 0.29 | 0.20 | 0.33 | |
| | | | serum | | | | | | | | | | |
| | gi\|32130518 | Apolipoprotein C-II precursor | LMW_c | 43 | 33 | 193 | 64 | 41 | 96 | 268 | 38 | 52 | 154 |
| | P02656 | Apolipoprotein C-III precursor | LMW_b | 32 | 29 | 36 | | | | | | | |
| | P02656 | Apolipoprotein C-III precursor | | | 10.00 | 12.40 | 12.42 | 1.00 | 1.00 | 1.00 | 1.00 | 1.00 | |
| | gi\|4557323 | Apolipoprotein C-III precursor | | | 35 | 49 | 33 | 43 | 39 | 65 | 38 | 48 | 55 |
| | P55056 | Apolipoprotein C-IV precursor | | | 2 | 7 | | | | | | | |
| | gi\|4502161 | whole serum | 15.00 | | 5 | 7 | 3 | 9 | 3 | 11 | 8 | 9 | 12 |
| | P05090 | LMW_c | 54 | | 0.25 | 0.40 | 0.50 | 0.67 | 0.25 | 0.43 | 0.40 | 0.42 | |
| | gi\|4502163 | LMW_b | 2 | | 1 | 0 | 0 | 0 | 0 | 0 | 0 | 0 | 0 |
| | P02649 | LMW_c | 5 | | 9 | 4 | | | | | | | |
| | P02649 | whole serum | 0.67 | | 1.75 | 2.20 | 3.00 | 1.00 | 0.75 | 0.86 | 1.00 | 0.92 | |
| | gi\|4557325 | LMW_c | 0 | | 4 | 23 | 14 | 6 | 17 | 17 | 17 | 12 | 15 |
| | gi\|4502165 | LMW_b | 6 | 1 | 1 | 0 | 0 | 0 | 0 | 0 | 0 | 1 | 0 |
| | P02749 | whole serum | 6.33 | 26.67 | 22.25 | 24.00 | 23.33 | 1.00 | 1.00 | 1.00 | 1.00 | 1.00 | |
| | gi\|4557327 | LMW_c | 12 | 2 | 0 | 0 | 1 | 2 | 1 | 0 | 1 | 1 | 1 |
| | O14791 | Apolipoprotein-L1 precursor | whole | 0.67 | 0.50 | 1.60 | 0.67 | 0.67 | 0.50 | 0.57 | 0.60 | 0.42 | |
| | | | serum | | | | | | | | | | |
| | O14791 | Apolipoprotein-L1 precursor | LMW_b | 0 | 0 | 0 | | | | | | | |
| | gi\|21735614 | Apolipoprotein L1 isoform a precursor | LMW_c | 2 | 3 | 1 | 2 | 3 | 3 | 1 | 0 | 1 | 0 |
| | Q9BQE5 | Apolipoprotein-L2 | whole | 0.00 | 0.00 | 0.20 | 0.08 | 0.00 | 0.00 | 0.00 | 0.20 | 0.08 | |
| | | | serum | | | | | | | | | | |
| | Q9BWW8 | Apolipoprotein-L6 | whole | 0.00 | 0.25 | 0.00 | 0.00 | 0.00 | 0.25 | 0.14 | 0.00 | 0.00 | |
| | | | serum | | | | | | | | | | |
| | O95445 | Apolipoprotein M | whole | 0.33 | 0.25 | 0.00 | 0.25 | 0.33 | 0.25 | 0.29 | 0.00 | 0.08 | |
| | | | serum | | | | | | | | | | |
| | gi\|22091452 | Apolipoprotein M | LMW_c | 4 | 1 | 6 | 5 | 4 | 4 | 2 | 2 | 1 | 0 |
| iron | gi\|4557871 | Transferrin | LMW_c | 61 | 61 | 70 | 34 | 61 | 78 | 81 | 68 | 104 | 101 |
| and | P02787 | Transferrin | LMW_b | 26 | 21 | 22 | | | | | | | |
| copper | P02787 | Transferrin | whole | 179.67 | 207.00 | 249.80 | 222.58 | 1.00 | 1.00 | 1.00 | 1.00 | 1.00 | |
| metabolism | | | serum | | | | | | | | | | |
| | P00450 | Ceruloplasmin precursor | LMW_b | 7 | 6 | 6 | | | | | | | |
| | P00450 | Ceruloplasmin precursor | whole | 51.33 | 31.50 | 48.60 | 41.67 | 1.00 | 1.00 | 1.00 | 1.00 | 1.00 | |
| | | | serum | | | | | | | | | | |
| | gi\|4557485 | Ceruloplasmin precursor | LMW_c | 17 | 12 | 17 | 13 | 11 | 18 | 16 | 20 | 22 | 13 |
| | P02790 | Hemopexin precursor | LMW_b | 2 | 5 | 3 | | | | | | | |
| | P02790 | Hemopexin precursor | whole | 46.33 | 53.75 | 73.40 | 66.33 | 1.00 | 1.00 | 1.00 | 1.00 | 1.00 | |
| | | | serum | | | | | | | | | | |
| | gi\|11321561 | Hemopexin | LMW_c | 10 | 8 | 9 | 6 | 6 | 11 | 15 | 7 | 10 | 7 |
| | P10109 | Adrenodoxin, mitochondrial precursor (Adrenal | whole | 0.00 | 0.00 | 0.00 | 0.08 | 0.00 | 0.00 | 0.00 | 0.00 | 0.08 | |
| | | ferredoxin) | serum | | | | | | | | | | |
| | gi\|20149498 | Ferritin, light polypeptide | LMW_c | 1 | 0 | 0 | 0 | 0 | 0 | 0 | 0 | 0 | 0 |
| | gi\|60499021 | Ferrochelatase isoform a precursor | LMW_c | 0 | 0 | 1 | 0 | 0 | 0 | 0 | 0 | 0 | 0 |
| | Q04656 | Copper-transporting ATPase 1 (ATP7A) | whole | 0.00 | 0.00 | 0.20 | 0.00 | 0.00 | 0.00 | 0.00 | 0.20 | 0.00 | |
| | | | serum | | | | | | | | | | |
| | Q9UJW0 | Dynactin subunit 4 | whole | 0.00 | 0.00 | 0.60 | 0.75 | 0.00 | 0.00 | 0.00 | 0.40 | 0.50 | |
| | | | serum | | | | | | | | | | |
| | Q92859 | Neogenin precursor | whole | 3.67 | 4.00 | 1.20 | 1.42 | 1.00 | 0.75 | 0.86 | 0.40 | 0.33 | |
| | | | serum | | | | | | | | | | |
| NMDA | Q5T2T0 | Novel protein (with guanylate kinase II | whole | 0.00 | 0.00 | 0.00 | 1.08 | 0.00 | 0.00 | 0.00 | 0.00 | 0.50 | |
| receptor | | domain) | serum | | | | | | | | | | |
| related | Q92796 | Presynaptic protein SAP10 | whole | 0.00 | 0.00 | 0.00 | 0.42 | 0.00 | 0.00 | 0.00 | 0.00 | 0.33 | |
| proteins | | | serum | | | | | | | | | | |
| | Q99996 | A-kinase anchor protein 9 | whole | 0.67 | 0.50 | 0.00 | 0.00 | 0.33 | 0.50 | 0.43 | 0.00 | 0.00 | |
| | | | serum | | | | | | | | | | |
| | Q13224 | Glutamate [NMDA] receptor subunit epsilon 2 | whole | 0.00 | 0.00 | 0.20 | 0.00 | 0.00 | 0.00 | 0.00 | 0.20 | 0.00 | |
| | | precursor | serum | | | | | | | | | | |
| | Q12879 | Glutamate [NMDA] receptor subunit epsilon | whole | 0.00 | 0.25 | 0.40 | 0.58 | 0.00 | 0.25 | 0.14 | 0.40 | 0.42 | |
| | | 1 precursor | serum | | | | | | | | | | |
| | gi\|33356128 | NMDA receptor regulated 1-like protein isoform 1 | LMW_c | 0 | 0 | 0 | 0 | 0 | 0 | 0 | 1 | 0 | 0 |

TABLE 11
Biomarker candidates for the differentiation between patients having microhemorrhages in the brain and patients without microhemorrhages
Proteins found in non-SH cases but not in SH cases

| | | | | found in x % of samples on average | | |
|---|---|---|---|---|---|---|
| Accession | Name | Probability | MW | SH 0 (n = 16) | SH 1 + 2 (n = 3) | SH 3 (n = 5) |
| P35542 | Serum amyloid A-4 protein precursor | 9.09E−004 | 14797.3 | 0.50 | 0.00 | 0 |

| | | | | found in x % of samples on average | |
|---|---|---|---|---|---|
| Accession | Name | Probability | MW | PMCI non-SH (n = 8) | PMCI SH (n = 3) |

Proteins found in PMCI SH cases but not in PMCI non-SH cases

| | | | | | |
|---|---|---|---|---|---|
| P21817 | Ryanodine receptor 1 | 8.02E−004 | 564813.8 | 0 | 1.00 |
| Q9NVE5 | Ubiquitin carboxyl-terminal hydrolase 40 | 3.65E−005 | 140041.1 | 0 | 0.67 |
| Q5NV79 | V5-4 protein (Fragment) | 4.56E−005 | 10672 | 0 | 0.67 |
| P51843 | Nuclear receptor 0B1 (Nuclear receptor | 4.77E−004 | 51683.9 | 0 | 0.67 |
| | DAX-1) | | | | |
| Q96SU4 | Oxysterol binding protein-related protein 9 | 4.96E−004 | 83132.4 | 0 | 0.67 |
| P17706 | Tyrosine-protein phosphatase, non-receptor | 5.85E−004 | 48497.5 | 0 | 0.67 |
| | type 2 | | | | |
| O15013 | Rho guanine nucleotide exchange factor 10 | 7.23E−004 | 127038.7 | 0 | 0.67 |
| O75129 | KIAA0634 protein (Fragment) | 7.87E−004 | 145332 | 0 | 0.67 |
| Q8N543 | Hypothetical protein FLJ10826 | 8.10E−004 | 63206.2 | 0 | 0.67 |
| Q96AE7 | Tetratricopeptide repeat protein 17 | 8.61E−004 | 129476.8 | 0 | 0.67 |

Proteins found in PMCI non-SH cases but not in PMCI SH cases

| | | | | | |
|---|---|---|---|---|---|
| P02747 | Complement C1q subcomponent | 8.22E−013 | 25757.1 | 0.88 | 0 |
| P02775 | Platelet basic protein precursor (PBP) | 4.10E−009 | 13885.4 | 0.88 | 0 |
| P35542 | Serum amyloid A-4 protein precursor | 9.09E−004 | 14797.3 | 0.88 | 0 |
| P20929 | Nebulin | 2.48E−004 | 772742.8 | 0.63 | 0 |
| Q4V312 | Colony stimulating factor 2 receptor, alpha, | 5.36E−005 | 46871.6 | 0.50 | 0 |
| | low-affinity (Granulocyte-macrophage) | | | | |
| P02671 | Fibrinogen alpha chain precursor | 9.49E−005 | 94914.3 | 0.50 | 0 |
| P13667 | Protein disulfide-isomerase A4 precursor | 2.62E−004 | 72887.1 | 0.50 | 0 |
| Q15643 | Thyroid receptor interacting protein 11 | 9.52E−004 | 227498.2 | 0.50 | 0 |

| | | | | found in x % of samples on average | | |
|---|---|---|---|---|---|---|
| | | | | Normal non-SH | PMCI non-SH | |
| Accession | Name | Probability | MW | (n = 5) | (n = 8) | PMCI SH (n = 3) |

Proteins found only in PMCI non-SH (but neither in Normal non-SH nor PMCI SH) cases

| | | | | | | |
|---|---|---|---|---|---|---|
| Q4V312 | Colony stimulating factor 2 receptor, alpha, | 5.36E−005 | 46871.6 | 0 | 0.50 | 0 |
| | low-affinity (Granulocyte-macrophage) | | | | | |
| P13667 | Protein disulfide-isomerase A4 precursor | 2.62E−004 | 72887.1 | 0 | 0.50 | 0 |

Proteins found only in PMCI SH (but neither in Normal non-SH nor PMCI non-SH) cases

| | | | | | | |
|---|---|---|---|---|---|---|
| P21817 | Ryanodine receptor 1 (Skeletal muscle-type | 8.02E−004 | 564813.8 | 0 | 0 | 1.00 |
| | ryanodine receptor) | | | | | |
| Q9NVE5 | Ubiquitin carboxyl-terminal hydrolase 40 | 3.65E−005 | 140041.1 | 0 | 0 | 0.67 |
| P51843 | Nuclear receptor 0B1 (Nuclear receptor | 4.77E−004 | 51683.9 | 0 | 0 | 0.67 |
| | DAX-1) | | | | | |
| Q96SU4 | Oxysterol binding protein-related protein 9 | 4.96E−004 | 83132.4 | 0 | 0 | 0.67 |
| | (OSBP-related protein 9) (ORP-9) | | | | | |
| O15013 | Rho guanine nucleotide exchange factor 10 | 7.23E−004 | 127038.7 | 0 | 0 | 0.67 |
| O75129 | KIAA0634 protein (Fragment) | 7.87E−004 | 145332 | 0 | 0 | 0.67 |
| Q8N543 | Hypothetical protein FLJ10826 | 8.10E−004 | 63206.2 | 0 | 0 | 0.67 |

SH 0 no hemorrhages
SH 1 + 2 insignificant hemorrhages
SH 3 significant hemorrhages

We claim:

1. A method for diagnosing a neurological condition in a patient comprising:
a) obtaining a biological sample from said patient; and
b) evaluating said sample for the abundance of at least one biomarker selected from the group consisting of a peptide having the amino acid sequence of SEQ ID NOs:1-440,
wherein the abundance of said at least one biomarker is indicative of a neurological condition.

2. The method of claim 1, wherein the abundance of said biomarker is greater than that of a control sample.

3. The method of claim 1, wherein said the abundance of said biomarker is less than that of a control sample.

4. The method of claim 1, further comprising, prior to the evaluation step, harvesting low molecular weight peptides from said sample to generate at least one fraction comprising said peptides.

5. The method of claim 1, wherein said biomarker is a low molecular weight protein complexed with a carrier protein.

6. The method of claim 5, wherein said low molecular weight protein is further purified from said carrier protein.

7. The method of claim 6, wherein said low molecular weight protein is digested and optionally sequenced.

8. The method of claim 1, wherein said biological sample is blood, serum or plasma.

9. The method of claim 1, wherein the evaluation step comprises an assay selected from the group consisting of mass spectrometry, immunoassay, immuno-mass spectrometry and suspension bead array.

10. The method of claim 9, wherein said immunoassay is an enzyme linked immunosorbent assay (ELISA).

11. The method of claim 9, wherein said mass spectrometry comprises tandem mass spectroscopy (MSMS).

12. The method of claim 1, wherein the neurodegenerative disease is selected from the group consisting of
a) Alzheimer's disease;
b) mild cognitive impairment;
c) stable mild cognitive impairment;
d) progressive mild cognitive impairment;
e) vascular dementia;
f) angiopathy black holes;
g) cerebral amyloid angiopathy; and
h) microhemorrages.

13. The method of claim 1, further comprising obtaining a neuroimage of brain microvasculopathy.

14. The method of claim 13, wherein said neuroimage is obtained by a method selected from the group consisting of
a) susceptibility weighted imaging; and
b) magnetic resonance spectroscopy.

15. A method for diagnosing Alzheimer's disease in a patient comprising:
a) obtaining a biological sample from said patient; and
b) evaluating said sample for the abundance of at least one biomarker selected from the group consisting of a peptide having the amino acid sequence of SEQ ID NOs:1, 3-13, 15, 16, 21, 22, 24-28, 31-33, 37-44, 56-59, 66-68, 93-101, 111-128, 143-153, 156-1170, 172-183, 263-279, 310-335, 348, 355-359, 362, 363, 365, 372, 373, 376-402, 406-426 and 436-44;
wherein the abundance of said at least one biomarker is indicative of Alzheimer's disease.

16. The method of claim 15, wherein the marker is a peptide:
(i) associated with inflammation and has the amino acid sequence of SEQ ID NOs:28, 368-373 and 390-401;
(ii) associated with vitamin D metabolism and bone mineralization and has the amino acid sequence of SEQ ID NOs:331 and 427-435;
(iii) associated with coagulation and platelet activity and has the amino acid sequence of SEQ ID NOs:24, 102-110, 157-165, 350-354 and 376-389;
(iv) associated with the complement cascade and has the amino acid sequence of SEQ ID NOs:117-120, 129-138, 139-142 and 330;
(v) associated with globin activity and has the amino acid sequence of SEQ ID NOs:76-92, 144-153;
(vi) are glycosylated /associated with glyscosylation and has the amino acid sequence of SEQ ID NOs: 23, 50-55, 46-49, 174-183, 193-201 and 376-389;
(vii) associated with protease inhibition and has the amino acid sequence of SEQ ID NOs:50-55, 193-201 and 376-389;
(viii) associated with keratins and related proteins and has the amino acid sequence of SEQ ID NOs:154-155, 202-319 and 336-338;
(ix) associated with heme degradation and has the amino acid sequence of SEQ ID NOs:93-98;
(x) associated with pyruvate metabolism and has the amino acid sequence of SEQ ID NO:363;
(xi) associated with calcium related proteins and has the amino acid sequence of SEQ ID NOs:14, 372-373 and 402;
(xii) associated with defensin and has the amino acid sequence of SEQ ID NOs:355-358;
(xiii) associated with gelsolin and has the amino acid sequence of SEQ ID NOs:166-170;
(xiv) associated with vitronectin and has the amino acid sequence of SEQ ID NOs:436-440;
(xv) associated with profilin and has the amino acid sequence of SEQ ID NO:360;
(xvi) associated with thrombospondin and has the amino acid sequence of SEQ ID NO:405;
(xvii) associated with peroxiredoxin or alcohol dehydrogenase and has the amino acid sequence of SEQ ID NOs:340-347;
(xviii) associated with apolipoproteins and has the amino acid sequence of SEQ ID NOs:66-75;
(xix) associated with iron and copper metabolism and has the amino acid sequence of SEQ ID NOs:406-425; or
(xx) associated with NMDA receptor-related proteins and has the amino acid sequence of SEQ ID NOs:35-36.

17. The method of claim 15, wherein said biomarker is a peptide having the amino acid sequence selected from the group consisting of SEQ ID NOs:93-98, 139-142, 166-170, 348, 355-358, 402 and 406-425.

18. The method of claim 15, wherein said the abundance of said biomarker is less than that of a control sample.

19. The method of claim 15, wherein the abundance of said biomarker is greater than that of a control sample.

20. The method of claim 15, further comprising obtaining a neuroimage of brain microvasculopathy associated with Alzheimer's Disease.

21. The method of claim 20, wherein said neuroimage is obtained by a method selected from the group consisting of
a) susceptibility weighted imaging; and
b) magnetic resonance spectroscopy.

22. The method of claim 15, further comprising, prior to the evaluation step, harvesting low molecular weight peptides from said sample to generate at least one fraction comprising said peptides.

23. The method of claim 15, wherein said biomarker is a low molecular weight protein complexed with a carrier protein.

24. The method of claim 23, wherein said low molecular weight protein is further purified from said carrier protein.

25. The method of claim 24, wherein said low molecular weight protein is digested and optionally sequenced.

26. The method of claim 15, wherein said biological sample is blood, serum or plasma.

27. The method of claim 15, wherein the evaluation step comprises an assay selected from the group consisting of mass spectrometry, immunoassay, immuno-mass spectrometry and suspension bead array.

28. The method of claim 27, wherein said immunoassay is an enzyme linked immunosorbent assay (ELISA).

29. The method of claim 27, wherein said mass spectrometry comprises tandem mass spectroscopy (MSMS).

30. A method for diagnosing mild cognitive impairment in a patient comprising:
a) obtaining a biological sample from said patient; and
b) evaluating said sample for the abundance of at least one biomarker selected from the group consisting of a peptide having the amino acid sequence of SEQ ID NOs:2, 4, 14, 17, 23, 29, 34, 45-55, 60-65, 69-92, 102-110, 129-142, 154, 155, 171, 184-191, 193-226, 248-279, 281-320, 333, 336-347, 349-354, 360, 361, 364, 366-371, 374, 375, 403-405 and 427-435;
wherein the abundance of said at least one biomarker is indicative of mild cognitive impairment.

31. The method of claim 30, wherein said the abundance of said biomarker is less than that of a control sample.

32. The method of claim 30, wherein the abundance of said biomarker is greater than that of a control sample.

33. The method of claim 30, further comprising obtaining a neuroimage of brain microvasculopathy associated with Alzheimer's Disease.

34. The method of claim 33, wherein said neuroimage is obtained by a method selected from the group consisting of
a) susceptibility weighted imaging; and
b) magnetic resonance spectroscopy.

35. The method of claim 30, further comprising, prior to the evaluation step, harvesting low molecular weight peptides from said sample to generate at least one fraction comprising said peptides.

36. The method of claim 30, wherein said biomarker is a low molecular weight protein complexed with a carrier protein.

37. The method of claim 36, wherein said low molecular weight protein is further purified from said carrier protein.

38. The method of claim 37, wherein said low molecular weight protein is digested and optionally sequenced.

39. The method of claim 30, wherein said biological sample is blood, serum or plasma.

40. The method of claim 30, wherein the evaluation step comprises an assay selected from the group consisting of mass spectrometry, immunoassay, immuno-mass spectrometry and suspension bead array.

41. The method of claim 40, wherein said immunoassay is an enzyme linked immunosorbent assay (ELISA).

42. The method of claim 40, wherein said mass spectrometry comprises tandem mass spectroscopy (MSMS).

43. A method for diagnosing brain microhemorrhages in a patient comprising:
a) obtaining a biological sample from said patient; and
b) evaluating said sample for the abundance of at least one biomarker selected from the group consisting of a peptide having the amino acid sequence of SEQ ID NOs:441-452
wherein the abundance of said at least one biomarker is indicative of microhemorrhages.

44. The method of claim 43, wherein said the abundance of said biomarker is less than that of a control sample.

45. The method of claim 43, wherein the abundance of said biomarker is greater than that of a control sample.

46. The method of claim 43, further comprising obtaining a neuroimage of brain microvasculopathy associated with Alzheimer's Disease.

47. The method of claim 46, wherein said neuroimage is obtained by a method selected from the group consisting of
a) susceptibility weighted imaging; and
b) magnetic resonance spectroscopy.

48. The method of claim 43, further comprising, prior to the evaluation step, harvesting low molecular weight peptides from said sample to generate at least one fraction comprising said peptides.

49. The method of claim 43, wherein said biomarker is a low molecular weight protein complexed with a carrier protein.

50. The method of claim 49, wherein said low molecular weight protein is further purified from said carrier protein.

51. The method of claim 50, wherein said low molecular weight protein is digested and optionally sequenced.

52. The method of claim 43, wherein said biological sample is blood, serum or plasma.

53. The method of claim 43, wherein the evaluation step comprises an assay selected from the group consisting of mass spectrometry, immunoassay, immuno-mass spectrometry and suspension bead array.

54. The method of claim 53, wherein said immunoassay is an enzyme linked immunosorbent assay (ELISA).

55. The method of claim 53, wherein said mass spectrometry comprises tandem mass spectroscopy (MSMS).

56. An antibody specific for a peptide selected from the group consisting of a peptide having the amino acid sequence of SEQ ID NOs:1-440.

57. The antibody of claim 56, wherein said antibody is a monoclonal antibody, polyclonal antibody or chimeric antibody.

58. A kit for detecting a neurological condition in a patient, comprising at least one antibody of claim 56 and instructions for the storage or use thereof.


SEQUENCE LISTING (452 sequences)

SEQ ID NO: 1 - protein, 20 aa, Homo sapiens
LYFCGLSDAVLKDGYFRNLK

SEQ ID NO: 2 - protein, 13 aa, Homo sapiens
DSADGTITRVTLR

SEQ ID NO: 3 - protein, 19 aa, Homo sapiens
ALRILQNEPLPERLEVNGR

SEQ ID NO: 4 - protein, 11 aa, Homo sapiens
AEDTAVYYCAK

SEQ ID NO: 5 - protein, 13 aa, Homo sapiens
AADDTWEPFASGK

SEQ ID NO: 6 - protein, 24 aa, Homo sapiens
ALGISPFHEHAEVVFTANDSGPRR

SEQ ID NO: 7 - protein, 13 aa, Homo sapiens
GSPAINVAVHVFR

SEQ ID NO: 8 - protein, 14 aa, Homo sapiens
GSPAINVAVHVFRK

SEQ ID NO: 9 - protein, 14 aa, Homo sapiens
KAADDTWEPFASGK

SEQ ID NO: 10 - protein, 24 aa, Homo sapiens
RYTIAALLSPYSYSTTAVVTNPKE

SEQ ID NO: 11 - protein, 28 aa, Homo sapiens
TSESGELHGLTTEEEFVEGIYKVEIDTK

SEQ ID NO: 12 - protein, 19 aa, Homo sapiens
VLDAVRGSPAINVAVHVFR

SEQ ID NO: 13 - protein, 23 aa, Homo sapiens
YTIAALLSPYSYSTTAVVTNPKE

SEQ ID NO: 14 - protein, 19 aa, Homo sapiens
HRPTAGAFNHSDLDAELRR

SEQ ID NO: 15 - protein, 14 aa, Homo sapiens
EDAIEHFMKLYEEK

SEQ ID NO: 16 - protein, 26 aa, Homo sapiens
MKLTLKGGAAVDPDSGLEHSAHVLEK

SEQ ID NO: 17 - protein, 11 aa, Homo sapiens
AASGTQNNVLR

SEQ ID NO: 18 - protein, 7 aa, Homo sapiens
FSSHGCK

SEQ ID NO: 19 - protein, 9 aa, Homo sapiens
MHVLHCQGR

SEQ ID NO: 20 - protein, 11 aa, Homo sapiens
SYTSHTNEIHK

SEQ ID NO: 21 - protein, 14 aa, Homo sapiens
LAPEYEKAAKELSK

SEQ ID NO: 22 - protein, 18 aa, Homo sapiens
FLQEAKILKQYDHPNIVK

SEQ ID NO: 23 - protein, 21 aa, Homo sapiens
EGVNITICFQIKSLYPQFQGR

SEQ ID NO: 24 - protein, 27 aa, Homo sapiens
TLNLAQNLLAQLPEELFHPLTSLQTLK

SEQ ID NO: 25 - protein, 8 aa, Homo sapiens
FRPDGLPK

SEQ ID NO: 26 - protein, 25 aa, Homo sapiens
EGLGPGDTTSTFCGTPNYIAPEILR

SEQ ID NO: 27 - protein, 9 aa, Homo sapiens
ILIGTVFHK

SEQ ID NO: 28 - protein, 17 aa, Homo sapiens
DKLNDNHEVEDEMGPQR

SEQ ID NO: 29 - protein, 9 aa, Homo sapiens
SAVQGPPDR

SEQ ID NO: 30 - protein, 9 aa, Homo sapiens
SAVQGPPDR

SEQ ID NO: 31 - protein, 9 aa, Homo sapiens
PRGSLATFK

SEQ ID NO: 32 - protein, 11 aa, Homo sapiens
ILIDGTLIIFR

SEQ ID NO: 33 - protein, 23 aa, Homo sapiens
KTVPSALLVQLIQERLAEEDCIK

SEQ ID NO: 34 - protein, 24 aa, Homo sapiens
GMGSDRTALSLQGAWGIFLSTFYK

SEQ ID NO: 35 - protein, 27 aa, Homo sapiens
CEVNAEDKENSGDYISENEDPELQDYR

SEQ ID NO: 36 - protein, 16 aa, Homo sapiens
KAYINTISSLKDLITK

SEQ ID NO: 37 - protein, 13 aa, Homo sapiens
HSQPATPTPLQSR

SEQ ID NO: 38 - protein, 18 aa, Homo sapiens
NCYSLLNLAEVRSKYLGK

SEQ ID NO: 39 - protein, 18 aa, Homo sapiens
NCYSLLNLAEVRSKYLGK

SEQ ID NO: 40 - protein, 11 aa, Homo sapiens
VEDTAVYYCAR

SEQ ID NO: 41 - protein, 26 aa, Homo sapiens
ENLEVGYEVLTIRASDRDSPINANLR

SEQ ID NO: 42 - protein, 9 aa, Homo sapiens
ETKETHVLR

SEQ ID NO: 43 - protein, 14 aa, Homo sapiens
QFVGCMRNLSVDGK

SEQ ID NO: 44 - protein, 25 aa, Homo sapiens
VPGGTRAFALRPGCTYAVGAACTPR

SEQ ID NO: 45 - protein, 16 aa, Homo sapiens
DGVYFLYEALHGPPKK

SEQ ID NO: 46 - protein, 11 aa, Homo sapiens
ATWSGAVLAGR

SEQ ID NO: 47 - protein, 20 aa, Homo sapiens
SLPAPWLSMAPVSWITPGLK

SEQ ID NO: 48 - protein, 22 aa, Homo sapiens
SWVPHTFESELSDPVELLVAES

SEQ ID NO: 49 - protein, 22 aa, Homo sapiens
TPGAAANLELIFVGPQHAGNYR

SEQ ID NO: 50 - protein, 10 aa, Homo sapiens
ADLSGITGAR

SEQ ID NO: 51 - protein, 19 aa, Homo sapiens
AVLDVFEEGTEASAATAVK

SEQ ID NO: 52 - protein, 20 aa, Homo sapiens
DYNLNDILLQLGIEEAFTSK

SEQ ID NO: 53 - protein, 11 aa, Homo sapiens
ITLLSALVETR

SEQ ID NO: 54 - protein, 10 aa, Homo sapiens
NLAVSQVVHK

SEQ ID NO: 55 - protein, 19 aa, Homo sapiens
YNLNDILLQLGIEEAFTSK

SEQ ID NO: 56 - protein, 9 aa, Homo sapiens
EWFSETFQK

SEQ ID NO: 57 - protein, 9 aa, Homo sapiens
IKQSELSAK

SEQ ID NO: 58 - protein, 11 aa, Homo sapiens
LKEFGNTLEDK

SEQ ID NO: 59 - protein, 10 aa, Homo sapiens
TPDVSSALDK

SEQ ID NO: 60 - protein, 11 aa, Homo sapiens
ESLSSYWESAK

SEQ ID NO: 61 - protein, 7 aa, Homo sapiens
LRDLYSK

SEQ ID NO: 62 - protein, 21 aa, Homo sapiens
STAAMSTYTGIFTDQVLSVLK

SEQ ID NO: 63 - protein, 24 aa, Homo sapiens
STAAMSTYTGIFTDQVLSVLKGEE

SEQ ID NO: 64 - protein, 9 aa, Homo sapiens
TAAQNLYEK

SEQ ID NO: 65 - protein, 9 aa, Homo sapiens
TYLPAVDEK

SEQ ID NO: 66 - protein, 7 aa, Homo sapiens
AWFLESK

SEQ ID NO: 67 - protein, 13 aa, Homo sapiens
DGWQWFWSPSTFR

SEQ ID NO: 68 - protein, 9 aa, Homo sapiens
ELLETVVNR

SEQ ID NO: 69 - protein, 8 aa, Homo sapiens
ILQADQEL

SEQ ID NO: 70 - protein, 9 aa, Homo sapiens
LNILNNNYK

SEQ ID NO: 71 - protein, 28 aa, Homo sapiens
EELATFDPVDNIVFNMAAGSAPMQLHLR

SEQ ID NO: 72 - protein, 20 aa, Homo sapiens
EFPEVHLGQWYFIAGAAPTK

SEQ ID NO: 73 - protein, 14 aa, Homo sapiens
KWIYHLTEGSTDLR

SEQ ID NO: 74 - protein, 9 aa, Homo sapiens
MKDGLCVPR

SEQ ID NO: 75 - protein, 9 aa, Homo sapiens
SLTSCLDSK

SEQ ID NO: 76 - protein, 12 aa, Homo sapiens
EFTPPVQAAYQK

SEQ ID NO: 77 - protein, 19 aa, Homo sapiens
FFESFGDLSTPDAVMGNPK

SEQ ID NO: 78 - protein, 21 aa, Homo sapiens
FFESFGDLSTPDAVMGNPKVK

SEQ ID NO: 79 - protein, 13 aa, Homo sapiens
GTFATLSELHCDK

SEQ ID NO: 80 - protein, 22 aa, Homo sapiens
GTFATLSELHCDKLHVDPENFR

SEQ ID NO: 81 - protein, 17 aa, Homo sapiens
KVLGAFSDGLAHLDNLK

SEQ ID NO: 82 - protein, 9 aa, Homo sapiens
LHVDPENFR

SEQ ID NO: 83 - protein, 16 aa, Homo sapiens
LLGNVLVCVLAHHFGK

SEQ ID NO: 84 - protein, 10 aa, Homo sapiens
LLVVYPWTQR

SEQ ID NO: 85 - protein, 29 aa, Homo sapiens
LLVVYPWTQRFFESFGDLSTPDAVMGNPK

SEQ ID NO: 86 - protein, 9 aa, Homo sapiens
SAVTALWGK

SEQ ID NO: 87 - protein, 22 aa, Homo sapiens
SAVTALWGKVNVDEVGGEALGR

SEQ ID NO: 88 - protein, 32 aa, Homo sapiens
SAVTALWGKVNVDEVGGEALGRLLVVYPWTQR

SEQ ID NO: 89 - protein, 16 aa, Homo sapiens
VLGAFSDGLAHLDNLK

SEQ ID NO: 90 - protein, 13 aa, Homo sapiens
VNVDEVGGEALGR

SEQ ID NO: 91 - protein, 12 aa, Homo sapiens
VVAGVANALAHK

SEQ ID NO: 92 - protein, 14 aa, Homo sapiens
VVAGVANALAHKYH

SEQ ID NO: 93 - protein, 24 aa, Homo sapiens
LPSEGPRPAHVVVGDVLQAADVDK

SEQ ID NO: 94 - protein, 7 aa, Homo sapiens
NIVAAMK

SEQ ID NO: 95 - protein, 17 aa, Homo sapiens
PAHVVVGDVLQAADVDK

SEQ ID NO: 96 - protein, 15 aa, Homo sapiens
TVAGQDAVIVLLGTR

SEQ ID NO: 97 - protein, 15 aa, Homo sapiens
VVACTSAFLLWDPTK

SEQ ID NO: 98 - protein, 25 aa, Homo sapiens
YVAVMPPHIGDQPLTGAYTVTLDGR

SEQ ID NO: 99 - protein, 7 aa, Homo sapiens
EISEAMK

SEQ ID NO: 100 - protein, 13 aa, Homo sapiens
EAQQYSEALASTR

SEQ ID NO: 101 - protein, 12 aa, Homo sapiens
QQKFYLETQAGK

SEQ ID NO: 102 - protein, 10 aa, Homo sapiens
ELLESYIDGR

SEQ ID NO: 103 - protein, 16 aa, Homo sapiens
HQDFNSAVQLVENFCR

SEQ ID NO: 104 - protein, 20 aa, Homo sapiens
IVEGSDAEIGMSPWQVMLFR

SEQ ID NO: 105 - protein, 19 aa, Homo sapiens
LAVTTHGLPCLAWASAQAK

SEQ ID NO: 106 - protein, 19 aa, Homo sapiens
SEGSSVNLSPPLEQCVPDR

SEQ ID NO: 107 - protein, 18 aa, Homo sapiens
SLEDKTERELLESYIDGR

SEQ ID NO: 108 - protein, 13 aa, Homo sapiens
TATSEYQTFFNPR

SEQ ID NO: 109 - protein, 17 aa, Homo sapiens
TFGSGEADCGLRPLFEK

SEQ ID NO: 110 - protein, 9 aa, Homo sapiens
YGFYTHVFR

SEQ ID NO: 111 - protein, 12 aa, Homo sapiens
DFALLSLQVPLK

SEQ ID NO: 112 - protein, 15 aa, Homo sapiens
DFALLSLQVPLKDAK

SEQ ID NO: 113 - protein, 9 aa, Homo sapiens
DHAVDLIQK

SEQ ID NO: 114 - protein, 14 aa, Homo sapiens
EPFLSCCQFAESLR

SEQ ID NO: 115 - protein, 13 aa, Homo sapiens
GLEEELQFSLGSK

SEQ ID NO: 116 - protein, 14 aa, Homo sapiens
GPEVQLVAHSPWLK

SEQ ID NO: 117 - protein, 12 aa, Homo sapiens
KADGSYAAWLSR

SEQ ID NO: 118 - protein, 26 aa, Homo sapiens
LHLETDSLALVALGALDTALYAAGSK

SEQ ID NO: 119 - protein, 9 aa, Homo sapiens
SCGLHQLLR

SEQ ID NO: 120 - protein, 8 aa, Homo sapiens
TYNVLDMK

SEQ ID NO: 121 - protein, 9 aa, Homo sapiens
VDFTLSSER

SEQ ID NO: 122 - protein, 21 aa, Homo sapiens
VGLSGMAIADVTLLSGFHALR

SEQ ID NO: 123 - protein, 15 aa, Homo sapiens
VLSLAQEQVGGSPEK

SEQ ID NO: 124 - protein, 23 aa, Homo sapiens
YVSHFETEGPHVLLYFDSVPTSR

SEQ ID NO: 125 - protein, 12 aa, Homo sapiens
FQNSAILTIQPK

SEQ ID NO: 126 - protein, 16 aa, Homo sapiens
MVETTAYALLTSLNLK

SEQ ID NO: 127 - protein, 7 aa, Homo sapiens
VFQFLEK

SEQ ID NO: 128 - protein, 16 aa, Homo sapiens
YIYPLDSLTWIEYWPR

SEQ ID NO: 129 - protein, 21 aa, Homo sapiens
AEATTLHVAPQGTAMAVSTFR

SEQ ID NO: 130 - protein, 22 aa, Homo sapiens
ANFDAQQFAGTWLLVAVGSACR

SEQ ID NO: 131 - protein, 22 aa, Homo sapiens
GAVHVVVAETDYQSFAVLYLER

SEQ ID NO: 132 - protein, 10 aa, Homo sapiens
KLDGICWQVR

SEQ ID NO: 133 - protein, 9 aa, Homo sapiens
LDGICWQVR

SEQ ID NO: 134 - protein, 11 aa, Homo sapiens
QLYGDTGVLGR

SEQ ID NO: 135 - protein, 12 aa, Homo sapiens
RPASPISTIQPK

SEQ ID NO: 136 - protein, 15 aa, Homo sapiens
SLPVSDSVLSGFEQR

SEQ ID NO: 137 - protein, 16 aa, Homo sapiens
VQEAHLTEDQIFYFPK

SEQ ID NO: 138 - protein, 17 aa, Homo sapiens
YGFCEAADQFHVLDEVR

SEQ ID NO: 139 - protein, 11 aa, Homo sapiens
EIMENYNIALR

SEQ ID NO: 140 - protein, 11 aa, Homo sapiens
KGEWVALNPLR

SEQ ID NO: 141 - protein, 23 aa, Homo sapiens
NTEILTGSWSDQTYPEGTQAIYK

SEQ ID NO: 142 - protein, 10 aa, Homo sapiens
SLGNVIMVCR

SEQ ID NO: 143 - protein, 17 aa, Homo sapiens
MAKESGISLKEIQVLAR

SEQ ID NO: 144 - protein, 12 aa, Homo sapiens
EFTPQMQAAYQK

SEQ ID NO: 145 - protein, 13 aa, Homo sapiens
GTFSQLSELHCDK

SEQ ID NO: 146 - protein, 22 aa, Homo sapiens
GTFSQLSELHCDKLHVDPENFR

SEQ ID NO: 147 - protein, 12 aa, Homo sapiens
LLGNVLVCVLAR

SEQ ID NO: 148 - protein, 10 aa, Homo sapiens
LLVVYPWTQR

SEQ ID NO: 149 - protein, 9 aa, Homo sapiens
TAVNALWGK

SEQ ID NO: 150 - protein, 16 aa, Homo sapiens
VLGAFSDGLAHLDNLK

SEQ ID NO: 151 - protein, 13 aa, Homo sapiens
VNVDAVGGEALGR

SEQ ID NO: 152 - protein, 12 aa, Homo sapiens
VVAGVANALAHK

SEQ ID NO: 153 - protein, 14 aa, Homo sapiens
VVAGVANALAHKYH

SEQ ID NO: 154 - protein, 11 aa, Homo sapiens
ANSSATETINK

SEQ ID NO: 155 - protein, 17 aa, Homo sapiens
GFFDPNTEENLTYLQLK

SEQ ID NO: 156 - protein, 17 aa, Homo sapiens
PGSPACSPVLGSGGRAR

SEQ ID NO: 157 - protein, 20 aa, Homo sapiens
TFPGFFSPMLGEFVSETESR

SEQ ID NO: 158 - protein, 10 aa, Homo sapiens
VTSGSTTTTR

SEQ ID NO: 159 - protein, 15 aa, Homo sapiens
ESSSHHPGIAEFPSR

SEQ ID NO: 160 - protein, 12 aa, Homo sapiens
EVDLKDYEDQQK

SEQ ID NO: 161 - protein, 13 aa, Homo sapiens
GLIDEVNQDFTNR

SEQ ID NO: 162 - protein, 11 aa, Homo sapiens
GSESGIFTNTK

SEQ ID NO: 163 - protein, 15 aa, Homo sapiens
HPDEAAFFDTASTGK

SEQ ID NO: 164 - protein, 17 aa, Homo sapiens
HRHPDEAAFFDTASTGK

SEQ ID NO: 165 - protein, 20 aa, Homo sapiens
TFPGFFSPMLGEFVSETESR

SEQ ID NO: 166 - protein, 15 aa, Homo sapiens
DSQEEEKTEALTSAK

SEQ ID NO: 167 - protein, 15 aa, Homo sapiens
EVQGFESATFLGYFK

SEQ ID NO: 168 - protein, 24 aa, Homo sapiens
FDLVPVPTNLYGDFFTGDAYVILK

SEQ ID NO: 169 - protein, 11 aa, Homo sapiens
HVVPNEVVVQR

SEQ ID NO: 170 - protein, 13 aa, Homo sapiens
PNSMVVEHPEFLK

SEQ ID NO: 171 - protein, 14 aa, Homo sapiens
RTTEMMPVYLDLNK

SEQ ID NO: 172 - protein, 14 aa, Homo sapiens [sequence omitted]

SEQ ID NO: 173 - protein, 12 aa, Homo sapiens
GTTVTVSSASTK

SEQ ID NO: 174 - protein, 8 aa, Homo sapiens
ALDLINKR

SEQ ID NO: 175 - protein, 9 aa, Homo sapiens
DGYLFQLLR

SEQ ID NO: 176 - protein, 14 aa, Homo sapiens
DSPVLIDFFEDTER

SEQ ID NO: 177 - protein, 20 aa, Homo sapiens
GEVLPLPEANFPSFPLPHHK

SEQ ID NO: 178 - protein, 21 aa, Homo sapiens
HPLKPDNQPFPQSVSESCPGK

SEQ ID NO: 179 - protein, 21 aa, Homo sapiens
KGEVLPLPEANFPSFPLPHHK

SEQ ID NO: 180 - protein, 5 aa, Homo sapiens
LPPLR

SEQ ID NO: 181 - protein, 10 aa, Homo sapiens
RDGYLFQLLR

SEQ ID NO: 182 - protein, 11 aa, Homo sapiens
RPSEIVIGQCK

SEQ ID NO: 183 - protein, 16 aa, Homo sapiens
SGFPQVSMFFTHTFPK

SEQ ID NO: 184 - protein, 12 aa, Homo sapiens
GTTTFAVTSILR

SEQ ID NO: 185 - protein, 9 aa, Homo sapiens
TPLTATLSK

SEQ ID NO: 186 - protein, 12 aa, Homo sapiens
VTVSSASPTSPK

SEQ ID NO: 187 - protein, 10 aa, Homo sapiens
AGVETTKPSK

SEQ ID NO: 188 - protein, 19 aa, Homo sapiens
ANPTVTLFPPSSEELQANK

SEQ ID NO: 189 - protein, 8 aa, Homo sapiens
VTVLGQPK

SEQ ID NO: 190 - protein, 9 aa, Homo sapiens
TGYYWGWIR

SEQ ID NO: 191 - protein, 9 aa, Homo sapiens
GLEWVANIK

SEQ ID NO: 192 - protein, 13 aa, Homo sapiens
FSGSGSGTDFTLK

SEQ ID NO: 193 - protein, 13 aa, Homo sapiens
AEAQAQYSAAVAK

SEQ ID NO: 194 - protein, 11 aa, Homo sapiens
AGFSWIEVTFK

SEQ ID NO: 195 - protein, 15 aa, Homo sapiens
ANTVQEATFQMELPK

SEQ ID NO: 196 - protein, 28 aa, Homo sapiens
FSSHVGGTLGQFYQEVLWGSPAASDDGR

SEQ ID NO: 197 - protein, 12 aa, Homo sapiens
GPDVLTATVSGK

SEQ ID NO: 198 - protein, 26 aa, Homo sapiens
LWAYLTIQQLLEQTVSASDADQQALR

SEQ ID NO: 199 - protein, 11 aa, Homo sapiens
MNFRPGVLSSR

SEQ ID NO: 200 - protein, 10 aa, Homo sapiens
NVHSGSTFFK

SEQ ID NO: 201 - protein, 11 aa, Homo sapiens
VTIGLLFWDGR

SEQ ID NO: 202 - protein, 10 aa, Homo sapiens
AEAESLYQSK

SEQ ID NO: 203 - protein, 9 aa, Homo sapiens
AQYEDIAQK

SEQ ID NO: 204 - protein, 9 aa, Homo sapiens
DYQELMNTK

SEQ ID NO: 205 - protein, 16 aa, Homo sapiens
EQIKSLNNQFASFIDK

SEQ ID NO: 206 - protein, 12 aa, Homo sapiens
FLEQQNQVLQTK

SEQ ID NO: 207 - protein, 20 aa, Homo sapiens
FSSCGGGGGSFGAGGGFGSR

SEQ ID NO: 208 - protein, 31 aa, Homo sapiens
GGGGGGYGSGGSSYGSGGGSYGSGGGGGGGR

SEQ ID NO: 209 - protein, 8 aa, Homo sapiens
IEISELNR

SEQ ID NO: 210 - protein, 11 aa, Homo sapiens
LALDLEIATYR

SEQ ID NO: 211 - protein, 12 aa, Homo sapiens
LNDLEDALQQAK

SEQ ID NO: 212 - protein, 26 aa, Homo sapiens
MSGECAPNVSVSVSTSHTTISGGGSR

SEQ ID NO: 213 - protein, 14 aa, Homo sapiens
NKLNDLEDALQQAK

SEQ ID NO: 214 - protein, 10 aa, Homo sapiens
NKYEDEINKR

SEQ ID NO: 215 - protein, 10 aa, Homo sapiens
NMQDMVEDYR

SEQ ID NO: 216 - protein, 10 aa, Homo sapiens
NMQDMVEDYR

SEQ ID NO: 217 - protein, 15 aa, Homo sapiens
QISNLQQSISDAEQR

SEQ ID NO: 218 - protein, 12 aa, Homo sapiens
SKAEAESLYQSK

SEQ ID NO: 219 - protein, 12 aa, Homo sapiens
SLDLDSIIAEVK

SEQ ID NO: 220 - protein, 16 aa, Homo sapiens
THNLEPYFESFINNLR

SEQ ID NO: 221 - protein, 9 aa, Homo sapiens
TLLEGEESR

SEQ ID NO: 222 - skipped

SEQ ID NO: 223 - protein, 11 aa, Homo sapiens
TNAENEFVTIK

SEQ ID NO: 224 - protein, 12 aa, Homo sapiens
WELLQQVDTSTR

SEQ ID NO: 225 - protein, 10 aa, Homo sapiens
YEELQITAGR

SEQ ID NO: 226 - protein, 27 aa, Homo sapiens
GSSGGGCFGGSSGGYGGLGGFGGGSFR

SEQ ID NO: 227 - protein, 18 aa, Homo sapiens
ADLEMQIESLTEELAYLK

SEQ ID NO: 228 - protein, 17 aa, Homo sapiens
AETECQNTEYQQLLDIK

SEQ ID NO: 229 - protein, 12 aa, Homo sapiens
ALEESNYELEGK

SEQ ID NO: 230 - protein, 9 aa, Homo sapiens
DAEAWFNEK

SEQ ID NO: 231 - protein, 17 aa, Homo sapiens
ELTTEIDNNIEQISSYK

SEQ ID NO: 232 - protein, 19 aa, Homo sapiens
GSLGGGFSSGGFSGGSFSR

SEQ ID NO: 233 - protein, 27 aa, Homo sapiens
GSSGGGCFGGSSGGYGGLGGFGGGSFR

SEQ ID NO: 234 - protein, 7 aa, Homo sapiens
IKEWYEK

SEQ ID NO: 235 - protein, 7 aa, Homo sapiens
LASYLDK

SEQ ID NO: 236 - protein, 9 aa, Homo sapiens
LENEIQTYR

SEQ ID NO: 237 - protein, 10 aa, Homo sapiens
LKYENEVALR

SEQ ID NO: 238 - protein, 27 aa, Homo sapiens
NVSTGDVNVEMNAAPGVDLTQLLNNMR

SEQ ID NO: 239 - protein, 19 aa, Homo sapiens
SKELTTEIDNNIEQISSYK

SEQ ID NO: 240 - protein, 14 aa, Homo sapiens
SLLEGEGSSGGGGR

SEQ ID NO: 241 - protein, 11 aa, Homo sapiens
SQYEQLAEQNR

SEQ ID NO: 242 - protein, 12 aa, Homo sapiens
SQYEQLAEQNRK

SEQ ID NO: 243 - protein, 27 aa, Homo sapiens
TIDDLKNQILNLTTDNANILLQIDNAR

SEQ ID NO: 244 - protein, 9 aa, Homo sapiens
VLDELTLTK

SEQ ID NO: 245 - protein, 9 aa, Homo sapiens
VTMQNLNDR

SEQ ID NO: 246 - protein, 23 aa, Homo sapiens
YCVQLSQIQAQISALEEQLQQIR

SEQ ID NO: 247 - protein, 8 aa, Homo sapiens
YENEVALR

SEQ ID NO: 248 - protein, 29 aa, Homo sapiens
DIENQYETQITQIEHEVSSSGQEVQSSAK

SEQ ID NO: 249 - protein, 23 aa, Homo sapiens
EIETYHNLLEGGQEDFESSGAGK

SEQ ID NO: 250 - protein, 13 aa, Homo sapiens
FSSSSGYGGGSSR

SEQ ID NO: 251 - protein, 32 aa, Homo sapiens
GGGGSFGYSYGGGSGGGFSASSLGGGFGGGSR

SEQ ID NO: 252 - protein, 16 aa, Homo sapiens
HGVQELEIELQSQLSK

SEQ ID NO: 253 - protein, 10 aa, Homo sapiens
IKFEMEQNLR

SEQ ID NO: 254 - protein, 8 aa, Homo sapiens
IQDWYDKK

SEQ ID NO: 255 - protein, 7 aa, Homo sapiens
LASYLDK

SEQ ID NO: 256 - protein, 25 aa, Homo sapiens
NYSPYYNTIDDLKDQIVDLTVGNNK

SEQ ID NO: 257 - protein, 18 aa, Homo sapiens
SDLEMQYETLQEELMALK

SEQ ID NO: 258 - protein, 16 aa, Homo sapiens
SGGGGGGGLGSGGSIR

SEQ ID NO: 259 - protein, 9 aa, Homo sapiens
STMQELNSR

SEQ ID NO: 260 - protein, 9 aa, Homo sapiens
TLLDIDNTR

SEQ ID NO: 261 - protein, 14 aa, Homo sapiens
VQALEEANNDLENK

SEQ ID NO: 262 - protein, 23 aa, Homo sapiens
YCGQLQMIQEQISNLEAQITDVR

SEQ ID NO: 263 - protein, 19 aa, Homo sapiens
AETECQNTEYQQLLDIKIR

SEQ ID NO: 264 - protein, 17 aa, Homo sapiens
GSLGGGFSSGGFSGGSF

SEQ ID NO: 265 - protein, 9 aa, Homo sapiens
ILLQIDNAR

SEQ ID NO: 266 - protein, 11 aa, Homo sapiens
IRLENEIQTYR

SEQ ID NO: 267 - protein, 12 aa, Homo sapiens
ISALEEQLQQIR

SEQ ID NO: 268 - protein, 7 aa, Homo sapiens
LAADDFR

SEQ ID NO: 269 - protein, 7 aa, Homo sapiens
LASYLDK

SEQ ID NO: 270 - protein, 9 aa, Homo sapiens
LENEIQTYR

SEQ ID NO: 271 - protein, 10 aa, Homo sapiens
LKYENEVALR

SEQ ID NO: 272 - protein, 12 aa, Homo sapiens
NQILNLTTDNAN

SEQ ID NO: 273 - protein, 21 aa, Homo sapiens
NQILNLTTDNANILLQIDNAR

SEQ ID NO: 274 - protein, 16 aa, Homo sapiens
NVQALEIELQSQLALK

SEQ ID NO: 275 - protein, 10 aa, Homo sapiens
RVLDELTLTK

SEQ ID NO: 276 - protein, 20 aa, Homo sapiens
SGGGGGGGGCGGGGGVSSLR

SEQ ID NO: 277 - protein, 19 aa, Homo sapiens
SKELTTEIDNNIEQISSYK

SEQ ID NO: 278 - protein, 14 aa, Homo sapiens
SLLEGEGSSGGGGR

SEQ ID NO: 279 - protein, 15 aa, Homo sapiens
VQALEIELQSQLALK

SEQ ID NO: 280 - protein, 9 aa, Homo sapiens
LEQEIATYR

SEQ ID NO: 281 - protein, 13 aa, Homo sapiens
EVATNSELVQSGK

SEQ ID NO: 282 - protein, 15 aa, Homo sapiens
GSCGIGGGIGGGSSR

SEQ ID NO: 283 - protein, 11 aa, Homo sapiens
ISSVLAGGSCR

SEQ ID NO: 284 - protein, 9 aa, Homo sapiens
VLDELTLAR

SEQ ID NO: 285 - protein, 10 aa, Homo sapiens
AEAESLYQSK

SEQ ID NO: 286 - protein, 11 aa, Homo sapiens
ELLQQVDTSTR

SEQ ID NO: 287 - protein, 28 aa, Homo sapiens
GGSGGGGGGSSGGRGSGGGSSGGSIGGR

SEQ ID NO: 288 - protein, 14 aa, Homo sapiens
GSGGGSSGGSIGGR

SEQ ID NO: 289 - protein, 16 aa, Homo sapiens
KQISNLQQSISDAEQR

SEQ ID NO: 290 - protein, 11 aa, Homo sapiens
LALDLEIATYR

SEQ ID NO: 291 - protein, 14 aa, Homo sapiens
NKLNDLEDALQQAK

SEQ ID NO: 292 - protein, 10 aa, Homo sapiens
NVEIDPEIQK

SEQ ID NO: 293 - protein, 8 aa, Homo sapiens
SEIDNVKK

SEQ ID NO: 294 - protein, 17 aa, Homo sapiens
SGGGFSSGSAGIINYQR

SEQ ID NO: 295 - protein, 8 aa, Homo sapiens
SISDAEQR

SEQ ID NO: 296 - protein, 12 aa, Homo sapiens
SISISVARGGGR

SEQ ID NO: 297 - protein, 12 aa, Homo sapiens
SKAEAESLYQSK

SEQ ID NO: 298 - protein, 12 aa, Homo sapiens
SLDLDSIIAEVK

SEQ ID NO: 299 - protein, 12 aa, Homo sapiens
SLNNQFASFIDK

SEQ ID NO: 300 - protein, 14 aa, Homo sapiens
SLNNQFASFIDKVR

SEQ ID NO: 301 - protein, 9 aa, Homo sapiens
SLVNLGGSK

SEQ ID NO: 302 - protein, 11 aa, Homo sapiens
THNLEPYFESF

SEQ ID NO: 303 - protein, 16 aa, Homo sapiens
THNLEPYFESFINNLR

SEQ ID NO: 304 - protein, 9 aa, Homo sapiens
TLLEGEESR

SEQ ID NO: 305 - protein, 11 aa, Homo sapiens
TNAENEFVTIK

SEQ ID NO: 306 - protein, 10 aa, Homo sapiens
VDQLKSDQSR

SEQ ID NO: 307 - protein, 9 aa, Homo sapiens
VEIDPEIQK

SEQ ID NO: 308 - protein, 12 aa, Homo sapiens
WELLQQVDTSTR

SEQ ID NO: 309 - protein, 10 aa, Homo sapiens
YEELQITAGR

SEQ ID NO: 310 - protein, 14 aa, Homo sapiens
GFSSGSAVVSGGSR

SEQ ID NO: 311 - protein, 8 aa, Homo sapiens
IEISELNR

SEQ ID NO: 312 - protein, 11 aa, Homo sapiens
LALDVEIATYR

SEQ ID NO: 313 - protein, 12 aa, Homo sapiens
LNDLEEALQQAK

SEQ ID NO: 314 - protein, 12 aa, Homo sapiens
NLDLDSIIAEVK

SEQ ID NO: 315 - protein, 12 aa, Homo sapiens
NVQDAIADAEQR

SEQ ID NO: 316 - protein, 12 aa, Homo sapiens
VDLLNQEIEFLK

SEQ ID NO: 317 - protein, 8 aa, Homo sapiens
YEDEINKR

SEQ ID NO: 318 - protein, 10 aa, Homo sapiens
YEELQVTVGR

SEQ ID NO: 319 - protein, 9 aa, Homo sapiens
YLDGLTAER

SEQ ID NO: 320 - protein, 20 aa, Homo sapiens
PFLSIISARYSPHQDADPLK

SEQ ID NO: 321 - protein, 17 aa, Homo sapiens
TFVPGCQPGEFTLGNIK

SEQ ID NO: 322 - protein, 15 aa, Homo sapiens
VPLQQNFQDNQFQGK

SEQ ID NO: 323 - protein, 12 aa, Homo sapiens
GISLANWMCLAK

SEQ ID NO: 324 - protein, 7 aa, Homo sapiens
LGMDGYR

SEQ ID NO: 325 - protein, 8 aa, Homo sapiens
QYVQGCGV

SEQ ID NO: 326 - protein, 8 aa, Homo sapiens
RLGMDGYR

SEQ ID NO: 327 - protein, 12 aa, Homo sapiens
STDYGIFQINSR

SEQ ID NO: 328 - protein, 28 aa, Homo sapiens
TPGAVNACHLSCSALLQDNIADAVACAK

SEQ ID NO: 329 - protein, 8 aa, Homo sapiens
WESGYNTR

SEQ ID NO: 330 - protein, 15 aa, Homo sapiens
VLATLCGQESTDTER

SEQ ID NO: 331 - protein, 11 aa, Homo sapiens
NANTFISPQQR

SEQ ID NO: 332 - protein, 13 aa, Homo sapiens
NPSELKGKFIHTK

SEQ ID NO: 333 - protein, 18 aa, Homo sapiens
ISFGTPAPGFSSMLYGMK

SEQ ID NO: 334 - protein, 7 aa, Homo sapiens
DKVNGDK

SEQ ID NO: 335 - protein, 11 aa, Homo sapiens
AGKRGYIIDTR

SEQ ID NO: 336 - protein, 11 aa, Homo sapiens
GRPAVCQPQGR

SEQ ID NO: 337 - protein, 10 aa, Homo sapiens
IEISSPCCPR

SEQ ID NO: 338 - protein, 9 aa, Homo sapiens
RPISSCSQR

SEQ ID NO: 339 - protein, 15 aa, Homo sapiens
LPEMEPLVPRVKEVK

SEQ ID NO: 340 - protein, 10 aa, Homo sapiens
ATAVVDGAFK

SEQ ID NO: 341 - protein, 17 aa, Homo sapiens
EGGLGPLNIPLLADVTR

SEQ ID NO: 342 - protein, 17 aa, Homo sapiens
EGGLGPLNIPLLADVTR

SEQ ID NO: 343 - protein, 8 aa, Homo sapiens
GLFIIDGK

SEQ ID NO: 344 - protein, 18 aa, Homo sapiens
KEGGLGPLNIPLLADVTR

SEQ ID NO: 345 - protein, 9 aa, Homo sapiens
LSEDYGVLK

SEQ ID NO: 346 - protein, 10 aa, Homo sapiens
RLSEDYGVLK

SEQ ID NO: 347 - protein, 8 aa, Homo sapiens
TDEGIAYR

SEQ ID NO: 348 - protein, 15 aa, Homo sapiens
IAADMSNIIENLDTR

SEQ ID NO: 349 - protein, 12 aa, Homo sapiens
DTGTYGFLLPER

SEQ ID NO: 350 - protein, 15 aa, Homo sapiens
AGPHCPTAQLIATLK

SEQ ID NO: 351 - protein, 9 aa, Homo sapiens
HITSLEVIK

SEQ ID NO: 352 - protein, 11 aa, Homo sapiens
ICLDLQAPLYK

SEQ ID NO: 353 - protein, 12 aa, Homo sapiens
ICLDLQAPLYKK

SEQ ID NO: 354 - protein, 12 aa, Homo sapiens
KICLDLQAPLYK

SEQ ID NO: 355 - protein, 31 aa, Homo sapiens
ADEVAAAPEQIAADIPEVVVSLAWDESLAPK

SEQ ID NO: 356 - protein, 9 aa, Homo sapiens
IPACIAGER

SEQ ID NO: 357 - protein, 10 aa, Homo sapiens
RYGTCIYQGR

SEQ ID NO: 358 - protein, 9 aa, Homo sapiens
YGTCIYQGR

SEQ ID NO: 359 - protein, 18 aa, Homo sapiens
HMKIKSLEEMYVFSLPMK

SEQ ID NO: 360 - protein, 22 aa, Homo sapiens
CVRADEYSLYAKNENTGVVVVK

SEQ ID NO: 361 - protein, 14 aa, Homo sapiens
YSSAGTVEFLVDSK

SEQ ID NO: 362 - protein, 10 aa, Homo sapiens
LGPGMADICK

SEQ ID NO: 363 - protein, 13 aa, Homo sapiens
IYVDDGLISLQVK

SEQ ID NO: 364 - protein, 17 aa, Homo sapiens
DLGQGIQNSVTDRPETR

SEQ ID NO: 365 - protein, 20 aa, Homo sapiens
LGMPQFLSPEAQSLLRMLFK

SEQ ID NO: 366 - protein, 17 aa, Homo sapiens
QTSGLQATSSWQNLDAK

SEQ ID NO: 367 - protein, 23 aa, Homo sapiens
PISSIGQVQSYMEHYCNSSTDRR

SEQ ID NO: 368 - protein, 12 aa, Homo sapiens
ENFPNFLSACDK

SEQ ID NO: 369 - protein, 11 aa, Homo sapiens
GTNYLADVFEK

SEQ ID NO: 370 - protein, 19 aa, Homo sapiens
IDFSEFLSLLGDIATDYHK

SEQ ID NO: 371 - protein, 20 aa, Homo sapiens
KIDFSEFLSLLGDIATDYHK

SEQ ID NO: 372 - protein, 19 aa, Homo sapiens
IDFSEFLSLLGDIATDYHK

SEQ ID NO: 373 - protein, 20 aa, Homo sapiens
KIDFSEFLSLLGDIATDYHK

SEQ ID NO: 374 - protein, 19 aa, Homo sapiens
LLLSSLGIPVNHLIEGSQK

SEQ ID NO: 375 - protein, 31 aa, Homo sapiens
PVAQPVAALESAAEAGAGTLANPLGTLNPLK

SEQ ID NO: 376 - protein, 8 aa, Homo sapiens
AVLTIDEK

SEQ ID NO: 377 - protein, 7 aa, Homo sapiens
FLENEDR

SEQ ID NO: 378 - protein, 15 aa, Homo sapiens
FNKPFVFLMIEQNTK

SEQ ID NO: 379 - protein, 22 aa, Homo sapiens
GTEAAGAMFLEAIPMSIPPEVK

SEQ ID NO: 380 - protein, 14 aa, Homo sapiens
ITPNLAEFAFSLYR

SEQ ID NO: 381 - protein, 10 aa, Homo sapiens
KLSSWVLLMK

SEQ ID NO: 382 - protein, 15 aa, Homo sapiens
LQHLENELTHDIITK

SEQ ID NO: 383 - protein, 10 aa, Homo sapiens
LSITGTYDLK

SEQ ID NO: 384 - protein, 9 aa, Homo sapiens
LSSWVLLMK

SEQ ID NO: 385 - protein, 8 aa, Homo sapiens
SASLHLPK

SEQ ID NO: 386 - protein, 7 aa, Homo sapiens
SPLFMGK

SEQ ID NO: 387 - protein, 10 aa, Homo sapiens
SVLGQLGITK

SEQ ID NO: 388 - protein, 18 aa, Homo sapiens
VFSNGADLSGVTEEAPLK

SEQ ID NO: 389 - protein, 10 aa, Homo sapiens
FMFDLFQQFR

SEQ ID NO: 390 - protein, 14 aa, Homo sapiens
DPNHFRPAGLPEKY

SEQ ID NO: 391 - protein, 14 aa, Homo sapiens
EANYIGSDKYFHAR

SEQ ID NO: 392 - protein, 17 aa, Homo sapiens
LTGHGAEDSLADQAANK

SEQ ID NO: 393 - protein, 16 aa, Homo sapiens
RGPGGAWAAEVISNAR

SEQ ID NO: 394 - protein, 14 aa, Homo sapiens
SFFSFLGEAFDGAR

SEQ ID NO: 395 - protein, 15 aa, Homo sapiens
AYWDIMISNHQNSNR

SEQ ID NO: 396 - protein, 11 aa, Homo sapiens
EALQGVGDMGR

SEQ ID NO: 397 - protein, 8 aa, Homo sapiens
FRPDGLPK

SEQ ID NO: 398 - protein, 9 aa, Homo sapiens
GPGGVWAAK

SEQ ID NO: 399 - protein, 15 aa, Homo sapiens
SFFKEALQGVGDMGR

SEQ ID NO: 400 - protein, 10 aa, Homo sapiens
SNEKAEEWGR

SEQ ID NO: 401 - protein, 23 aa, Homo sapiens
VYLQGLIDYYLFGNSSTVLEDSK

SEQ ID NO: 402 - protein, 13 aa, Homo sapiens
EMVSQLQRECYLK

SEQ ID NO: 403 - protein, 21 aa, Homo sapiens
GGTLSTPQTGSENDALYEYLR

SEQ ID NO: 404 - protein, 12 aa, Homo sapiens
LDTLAQEVALLK

SEQ ID NO: 405 - protein, 25 aa, Homo sapiens
LNPGALLPVLTDPALNDLYVISTFK

SEQ ID NO: 406 - protein, 38 aa, Homo sapiens
AIAANEADAVTLDAGLVYDAYLAPNNLKPVVAEFYGSK

SEQ ID NO: 407 - protein, 8 aa, Homo sapiens
ASYLDCIR

SEQ ID NO: 408 - protein, 13 aa, Homo sapiens
CSTSSLLEACTFR

SEQ ID NO: 409 - protein, 8 aa, Homo sapiens
DDTVCLAK

SEQ ID NO: 410 - protein, 10 aa, Homo sapiens
DGAGDVAFVK

SEQ ID NO: 411 - protein, 13 aa, Homo sapiens
DLLFRDDTVCLAK

SEQ ID NO: 412 - protein, 10 aa, Homo sapiens
DSGFQMNQLR

SEQ ID NO: 413 - protein, 11 aa, Homo sapiens
DYELLCLDGTR

SEQ ID NO: 414 - protein, 14 aa, Homo sapiens
EDPQTFYYAVAVVK

SEQ ID NO: 415 - protein, 11 aa, Homo sapiens
HSTIFENLANK

SEQ ID NO: 416 - protein, 9 aa, Homo sapiens
KASYLDCIR

SEQ ID NO: 417 - protein, 14 aa, Homo sapiens
KCSTSSLLEACTFR

SEQ ID NO: 418 - protein, 11 aa, Homo sapiens
KDSGFQMNQLR

SEQ ID NO: 419 - protein, 13 aa, Homo sapiens
KPVEEYANCHLAR

SEQ ID NO: 420 - protein, 16 aa, Homo sapiens
NLNEKDYELLCLDGTR

SEQ ID NO: 421 - protein, 7 aa, Homo sapiens
NPDPWAK

SEQ ID NO: 422 - protein, 19 aa, Homo sapiens
SAGWNIPIGLLYCDLPEPR

SEQ ID NO: 423 - protein, 14 aa, Homo sapiens
SVIPSDGPSVACVK

SEQ ID NO: 424 - protein, 11 aa, Homo sapiens
WCAVSEHEATK

SEQ ID NO: 425 - protein, 8 aa, Homo sapiens
YLGEEYVK

SEQ ID NO: 426 - protein, 16 aa, Homo sapiens
FLPEGCQPLVSSAVDR

SEQ ID NO: 427 - protein, 13 aa, Homo sapiens
EDFTSLSLVLYSR

SEQ ID NO: 428 - protein, 20 aa, Homo sapiens
EFSHLGKEDFTSLSLVLYSR

SEQ ID NO: 429 - protein, 8 aa, Homo sapiens
ELSSFIDK

SEQ ID NO: 430 - protein, 14 aa, Homo sapiens
FPSGTFEQVSQLVK

SEQ ID NO: 431 - protein, 15 aa, Homo sapiens
KFPSGTFEQVSQLVK

SEQ ID NO: 432 - protein, 11 aa, Homo sapiens
LPDATPTELAK

SEQ ID NO: 433 - protein, 11 aa, Homo sapiens
RTHLPEVFLSK

SEQ ID NO: 434 - protein, 22 aa, Homo sapiens
VPTADLEDVLPLAEDITNILSK

SEQ ID NO: 435 - protein, 7 aa, Homo sapiens
YTFELSR

SEQ ID NO: 436 - protein, 9 aa, Homo sapiens
IYISGMAPR

SEQ ID NO: 437 - protein, 22 aa, Homo sapiens
MDWLVPATCEPIQSVFFFSGDK

SEQ ID NO: 438 - protein, 11 aa, Homo sapiens
RVDTVDPPYPR

SEQ ID NO: 439 - protein, 15 aa, Homo sapiens
SIAQYWLGCPAPGHL

SEQ ID NO: 440 - protein, 10 aa, Homo sapiens
VDTVDPPYPR

SEQ ID NO: 441 - protein, 22 aa, Homo sapiens
EEQNFVVQNEINNMSFLTADNK

SEQ ID NO: 442 - protein, 8 aa, Homo sapiens
IGISWNKK

SEQ ID NO: 443 - protein, 10 aa, Homo sapiens
FSGSSSGADR

SEQ ID NO: 444 - protein, 18 aa, Homo sapiens
YLPCFQVLPLDQQLVLVR

SEQ ID NO: 445 - protein, 17 aa, Homo sapiens
HCIVLLQIAKDQSNAEK

SEQ ID NO: 446 - protein, 23 aa, Homo sapiens
VKLQNAENDYINASLVDIEEAQR

SEQ ID NO: 447 - protein, 19 aa, Homo sapiens
YILGSVVDSEKNYVDALKR

SEQ ID NO: 448 - protein, 16 aa, Homo sapiens
EAFKSALMSSYWCSGK

SEQ ID NO: 449 - protein, 16 aa, Homo sapiens
EPHISTLRKILFEDFR

SEQ ID NO: 450 - protein, 19 aa, Homo sapiens
IHIEENEDRDTGLEQRHNK

SEQ ID NO: 451 - protein, 29 aa, Homo sapiens
GLRIHELSSDDYSTEEEAQTPDCSITDFR

SEQ ID NO: 452 - protein, 12 aa, Homo sapiens
IQQQVDSPMNLK